(19) United States
(12) Patent Application Publication
(10) Pub. No.: US 2026/0272852 A1
(43) Pub. Date: Sep. 17, 2026

(54) COAGULATION PATHWAY AND NICOTINAMIDE-ADENINE DINUCLEOTIDE PATHWAY MODULATING COMPOSITIONS AND METHODS OF THEIR USE

(71) Applicant: L'OREAL, Paris (FR)

(72) Inventors: Jacob WAUGH, Las Vegas, NV (US); Jonah SHACKNAI, Santa Barbara, CA (US)

(21) Appl. No.: 18/723,005

(22) PCT Filed: Dec. 22, 2022
(86) PCT No.: PCT/IB2022/062694

§ 371 Date: Jun. 21, 2024

Related U.S. Application Data
(60) Provisional application No. 63/292,960, filed on Dec. 22, 2021.

(51) Int. Cl. A61K 31/122; A61K 9/00; A61K 31/455; A61K 47/36; A61K 47/42

(52) U.S. Cl. CPC A61K 31/122; A61K 9/0014; A61K 31/455; A61K 47/36; A61K 47/42

(57) ABSTRACT

Provided are compositions comprising (1) a physiologically effective amount (e.g., a cosmetically effective amount or therapeutically effective amount) of a coagulation pathway modulation component (CP-MC), which modulates a protein C activity (either directly, indirectly, such as through modulating protein S activity, or both), and (2) a physiologically effective amount (e.g., a cosmetically effective amount or therapeutically effective amount) of a nicotinamide-adenine dinucleotide (NAD) pathway modulation component (NADP-MC). The CP-MC can comprise vitamin K compounds, such as vitamin K1. The NADP-MC can comprise a nicotinate compound component, a niacinamide compounds component, or both. In cases, compositions include additional components/ingredients, such as a penetration enhancing component comprising one or more penetration enhancers. Also provided are methods of modulating conditions in subjects, such as the appearance of skin or the treatment/prevention of skin diseases, comprising administering such compositions or compositions comprising such components in a manner effective to achieve desired effects.

### RELATED APPLICATIONS AND PRIORITY

This application claims priority to Application No. PCT/IB2022/062694 filed Dec. 22, 2022, which claims the benefit of and priority to U.S. Provisional Patent Application No. 63/292,960, filed Dec. 22, 2021, each of which is incorporated herein by reference.

### FIELD OF THE INVENTION

The invention relates to compositions that exhibit pharmaceutical or cosmetic properties in individuals and methods of using such compositions to treat or prevent diseases, to change the appearance of recipients, or otherwise modulate the physiological state of subjects.

### BACKGROUND OF THE INVENTION

The skin is the largest organ of the human body. Unfortunately, skin diseases and undesirable skin conditions are very common and, according to studies, affect as many as one in three Americans at any given time. Treatment of conditions of the skin is important for both overall health, but also for appearance, the latter being associated with self-esteem and other important aspects of mental health. Such conditions can include, e.g., hyperkeratotic skin, discoloration from bruising and other injuries, and the like. Additional medical skin conditions include actinic keratosis, psoriasis, rosacea, and seborrheic dermatoses. Common treatment methods for skin conditions include medicated creams and ointments, including agents such as antibiotics, antioxidants, and vitamins. Surveys indicate that the average American spends over $300 per year on skincare (possibly result in expenditures of more than $15,000 over a lifetime). The overall global market for skin care products is over 145.3 billion dollars per year. Clearly, while there are many products available to treat skin-related conditions, no single solution currently exists for even standard and well-studied skin conditions, such as treatment of bruising.

As noted above, vitamins are one common component of many skin products, especially cosmetic skin products. The primary vitamins used by cosmetic formulators reportedly include forms of vitamins classified as A, B, C, D, E, F, and K vitamins. In humans, there are 13 “letter” vitamins that can be classified as either fat- or water-soluble. Vitamins A, D, E, and K are fat-soluble, whereas the B-complex group and vitamin C are mostly water-soluble. The letter naming system can sometimes lead to oversimplification and, thus, confusion as, for example, there are numerous types of compounds classified under each “letter” of vitamin (e.g., vitamin B can refer to vitamin B-1 (thiamine), vitamin B-2 (riboflavin), vitamin B-3 (niacin), vitamin B-5 (pantothenic acid/panthenol), vitamin B-6 (pyridoxine), vitamin B-7 (biotin), vitamin B-9 (folic acid), and vitamin B-12 (cobalamin).

While there is evidence that some vitamin-based products, particularly products including combinations of vitamins, may be effective in skin health, the limited amount of such efficacy data does not support the widespread use of such products, generally, especially when such products are delivered topically. Moreover, it is well known in the art that formulation of topically applied vitamin products is essential to effectiveness in many or most situations. An August 2020 brief review by Perry Romanowski, published on chemistscorner.com (a website focused on the science of cosmetic chemistry/formulation), for example, explores the evidence for commonly used vitamins in cosmetic formulations, with reference to numerous published peer review papers. The Romanowski article reports, e.g., that vitamin B-5 is of questionable efficacy in skin care and vitamin B-3 provides only a “slight effective” in skin care. In contrast, some forms of vitamin A are known to be effective for skin conditions including reducing photoaging, wrinkles, and acne, and, in fact, are regulated as drugs by the US Food and Drug Administration (FDA). Studies of vitamin E reportedly demonstrated that it at least sometimes is not effective against UV-induced skin damage and there are inconsistent results with respect to vitamin E's ability to assist in reduction of scar tissue. Historically, vitamin C formulations were only possibly considered effective in remarkably high concentrations of certain forms given the poor stability of such compounds. Vitamin D, while showing some efficacy for treating psoriasis, does not appear to have “any notable antiaging effects.” Further according to the Romanowski article, use of vitamin F and vitamin K also reportedly was not supported by meaningful evidence. Another article by Dr. Gary D. Vogin, MD, posted on WebMd (2002) similarly reports that, according to dermatologists, while “many face creams contain vitamins known as antioxidants, very few are actually effective in preventing or reversing skin damage” due to poor absorption, low concentrations, and other issues limiting or negating efficacy, with possible exceptions including vitamin E and vitamin C.

Other reviewers take a slightly more favorable view. An article by Zawn Villines, reviewed by Cynthia Cobb, in Medical News Today (Apr. 11, 2019) reports, “Research into the role of vitamin B-complex supplements is promising, though inconclusive” and also notes that “niacinamide, may help some signs of skin aging” including reducing the appearance of age spots and discoloration and folic acid also may be associated with positive skin effects. Pantothenic acid dietary supplementation also has been reported in some studies to aid with acne and skin inflammation.

Nicotinic acid and nicotinamide are the two most common forms of niacin in dietary supplements and topically applied cosmetic products. Nicotinic acid in supplemental amounts beyond nutritional needs can cause skin flushing, so some formulations are manufactured and labeled as prolonged, sustained, extended, or timed release to minimize this unpleasant side effect. Due to the decreased number of side effects of topical niacinamide compared to nicotinic acid, the effects of niacinamide as a topical cosmeceutical agent have been more studied to date. See, e.g., Levin J, et al. J Clin Aesthet Dermatol. 2010; 3 (2):22-41. Nicotinamide does not produce skin flushing because of its slightly different chemical structure, but also shows a different therapeutic/physiologic profile from nicotinic acid/nicotinate.

The NIH reports at least some research supports the use of niacinamide for aiding some skin conditions, such as eczema, reduction of isotretinoin-induced skin irritation/inflammation, and lightening of dark patches (melasma), and early-stage research suggests that applying niacinamide can reduce redness and scaling of the skin in people with seborrheic dermatitis. Other authors report that “although the existing data are not sufficient for a scientifically founded evaluation, it can be stated that the use of niacinamide in galenic preparations for epicutaneous application offers most interesting prospects.” See, e.g., Wohlrab J, et al. Skin Pharmacol Physiol 2014; 27:311-315. One supportive study involved 50 women applying a moisturizer containing 5% niacinamide to one half of their face and a placebo to the other half for 12 weeks, leading to a finding of significant improvements in hyperpigmentation spots, fine lines, and wrinkles, in the treated side as compared to the control side. Another split-face study, published in 2011 in Dermatology Research and Practice, found that a topical 4% niacinamide treatment resulted in good-to-excellent results in 44% of treated patients, which was less effective than 4% hydroquinone, but associated with less adverse events.

As noted, the amount of data relating to the use of nicotinic acid/nicotinate topical products is even less well developed. Moreover, where nicotinic acid/nicotinate products are used in topical skincare products, they are often in the form of vitamin derivatives, such as methyl nicotinate or ϵ-tocopheryl nicotinate. Methyl nicotinate causes reddening of skin and is actually used as a rubefacient (a product used to induce redness of the skin) or, as a supplement, in the treatment of muscle/tendon aches and pains, such as arthritis pain. Reports indicate that α-tocopheryl nicotinate is used “infrequently” in cosmetic applications, which may be due to the lower uptake of α-tocopherol by skin compared with, e.g., an acetate ester or the lower UV protection afforded by the nicotinate esterified form relative to the acetate-esterified form. See, e.g., Zondlo Fiume M, Int J Toxicol. 2002; 21 Suppl 3( ):51-116 as reported in Duncan K R et al. Antioxidants (Basel). 2017; 6 (1):20.

As noted, other vitamins widely used in cosmetic/skincare products similarly appear to be associated with limited or conflicting evidence regarding efficacy and various forms of “letter” vitamins are associated with different physiological effects. For example, the two known different forms of vitamin K (K1/phylloquinone and K2) are obtained from different natural sources and have different chemistries. Vitamin K2 includes several subtypes called menaquinones (MKs) that are named by the length of their side chain (MK-4 to MK-13). Because of differences in absorption and transport to tissues, vitamin K1 and K2 reportedly “could have profoundly different effects on your health.” See, e.g., Pearson, K. Healthline. Sep. 15, 2017. Both forms of vitamin K appear to have an impact on blood clotting. E.g., reportedly one study showed that a single serving of natto rich in vitamin K2 altered measures of blood clotting for up to four days. Vitamin K2 is associated with a significantly longer half-life than vitamin K1 and vitamin K2 also is believed to be better absorbed than vitamin K1.

In some scientific literature and reports on the internet, vitamin K has been suggested to be associated with wound healing activity. E.g., a 1% concentration of vitamin K1 was allegedly shown in animal studies to positively influence wound healing (as reported on the Paula's Choice review of vitamin K). Recent studies have also shown positive results in humans, but the mechanism for vitamin K1's effects in wound healing is not clear. See, e.g., Pazyar N et al. Indian J Pharmacol. 2019; 51 (2):88-92. However, another report found that pretreatment with vitamin K had no effect on 585-nm pulsed dye laser-induced purpuras, but the same report identified a slightly significant difference (P<0.0489) between the mean scores of vitamin K and placebo treated sites in the posttreatment group; measures were based on a subjective visual analog scale. Still another double-blind, placebo-controlled study on ten healthy individuals treated twice daily with 0.5% vitamin K1 cream or placebo (vehicle only) for one week, resulted in no influence on suction induced petechia formation (Kovacs, R K, Journal of the American Academy of Dermatology, 50 (6): P982-983, Jun. 1, 2004).

In cosmetics, vitamin K has been promoted for the reduction of dark circles around the eyes in connection with various products. However, critical reviews of such use indicate that in most cases such products do not contain any amount of vitamin K that has been the subject of any clinical testing. What testing has been performed on vitamin K in such contexts appears to be inconclusive regarding supporting a claim of efficacy. For example, a study published in the Journal of Cosmetic Dermatology (April 2004, page 73) examined the effect of applying a gel containing 2% vitamin K plus 0.1% retinol, vitamin E, and vitamin C in 57 adults on dark circles over an 8-week study and the results, were inconclusive, with only 47% of the testers noting a “fair to moderate” improvement in dark circles. Also, it remains unclear whether the results seen in the study were from vitamin K or from other vitamins present in the test product. Reportedly a 1% vitamin K1 product was used in another study to test its results on improving the look of dark circles, but although some improvement was noted, the formula in testing also contained caffeine and emu oil, making it difficult to conclude whether vitamin K1 caused any effect (also reported on the Paula's Choice review of vitamin K on the internet).

Vitamin K is an essential co-factor for coagulation factor Protein C. Activated protein C (“APC”) has been linked to effects in sepsis, wound healing, central nervous system injury, ischemic stroke, Alzheimer's disease, acute kidney injury, lung disorders, acute pancreatitis, type I diabetes, rheumatoid arthritis, and cancer. See Zhao R, et al. Int J Mol Sci. 2019; 20 (4):903. However, to date, APC has only been approved for severe sepsis (Xigris™—Eli Lilly and Co.). Id. A 2012 Cochrane review found that rhAPC's use cannot be recommended since it does not improve survival and increases bleeding risk and in October 2011 Xigris was withdrawn from the market by Eli Lilly due to a higher mortality in a trial among adults, evidencing that safe and effective modulation of coagulation pathways is not a simple matter.

Numerous patent disclosures are directed to topically applied products comprising combinations of several (e.g., ≥5, ≥10, or more) agents, many or most of which may be various vitamin compounds. For example, US20180344856 discloses compositions for skin conditions including regulating dryness in urogenital skin and reduction of wrinkles, comprising compounds relating to vitamin A, B, C, D, E, F, and K compounds, salts thereof, and mixtures thereof, in combination with hyaluronic acid, hyaluronic acid compositions, and optionally other ingredients such as silicone oil. US20040146539 discloses compositions for body slimming, skin aging, and other conditions also possibly including numerous ingredients (including for example, forskohlin extract (from Coleus forskolii), human growth hormone (HGH), Roselle tea extract, Laminaria extract, extract from berries of Panax genus plants, extract of Sage (Salvia officinalis), and extract of Orthosiphon (Ortosifon stamincus), as well as, e.g., vitamin K, nicotinate, and hyaluronic acid. WO2008065451 similarly discloses compositions for treatment of skin conditions including, e.g., wrinkles, that can comprise a wide variety of numerous unrelated ingredients including, e.g., titrated extract of Centella, extract of Ginkgo biloba, Devil's claw extract, extract of Boswellia serrata, Echinacea purpurea, hydrocortisone 17-butyrate, Japanese pepper extract rosemary extract CG (extract of Rosmarinus officinalis longa), and vitamin C palmitate, vitamin K, nicotinate, hyaluronic acid, vitamin D3, or vitamin E. US20040161435 discloses treatment of a variety of conditions, such as fat reduction, cellulite control, etc., which compositions optionally comprise hyaluronic acid, vitamin K, and nicotinate, forskohlin extract (from Coleus forskohlii), human growth hormone (HGH), theobromine (or salts thereof such as aminophylline), roselle tea extract, Gymnema sylvestre extract, 9-cis, extract of St. Johns-wart (Hypericum perforatum), astaxanthin (from Haematococcus algae), and a large number of different vitamins (such as vitamin A, members of vitamins B group, vitamin C, vitamin D, vitamin E, carotenes, biotin, and/or folic acid). US20190365735 discloses treatment of varicose veins with compositions that can optionally comprise vitamin K and nicotinate among many other unrelated ingredients (including for example, glycerin; caprylic triglycerides; polysorbate; methyl nicotinate; pyridoxal-5-phosphate; and Ginkgo biloba). US20210077375 discloses compositions for treatment of e.g., wrinkles, which can comprise any number of a large group of unrelated ingredient options (including for example, vitamin B1 to B12, with the exception of vitamin B3, low molecular weight hyaluronic acid, vitamin K, vitamin E, vitamin D, and/or vitamin A). Additional similar disclosures of mixtures of various vitamins and other allegedly active agents for treatment of various skin conditions are provided in, e.g., WO2013149323, U.S. Pat. No. 7,455,319, WO200001351, US20060193789, US20070243146, US20180333367, US20060110415, U.S. Pat. No. 6,193,985, US20130022687, and US20040156873.

These and similar broad patent disclosures including numerous (e.g., combinations of 10 or more) active agents, taken in view of the large number of marketed products in the field and internet disclosures that are unsupported by actual clinical data, evidence that while numerous agents have been proposed/suggested for modulation of skin conditions, there is actually a lack of reliable teaching in the art with respect to the selection, combination, and formulation of effective combinations of such ingredients for the treatment of skin conditions. Indeed, such facts evidence that the generation of truly effective compositions and methods for treating skin conditions requires an application of inventive ingenuity.

### Construction, Terms, and Acronyms

This section offers guidelines for reading this disclosure. The intended audience for this disclosure (“readers”) are persons having ordinary skill in the practice of technologies discussed or used herein. Readers may also be called “skilled persons,” and such technologies called “the art.” Terms such as “understood,” “known,” and “ordinary meaning,” refer to the general knowledge of skilled persons.

The term “uncontradicted” means not contradicted by this disclosure, logic, or plausibility based on knowledge of skilled persons.

Disclosed here are several different but related exemplary aspects of the invention (“aspects”) (“cases,” “facets,” or “embodiments”). The invention encompasses all aspects, as described individually and as can be arrived at by any combination of such individual aspects. The breadth and scope of the invention should not be limited by any exemplary embodiments. No language in this disclosure should be construed as indicating any element/step is essential to the practice of the invention unless such a requirement is explicitly stated. Uncontradicted, any aspect(s) can be combined with any other aspect(s).

Uncontradicted, all technical/scientific terms used here generally have the same meanings as commonly understood by skilled persons, regardless of any narrower examples or descriptions provided here (including any term introduced initially in quotations). However, aspects characterized by the inclusion of elements, steps, etc., associated with specific descriptions provided here are distinct embodiments of the invention. Uncontradicted, disclosure of any aspect using known terms, which terms are narrowed by example or otherwise in this disclosure, implicitly discloses related aspects in which such terms are alternatively interpreted using the broadest reasonable interpretation of skilled persons.

Uncontradicted, “or” means “and/or” here, regardless of any occasional inclusion of “and/or” (e.g., phrases such as “A, B, or C” and “A, B, and/or C” simultaneously disclose aspects including (1) all of A, B, and C; (2) A and C; (3) A and B; (4) B and C; (5) only A; (6) only B; and (7) only C (and also support sub-groupings, such as “A or B,” “A or C,” etc.)).

Uncontradicted, “also” means “also or alternatively.” Uncontradicted, “here” & “herein” mean “in this disclosure”. The term “i.a.” (“ia” or “ia”) means “inter alia” or “among other things.” “Also known as” is abbreviated “aka” or “AKA” (and can mean is otherwise referred to, even if the relationship between the terms is not well known). “Elsewhere” means “elsewhere herein.”

For conciseness, symbols are used where appropriate. E.g., “&” is used for “and” & “~” for “about.” Symbols such as < and > are given their ordinary meaning (e.g., “≤” means “less than or equal to” & “≥” means “greater than or equal to”). A slash “/” can represent “or” (“A/B” means “A or B”) or identify synonyms of an element, as will be clear from context.

The inclusion of “(s)” after an element or a step indicates that ≥1 of such an element is present, step performed, and the like. E.g., “element(s)” means both 1 element or ≥2 elements, with the understanding that each thereof is an independent aspect of the invention.

Use of the abbreviation “etc.” (or “et cetera”) in association with a list of elements/steps means any or all suitable combinations of the recited elements/steps or any known equivalents of such recited elements/steps for achieving the function(s) of such elements/steps that are known in the art. Terms such as “and combinations,” or “or combinations” regarding listed elements/steps means combinations of any or all such elements/steps. “Suitability” means acceptable or appropriate for performing a particular function/achieving particular state(s)/outcome(s), and typically means effective, practical, and non-deleterious/harmful. Additional characteristics of suitability of certain aspects of the invention may be described below.

Uncontradicted, heading(s) (e.g., “Construction, Terms . . . ”) and subheadings are included for convenience and do not limit the scope of any aspect(s). Uncontradicted, aspect(s), step(s), or element(s) described under one heading can apply to other aspect(s) or step(s)/element(s) here.

Ranges of values are used to represent each value falling within such range that are within an order of magnitude of the smallest endpoint of the range without having to explicitly write each value of the range. E.g., a recited range of 1-2 implicitly discloses each of 1.0, 1.1, 1.2, . . . 1.9, and 2.0 and 10-100 implicitly discloses each of 10, 11, 12, . . . 98, 99, and 100). Uncontradicted, all ranges include the range's endpoints, regardless of how a range is described. E.g., “between 1-5” includes 1 and 5 in addition to 2, 3, and 4 (and all numbers between such numbers within an order of magnitude of such endpoints, e.g., 1.0, 1.1, . . . 4.9, and 5.0). For the avoidance of doubt, any number within a range, regardless of the order of magnitude of the number, is covered by the range (e.g., a range of 2-20 covers 18.593).

Terms of approximation (e.g., “about,” “~,” or “approximately”) are used (1) to refer to a set of related values or (2) where a precise value is difficult to define (e.g., due to limits of measurement). Uncontradicted, all exact values provided here simultaneously/implicitly disclose corresponding approximate values and vice versa (e.g., disclosure of “about 10” provides explicit support for the use of 10 exactly in such aspect/description). Ranges described with approximate value(s) include all values encompassed by each approximate endpoint, regardless of presentation (e.g., “about 10-20” has the same meaning as “about 10-about 20”). The scope of value(s) encompassed by an approximate term typically depends on the context of the disclosure, criticality or operability, statistical significance, understanding in the art, etc. In the absence of guidance here or in the art, terms such as “about” should be interpreted as +/−10% of the indicated value(s).

Lists of aspects, elements, steps, and features are sometimes employed for conciseness. Unless indicated, each member of each list should be viewed as an independent aspect. Each aspect defined by any individual member of a list can have, and often will have, nonobvious properties vis-a-vis aspects characterized by other members of the list.

Uncontradicted, the terms “a” and “an” and “the” and similar referents encompass both the singular and the plural form of the referenced element, step, or aspect. Uncontradicted, terms in the singular implicitly convey the plural and vice versa herein (in other words, disclosure of an element/step implicitly discloses corresponding use of such/similar elements/steps and vice versa). Hence, e.g., a passage regarding an aspect including X step supports a corresponding aspect including several X steps. Mixed usage of a referent such as “a” in respect of one element and “one or more of” with respect to another element in a paragraph, sentence, aspect, or claim, does not change the meaning of such referents.

Uncontradicted, the term “suitable” means appropriate, acceptable, or in contexts sufficient, or providing at least generally or substantially all of an intended function, without causing or imparting significant negative/detrimental impact.

“Significant” and “significantly” mean results/characteristics that are statistically significant using ≥1 appropriate test(s)/trial(s) in the given context (e.g., p≤0.05/0.01). “Detectable” means measurably present/different using known detection tools/techniques. The acronym “DOS” (or “DoS”) means “detectable(ly) or significant(ly).”

Uncontradicted, for any value here that is not accompanied by a unit of measurement (e.g., a weight of 50 or a length of 20), any previously provided unit for the same element/step or the same type of element/step will apply, or, in cases where no such disclosure exists, the unit most commonly used in association with such an element/step in the art applies.

Uncontradicted, the terms “including,” “containing,” “comprising,” and “having” mean “including, but not limited to” or “including, without limitation.” Uncontradicted, use of terms such as comprising and including regarding elements/steps means including any detectable number or amount of an element or including any detectable performance of a step/number of steps (with or without other elements/steps).

For conciseness, description of an aspect “comprising” or “including” an element, with respect to a collection/whole (e.g., a system, device, or composition), implicitly provides support for any detectable amount/number or ≥~1%, ≥~5%, ≥~10%, ≥~20%, ≥~25%, ≥~33%, ≥~50%, ≥~51%, ≥~66%, ≥~75%, ≥~90%, ≥~95%, ≥~99%, or ~100% of the whole/collection being made up of the element, or essentially all of the whole/collection being made up of the element (i.e., that the collection consists essentially of the referenced element). Similarly, a method described as including a step with respect to an effect/outcome implicitly provides support for the referenced step providing ≥~1%, ≥~5%, ≥~10%, ≥~20%, ≥~25%, ≥~33%, ≥~50%, ≥~51%, ≥~66%, ≥~75%, ≥~90%, ≥~95%, ≥~99%, or ~100% of the effect/outcome, of ≥~1%, ≥~5%, ≥~10%, ≥~20%, ≥~25%, ≥~33%, ≥~50%, ≥~51%, ≥~66%, ≥~75%, ≥~90%, ≥~95%, ≥~99%, or ~100% of the steps/effort performed, or both. Explicit listing of percentages of elements/steps in connection with aspects does not limit or contradict such implicit disclosure.

Uncontradicted, terms such as “comprising” when used in connection with a step of a method provide implicit support for performing the step once, ≥2 times, or until an associated function/effect is achieved.

Uncontradicted, the term “one” means a single type, single iteration/copy/thing, of a recited element or step, or both. For example, the referent “one” with respect to a component of a composition can refer to one type of element (which may be present in numerous copies/units/molecules, as in the case of an ingredient in a composition), one such unit of the element, or both. Similarly, “one” component, a “single” component, or the “only component” of a system typically means 1 type of element (which may be present in numerous copies), one instance/unit of the element, or both. Further, “one” step of a method typically means performing one type of action (step), one iteration of a step, or both.

Uncontradicted, the term “some” means ≥2 copies/instances or ≥5% of a listed collection/whole is, or is made up of, an element. Regarding methods, some means ≥5% of an effect, effort, or both, is made up of or is attributable to a step (e.g., as in “some of the method is performed by step Y”) or indicates a step is performed ≥2 times (e.g., as in “step X is repeated some number of times”). “Predominately,” “most,” or “mostly,” means detectably ≥50% (e.g., mostly comprises, predominately includes, etc., mean ≥50%) (e.g., a system that mostly includes element X is composed of ≥50% of element X). The term “generally” means ≥75% (e.g., generally consists of, generally associated with, generally comprises, etc., means ≥75%) (e.g., a method that generally consists of step X means that 75% of the effort or effect of the method is attributable to step X). “Substantially” or “nearly” means ≥95% (e.g., nearly all, substantially consists of, etc., mean ≥95%) (e.g., a collection that nearly entirely is made up of element X means that at least 95% of the elements in the collection are element X).

Uncontradicted, any aspect described with respect to an optionally present element(s)/step(s) also provides implicit support for corresponding aspect(s) in which one, some, most, generally all, nearly all, essentially all, or all of such element(s) are lacking/step(s) not performed, in respect of the relevant aspect. E.g., disclosure of a system comprising element X implicitly also supports a system lacking element X.

Uncontradicted, changes to tense or presentation of terms (e.g., using “comprises predominately” in place of “predominately comprises”) do not change the meaning of the corresponding term/phrase.

Uncontradicted, all methods provided here can be performed in any suitable order regardless of presentation (e.g., a method comprising steps A, B, and C, can be performed in the order C, B, and A; B and A and C, simultaneously, etc.) given the described/intended outcome is achieved (e.g., a stable composition results, ingredients are not subjected to conditions or combinations affecting their efficacy, etc.). Uncontradicted, elements of a composition, or, e.g., groups of ingredients such as groups of ingredients compiled in phases described herein, can be assembled in any suitable manner by any suitable method given a particular outcome is achieved (e.g., a stable composition results, ingredients are not subjected to conditions or combinations affecting their efficacy, etc.). In general, any methods and materials similar or equivalent to those described here can be used in the practice of embodiments. Uncontradicted, the use of ordinal numbers such as “first,” “second,” “third,” and so on is to distinguish respective elements rather than to denote a particular order. Uncontradicted, any elements, steps, components, or features of aspects and all variations thereof, etc., are within the scope of the invention.

Elements associated with a function can be described as “means for” performing a function in a composition/device/system or a “step for” performing a part of a method, and parts of this disclosure refer to “equivalents,” which means equivalents known in the art for achieving a referenced function associated with disclosed mean(s)/step(s). However, no element of this disclosure or claim should be interpreted as limited to a “means-plus-function” or “step-plus-function” construction unless such intent is clearly indicated by the use of the terms “means for” or “step for.” Terms such as “configured to” or “adapted to” do not indicate “means-plus-function” interpretation, but, rather, describe element(s)/step(s) configured to, designed to, selected to, or adapted to achieve a certain performance, characteristic, property, or the like using teachings provided here or in the art.

All references (e.g., publications, patent applications, and patents) cited herein are hereby incorporated by reference as if each reference were individually and specifically indicated to be incorporated by reference and set forth in its entirety herein. Uncontradicted, any suitable principles, methods, or elements of such references (collectively “teachings”) can be combined with or adapted to aspects. However, citation/incorporation of patent documents is limited to the technical disclosure thereof and does not reflect any view regarding the validity, patentability, etc., thereof. In the event of any conflict between this disclosure and the teachings of such documents, the content of this disclosure controls regarding aspects of the invention. Numerous references are cited here to concisely incorporate known information and aid skilled persons in putting aspects into practice. While efforts have been made to include the most relevant references for such purposes, readers will understand that not every aspect of every cited reference will apply to every aspect of the invention.

### Additional Terms, Concepts, and Acronyms

The following description of certain terms and acronyms is provided to assist readers in understanding the invention. Additional acronyms may be only provided in other parts of this disclosure and acronyms that are well known in the art may not be provided here.

Uncontradicted, any description of weight of compounds, ingredients, etc., of a composition described here is in reference to the weight percent (“wt. %”).

Except where explicitly indicated or clearly indicated by context, “improved” herein means “increased.” In aspects, “improved” means “reduced,” such as with respect to the toxicity of a composition adverse events related to use of a composition, or the like. Uncontradicted, terms such as “enhanced,” “improved,” and the like are used synonymously. For example, “enhanced activity” means an improvement in activity of a compound (e.g., an increased level of activity), and “enhanced stability” means an improvement in stability (e.g., an increase in the amount of time a compound is stable).

An “effective amount” of an ingredient, component, composition, etc., means an amount that is effective for carrying out an intended function/outcome or detectably or significantly inducing, promoting, causing, preventing, enhancing, etc., as the case may be, an intended function/outcome. For example, an effective amount of a preservative ingredient is an amount that detectably or significantly enhances the preservation of other ingredient(s) in a composition. A “physiologically effective amount” is an amount of an ingredient, composition, component, etc., that causes or induces, promotes, enhances, etc., an effect in a subject, such as in a human patient. A “cosmetically effective amount” is an amount that detectably or significantly enhances the appearance of the skin of a subject. A “therapeutically effective amount” typically means an amount of a compound, composition, component, ingredient, etc. that elicits, promotes, enhances, etc., an intended (typically significant) biological or medical response of a tissue, system, animal, or human. In aspects, a therapeutically effective amount is demonstrated by at least one or at least two well controlled and adequate clinical studies in human subjects/patients (e.g., as would be considered sufficient for pharmaceutical approval by leading regulatory agencies, such as US FDA). Readers will understand that an effective amount of a compound/ingredient can, if combined with other compounds/ingredients providing the intended effect, be less than what would be effective if such compound/ingredient was/were administered/delivered in such an amount alone. E.g., where ≥2 compounds of a type are provided, such as ≥2 NADP-MC constituents, an effective amount of one of such compounds can mean the amount of that compound that, combined with the amount of the other compound, is effective for the combination of compounds to be effective. In other aspects, some, most, or each of ≥2 compounds/ingredients in a composition are independently present in amounts that would be effective even if such compound(s) were administered/delivered alone. Uncontradicted, both variations are implicitly provided with respect to ingredients/compositions described herein. Uncontradicted, any composition described herein is understood as being administered or otherwise delivered (e.g., by DNA expression, where applicable) to subject(s) in an effective amount, which, uncontradicted implicitly provides support for in a pharmaceutically/therapeutically effective amount or in a cosmetically effective amount.

As noted above, “suitability” is determined based on intended use or context for intended use. For example, a composition that is “suitable” for topical administration means a formulation that is at least substantially absorbed by the skin under the conditions of application. A composition that is “suitable” for cosmetic or therapeutic/pharmaceutical use will be sufficiently safe (e.g., non-toxic), and have sufficient strength, purity, potency, etc., for its ended use and effect. Uncontradicted, any compositions, ingredients, components, etc., described herein are suitable for their intended use.

Uncontradicted, references to “compositions” herein mean compositions of this invention. In aspects, compositions can be characterized by, i.a., the elements of which they are comprised, which can be described herein as ingredients or “components.” Uncontradicted, a “component’ is a compound, collection of compounds, or a composition containing compound(s) (or constituents) that is related by exhibiting/providing/imparting a specific function and that can be considered a discrete part/ingredient of a composition. For example, a “coagulation pathway modulation component” (CP-MC) is a component of a composition comprising constituent(s) which provide(s) DOS coagulation pathway modifying effect(s); an “NAD pathway modulation component” (NADP-MC) is similarly a component of a composition comprising one or more constituents which each alone or together provide(s) an NAD pathway modifying effect, etc. Uncontradicted, the term “constituents” means a compound that is an ingredient of a composition. An ingredient or a constituent can be a single compound, molecule, etc., or can be a composition that is related in one or more physical or functional ways (e.g., sodium hyaluronate, a sodium salt of hyaluronic acid, can be an ingredient/constituent).

As used herein, the terms “administering” or “administration” of an agent, drug, or peptide to a subject includes any route of introducing or delivering to a subject a compound to perform its intended function. The administering or administration can be carried out by any suitable route, including orally, intranasally, parenterally (intravenously, intramuscularly, intraperitoneally, or subcutaneously), rectally, or topically. Uncontradicted, administering or administration includes self-administration and the administration under the direction/supervision of or by another (e.g., under the direction/supervision of a licensed healthcare professional).

A table of some acronyms frequently used in this disclosure follows:

TABLE 1
Select Acronyms

| Acronym | Full Term | Brief Description |
|---|---|---|
| APC | Activated Protein C | An abbreviation known in the art |
| CP-MC | Coagulation | A component of a composition comprising |
| | Pathway Modulating | constituent(s) which provide(s) DOS coagulation |
| | Component | pathway modifying effect(s) when |
| | | delivered/administered to subject(s) in effective |
| | | amount(s) |
| NAD | Nicotinamide- | An abbreviation known in the art |
| | adenine dinucleotide | |
| NADP-MC | NAD Pathway | A component of a composition comprising |
| | Modulating | constituent(s) which provide(s) DOS NAD |
| | Component | pathway modifying effect(s) when |
| | | delivered/administered in effective amount(s) to |
| | | subject(s) |
| PC-MC | Protein C | A component of a composition comprising |
| | Modulating | constituent(s) which comprise(s) protein C |
| | Component | activity modulator(s) (PCAMs) |
| PCAM | Protein C activity | A constituent (compound/molecule) of a protein C |
| | modulator | modulating component (PC-MC) that, alone or in |
| | | combination with other PCAM(s), causes a DOS |
| | | modulation of Protein C activity in subject(s) |
| | | when administered/delivered in effective |
| | | amount(s). |
| PCPSAM (or | Protein C/S activity | A compound/molecule of a composition |
| PCAM/PSAM)/ | modulator / | comprising constituent(s) which, individually or |
| PCPSA-MC | PCPSAM | in combination, provide(s) DOS Protein C/S |
| | component | modulating activity when administered/delivered |
| | | in effective amount(s) in subject(s). A PCPSA- |
| | | MC is a component of a composition comprising |
| | | PCPSAM(s). The abbreviation “PCAM/PSAM” is |
| | | interchangeable with PCPSAM. |
| PEC / PECC | Penetration enhancer | A component (PEC) of a composition comprising |
| | component / | constituent(s) (PECC(s)) which allow(s) |
| | Penetration enhancer | therapeutically effective or cosmetically effective |
| | component | amount(s) of other associated (co-delivered/co- |
| | constituent | administered) compound(s)/molecule(s) to |
| | | penetrate the skin with DOS improvement in the |
| | | amount of uptake of such other constituent(s), rate |
| | | of uptake of such other constituent(s), or both |
| PSAM | Protein S activity | A constituent of a protein S modulating |
| | modulator and | component (PS-MC) that DOS modulates protein |
| | PSAM component | S activity when delivered/administered in |
| | | effective amount(s) in subject(s). |
| PS-MC | Protein S | A component of a composition that DOS |
| | Modulating | modulate(s) protein S activity when delivered |
| | component | administered in effective amount(s) and that |
| | | comprises one or more PSAM(s) |
| TSD-PEC | Temporary skin | A type of penetration enhancer component |
| | disruption | comprising one or more agent(s) that are believed |
| | penetration enhancer | to temporarily disrupt skin components and that, |
| | component | when present in effective amount(s) and under |
| | | suitable condition(s) allow for passage/penetration |
| | | of large molecules into the skin (e.g., a compound |
| | | of ≥~500 Da, ≥~750 Da, ≥~1 kDa, ≥~1.5 kDa, |
| | | ≥2 kDa, ≥~2.5 kDa, or ≥~5 kDa) or |
| | | penetration/passage of molecule(s) that do not |
| | | significantly penetrate the skin without the |
| | | presence of the TSD-PEC or similar/equivalent |
| | | skin penetration means |
| VKC | Vitamin K | A vitamin K compound, an exemplary form of |
| | Compound | which is Vitamin K1 |

In cases, additional definitions/explanations of terms are provided, and the meaning of acronyms repeated in the following portions of the disclosure to aid readability.

### SUMMARY OF THE INVENTION

A summary of notable aspects of the invention is provided in this section. This Summary is not intended to be all-inclusive, and the scope of the invention is not limited to or by the aspects, features, elements, or embodiments provided in this Summary, which is included for illustration, rather than restriction. Any of the aspects described under this section can be combined with any other aspect described in any part of this disclosure.

This invention provides compositions that are suitable for cosmetic use, pharmaceutical use, or both, which comprise a coagulation pathway modulation component and a therapeutically effective amount of a nicotinamide-adenine dinucleotide (NAD) pathway modulation component (NADP-MC). In aspects, compositions of the invention (“compositions”) are suitable for topical application to mammalian skin, e.g., to humans. In aspects, compositions comprise a physiologically effective amount, a cosmetically effective amount, or a therapeutically effective amount, of a coagulation pathway modulation component and a NAD pathway modulation component. In aspects, compositions are provided as water-based cream compositions. In aspects, compositions are suitable for/adapted to topical application and are mostly, generally, or substantially entirely absorbed into the skin within about 4 hours, such as within about 2 hours, within about 1 hour, within about 0.5 hours, within about 0.25 hours, within about 0.125 hours, or within about 0.1 hours of topical application.

In embodiments, a coagulation pathway modulation component (“CP-MC”) in a composition detectably or significantly (“DOS”) enhancing protein C function(s), protein S function(s), or both, in a subject to which the composition is administered. In aspects, such enhancement is the result of DOS enhancement of activity of protein C, protein S, or both; increasing the stability of protein C, protein S, or both; or, by both increasing the activity and stability of protein C, protein S, or both. In aspects, compositions provided by the invention comprising a coagulation pathway modulating component which detectably or significantly affects one or more skin characteristics, such as, e.g., bruising. In aspects, the coagulation pathway modulation component comprises a vitamin K compound, e.g., vitamin K1.

In aspects, the NAD pathway modulation component (“NADP-MC”) of compositions detectably or significantly increase NAD functioning in a subject. In aspects, compositions DOS enhance the amount of bioavailable nicotinamide-adenine dinucleotide (NAD) in a recipient. In aspects, compositions provided by the invention DOS affect one or more skin characteristics, such as, e.g., smoothing of skin (e.g., ameliorating hyperkeratosis). In common aspects, the NAD coagulation pathway modulation component comprises a nicotinate compound, e.g., a non-irritating nicotinate compound, such as tocopheryl nicotinate, a niacinamide compound, or both.

In facets, the invention provides compositions comprising a coagulation pathway modulation component (“CP-MC”), an NADP-MC, and one or more penetration enhancer components, which can, in aspects, comprise an effective amount of a temporary skin disruption penetration enhancer component (TSD-PEC) (an amount that, e.g., DOS enhances the transport of another/other constituent(s) of the composition through the skin, typically one or more constituent(s) that otherwise would not be able to penetrate the skin in significant amount(s)). In specific aspects, the TSD-PEC detectably or significantly enhances the penetration of multiple compounds of the composition into/through the skin. In aspects, a TSD-PEC has an average molecular weight of about 2,000 Daltons to 20,000 Daltons. In aspects, a DPEC is a sodium hyaluronate composition. In aspects, a DPEC comprises fragment(s) of a hyaluronic acid compound, collagen compound, fibronectin compound, elastin compound, or a combination of any or all thereof. In aspects, the TSD-PEC comprises an effective amount of a hyaluronate component (e.g., a sodium hyaluronate compound (a physiologically suitable, typically topically suitable, salt of a hyaluronic acid composition) or comprises a fragment of a hyaluronic acid compound.

In embodiments, compositions comprise (1) an effective amount of a coagulation pathway modulation component (CP-MC) comprising ingredient(s) which modulate(s), typically enhance(s), protein C functions/activity(ies) (a protein C activity modulator) (PCAM) or PCAM component, or protein C and protein S functions (a protein C/protein S modulator (PCPSAM) or PCPSAM component) and (2) an effective amount of an NAD pathway modulation component (NADP-MC) comprising a nicotinate compound, wherein the ratio of the nicotinate compound to the PCAM is about 1:5-about 1:3 (on a weight percent to weight percent basis, as is, uncontradicted, the basis of all ratios described here). In aspects, the ratio of the NAD pathway enhancer component to a PCPSAM (PCAM/PSAM) component is about 2.5:1-about 4:1. In aspects, a NADP-MC comprises a nicotinate compound component and a niacinamide compound component wherein the ratio of the nicotinate compound component to the niacinamide compound component is about 1:5 to about 1:20, e.g., about 1:7.5 to about 1:17.5, e.g., about such as about 1:10-about 1:15.

In aspects, a coagulation pathway modulation component is present in an amount of about 0.75 wt. %-about 1.5 wt. % of a composition. In aspects, the NAD pathway modulation component is present in an amount of about 1 wt. %-about 5 wt. % of a composition. In aspects, the penetration enhancer component is present in a composition in an amount of about 1 wt. %-about 3 wt. % of a composition.

In embodiments, a nicotinate compound is present in a composition in an amount of about 0.15 wt. %-about 0.35 wt. % of the compositions. In aspects, a niacinamide compound is present in the compositions in an amount of about (~) 1 wt. %-~4 wt. % of the compositions. In aspects, a vitamin K compound is present in the compositions in an amount of about 0.75 wt. %-about 1.5 wt. % of a composition. In aspects, a TSD-PEC is present in the compositions in an amount of about 0.15 wt. %-about 0.35 wt. %. In aspects, a non-TSD-PEC is present in the compositions in an amount of about 1.5 wt. %-about 2 wt. %.

In aspects, the invention provides a composition having any of or any combination of the characteristics of any of the compositions described above, wherein the composition further comprises an effective number of amino acid(s), such as, a basic amino acid, e.g., arginine, histidine, or lysine, or a tryptophan, or a combination of any or all thereof. In aspects, an amino acid component of a composition detectably or significantly increases collagen levels, exhibits antioxidant activity, increases skin hydration, enhances skin healing, reduces wrinkles/fine lines, protects against UV damage, enhances activity of the coagulation pathway modulating component, or results in/performs a combination of any or all thereof.

In aspects, the invention provides a composition, having the features of any of or any combination of the compositions described above, wherein the composition further comprises an effective amount of one or more neurotransmitter-inhibiting peptides.

In aspects, the invention provides a composition, having the features of any of or any combination of the compositions described above, wherein the composition further comprises an effective amount or effective amounts of one or more antioxidants, one or more preservatives, one or more exfoliants, or (i.e., and/or) one or more emollients.

In certain aspects, compositions having any of the features or combination of features found in any of the compositions described above further comprise an effective amount of one or more fatty acid compounds in a fatty acid component. In aspects, compositions comprise at least about 2.5 wt. % compounds characterizable as fatty acid compounds.

In embodiments, the invention provides cosmetic compositions which modify the appearance of skin, e.g., result in a detectable or significant improvement in one or more conditions of the skin, but do not result in any physiological effect that would be considered treating or preventing a disease. In aspects, the therapeutic skin effect is the improvement of hyperkeratosis, the improvement of visible bruising, or both. In aspects, improvements in hyperkeratosis are measurable within about 10 hours of application of the compositions provided by the invention.

In aspects, the invention provides methods of modulating the condition of the skin of a subject comprising the administration of an effective amount of any one or more of the compositions described above, administered a sufficient number of times, to detectably or significantly modulate one or more conditions of the skin of the subject, such as, e.g., the appearance of hyperkeratotic skin, visible bruising, or both. In aspects, methods provided by the invention provide measurable improvement in hyperkeratosis (or, e.g., skin smoothness), within, e.g., one week, two days, one day, or even about 10 hours or less of topical application of the composition(s).

In aspects, the invention provides methods of treating or preventing a skin-associated disease or medical condition, such as, e.g., seborrheic dermatitis, psoriasis, or actinic keratosis. In aspects, the skin-associated disease or medical condition comprises bruising or wound healing.

In aspects, the invention provides methods such as any of the methods above wherein the skin-associated disease or medical condition is associated with the performance of a medical or aesthetic procedure on the subject. In aspects, composition(s) are provided before such a procedure, after such a procedure, or both. In aspects, composition(s) are provided promptly following the formation of a wound or bruise on the skin of a subject. In aspects, compositions are topically applied 2 or more times over a period of time, such as, 1-3 times per day for, e.g., 2 or more days, e.g., at least once per day for a period of about 1 week or more. In certain aspects, methods of the invention comprise initiation of administration of a composition provided by the invention only under the supervision of a licensed medical professional.

### BRIEF DESCRIPTION OF THE DRAWINGS/FIGURES

The drawings/figures provided here, and the associated following brief description of such drawings/figures, are intended to exemplify certain aspects and principles of the invention without limiting its scope.

FIG. 1 is a photograph of a deep hematoma and related bruises prior to treatment with a composition of the invention.

FIG. 2 is a photograph of the treated deep hematoma and related bruises of FIG. 1, 3 days after the start of treatment with a composition of the invention.

FIG. 3 is a photograph of the treated deep hematoma and related bruises of FIG. 1, 5 days after the start of treatment with a composition of the invention.

FIG. 4 is a photograph of bruising resulting from Qwo® treatment, two days after the start of treatment with a composition of the invention (left: treated; right: untreated).

FIG. 5 is a photograph of a bruise prior to treatment with a composition of the invention.

FIG. 6 is a photograph of the bruise of FIG. 5, approximately 4 days after the start of treatment with a composition of the invention.

FIG. 7 is a photograph of purpura prior to treatment with a composition of the invention.

FIG. 8 is a photograph of the purpura of FIG. 8, approximately 4 days after the start of treatment with a composition of the invention.

FIG. 9 is a photograph of a bruise approximately 2 hours post-first treatment with a composition of the invention.

FIG. 10 is a photograph of the bruise of FIG. 9, approximately 72 hours after the start of treatment with a composition of the invention.

FIG. 11 is a photograph of the bruise of FIG. 9, approximately 96 hours after the start of treatment with a composition of the invention.

FIG. 12 is a photograph of the bruise of FIG. 9, approximately 7 days after the start of treatment with a composition of the invention.

FIG. 13 is a photograph of untreated (left) and treated (right) bruising resulting from a cupping procedure, 24-hours post-procedure.

FIG. 14 is a photograph of the untreated (left) and treated (right) cupping-related bruising shown in FIG. 13, 36-hours post-procedure.

FIG. 15 is a photograph of the untreated (left) and treated (right) cupping-related bruising shown in FIG. 13, 48-hours post-procedure.

### DETAILED DESCRIPTION OF THE INVENTION

For convenience, combinations and methods of the invention (as a whole) and individual elements/steps thereof are described in this section of this disclosure. Despite the inclusion of sections or passages focused on specific elements/steps, any aspect, facet, embodiment, or other description of particular step(s) or element(s) can be applied to any general description of the compositions/methods of the invention, or any other recited element(s)/step(s) thereof, which are provided in any part of this disclosure.

### Compositions for Modulating Conditions in Subject(s)

In aspects, the invention provides compositions comprising an effective amount (e.g., a physiologically effective amount) of a coagulation pathway modulation component (CP-MC) and an effective amount of a nicotinamide-adenine dinucleotide (NAD) pathway modulation component (NADP-MC). Ingredients/constituents of compositions provided by the invention and certain of their relevant characteristics and uses thereof are described here in detail, as are methods of applying such compositions for achieving various effects in subjects.

In aspects, compositions of the invention comprise a therapeutically effective amount or cosmetically effective amount of a NADP-MC and a therapeutically effective amount or cosmetically effective amount of a CP-MC. In aspects, the NADP-MC comprises an effective amount of a nicotinate compound component. In aspects, the CP-MC comprises a non-polypeptide modulator of protein C. In aspects, the CP-MC comprises a vitamin K compound. In aspects, the NADP-MC comprises a mixture of a nicotinate compound component and a nicotinamide compound component. In aspects, compositions are suitable for topical application to mammalian skin (e.g., to a human). In aspects, compositions further comprise a penetration enhancing component, comprising, e.g., sodium hyaluronate (e.g., hydrolyzed sodium hyaluronate).

### Ingredients

Compositions of this invention typically, but not necessarily always, comprise a coagulation pathway modulation component (CP-MC), an NAD pathway modulation component (NADP-MC), or both a CP-MC and a NADP-MC, each of which can comprise one or more constituents. Compositions also can comprise additional ingredients, including, e.g., a penetration enhancer component, and optionally further ingredients such as antioxidant(s), emollient(s), or other physiologically active ingredients (e.g., skin modulating peptides, amino acids, or both). In aspects, some or most ingredients can, in aspects, provide one or more measurable, e.g., detectable or significant (DOS) effects in a subject, such as in one or more target tissues, organs, body parts, etc., such as on the skin of individual, parts of the skin (e.g., facial skin), or even on individual areas within the skin of a part of the body (such as, e.g., in individual lesions, bruises, and the like). In aspects, additional ingredients can be provided to, i.a., provide various defined functions, such as to aid in product stability, product application, appearance, or the like which, in aspects, are not directly related to the provision of a physiological, cosmetic, or therapeutic effect in a subject. A description of the primary components of the composition (a CP-MC, NADP-MC, or both), constituents thereof, and other possible components of compositions follows.

### Coagulation Pathway Modulation Component (CP-MC)

In aspects, compositions of the invention comprise an effective amount of a coagulation pathway modulating component (CP-MC). In aspects, a composition comprises a CP-MC that directly or indirectly modulates a coagulation pathway or the activity of one or more endogenous factors of a coagulation pathway. In aspects, a CP-MC or CP-MC constituent(s) increase(s) (e.g., detectably or significantly raises an amount/level of), directly or indirectly stabilizes (e.g., decreases the rate of degradation of or breakdown of) one or more factors of a coagulation pathway of a subject, or increases the activity of one or more endogenous factors of a coagulation pathway, such that, in aspects, a CP-MC improves/enhances one or more aspects of a coagulation pathway or components thereof, e.g., activity of coagulation factor(s). In aspects, a CP-MC and at least one constituent of a CP-MC modulates ≥2 coagulation pathways/factors. For example, a CP-MC/CP-MC constituent, can promote synthesis, activation, or stability of coagulation factors (blood/clotting factors), such as Factor II, VII, IX, and X, which are well known components of the human/mammalian coagulation cascade/pathway.

In aspects, a CP-MC or a constituent thereof also or alternatively, imparts DOS effects/activity upon other physiological processes that are separate from a coagulation pathway/cascade/process (i.e., non-coagulation pathway modulation activity). E.g., in aspects a CP-MC constituent DOS induces, enhances, causes, improves, etc., one or more physiological effects related to inflammation, bone metabolism (e.g., by affecting osteocalcin (e.g., significantly similar to vitamin K1/phytonadione), affecting matrix Gla protein (MGP) (significantly similar to vitamin K), or affecting (e.g., inhibiting) mineralization (significantly similar to vitamin K)). In aspects, a CP-MC or a constituent thereof DOS affects cell proliferation (e.g., DOS modulating cell growth, apoptosis, and in aspects, anti-proliferative effects in cancerous cells) (e.g., in a manner that is significantly similar to vitamin K1).

In aspects, a CP-MC comprises ≥2 constituents. In other aspects, the coagulation pathway modulation component comprises only a single CP-MC constituent. In aspects, a CP-MC comprises 3, 4, 5, or more constituents, which each alone, together, or both, impart a coagulation pathway modulation effect when delivered to a target subject in a physiologically effective amount. In aspects, two or more constituents of a coagulation pathway modulation component can act additively/cooperatively or even synergistically in their provision of a coagulation pathway modulation effect. In aspects, a CPMC comprises two or more constituents that together impart an effect which is DOS greater than the effect either constituent would provide alone. In aspects where a CP-MC comprises ≥2 constituents, any 1st constituent does not significantly negatively impede the activity of any 2nd constituent, such as, e.g., it does not DOS reduce or otherwise negatively impact the activity of any 2nd constituent whereby the second constituent fails to demonstrate a detectable effect in a majority of subjects in an appropriately conducted and controlled clinical trial due to the interference of the first constituent. Further, in aspects, constituent(s) of a CP-MC does/do not significantly negatively impede the activity of any other component or ingredient of a composition, such as, e.g., an NAD pathway modulation component (NADP-MC). In aspects, the CP-MC is dermatologically acceptable and suitable for topical application in the context of the overall composition.

In aspects, a CP-MC or constituent(s) thereof detectably or significantly modulates one or more of protein C, protein S, and antithrombin, when administered to a subject in an effective manner and in effective amounts.

In aspects, a CP-MC or constituent thereof comprises, mostly comprises, generally only comprises, consists essentially of, or consists of compound(s) that modulate thrombosis-related conditions. In other aspects, a CP-MC also or alternatively comprises compound(s) that modulate both bleeding and thrombosis-related conditions (e.g., modulators of Factor II, Factor VII, or Factor IX).

In aspects, a CP-MC does not comprise any constituent having a molecular weight of more than about 50 kDa, more than ~30 kDa, more than ~20 kDa, more than ~15 kDa, more than ~10 kDa, more than ~5 kDa, more than ~4 kDa, more than ~3 kDa, more than ~2 kDa, or ≥than 1 kDa.

In aspects, most, generally all, or all of a CP-MC does not comprise any multimeric proteins/polypeptides (e.g., does not comprise any dimeric proteins). In other aspects, most, generally all, substantially all, or all of a CP-MC does not comprise any polypeptide/protein component. In aspects, a CP-MC constituent comprises a mineral or a mineral-containing compound (e.g., a zinc-containing compound or zinc ions). In aspects, a CP-MC comprises ≥2 compounds classified in the art as vitamins (which can include vitamers, provitamins, and the like) or that are structurally and functionally related to a vitamin that classification as a vitamin is appropriate (e.g., a compound that is known to be or would otherwise be recognized as a vitamin analog or derivative). Examples of such compounds include, e.g., vitamin C, vitamin K, and vitamin Q (co-enzyme Q/ubiquinone). In aspects, a CP-MC comprises a vitamin K compound (including, e.g., two or more vitamin K compounds) (which are discussed in detail below). In aspects, a CP-MC comprises a vitamin K compound and one or more additional vitamins. For example, in aspects, a vitamin D constituent is present in a CP-MC along with a vitamin K compound to accentuate bone and tissue calcification activity demonstrated by vitamin K, activity supplementing the coagulation pathway modulation activity of the CP-MC. In aspects, a vitamin A constituent is present in a CP-MC along with a vitamin K compound to further augment the modulation effect of vitamin K on one or more vitamin K-dependent enzymes. As another example, in aspects, a vitamin E compound is present in a composition as part of one or more other composition ingredients, such as, e.g., a component of an NAD pathway modulation component (such as, e.g., a tocopheryl nicotinate). In aspects, a composition comprises no more than 6, e.g., no more than 2, 3, 4, or 5, types of vitamins/vitamin compounds. In aspects, any vitamin(s) that act as CP-MC constituent(s), NADP-MC constituent(s), or both CP-MC and NADP-MC constituent(s) make up most, generally all, or substantially all of the vitamin content of the composition. In aspects, vitamin CP-MC constituent(s) are present in an amount that places the vitamin CP-MC constituent(s) among the top 50%, top 40%, or top 33% of ingredients in the composition in terms of amount/concentration. In aspects, a CP-MC constituent(s) is composed of vitamin compounds that make up at least 1.5%, such as at least about 2% of the non-carrier (e.g., non-water) ingredients of a composition. In aspects, at least about 25%, ≥~33%, or most of any non-CP-MC constituent vitamin(s) in a composition are conjugated or complexed with other compounds of a composition (e.g., as in the case of tocopheryl nicotinate or methyl nicotinate). In aspects, most, generally all, or all of the vitamin ingredient(s)/compound(s) of a composition are not conjugated. In aspects, the ratio of non-conjugated to conjugated vitamin compounds in the composition is at least about 5:1, e.g., ≥~8:1, ≥~10:1, ≥~12:1, or ≥~15:1. In aspects, generally all, essentially all, or all of a CP-MC vitamin compound component is composed of non-conjugated vitamins. In aspects, vitamin(s) that act as CP-MC constituent(s), NADP-MC constituent(s), or that make up both CP-MC and NADP-MC constituent(s) are present in greater amounts than other vitamin(s) in the composition (e.g., a CP-MC vitamin is present in a greater concentration/amount than any non-CP-MC vitamin, the total amount of CP-CM vitamins are present in a greater concentration/amount than any non-CP-MC vitamins (collectively), or both). In aspects, CP-MC vitamin compound(s) make up at least about 0.5%, at least about 0.75%, or at least about 0.9% of an overall composition. In aspects, vitamin(s) in the CP-MC and NADP-MC make up at least about 1 wt. %, ~1.5 wt. %, ~2 wt. %, ~2.5 wt. %, ~3 wt. %, at least ~3.5 wt. %, or at least ~4 wt. % of a composition.

According to aspects, a CP-MC comprises coagulation factors/peptides, or coagulation factor co-factors, regulators, or modulators. In aspects, a CP-MC causes DOS modulation of, e.g., platelet activation, fibrinolysis, or a specific coagulation cascade, such as, e.g., a tissue factor pathway, a contact activation pathway, a common pathway scheme, a cell-based scheme of coagulation, or other such models of coagulation. Examples of such coagulation factors can include, e.g., fibrinogen (Factor I), prothrombin (Factor II), Tissue Factor or Tissue Thromboplastin (Factor III), Factor IV, proaccelerin/labile factor (Factor V), Factor VI, stable factor/proconvertin (Factor VII), antihemophilic factor A (Factor VIII), antihemophilic factor B/Christmas factor (Factor IX), Stuart-Prower factor (Factor X), plasma thromboplastin antecedent (Factor XI, Hageman factor (Factor XII), fibrin-stabilizing factor (Factor XII), von Willebrand Factor, Fletcher Factor (prekallikrein, Fitzgerald Factor (high-molecular-weight kininogen/HMWK), fibronectin, antithrombin III, heparin cofactor II, protein C, protein S, protein Z, protein Z-related protease inhibitor (ZPI), plasminogen, alpha 2-antiplasmin, tissue plasminogen activator (tPA), urokinase, plasminogen activator inhibitor-1 (PAI1), plasminogen activator inhibitor-2 (PAI2), cancer procoagulant, platelet-activating factor, platelet factor 4, thromboxane A2, protein kinase C, phospholipase A2, integrin membrane glycoprotein IIb/IIIa, serine protease(s), Factor V, Factor III, Factor XIII (a transglutaminase), calcium, phospholipid(s), antithrombin, tissue factor pathway inhibitor (TFPI), plasmin, or, e.g., prostacyclin. In aspects, the active ingredient(s) of a CP-MC is/are procoagulant(s). In aspects, such compound(s) are or comprise anticoagulant(s). In aspects, a CP-MC comprises one or more heparin compounds (e.g., heparin, liposomal heparin, heparinoid mucopolysaccharides, etc.), one or more thrombin compounds, etc.

In aspects, a CP-MC, when provided in sufficient amounts, DOS impacts platelet adhesion. In aspects, a CP-MC constituent is, comprises, or mostly comprises, an herbal ingredient or other natural/plant-derived ingredient such as feverfew, meadowsweet, white willow, turmeric, ginger, cayenne peppers, garlic, cassia cinnamon, Gingko biloba, omega-3 fatty acid(s), chamomile, fenugreek, red clover, dong quai, evening primrose oil, etc. In aspects, most or all of a CP-MC is not an herbal ingredient (such as an extract or other herbal-derived mixture) but is one or more substantially purified/purified compound(s).

In aspects, compositions incorporate nucleic acid constructs encoding for one or more CP-MC constituents, such as one or more coagulation factors, functional fragments thereof, or functional variants thereof (in DNA form, such as one or more plasmid(s)/viral vector(s), RNA form, such as one or more mRNA vector(s), or both). In aspects, a CP-MC comprises a DNA plasmid wherein the DNA plasmid comprises a nucleotide sequence encoding one or more proteins which, when present in sufficient amounts, can modulate one or more coagulation-related physiological processes, such as, e.g., protein C or protein S. In aspects, a CP-MC comprises one or more RNA molecule(s) (e.g., mRNA(s)), wherein each such RNA molecule comprises a nucleotide sequence encoding one or more proteins which, when present in sufficient amounts, can modulate one or more coagulation-related physiological processes, such as, e.g., protein C or protein S. In aspects, a coagulation pathway component comprises one or more DNA constituents, one or more RNA constituents, or a combination thereof. In aspects, the DNA plasmid, RNA molecule, or both encode a protein having at least an equivalent physiological modulation effect or an improved modulation effect on one or more coagulation pathway constituents as, e.g., an effective amount of a vitamin K compound. In aspects, the DNA plasmid, RNA molecule, or both encode a protein having at least an equivalent physiological modulation effect or an improved modulation effect on one or more coagulation pathway constituents as vitamin K1.

In aspects, any of the above coagulation pathway modification components is provided in a variant form, such as a derivative or analogue/analog/mimetic which provides at least substantially similar or effectively equivalent physiological activity as the naturally occurring constituent listed here. In one aspect, a variant/derivative is a compound comprising one or more modifications which detectably or significantly (1) enhance the ability of the constituent to pass through the skin, e.g., DOS increase the amount of compound which can pass through the skin during any given period of time compared to the native compound, improves the speed upon which an amount of compound can pass through the skin, or any combination thereof; (2) enhance the ability of the compound to modulate coagulation pathway activity in one or more respects, in cases DOS enhancing one or more cosmetic or therapeutic outcomes associated with administration of a composition comprising an effective amount of the variant; or (3) DOS demonstrate the effects of both (1) and (2).

In aspects, a CP-MC is present in an amount of about 0.05 wt. %-about 2.5 wt. % of the composition, such as ~0.05 wt. % to ~1 wt. %, ~0.05 wt. % to ~0.75 wt. %, ~0.05 wt. % to ~0.5 wt. %, ~0.05 wt. % to ~0.25 wt. %, ~0.1 wt. % to ~1.5 wt. %, ~0.15 wt. % to ~1.5 wt. %, ~0.25 wt. % to ~1.5 wt. %, or ~0.5 wt. % to ~1.5 wt. %, e.g., 0.75 wt. %-about 1.5 wt. %, or ~1 wt. % of the composition. In aspects, a CP-MC consists essentially of a PCAM/PSAM (PCPSAM). In aspects, the CP-MC consists essentially of a vitamin K compound, e.g., a vitamin K1 compound.

### Protein C Modulators

In aspects, a CP-MC, constituent(s) of a CP-MC, or both, imparts, enhances, induces, causes, or modulates a measurable/detectable effect or significant effect on protein C activity in a subject (or in a significant number of subjects, as determined, e.g., by suitable clinical testing). Constituent(s) that cause such DOS effects can be referred to as protein C activity modulators (PCAMs) and a component of a CP-MC that causes such DOS effects can be called a protein C modulating component (PC-MC). For sake of clarity, a PC-MC can be a constituent of a CP-MC, and a PCAM can be a constituent of a PC-MC, making it also a constituent of a CP-MC. In aspects, a CP-MC, CP-MC constituent (e.g., a PC-MC, a PCAM, or both), or both, can enhance, modulate, promote, cause, induce, increase, etc., any aspect(s) of protein C activity in subject(s). In aspects, a CP-MC or CP-MC constituent DOS increases, sustains, or otherwise enhances/increases the conversion of protein C to “activated protein C” (“APC”), the activity of APC, the amount of APC, the duration of APC activity, the extent of APC activity, etc.

In aspects, a PC-MC/PCAM increases/enhances/improves levels of Protein C activity in subject(s). Protein C activity in a subject can be enhanced by, e.g., increasing protein C level(s) in a subject, by promoting the stability of endogenous protein C, by enhancing the activity of protein C (either directly or indirectly through enhancing other factors that enhance protein C activity or reducing factors that reduce protein C activity), or any combination thereof.

Protein C (also referred to as, e.g., autoprothrombin IIA and blood coagulation Factor XIX) is an endogenous constituent of the human blood anticoagulation system, and a vitamin K-dependent, protein zymogen. The biology/chemistry of Protein C is known in the art (see, e.g., Dhalback, et. al. “The Protein C Anticoagulant System” in Stamatoyannopoulos G, Majerus P W, Perlmutter R M, Varmus H, eds. The Molecular Basis of Blood Disease, third ed. Philadelphia: WB Saunders Company; 2000:614-656), DAHLBACK in, “Blood coagulation and its regulation by anticoagulant pathways: genetic pathogenesis of bleeding and thrombotic disease”, Journal of Internal Medicine, Volume 257, Issue 3, pp. 209-223 (March 2005), and citations contained in either reference).

In aspects, a PCAM comprises a protein C polypeptide or an active fragment, variant, or derivative of a protein C polypeptide. Such PCAMs increase protein C activity by directly increasing the level of protein C or by providing an amount of a protein C derivative, fragment, analog, etc., which acts, in one or more respects, similar to protein C in modulating coagulation pathway(s), albeit possibly in different respects in some ways (e.g., at an enhanced level or in a modified manner). A variant of a protein/polypeptide in this and other respects typically is understood to be a polypeptide/protein that (1) is substantially similar in structure and (2) at least similar in one or more respects in terms of biological activity to a referenced protein, domain, or sequence (e.g., here, a protein C, such as human protein C). Variants differ from a native/endogenous/wild-type polypeptide/protein based on one or more deletions, insertions, or substitutions of amino acid residues. Substitutions may further be defined as conservative or unconservative substitutions. Uncontradicted, a variant herein means a variant that (1) exhibits at least about 80% amino acid sequence identity, typically at least about 85%, ~90%, ~93%, or at least ~95% identity to a referenced sequence, and (2) exhibits significantly similar or detectably or significantly improved biological activity in one or more respects as compared to the endogenous/native counterpart (e.g., in terms of one or more physiological effects, such as the modulation of the coagulation cascade, reduction of bruising, or other effects associated with the native/endogenous protein). Methods of determining identity between variants are described in, e.g., US20200325182. In aspects, a protein C modulator is a derivative of a protein C (e.g., a PEGylated derivative). Derivatives of polypeptides are molecules in which one or more non-amino acid moieties/groups and the like are bound to an amino acid chain of a polypeptide/protein. Derivatives also are described in, e.g., US20200325182. In aspects, a protein C modulator comprises a nucleic acid that expresses a protein C polypeptide or a variant thereof. A large variety of nucleic acid expression delivery systems including DNA constructs and RNA constructs; viral vectors comprising such constructs; cellular delivery systems comprising such constructs; and non-viral vector systems are also provided in US20200325182, which can be adapted to such aspects of the invention. A variety of endogenous protein C polypeptides/proteins are known in the art (see, e.g., UniProtKB-accession/entry P04070 (PROC_HUMAN)). Examples of protein C derivatives, variants/analogs, nucleic acid delivery systems, and related compositions, etc., are provided in, e.g., U.S. Patent Applications 20050176083, 20050143283, 20050095668, 20050059132, 20040028670, 20030207435; 20030027299, 20030022354, 20060204489, and 20030018175; U.S. Pat. Nos. 6,933,367; 6,841,371; 6,815,533; 6,630,138; 6,630,137; 6,436,397; 6,395,270; 6,162,629; 6,159,468; 5,837,843; 5,453,373; 5,330,907; 5,766,921; 5,753,224; 5,516,650; 5,358,932; 5,766,921 4,775,624; 4,968,626; 5,151,268; and 5,270,178; AU1989038906; WO1998044000, WO2010020677, WO2008048646, WO2004113385, WO2004044190, and WO2003073980; and Cai S R et al. J Clin Invest. 1998; 101 (12):2831-2841 and Quinn L M et al. Biochem Soc Trans. 2015 August; 43 (4):691-5. For example, a protein C variant can comprise a cytoprotective-selective APC variant, such as protein 3K3A-APC (see, e.g., Mosnier L O et al. Thromb Haemost. 2014 November; 112 (5):883-92). Aspects of cryoprotective effects of protein C/the protein C pathway are known (see, e.g., L O Mosnier et al. Blood 2007 109:3161-3172; Griffin J H, et al. Int J Hematol. 2012; 95 (4):333-345; and Rezaie A R. Curr Med Chem. 2010; 17 (19):2059-69). In aspects, a CP-MC comprises a PCAM that is specifically adapted for subcutaneous administration (see, e.g., U.S. Pat. No. 5,827,818). In aspects, a PCAM comprises a protein C polypeptide, active fragment thereof, or a fragment thereof (or a nucleic acid that expresses any one or more thereof), or derivative of any thereof, wherein the composition further comprises a temporary skin disrupting/disruption penetration enhancer (TSD-PEC) that significantly promotes the uptake of the protein C polypeptide or protein C-related compound (derivative, variant, etc.) into the skin. In aspects, a PCAM in a composition is a protein C precursor/protein C zymogen.

In aspects, some, most, generally all, or all, of a PC-MC is not made up of a protein C polypeptide/protein or related compound. In aspects, some, most, generally all, or all of a PC-MC is composed of one or more polypeptides/proteins (or protein/polypeptide expressing vectors/constructs) that are not protein C polypeptides/proteins or related compounds. In aspects, some, most, generally all, or all of a PC-MC comprises one or more non-protein-C polypeptides/proteins that are not protein C variants, derivatives, fragments, etc. For example, in one aspect, a PC-MC comprises an effective amount of a protein S polypeptide or related compound (e.g., a variant or derivative thereof) (related aspects are discussed further below), a thrombomodulin polypeptide or related compound, or an endothelial protein C receptor (EPCR) or related compound. In aspects, a PC-MC comprises an effective amount of a Factor V polypeptide or a related compound. In aspects, a PC-MC comprises a protein C activity-modulating integrin (e.g., Mac-1 (CD11b/CD18), β1 integrins, or β3 integrins). In aspects, a PC-MC comprises an effective amount of Sphingosine-1-Phosphate Receptor 1 (S1P1) or a related compound. In aspects, a PC-MC comprises an effective amount of apolipoprotein E receptor 2 or a related compound. In aspects, a PC-MC comprises an effective amount of an anionic phospholipid (e.g., phosphatidylserine, cardiolipin, etc.) or a glycosphingolipid (e.g., glycosylceramide.) In aspects, a PC-MC also or alternatively comprises an effective amount of one or more of glycoprotein Ib, Tie2 Tyrosine-protein kinase, vitamin K 2,3-epoxide reductase, apolipoprotein A-I, a gamma-glutamyl carboxylase, platelet factor 4 (PF4) (see, e.g., WO2013086493), and an epidermal growth factor receptor (EGFR). In aspects, a PC-MC comprises a combination of ≥1 protein C polypeptide(s)/protein(s) or related compound(s) and one or more non-protein C PC-MC(s), such as ≥1 non-protein C polypeptides/proteins described in this paragraph. In aspects, a PC-MC comprises ≥2 non-protein C polypeptide/protein/related compound polypeptides/proteins, such as ≥2 of the types of polypeptides/proteins described in this paragraph.

Examples of non-protein/polypeptide PCAMs include, e.g., protein C activity/pathway modulating lipid/lipid-related compound(s)/composition(s). Examples of such lipid/lipid-related compound(s)/composition(s) include, e.g., negatively charged phospholipid PCAMs, such as phosphatidylserine and cardiolipin; protein C-modulating sphingolipids (e.g., glycosphingolipids and sphingosine), e.g., glucosylceramide; and high-density lipoprotein.

In aspects, some, most, generally all, or all of the PC-MC comprises one or more mineral-related compound(s)/composition(s). In aspects, a PC-MC comprises an effective amount of one or more copper chelator compound(s)/composition(s) that inhibit inactivation of protein C (see, e.g., US20030055003). In aspects, a PC-MC comprises an effective amount of a zinc ion (e.g., Zn(2+)) or zinc-containing compound/composition, that DOS enhances protein C pathway activity (e.g., protein C: EPCR binding, PAR1 activity, and p44/42 MAPK activity) (see, e.g., Sen P, et al. J Biol Chem. 2010 Jun. 25; 285 (26):20410-20).

In aspects, a PC-MC comprises cells that modulate protein C activity/pathway(s) in subjects (e.g., in a subject or a significant number of subjects). Examples of such cells include dendritic cells and other protein C pathway-modulating lymphocytes.

In aspects, a PC-MC detectably or significantly modulates (e.g., enhances/improves) Protease Activated Receptor 1 (PAR1) activity in a subject or in a significant number of subjects (e.g., as determined through one or more clinical studies) (uncontradicted, any reference to modulation herein implicitly discloses both enhancement/improvement and reduction/inhibition). In aspects, a PC-MC DOS modulates PAR3 activity in subjects or in a significant number of subjects. In aspects, a PC-MC DOS modulates one or more aspects of a subject's phosphatidylinositol 3-kinase (PI3K-PKB)/Akt/mTOR (mammalian target of rapamycin) pathway(s). In aspects, a PC-MC DOS promotes/enhances/causes carboxylation of glutamic acid residues in the Gla domain of endogenous coagulation factors, such as protein C. In aspects, a PC-MC DOS modulates activation of Src family kinases, associated with anti-inflammatory effects. In aspects, a CP-MC, CP-MC constituent, or both, such as, e.g., a PC-MC or a PCAM, can enhance, modulate, promote, cause, induce, increase, etc., thrombin/thrombomodulin proteolytic cleavage.

In aspects, at least one, some, most, generally all, or all of the PCAMs of a PC-MC DOS modulate the activity of at least two endogenous coagulation factors in subject(s). In aspects, such a PCAM DOS modulates ≥~2, ≥~3, ≥~4, ≥~5, or all of Factors I, VII, IX, and X, protein C, and protein S in subjects or a significant number of subjects.

In aspects, a coagulation pathway modulating component, e.g., a PC-MC, e.g., a PCAM, DOS enhances the activity of endogenous protein C by providing sufficient amount(s) of element(s) necessary in the formation of protein C, the activation of protein C, or both. For example, in aspects, a CP-MC provides element(s) required by protein C to develop or maintain proper form, such as, e.g., providing element(s) which aid in the formation of residues which are involved in the establishment of a structure of protein C which can effectively bind EPCR (such as, e.g., the formation of Gla residues formed as the result of vitamin K-dependent post-translational carboxylation of glutamic acid residues on the Gla domain, such residues binding calcium and important in the proper folding of the domain, which binds EPCR, as described above). In aspects, a PCAM acts to DOS enhance the catalytic activity of protein C indirectly, by affecting one or more co-factors such as protein S (described elsewhere herein), a coagulation pathway modulation component affecting the activity of protein S can belong to a protein S modulation component (PS-MC) and can be a protein S activity modulator (PSAM), hence, e.g., a CP-MC can comprise a PS-MC, and PSAMs can be constituents of a PS-MC and a CP-MC.

In aspects, a PCAM or PC-MC DOS enhances the activity of, and DOS stabilizes, endogenous protein C in subject(s). In aspects, a CP-MC component consists generally of, essentially of, or substantially of, a PCAM. In aspects, a CP-MC component consists generally of, essentially of, or substantially of, a PSAM. In aspects, a CP-MC component consists generally of, essentially of, or substantially of, a PCAM/PSAM (also referred to as a PCPSAM, e.g., a compound capable modulating the activity, either directly or indirectly, of both protein C and protein S).

In aspects, the presence of effective amounts of a CP-MC, e.g., of one or more PCAMs, increases the amount of protein C available for effective binding to EPCR at a given time during a period in which composition(s) provided by the invention are in use by at least about 0.5%, ≥~0.75%, ≥~1%, ≥~2%, ≥~5%, ≥~10%, ≥~15%, or even ≥~20% or more, or any other significant or detectable amount, compared to the amount of protein C available for effective finding to EPCR when composition(s) are not provided, when similar or equivalent environments exist (e.g., the presence or absence of a skin condition, such as e.g., hyperkeratosis or bruising, etc.) as measured by appropriate in-vitro or in-vivo techniques.

In aspects, a PCAM/CP-MC/PC-MC increase(s) the percentage of protein C which can effectively bind an EPCR (or bind to and, when activated, detach from, an EPCR) over a period, e.g., by at least about 0.5%, ≥~0.75%, ≥~1%, ≥~2%, ≥~5%, ≥~10%, ≥~15%, or even ≥~20% or more, or any other significant or detectable amount, compared to similar untreated subjects or the subject in a similar state when untreated with the composition or other control.

In aspects, effective amounts of a CP-MC, e.g., of one or more PCAMs, increases the percentage of protein C circulating as APC, e.g., by at least about 0.5%, ≥~0.75%, ≥~1%, ≥~2%, ≥~5%, ≥~10%, ≥~15%, or even ≥~20% or more, or any other significant or detectable amount, compared to the percentage of protein C circulating as non-activated protein C when composition(s) are not provided, when similar or equivalent environments exist (e.g., the presence or absence of a skin condition, such as e.g., hyperkeratosis or bruising, etc.) as measured by appropriate in-vitro or in-vivo techniques, or in other suitable control.

In aspects, effective amount(s) of a CP-MC, e.g., a PC-MC comprising one or more PCAMs, increases the average stability of circulating protein C at a given time during a period in which composition(s) provided by the invention are in use, e.g., by at least about 0.5%, ≥~0.75%, ≥~1%, ≥~2%, ≥~5%, ≥~10%, ≥~15%, or even ≥~20% or more, or any other detectable or significant amount, as compared to a suitable control in the subject or similar subjects (e.g., in a test population). For example, if the average half-life of activated protein C is about 20 minutes (see DHALBACK, 2005, supra), the presence of effective amounts of one or more PCAMs can increase the average stability of circulating protein C at a given time during a period in which composition(s) provided by the invention are used by at least about 0.5%, ≥~0.75%, ≥~1%, ≥~2%, ≥~5%, ≥~10%, ≥~15%, or even ≥~20% or more (e.g., to an average half-life of detectably ≥~20 minutes, e.g., ~21 minutes, 22 minutes, 23 minutes, 24 minutes or more).

In aspects, effective amounts of a CP-MC, e.g., of one or more PCAMs, as provided by composition(s), increases the relative amount of inactivated Factor V, Factor VIII, or both (or, stated in the alternative, decreases the amount of activated Factor V, Factor VIII, or both) in circulation at a given time, e.g., by at least about 0.5%, ≥~0.75%, ≥~1%, ≥~2%, ≥~5%, ≥~10%, ≥~15%, or even ≥~20%, or another detectable or significant amount, as measured by appropriate in-vitro or in-vivo techniques in an appropriate control state/population. In aspects, a PC-MC/PCAM increases the ratio of inactivated Factor V, Factor VIII, or both to activated Factor V, Factor VIII, or both (respectively) in circulation at a given time during a period in which composition(s) provided by the invention are in use by, e.g., at least about 0.5%, ≥~0.75%, ≥~1%, ≥~2%, ≥~5%, ≥~10%, ≥~15%, or even ≥~20% or more, or other detectable or significant amount, compared to the ratio of inactivated-to-activated Factor V, Factor VIII, or both when composition(s) are not provided, in a suitable control state/population.

In aspects, a PC-MC comprises one or more agents that block agents that block protein C activity (e.g., resulting in an increase in protein C activity). E.g., in one aspect a PC-MC comprises a brodifacoum antagonist, a warfarin antagonist, a Factor VIII antagonist, etc.

In aspects, a PC-MC/PCAM in effective amount DOS modulates inactivation of Factor Va. In aspects, a PC-MC/PCAM in effective amount DOS modulates inactivation of Factor VIIIa, causes DOS degradation of Factor VIIa, or both. In aspects, a PC-MC/PCAM in effective amount DOS modulates subject inflammation, apoptosis levels, or both (in a subject or a portion of a population of subjects).

In certain aspects, compositions comprise an effective amount of one or more compounds capable of performing one or more activities of that performed by endogenous activated protein C, e.g., an “activated protein C variant”. In aspects, such a variant can target specific elements of various pathways, e.g., such a variant may have a specificity for factor Va versus PAR-1.

In aspects, compositions comprise an effective amount of one or more ingredients, e.g., one or more compounds, which provide a detectably or significantly similar effect as activated protein C by way of up-regulating a gene which is or can be upregulated by activated protein C. A list of such genes is provided in Table 2 below. In aspects, compositions comprise one or more, such as 1, 2, 3, 5 or more of the proteins provided in Table 2 below, e.g., for example 1, 2, 3, 5 or more of the proteins provided in Table 2 below under the section header “Up-Regulation” (such as, for example, CXCL2, F3, IL1R1, IL6, IL8, etc.).

| | | | | |
|---|---|---|---|---|
| | | | Reference | Other Protein |
| Gene (*) | Official Gene Name | Pathway Affected | Sequence | Name |

Up-Regulation

| | | | | |
|---|---|---|---|---|
| BCL2A1 | BCL2-related protein A1 | Apoptosis | NM_004049 | A1 |
| BMP2 | Bone morphogenetic | Differentiation | NM_001200 | BMP-2 |
| | protein 2 | | | |
| CCL2 | Chemokine (C-C motif) | Inflammation | NM_002982 | MCP-1 |
| | ligand 2 | | | |
| CX3CL1(**) | Chemokine (C-X3-C | Adhesion | NM_002996 | Fractalkine |
| | motif) ligand 1 | | | |
| CXCL2 | Chemokine (C-X-C motif) | Inflammation | NM_002089 | MIP-2, GRO |
| | ligand 2 | | | |
| DDX21 | DEAD (Asp-Glu-Ala-Asp) | Transcription | NM_004728 | RNA helicase |
| | box polypeptide 21 | | | II/Gu-α |
| F3 | Coagulation factor III | Coagulation | NM_001993 | TF, CD142 |
| | (thromboplastin, tissue | | | |
| | factor) | | | |
| HBEGF | Heparin-binding EGF-like | Proliferation | NM_001945 | HBEGF |
| | growth factor | | | |
| IL1R1(**) | Interleukin 1 receptor, type | Inflammation | NM_000877 | IL-R1, CD121a |
| | 1 | | | |
| IL6 | Interleukin 6 (interferon, | Inflammation | NM_000600 | IL-6 |
| | beta 2) | | | |
| IL8 | Interleukin 8 | Inflammation | NM_000584 | IL-8, CXCL-8 |
| NFKB1(**) | Nuclear factor of kappa | Transcription | NM_003998 | NF-κB |
| | light polypeptide gene | | | |
| | enhancer 1 | | | |
| NFKB2(**) | Nuclear factor of kappa | Transcription | NM_002502 | NF-κB2 p100 |
| | light polypeptide gene | | | |
| | enhancer 2 | | | |
| NOS3 | Nitric oxide synthase 3 | Inflammation | NM_000603 | eNOS |
| | (endothelial cell) | | | (endothelial nitric |
| | | | | oxide synthase) |
| NR4A1 | Nuclear receptor subfamily | Apoptosis | NM_173157 | TR3, Nur77, |
| | 4, group A, member 1 | | | NA4A1 |
| NR4A2 | Nuclear receptor subfamily | Transcription | NM_006186 | NURR1, NA4A2 |
| | 4, group A, member 2 | | | |
| NR4A3 | Nuclear receptor subfamily | Transcription | NM_006981 | NOR-1, NA4A3 |
| | 4, group A, member 3 | | | |
| PCNA | Proliferating cell nuclear | Cell Cycle | NM_002592 | PCNA |
| | antigen | | | |
| PTGS2 | Prostaglandin- | Inflammation | NM_000963 | COX-2 |
| | endoperoxide synthase 2 | | | |
| SMAD3 | SMAD, mothers against | Apoptosis | NM_005902 | SMAD-3 |
| | DPP homolog 3 | | | |
| | (Drosophila) | | | |
| TNFAIP3 | Tumor necrosis factor, | Apoptosis | NM_006290 | A20 |
| | alpha-induced protein 3 | | | |

Down-Regulation

| | | | | |
|---|---|---|---|---|
| B2M | Beta-2-microglobulin | Inflammation | NM_004048 | B2M |
| BIRC5 | Baculoviral IAP repeat- | Apoptosis | NM_001168 | Survivin |
| | containing 5 (survivin) | | | |
| CALR | Calreticulin | Inflammation | NM_004343 | Ro, cC1qR |
| CMKOR1 | Chemokine orphan | Cell signaling | NM_020311 | RDC1 |
| | receptor 1 | | | |
| CX3CL1(**) | Chemokine (C-X3-C | Adhesion | NM_002996 | Fractalkine |
| | motif) ligand 1 | | | |
| EFNA1 | Ephrin-A1 | Chemoattract | NM_004428 | Ephrin-A1 |
| ICAM1 | Intercellular adhesion | Adhesion | NM_000201 | ICAM-1, CD54 |
| | molecule 1 (CD54) | | | |
| ILR1(**) | Interleukin 1 receptor, type | Inflammation | NM_000877 | IL-1R1, DC121a |
| | I | | | |
| LTB | Lymphotoxin beta (TNF | Inflammation | NM_002341 | LT-β |
| | superfamily, member 3) | | | |
| MMP10 | Matrix metallopeptidase 10 | Proteolysis | NM_002425 | MMP-10 |
| | (stromelysin 2) | | | |
| NFKB1(**) | Nuclear factor of kappa | Transcription | NM_003998 | NF-κB |
| | light polypeptide gene | | | |
| | enhancer 1 | | | |
| NFKB2(**) | Nuclear factor of kappa | Transcription | NM_002502 | NF-κB2 p100 |
| | light polypeptide gene | | | |
| | enhancer 2 | | | |
| SELE | Selectin E (endothelial | Adhesion | NM_000450 | E-selectin, |
| | adhesion molecule 1) | | | CD62E |
| SOD2 | Superoxide dismutase 2, | Oxidation | NM_000636 | MNSOD |
| | mitochondrial | | | |
| THBS1 (***) | Thrombospondin 1 | Adhesion | NM_003246 | TSP-1 |
| TP53 (***) | Tumor protein p53 (Li- | Apoptosis | NM_000546 | P53 |
| | Fraumeni syndrome) | | | |
| VCAM1 | Vascular cell adhesion | Adhesion | NM_080682 | VCAM-1, CD106 |
| | molecule 1 | | | |

Table 2 has been extracted in part from Mosnier, et. al. in Blood, 15 Apr. 2007, volume 109, number 8, pp. 3161-3172; a reference incorporated by reference herein in its entirety. Per this reference: (*) Selected genes whose expression is modulated by activated protein C. Nomenclature in the above-referenced table is according to the HUGO gene nomenclature committee. (**) Genes down-regulated by activated protein C in TNF-α-simulated human umbilical vein endothelial cells (HUVECs) but up-regulated by activated protein C when human coronary artery endothelial cells (HCAECs) are stimulated with a mixture of interleukin-1β, TNFα, and interferon-γ. (***) Modulation of the expression of thrombospondin-1 and p53 by activated protein C in the microarray analysis was less than 2-fold. Nevertheless, activated protein C-induced changes in these genes shown to be functionally significant.)

In aspects, compositions comprise an effective amount of one or more compounds capable of up-regulating a gene affecting an apoptosis pathway. In aspects, such a gene includes, e.g., BCL2A1, NR4A1, SMAD3, or, e.g., TNFAIP3. In aspects, compositions comprise an effective amount of 1, 2, 3, or all of BCL2A1, NR4A1, SMAD3, or TNFAIP3.

In aspects, compositions comprise an effective amount of one or more compounds capable of up-regulating a gene affecting a differentiation pathway. In aspects, such a gene includes, e.g., BMP2. In aspects, compositions comprise an effective amount of BMP2.

In aspects, compositions comprise an effective amount of one or more compounds capable of up-regulating a gene affecting an inflammatory (inflammation) pathway. In aspects, such a gene includes, e.g., CCL2, CXCL2, ILIR1, IL6, IL8, NOS3, or, e.g., PTGS2. In aspects, compositions comprise an effective amount of 1, 2, 3, 4, or more or all of CCL2, CXCL2, IL1R1, IL6, IL8, NOS3, or PTGS2.

In aspects, compositions comprise an effective amount of one or more compounds capable of up-regulating a gene affecting an adhesion pathway. In aspects, such a gene includes, e.g., CX3CL1. In aspects, compositions comprise an effective amount of CX3CL1.

In aspects, compositions comprise an effective amount of one or more compounds capable of up-regulating a gene affecting a transcription pathway. In aspects, such a gene includes, e.g., DDX21, NFKB1, NFKB2, NR4A2, or, e.g., NR4A3. In aspects, compositions comprise an effective amount of 1, 2, 3, 4, or each of DDX21, NFKB1, NFKB2, NR4A2, or NR4A3.

In aspects, compositions comprise an effective amount of one or more compounds capable of up-regulating a gene affecting a coagulation pathway. In aspects, such a gene includes, e.g., F3. In aspects, compositions comprise an effective amount of F3. In aspects, compositions comprise an effective amount of F3.

In aspects, compositions comprise an effective amount of one or more compounds capable of up-regulating a gene affecting a proliferation pathway. In aspects, such a gene includes, e.g., HBEGF. In aspects, compositions comprise an effective amount of HBEGF.

In aspects, compositions comprise an effective amount of one or more compounds capable of up-regulating a gene affecting a cell cycle pathway. In aspects, such a gene includes, e.g., PCNA. In aspects, compositions comprise an effective amount of PCNA.

In aspects, compositions comprise an effective amount of one or more compounds capable of down-regulating a gene affecting an inflammatory (inflammation) pathway. In aspects, such a gene includes, e.g., B2M, CALR, ILR1, or, e.g., LTB. In aspects, compositions comprise an effective amount of 1, 2, 3, or all of B2M, CALR, ILR1, or LTB.

In aspects, compositions comprise an effective amount of one or more compounds capable of down-regulating a gene affecting an apoptosis pathway. In aspects, such a gene includes, e.g., BIRC5, or, e.g., TP53. In aspects, compositions comprise an effective amount of 1 or both of BIRC5 or TP53.

In aspects, compositions comprise an effective amount of one or more compounds capable of down-regulating a gene affecting a cell signaling pathway. In aspects, such a gene includes, e.g., CMKOR1. In aspects, compositions comprise an effective amount of CMKOR1.

In aspects, compositions comprise an effective amount of one or more compounds capable of down-regulating a gene affecting an adhesion pathway. In aspects, such a gene includes, e.g., CX3CL1, ICAM1, SELE, THBS1, or, e.g., VCAM1. In aspects, compositions comprise an effective amount of 1, 2, 3, 4, or each of CX3CL1, ICAM1, SELE, THBS1, or, VCAM1.

In aspects, compositions comprise an effective amount of one or more compounds capable of down-regulating a gene affecting a chemoattraction pathway. In aspects, such a gene includes, e.g., EFNA1. In aspects, compositions comprise an effective amount of EFNA1.

In aspects, compositions comprise an effective amount of one or more compounds capable of down-regulating a gene affecting a proteolysis pathway. In aspects, such a gene includes, e.g., MMP10. In aspects, compositions comprise an effective amount of MMP10.

In aspects, compositions comprise an effective amount of one or more compounds capable of down-regulating a gene affecting transcription. In aspects, such a gene includes, e.g., NFKB1, or, e.g., NFKB2. In aspects, compositions comprise an effective amount of 1 or both of NFKB1 or NFKB2.

In aspects, compositions comprise an effective amount of one or more compounds capable of down-regulating a gene affecting oxidation. In aspects, such a gene includes, e.g., SOD2. In aspects, compositions comprise an effective amount of SOD2.

In aspects, compositions comprise an effective amount of one or more compounds capable of up-regulating one or more of the genes noted above, and, further, comprise an effective amount of one or more compounds capable of down-regulating one or more of the genes noted above. In aspects, compositions comprise an effective amount of one or more compounds capable of detectably or significantly mimicking, e.g., demonstrating at least generally, at least substantially, at least essentially, or, e.g., statistically the same effect as activated protein C in its anti-inflammatory vascular effect(s) on endothelial cells, leukocytes, or both. Such effects are described in, e.g., Mosnier (supra). In aspects, compositions comprise an effective amount of one or more compounds capable of detectably or significantly mimicking, e.g., demonstrating at least generally, at least substantially, at least essentially, or, e.g., statistically the same effect as activated protein C in its antiapoptotic activity via an intrinsic apoptotic pathway, extrinsic apoptotic pathway, or both. Such activity is described in, e.g., Mosnier (supra). In aspects, compositions comprise an effective amount of one or more compounds capable of detectably or significantly mimicking, e.g., demonstrating at least generally, at least substantially, at least essentially, or, e.g., statistically the same effect as activated protein C in its effect on endothelial barrier stabilization. Such effects are described in, e.g., Mosnier (supra). In aspects, compositions comprise an effective amount of one or more compounds capable of detectably or significantly mimicking, e.g., demonstrating at least generally, at least substantially, at least essentially, or, e.g., statistically the same effect as activated protein C in its anticoagulant activity or effect, its cytoprotective activity or effect, or both. Such effects are described in, e.g., Mosnier (supra). In aspects, compositions comprise an effective amount of one or more compounds capable of detectably or significantly mimicking, e.g., demonstrating at least generally, at least substantially, at least essentially, or, e.g., statistically the same effect as activated protein C in its angiogenesis effect, wound healing effect, or both. Such effects are described in, e.g., Mosnier (supra).

### Protein S Modulators

In aspects, compositions comprise a coagulation pathway modulating component which DOS enhances protein S (a “protein S activity modulating component” PS-MC) which can comprise one or more “protein S activity modulators” or “PSAMs”. In aspects, a PSAM (in effective amount, which is implicit in regard to any description of ingredients herein) imparts a measurable or significant effect on protein S. In aspects, a PSAM in a composition DOS enhances the activity of endogenous protein S. In aspects, a PSAM, DOS enhances the activity of endogenous protein S by providing sufficient amount(s) of element(s) necessary in the formation of protein S, maintenance of an appropriate conformation of protein S, or both, such as, e.g., providing element(s) required by protein S to develop or maintain proper form, such as, e.g., providing element(s) which aid in the formation of residues which are involved in the establishment of a structure of protein S which can optimally align protein S with phospholipid or cell surfaces and efficiently participate in phospholipid binding (such as, e.g., the formation of Gla residues formed as the result of vitamin K-dependent post-translational carboxylation of glutamic acid residues on the Gla domain, the domain, as described above, comprising an aromatic stack which is believed to participate in the same). In aspects, a PSAM acts to DOS enhance the catalytic activity of protein C indirectly, by affecting one or more co-factors such as protein S. Protein S is known as an essential co-factor of protein C and an inhibitor of Factor IXa. See, e.g., WIJNEN. Throm Haemost, 76 (3):397-403 (September 1996) and Uniprot Accession P07225 (PROS_HUMAN). Variants and derivatives of protein S have also been described (see, e.g., WO1998044000 and U.S. Pat. No. 11,040,109). In aspects, a PSAM increases the amount of protein S in a subject. In aspects, a PSAM comprises protein S or a suitable variant or active fragment thereof, or a derivative of either thereof. In aspects, a PSAM comprises a nucleic acid encoding such a factor. In aspects, a PSAM is a nucleic acid construct or related composition (vector, cell, etc.), encoding a factor that causes increases in the expression of protein S or of other polypeptides/proteins that increase the stability or activity of protein S. This principle also can be applied to protein C modulators (i.e., a PCAM can comprise such a nucleic acid construct or related composition).

In aspects, a PSAM DOS inhibits Factor Xa from diminishing activated protein C's inactivation of Factor Va (see, e.g., MOSNIER, et al. Frontiers in Bioscience, 11:2381-99 (2006)). In aspects, a PSAM DOS inhibits inflammation, modulates apoptosis, modulates angiogenesis, or modulates cell viability in subject(s). In aspects, a PSAM DOS modulates activity of Factor IXa in subject(s).

In aspects, PSAM(s) as provided by composition(s), increase(s) the amount of protein S available in subject(s) by, e.g., at least about 0.5%, ≥~0.75%, ≥~1%, ≥~2%, ≥~5%, ≥~10%, ≥~15%, or even ≥~20% or more, or any other detectable or significant amount, compared to suitable control(s) (e.g., a state or subjects in which the PSAM(s) are not administered).

In aspects, the presence of effective amount(s) of PSAM(s), increase(s) the percentage of protein S which effectively binds to phospholipids within/associated with the coagulation system at a given time during a period in which composition(s) provided by the invention are in use by, e.g., at least about 0.5%, ≥~0.75%, ≥~1%, ≥~2%, ≥~5%, ≥~10%, ≥~15%, or even ≥~20% or more, or another significant or detectable amount, as compared to the percentage of protein S which effectively binds to phospholipids in a control (e.g., within the coagulation system when composition(s) are not provided, when similar or equivalent environments exist (e.g., the presence or absence of a skin condition, such as e.g., hyperkeratosis or bruising, etc.) as measured by appropriate in-vitro or in-vivo techniques).

In aspects, a PSAM DOS stabilizes endogenous protein S. In aspects, a PSAM DOS enhances the stability of endogenous protein S. In aspects, a coagulation pathway modulating component DOS enhances the stability of endogenous protein S by ensuring sufficient amount(s) of element(s) required for the proper formation of protein S, sufficient amount(s) of element(s) required for the development of proper structural form of protein S, or both are present in circulation for use by processes participating in the same. For example, the same process described above (e.g., the vitamin K-dependent post-translational carboxylation of glutamic acid residues on the Gla domain of protein C) aids in the stability of protein S.

In aspects, effective amount(s) of PSAM(s) increase(s) the average stability of circulating protein S at a given time during a period in which composition(s) provided by the invention are in use by, e.g., at least about 0.5%, ≥~0.75%, ≥~1%, ≥~2%, ≥~5%, ≥~10%, ≥~15%, or even ≥~20% or more, or by any other detectable or significant amount, as compared to an adequate control in subject(s).

In aspects, effective amount(s) of PSAM(s) increase(s) the relative amount of inactivated Factor V, Factor VIII, or both (or, stated in the alternative, decrease(s) the amount of activated Factor V, Factor VIII, or both) in circulation in subject(s) at a given time by, e.g., about 0.5%, ≥~0.75%, ≥~1%, ≥~2%, ≥~5%, ≥~10%, ≥~15%, or even ≥~20% or more, or any other detectable or significant amount, compared to the relative amount of inactivated Factor V, Factor VIII, or both when composition(s) are not provided. In aspects, effective amounts of PSAM(s), increase(s) the ratio of inactivated Factor V, Factor VIII, or both to activated Factor V, Factor VIII, or both (respectively) in circulation at a given time during a period in which composition(s) provided by the invention are in use by at least about 0.5%, ≥~0.75%, ≥~1%, ≥~2%, ≥~5%, ≥~10%, ≥~15%, or even ≥~20% or more compared to the ratio of inactivated-to-activated Factor V, Factor VIII, or both when composition(s) are not provided, when similar or equivalent environments exist (e.g., the presence or absence of a skin condition, such as e.g., hyperkeratosis or bruising, etc.) as measured by appropriate in-vitro or in-vivo techniques.

In aspects, a modulator of a referenced protein/polypeptide (e.g., a protein S modulator or protein C modulator) can comprise/be a mimetic of the protein/polypeptide. Mimetics of various biological molecules, particularly peptides/proteins, are known in the art (see, e.g., WO 00/68185 with respect to mimetics of helical portions of proteins). Other mimetics and relevant methods for generating such mimetics are described in, e.g., Moore G J. Trends Pharmacol Sci. 1994 April; 15 (4):124-9; Murali R, et al. Pharmaceuticals (Basel). 2012 Feb. 16; 5 (2):209-35; Mason J M. Future Med Chem. 2010 December; 2 (12):1813-22; Sun H, et al. Acc Chem Res. 2008 October; 41 (10):1264-77; Rai J. Chem Biol Drug Des. 2019 May; 93 (5):724-736; Davis J M, et al. Chem Soc Rev. 2007 February; 36 (2):326-34; Graham J. Moore, et al. Advances in Pharmacology, Academic Press, Volume 33, 1995, Pages 91-141; Gary L. Olson, et al. Journal of Medicinal Chemistry 1993 36 (21), 3039-3049; and Robinson J A. Acc Chem Res. 2008 October; 41 (10):1278-88, as well as US20110040087, US20100216992, US20070197772, US20100216992, and US20060084655. A mimetic may be a peptide mimetic or a non-peptide mimetic (in the latter case a small molecule that acts as a suitable substitute for a referenced polypeptide/protein). Typically, in a mimetic, the 3-dimensional placement of atoms of the mimetic such that similar ionic forces, covalent forces, van der Waal's, or other forces, and a similar charge complementarity, or electrostatic complementarity, exist between the atoms of the mimetic and the atoms of a binding site/target, such that the mimetic has a similar binding affinity as a referenced polypeptide (e.g., as determined by significantly similar or improved affinity in relevant test(s)). Methods for isolating or screening for compounds to generate potential mimetics which mimic a binding site are well known. For example, it is possible to use an antibody/anti-idiotypic antibody, such as an antibody that blocks binding of the referenced polypeptide to target, to identify binding determinates, if not already known (in the case of protein C and protein S several key binding determinates are known). Also, using known methods, compounds or mimetics may also be designed in light of the nucleic acid and amino acid sequences of binding molecules disclosed herein and the three-dimensional array or conformations of the amino acids of the binding molecules, as determined X-ray crystallography or NMR of the binding molecules (see e.g., U.S. Pat. No. 5,648,379; Colman et al., Protein Science, 3:1687-1696 (1994); Malby et al., Structure et al., 2:733-746 (1994); McCoy et al., J. Mol. Biol., 268:570-584 (1997); Pallaghy et al., Biochemistry, 34:3782-3794 (1995)). Thus, a mimetic or molecule which mimics the 3-dimensional structure of a binding site or moiety described herein (e.g., a paratope) may be designed from the analysis of the interaction of the binding site and ligand in crystals of the two molecules, or in solutions containing the two molecules. Purely synthetic binding molecules may be designed by the 3-dimensional placement of atoms, such that similar ionic forces, covalent forces, van der Waals, or other forces, and similar charge complementarity, exist between the atoms of the mimetic and the atoms of the binding or moiety. Mimetic candidates once generated may then be screened for high affinity binding to target site(s) and tested in in vitro or in vivo applications. In silico methods for the design and testing of mimetic compounds also have been developed and may be adapted to the generation of peptide modulators. Examples of such methods are provided in, e.g., Tzoupis H, Tselios T. Methods Mol Biol. 2018; 1824:33-47; Silva D A et al. Nature. 2019 January; 565 (7738):186-191; Bonadio A, et al. Protein Eng Des Sel. 2021 Feb. 15; 34: gzab020; Viswanath A N I et al. Chem Biol Drug Des. 2017 December; 90 (6):1041-1055; and Akhoon B A et al. J Mol Graph Model. 2010 April; 28 (7):664-9 and also WO2001009191. Methods relating to the specific design of Gla domain peptides, which may be adaptable for use as CP-MC constituents (e.g., PCAM(s)) are described in, e.g., U.S. Pat. No. 10,894,075.

In aspects, a PSAM modulates activity of protein C/protein S pathway(s) in subject(s) in a detectably or significantly different manner than the state of the protein C/protein S pathway(s) in subject(s) when treatment is not provided (e.g., without administration of composition(s). In aspects, a PSAM results in an increase of protein S/protein C pathway activities that correspond in one or more aspects to a level of activity associated with detectably or significantly more than 40% (e.g., more than ~45%, ~50%, or more than about 60%) free endogenous protein S in a subject than when composition(s) are not administered.

In aspects, a PSAM comprises a C4b-binding protein, an active fragment thereof, a variant thereof, or a derivative of any thereof. In aspects, a PSAM comprises a C4b-binding protein antagonist, e.g., an antibody that competes with protein S for binding to C4b-binding protein or a compound that blocks C4b-binding protein expression (e.g., an siRNA).

In aspects, a PSAM DOS modulates inhibition of the formation of the prothrombinase (Factor Va/Factor Xa) complex, the Factor X activating complex, or both. In aspects, a PSAM DOS inhibits binding of prothrombin to Factor Va, the amidolytic activity of Factor Xa, or both. In aspects, a PSAM modulates formation of one or more “tenase” complexes (Factor IXa/Factor VIIIa or Factor VII/tissue factor). In aspects, a PSAM DOS modulates anticoagulant activity, fibrinolysis, or both in subject(s). In aspects, a PSAM DOS modulates phagocytosis, apoptosis, or both, in subject(s). In aspects, effective amount(s) of PSAM(s) result in ≥2, ≥3, ≥4, or more of such effects in subject(s).

### Protein C and Protein S Modulators

In aspects, a CP-MC constituent detectably or significantly (DOS) modulates (e.g., enhances) both protein C activity and protein S activity. Compounds with such properties can be described as protein C/protein S activity modulators (PCAM/PSAMs or PCPSAMs) and a composition comprising ≥1 PCPSAMs as an ingredient of a composition can be described as a PCPSAM component (or PCPSA-MC). Uncontradicted, PCPSAMs can exhibit one, some, most, generally all, or all of the activities/effects or properties described above separately with respect to PCAMs and PSAMs. E.g., a PCPSA-MC, e.g., comprising PCPSAM(s) can comprise, mostly comprise, generally consist of, consist essentially of, or consist of non-polypeptide/non-protein compound(s), can increase both protein S-associated and protein C-associated activities, etc. CP-MCs can comprise a single (i.e., a single type) of PCPSAM, or 2 or more PCPSAMs. CP-MCs also can comprise, e.g., a PCPSAM and a compound/composition that might be classifiable only as (or only currently known to be) a PCAM or a PSAM, though, as protein S serves as an activated protein C binding protein that is essential for assembly of the anticoagulant complex on cell surfaces, most PSAMs also can be considered, at least indirectly, also to be PCAMs (and, thus, PCPSAMs). In aspects, PSAM(s) comprise/are or consist essentially of a compound/molecule that exerts effects either primarily/mostly on protein S, on protein S activities not associated with protein C, or both.

In aspects, a PCPSAM is a vitamin K compound (“VKC”).

The name “vitamin K” is understood in the art to refer to a series of fat-soluble compounds, typically having a common 2-methyl-1,4-naphthoquinone nucleus, but which differ in terms of the structure of any side chain at the 3 position, as exemplified in Formulas I-III below, reflecting the structures of vitamin K1, MK-4, and MK-7 (the latter two being both subtypes of vitamin K2).

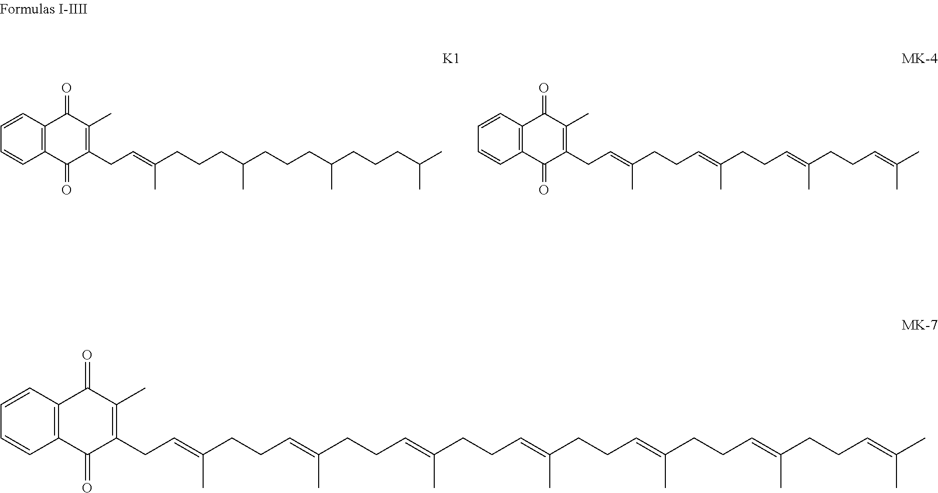 [SMILES: CC1=C(C/C=C(\C)CCCC(C)CCCC(C)CCCC(C)C)C(=O)c2ccccc2C1=O ; CC(C)=CCC/C(C)=C/CC/C(C)=C/CC/C(C)=C/CC1=C(C)C(=O)c2ccccc2C1=O ; CC(C)=CCC/C(C)=C/CC/C(C)=C/CC/C(C)=C/CC/C(C)=C/CC/C(C)=C/CC/C(C)=C/CC1=C(C)C(=O)c2ccccc2C1=O]

Vitamin K compounds can include 2-methyl-1,4-naphthoquinone (3-) derivatives. In aspects, a VKC that is included in a composition is a naturally occurring vitamin K compound or a synthetic version thereof that is substantially identical (e.g., vitamin K1 (phylloquinone, the natural form of K1 and phytonadione, the synthetic type of K1) or vitamin K2 (menaquinone)). VKCs typically comprise an isoprene side chain. In aspects, a VKC comprises a saturated isoprene side chain (e.g., similar to phylloquinone). In aspects, a VKC comprises an unsaturated isoprene side chain (similar to vitamin K2 compounds, such as MK-4 and MK-7). In aspects, a vitamin K compound lacks any side chain (as in the case of menadione/vitamin K3). In aspects, the vitamin K compound content of compositions lacks any menadione or other VKC that lacks a side chain. In aspects, the VKC(s) of a composition are mostly vitamin K1 compound(s). In aspects, generally all, essentially all, or substantially all of the VKC(s) of a composition are vitamin K compound(s).

In aspects, a vitamin K compound of a CP-MC is any pharmaceutically acceptable, cosmetologically acceptable vitamin K compound, including naturally occurring vitamin K compound(s) and synthetically produced versions of the same, capable of DOS modulating a coagulation pathway, such as, e.g., modulating one or more coagulation pathway constituents. In aspects, the vitamin K compound is suitable for topical application (examples of vitamin K compound-containing compositions suitable for topical administration are exemplified elsewhere herein and described in the art, see, e.g., WO1997039746 and U.S. Pat. No. 5,510,391).

In aspects, a vitamin K compound includes vitamin K1 (phylloquinone), vitamin K2 (menaquinone) (including all vitamin K2 subtypes including shorter subtypes such as MK-4 (4 isoprene groups) and longer subtypes such as MK-13, and including MK-7 shown above), or menadione (infrequently referred to as vitamin K3, a vitamin precursor converted into menaquinone by the liver).

A vitamin K compound can comprise any possible and suitable pharmaceutically acceptable isomer, prodrug (see, e.g., U.S. Pat. No. 10,159,687), hydrate, solvate, or polymorph of a naturally occurring vitamin K compound or synthetic vitamin K compound (examples of which are known in the art, see, e.g., US20200237710, WO2016038626, and U.S. Pat. No. 11,096,907, which describe a variety of vitamin K compounds/VKC-related compounds and related compositions, which may be adaptable to/applied in aspects, and, e.g., Cook et al, Journal of AOAC INTERNATIONAL, Volume 85, Issue 4, 1 Jul. 2002, Pages 832-840; Koehn et al, ACS Omega 2018, 3, 14889-14901; and Lowenthal J et al. J Pharmacol Exp Ther. 1979 June; 209 (3):330-3, describing vitamin K isomers.) Some vitamin K compounds, such as derivatives of natural vitamin K compounds, may include salt forms (see, e.g., US20160184254). Uncontradicted, any possible and suitable salt forms of VKCs also are within the scope of VKCs described here. Uncontradicted, any compound(s) of this disclosure can include any suitable related compound(s) such as esters, salts, prodrugs, hydrates, solvates, active isomers (e.g., enantiomers, racemates, etc.), etc. Acceptable salts for compounds can include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, metliylsulfonates, propylsulfonates, besylates, xylenesulfonates, naphthalene-1-sulfonates, naphthalene-2-sulfonates, phenylacetates, phenylpropionates, phenylbutyrates, citrates, lactates, g-hydroxybutyrates, glycolates, tartrates, mandelates, and the like. Additional suitable salt forms are provided in references cited herein and, more generally, in Remington's Pharmaceutical Sciences, 17th Edition, Mack Publishing Company, Easton, Pa., 1985 and Berge S M et al. J Pharm Sci. 1977 January; 66 (1):1-19. Uncontradicted, the term “salts” also includes solvates of addition salts, such as hydrates, as well as polymorphs of addition salts, wherever applicable and suitable. Such aspects are applicable to compound(s) of the invention, generally.

In aspects, a coagulation pathway modulating component comprises a vitamin K compound mimetic, which shares at least similarity in terms of binding to vitamin K targets and modulation thereof (e.g., modulation of protein C and protein S) and exerts a physiological functionally similar or equivalent effect as a vitamin K compound. Examples of vitamin K mimetics can include, e.g., relatively smaller, simplified naphthoquinones which share anticoagulative properties of a natural vitamin K compound, such as, e.g., 2-methyl-1,4naphthoquinone and other compounds disclosed in, e.g., RHODES, et. al., JAMA 114 (5):400-401. Other vitamin K compounds, vitamin K-related compounds, and other compositions/principles relevant thereto that may be adaptable/applicable to aspects of the invention are described in, e.g., US20140336268, US2010013061, U.S. Pat. No. 9,212,214, US20160184254, US20110028499, and US20140336268.

Many vitamin K compounds are known, a further exemplary selection of which are provided in Table 3 as illustrative of the breadth and diversity of such compounds, some of which compounds can, with no more than routine experimentation, be screened for suitability in use in compositions/methods of the invention. Compounds of Table 3 are exemplary vitamin K compounds provided by the publicly available ClassyFire database (available at classyfire.wishartlab.com) and are each identified by their SMILES and InchIKey identifiers, with compound names and structures listed as provided in the publicly available ChemSpider database (available at chemspider.com). Such compound(s) may comprise stereoisomer(s) and similar alternative forms (salts, etc.), each of which should be considered as being disclosed here and each of which may be suitable for screening for use in compositions/methods of the invention. Specific structures of compounds presented in Table 3 are shown according to the 2-dimensional style provided by chemspider.com, wherein specific characteristics are used in the 2-dimensional representation to indicate a 3 -dimensional shape (see for example the crossed connecting lines of, e.g., compounds IX, X, etc.) Such visual representation should not be interpreted as limiting the disclosure of a specific conformation, e.g., a specific stereoisomer of the compound.

TABLE 3
(Additional Exemplary Vitamin K Compounds)

| Formula | | | | |
|---|---|---|---|---|
| (ID) | Formula | Mass | SMILES | InChIKey |
| IV | C26H30Cl2N2O5 | 521.44 | CC(C)═CCCC(C)═CCC1 | LLEHFPOFPPKPQB- |
| | | | 2OC(C)(C)C(Cl)CC1(Cl) | UHFFFAOYSA-N |
| | | | C(═O)C1═C(C2═O)C([N+]#N)═ | |
| | | | C(O)C(C)═C1[O−] | |

3,4a-Dichloro-9-diazonio-10a-[(2E)-3,7-dimethyl-2,6-octadien-1-yl]-8-hydroxy-2,2,7-
trimethyl-5,10-dioxo-3,4,4a,5,10,10a-hexahydro-2H-benzo[g]chromen-6-olate

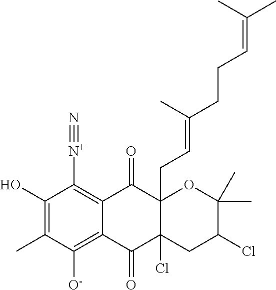 [SMILES: CC(C)=CCC/C(C)=C/CC12OC(C)(C)C(Cl)CC1(Cl)C(=O)c1c([O-])c(C)c(O)c([N+]#N)c1C2=O]

| | | | | |
|---|---|---|---|---|
| V | C26H31Cl2N2O5 | 522.44 | CC(C)═CCCC(C)═CCC1 | LLEHFPOFPPKPQB- |
| | | | 2OC(C)(C)C(Cl)CC1(Cl) | UHFFFAOYSA-O |
| | | | C(═O)C1═C(C2═O)C([N+]#N)═ | |
| | | | C(O)C(C)═C1O | |

3,4a-Dichloro-10a-[(2E)-3,7-dimethyl-2,6-octadien-1-yl]-6,8-dihydroxy-2,2,7-trimethyl-5,10-
dioxo-3,4,4a,5,10,10a-hexahydro-2H-benzo[g]chromene-9-diazonium

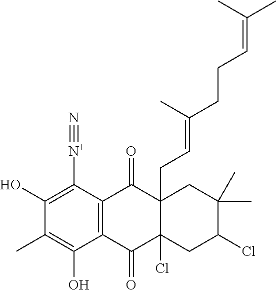 [SMILES: CC(C)=CCC/C(C)=C/CC12CC(C)(C)C(Cl)CC1(Cl)C(=O)c1c(O)c(C)c(O)c([N+]#N)c1C2=O]

| | | | | |
|---|---|---|---|---|
| VI | C30H43ClO2 | 471.12 | CC(C)CCCC(C)CCCC(C) | BFMNXKXZDPXHIG- |
| | | | CCCC(C)═CCC1═C(Cl) | UHFFFAOYSA-N |
| | | | C(═O)C2═CC═CC═C2C1═O | |

2-Chloro-3-(3,7,11,15-tetramethyl-2-hexadecen-1-yl)-1,4-naphthalenedione

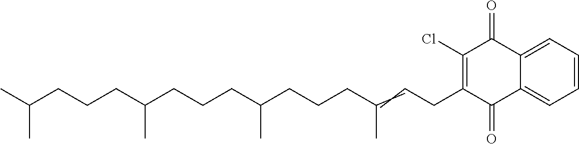 [SMILES: CC(=CCC1=C(Cl)C(=O)c2ccccc2C1=O)CCCC(C)CCCC(C)CCCC(C)C]

| | | | | |
|---|---|---|---|---|
| VII | C51H74O2 | 719.151 | CC(CCCC(C)═CCC1═C(C) | ROENOAFLUDRPDJ- |
| | | | C(═O)C2═CC═CC═C2 | UHFFFAOYSA-N |
| | | | C1═O)CCC═C(C)CCC═ | |
| | | | C(C)CCC═C(C)CCC═C(C) | |
| | | | CCC═C(C)CCC═C(C)C | |

2-Methyl-3-[(2E,10E,14E,18E,22E,26E)-3,7,11,15,19,23,27,31-octamethyl-
2,10,14,18,22,26,30-dotriacontaheptaen-1-y1]-1,4-naphthoquinone

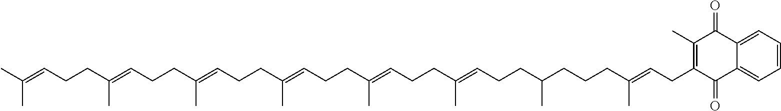 [SMILES: CC(C)=CCC/C(C)=C/CC/C(C)=C/CC/C(C)=C/CC/C(C)=C/CC/C(C)=C/CCC(C)CCC/C(C)=C/CC1=C(C)C(=O)c2ccccc2C1=O]

| | | | | |
|---|---|---|---|---|
| VIII | C15H16O2 | 228.291 | CC(C)CCC1═CC(═O) | LTDXMRHTAVBPHX- |
| | | | C2═CC═CC═C2C1═O | UHFFFAOYSA-N |

2-(3-Methylbutyl)-1,4-naphthoquinone

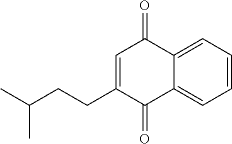 [SMILES: CC(C)CCC1=CC(=O)c2ccccc2C1=O]

| | | | | |
|---|---|---|---|---|
| IX | C25H30Cl2O5 | 481.41 | CC(C)═CCCC(C)═CCC1 | BCPWMPCOEAOEDD- |
| | | | 2OC(C)(C)C(Cl)CC1(Cl) | UHFFFAOYSA-N |
| | | | C(═O)C1═C(O)C═C(O)C═C1C2═O | |

3,4a-Dichloro-10a-(3,7-dimethyl-2,6-octadien-1-yl)-6,8-dihydroxy-2,2-dimethyl-3,4,4a, 10a-
tetrahydro-2H-benzo[g]chromene-5,10-dione

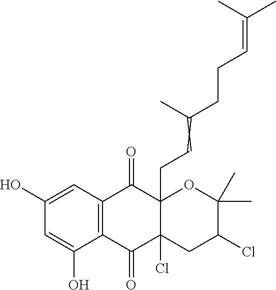 [SMILES: CC(C)=CCCC(C)=CCC12OC(C)(C)C(Cl)CC1(Cl)C(=O)c1c(O)cc(O)cc1C2=O]

| | | | | |
|---|---|---|---|---|
| X | C21H24O2 | 308.421 | CC(C)═CCCC(C)═CCC1═ | PTWSIIUEOFZCHW- |
| | | | C(C)C(═O)C2═CC═CC═C2C1═O | UHFFFAOYSA-N |

2-(3,7-Dimethyl-2,6-octadien-1-yl)-3-methyl-1,4-naphthoquinone

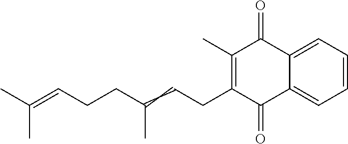 [SMILES: CC(C)=CCCC(C)=CCC1=C(C)C(=O)c2ccccc2C1=O]

| | | | | |
|---|---|---|---|---|
| XI | C31H48O3 | 468.722 | CC(C)CCC[C@@H](C) | OOJSROHRRKVBFW- |
| | | | CCC[C@@H](C)CCCC(C)═ | MKHVJUGASA-N |
| | | | CCC1(O)C(C)C(═O) | |
| | | | C2═CC═CC═C2C1═O | |

3-hydroxy-2-methyl-3-phytyl-2,3-dihydro-1,4-naphthoquinone

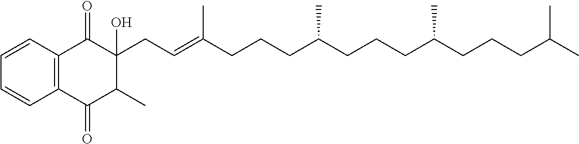 [SMILES: C/C(=C\CC1(O)C(=O)c2ccccc2C(=O)C1C)CCC[C@H](C)CCC[C@H](C)CCCC(C)C]

| | | | | |
|---|---|---|---|---|
| XII | C20H22O2 | 294.394 | CC(C)═CCCC(C)═CCC1═ | WALCBDVYDNDULV- |
| | | | CC(═O)C2═CC═CC═C2Cl═O | UHFFFAOYSA-N |

2-(3,7-Dimethyl-2,6-octadien-1-yl)-1,4-naphthoquinone

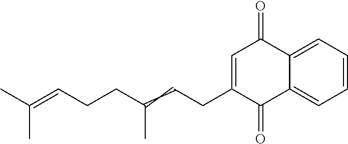 [SMILES: CC(C)=CCCC(C)=CCC1=CC(=O)c2ccccc2C1=O]

| | | | | |
|---|---|---|---|---|
| XIII | C31H48O3 | 468.722 | CC(C)CCCC(C)CCCC(C) | IGFQNNFYQDCUDR- |
| | | | CCCC(C)═CCC1C(═O) | UHFFFAOYSA-N |
| | | | C2═CC═CC═C2C(═O)C1(C)O | |

2-Hydroxy-2-methyl-3-(3,7,11,15-tetramethyl-2-hexadecen-1-yl)-2,3-dihydro-1,4-naphthalenedione

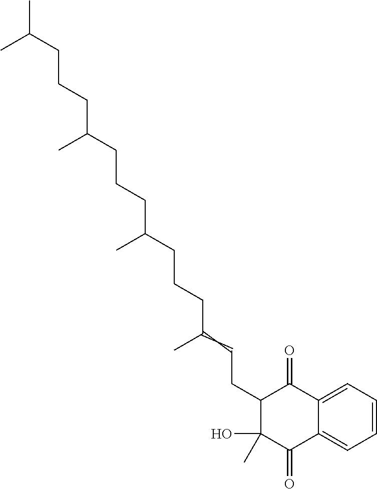 [SMILES: CC(=CCC1C(=O)c2ccccc2C(=O)C1(C)O)CCCC(C)CCCC(C)CCCC(C)C]

| | | | | |
|---|---|---|---|---|
| XIV | C31H48O3 | 468.722 | CC(C)CCCC(C)CCCC(C) | OOJSROHRRKVBFW- |
| | | | CCCC(C)═CCC1(O)C(C) | UHFFFAOYSA-N |
| | | | C(═O)C2═CC═CC═C2C1═O | |

2-Hydroxy-3-methyl-2-(3,7,11,15-tetramethyl-2-hexadecen-1-yl)-2,3-dihydro-1,4- naphthalenedione

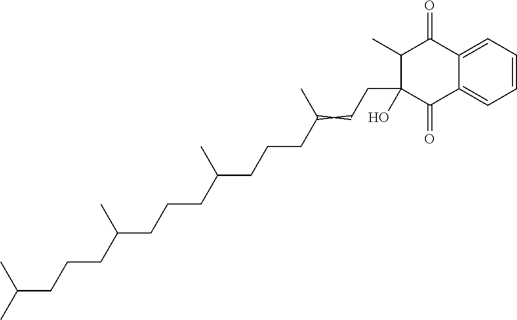 [SMILES: CC(=CCC1(O)C(=O)c2ccccc2C(=O)C1C)CCCC(C)CCCC(C)CCCC(C)C]

| | | | | |
|---|---|---|---|---|
| XV | C31H46O2 | 450.707 | CC(C)CCC[C@H](C)CC | MBWXNTAXLNYFJB- |
| | | | CC(C)CCC1C(C)═C\CC1═ | JXAKDHTRSA-N |
| | | | C(C)C(═O)C2═CC═CC═C2C1═O | |

2-Methyl-3-[(2E,11S)-3,7,11,15-tetramethyl-2-hexadecen-1-y1]-1,4-naphthoquinone

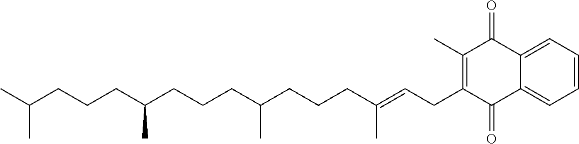 [SMILES: CC1=C(C/C=C(\C)CCCC(C)CCC[C@@H](C)CCCC(C)C)C(=O)c2ccccc2C1=O]

| | | | | |
|---|---|---|---|---|
| XVI | C20H22O6 | 358.39 | CCC(C)C(═O)O[C@@H] | InChIKey═XTGDSJYAIZDV |
| | | | (C═C(C)C)C1═CC(═O) | KJ-NBFOKTCDSA-N |
| | | | C2═C(O)C═CC(O)═C2C1═O | |

(1S)-1-(5,8-Dihydroxy-1,4-dioxo-1,4-dihydro-2-naphthalenyl)-3-methyl-2-buten-1-yl 2-methylbutanoate

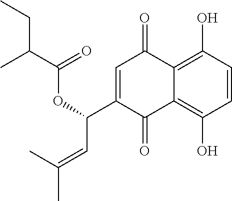 [SMILES: CCC(C)C(=O)O[C@@H](C=C(C)C)C1=CC(=O)c2c(O)ccc(O)c2C1=O]

| | | | | |
|---|---|---|---|---|
| XVII | C15H16O2 | 228.297 | CC(C)═CCC1CC(═O)C2═ | CWIWSAPEOCRNMD- |
| | | | CC═CC═C2C1═O | UHFFFAOYSA-N |

2-(3-Methyl-2-buten-1-yl)-2,3-dihydro-1,4-naphthalenedione

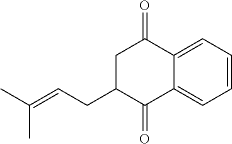 [SMILES: CC(C)=CCC1CC(=O)c2ccccc2C1=O]

| | | | | |
|---|---|---|---|---|
| XVIII | C26H34O2 | 378.556 | CC(CCC\C(C)═C\CC1═C | UOZVJFJPUVXXIY- |
| | | | (C)C(═O)C2═CC═CC═C2C1═O) | CAPFRKAQSA-N |
| | | | CCC═C(C)C | |

2-Methyl-3-[(2E)-3,7,11-trimethyl-2,10-dodecadienyl]naphthoquinone

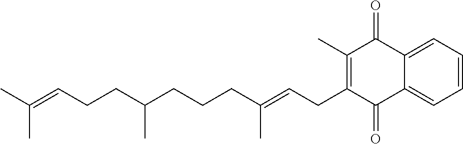 [SMILES: CC(C)=CCCC(C)CCC/C(C)=C/CC1=C(C)C(=O)c2ccccc2C1=O]

| | | | | |
|---|---|---|---|---|
| XIX | C26H31C1N2O5 | 486.99 | CC(C)═CCC1C(C)═C1C[C@] | LPYVLCLBZOVMKA- |
| | | | 1(O)C(═O)C2═C(C([O-])═ | UDONJXMRSA-N |
| | | | C(C)C(O)═C2[N+]#N) | |
| | | | C(═O)C1(Cl)CC═C(C)C | |

(6S)-7-Chloro-4-diazonio-6-[(2E)-3,7-dimethyl-2,6-octadien-1-yl]-3,6-dihydroxy-2-methyl-7-
(3-methyl-2-buten-1-yl)-5,8-dioxo-5,6,7,8-tetrahydro-1-naphthalenolate

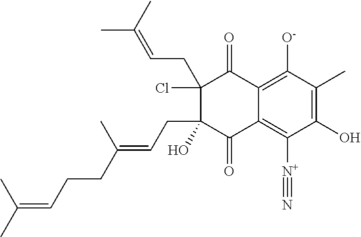 [SMILES: CC(C)=CCC/C(C)=C/C[C@]1(O)C(=O)c2c([N+]#N)c(O)c(C)c([O-])c2C(=O)C1(Cl)CC=C(C)C]

| | | | | |
|---|---|---|---|---|
| XX | C26H33C1O5 | 461.0 | CC(C)═CCC1C(C)═C\CC | VGHMRYUOZDKQTD- |
| | | | 1(O)C(═O)C2═CC(O)═C(C) | GMYRNWJMSA-N |
| | | | C(O)═C2C(═O)[C@@] | |
| | | | 1(Cl)CC═C(C)C | |

(3R)-3-Chloro-2-[(2E)-3,7-dimethyl-2,6-octadien-1-yl]-2,5,7-trihydroxy-6-methyl-3-(3-methyl-
2-buten-1-yl)-2,3-dihydro-1,4-naphthalenedione

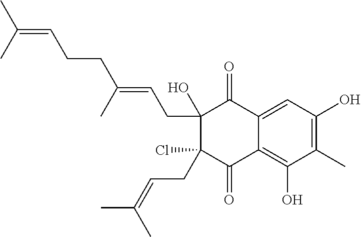

| | | | | |
|---|---|---|---|---|
| XXI | C56H84O2 | 789.286 | CC(CCCC(C)CC1C═C(IC) | NXKRRNUXCLRIHG- |
| | | | CC1C═C(\C)CC1C═C(IC) | BKYDCOGOSA-N |
| | | | CC1C═C(\C)CC1C═C(IC) | |
| | | | CCC═C(C)C)CCC\C(C)═ | |
| | | | C/CC1═C(C)C(═O)C2═ | |
| | | | CC═CC═C2C1═O | |

2-Methyl-3-[(2Z,14Z,18Z,22Z,26Z,30Z)-3,7,11,15,19,23,27,31,35-nonamethyl-
2,14,18,22,26,30,34-hexatriacontaheptaen-1-yl]-1,4-naphthoquinone

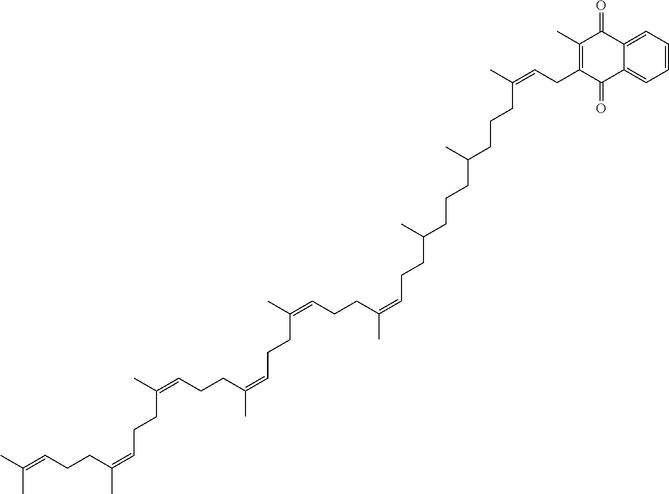 [SMILES: CC(C)=CCC/C(C)=C\CC/C(C)=C\CC/C(C)=C\CC/C(C)=C\CC/C(C)=C\CCC(C)CCCC(C)CCC/C(C)=C\CC1=C(C)C(=O)c2ccccc2C1=O]

| | | | | |
|---|---|---|---|---|
| XXII | C45H58N4O4S | 751.O4 | NC1═CC═C(C═C1)S(═O) | UYJOQYNTPXNDFP- |
| | | | (═O)NC1═NC2═CC═CC═ | UDLBSFQBSA-N |
| | | | C2N═C1.CC(C)CCCC(C) | |
| | | | CCCC(C)CCC\C(C)═C\CC1═C | |
| | | | (C)C(═O)C2═CC═CC═C2C1═O | |

4-Amino-N-(2-quinoxalinyl)benzenesulfonamide - 2-methyl-3-[(2E)-3,7,11,15-tetramethyl-2-
hexadecen-1-y1]-1,4-naphthoquinone (1:1)

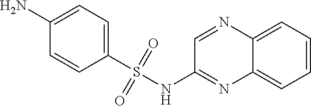 [SMILES: Nc1ccc(S(=O)(=O)Nc2cnc3ccccc3n2)cc1]

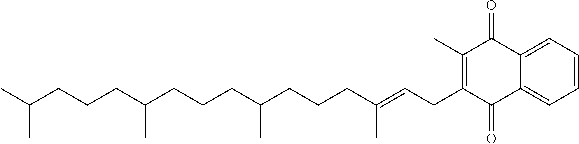 [SMILES: CC1=C(C/C=C(\C)CCCC(C)CCCC(C)CCCC(C)C)C(=O)c2ccccc2C1=O]

| | | | | |
|---|---|---|---|---|
| XXIII | C26H32C1N2O5 | 488.0 | CC(C)═CCC1C(C)═C1C[ | LPYVLCLBZOVMKA- |
| | | | C@]1(O)C(═O)C2═C(C(O)═ | UDONJXMRSA-O |
| | | | C(C)C(O)═C2[N+]#N)C(═O) | |
| | | | C1(Cl)CC═C(C)C | |

(7S)-6-Chloro-7-[(2E)-3,7-dimethyl-2,6-octadien-1-yl]-2,4,7-trihydroxy-3-methyl-6-(3-methyl-
2-buten-1-yl)-5,8-dioxo-5,6,7,8-tetrahydro-1-naphthalenediazonium

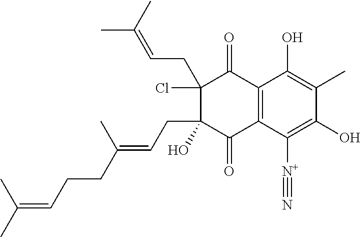 [SMILES: CC(C)=CCC/C(C)=C/C[C@]1(O)C(=O)c2c([N+]#N)c(O)c(C)c(O)c2C(=O)C1(Cl)CC=C(C)C]

| | | | | |
|---|---|---|---|---|
| XXIV | C31H42O2 | 446.675 | C[C@@H]1[C@H](C\C═ | JKOZDGHYJBZMGU- |
| | | | C(/C)CC1C═C(/C)CC1C═ | MVFADMRVSA-N |
| | | | C(/C)CCC═C(C)C)C(═O) | |
| | | | C2═CC═CC═C2C1═O | |

(2R,3S)-2-Methyl-3-[(2E,6E,10E)-3,7,11,15-tetramethyl-2,6,10,14-hexadecatetraen-1-y1]-2,3-
dihydro-1,4-naphthalenedione

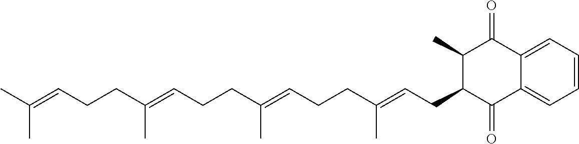 [SMILES: CC(C)=CCC/C(C)=C/CC/C(C)=C/CC/C(C)=C/C[C@@H]1C(=O)c2ccccc2C(=O)[C@@H]1C]

| | | | | |
|---|---|---|---|---|
| XXV | C31H42O2 | 446.675 | CC1C(CC═C(C)CCC═C(C) | JKOZDGHYJBZMGU- |
| | | | CCC═C(C)CCC═C(C)C) | UHFFFAOYSA-N |
| | | | C(═O)C2═CC═CC═C2C1═O | |

2-Methyl-3-(3,7,11,15-tetramethyl-2,6,10,14-hexadecatetraen-1-yl)-2,3-dihydro-1,4-
naphthalenedione

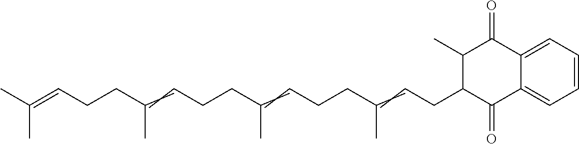 [SMILES: CC(C)=CCCC(C)=CCCC(C)=CCCC(C)=CCC1C(=O)c2ccccc2C(=O)C1C]

| | | | | |
|---|---|---|---|---|
| XXVI | C31H46O2 | 450.707 | CC(C)CCC[C@H](C)CC | MBWXNTAXLNYFJB- |
| | | | C[C@H](C)CCC\C(C)═C\CC1═ | JHBCSKSVSA-N |
| | | | C(C)C(═O)C2═CC═CC═C2C1═O | |

2-Methyl-3-[(2E,7S,11S)-3,7,11,15-tetramethyl-2-hexadecen-1-y1]-1,4-naphthoquinone

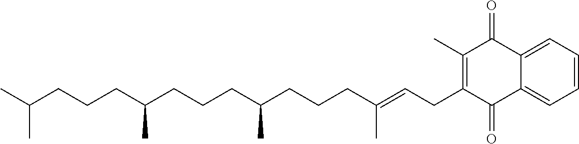 [SMILES: CC1=C(C/C=C(\C)CCC[C@@H](C)CCC[C@@H](C)CCCC(C)C)C(=O)c2ccccc2C1=O]

| | | | | |
|---|---|---|---|---|
| XXVII | C31H46O2 | 450.707 | CC(C)CCC[C@H](C)CC | MBWXNTAXLNYFJB- |
| | | | C[C@@H](C)CCC\C(C)═ | UDCSOKOMSA-N |
| | | | C\CC1═C(C)C(═O)C2═ | |
| | | | CC═CC═C2C1═O | |

2-Methyl-3-[(2E,7R,11S)-3,7,11,15-tetramethyl-2-hexadecen-1-yl]-1,4-naphthoquinone

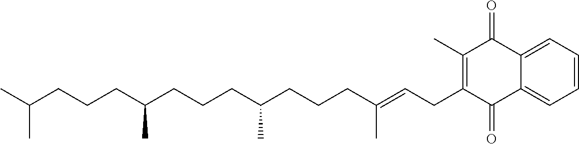 [SMILES: CC1=C(C/C=C(\C)CCC[C@H](C)CCC[C@@H](C)CCCC(C)C)C(=O)c2ccccc2C1=O]

| | | | | |
|---|---|---|---|---|
| XXVIII | C31H46O2 | 450.707 | CC(C)CCC[C@@H](C) | MBWXNTAXLNYFJB- |
| | | | CCC[C@H](C)CCC\C(C)═ | KSVLJPARSA-N |
| | | | C\CC1═C(C)C(═O)C2═ | |
| | | | CC═CC═C2C1═O | |

2-Methyl-3-[(2E,7S,11R)-3,7,11,15-tetramethyl-2-hexadecen-1-yl]-1,4-naphthoquinone

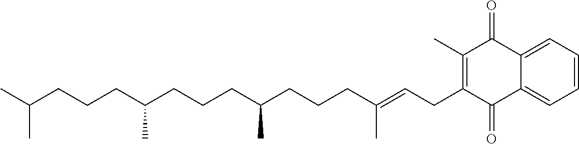 [SMILES: CC1=C(C/C=C(\C)CCC[C@@H](C)CCC[C@H](C)CCCC(C)C)C(=O)c2ccccc2C1=O]

In aspects, VKCs comprise, mostly comprise, or only comprise a 1,4-naphthoquinone having methyl or phytyl groups (or both) at positions 2 and 3, respectively. In aspects, VKCs comprise, mostly comprise, or only comprise prenylated naphthoquinone compound(s). In aspects, compositions provided by the invention comprise a form of vitamin K1 (phylloquinone or phytonadione).

In aspects, the vitamin K1 compound is a derivative or analogue of phylloquinone which provides at least substantially similar or effectively equivalent, if not improved, physiological activity as phylloquinone. In aspects, such vitamin K1 compound derivatives can comprise, mostly comprise, or be a vitamin K derivative, such as exemplified by CHEN et. al. Journal of Thrombosis and Haemostasis, Volume 19, Issue 3, pp. 689-700 (March 2021). In aspects, the vitamin K1 compound is a vitamin K1 mimetic which provides at least substantially similar or effectively equivalent physiological activity as phylloquinone/phytonadione. In aspects, some, most, generally all, or all of the VKCs of a composition exhibit skin penetration corresponding to or being detectably or significantly better than the skin penetration of vitamin K1 in the context of the relevant composition.

In aspects, compositions comprise a CP-MC constituent, such as VKC(s), present in the composition in an amount of about 0.1 wt. %-about 2 wt. %, such as, e.g., ~0.1-~1.8 wt. %, ~0.1-~1.6 wt. %, ~0.1-~1.4 wt. %, ~0.1-~1.2 wt. %, ~0.1-~1 wt. %, ~0.2-~2 wt. %, ~0.4-~2 wt. %, ~0.6-~2 wt. %, ~0.8-~2 wt. %, ~1-~2 wt. %, such as, e.g., ~0.2-~1.9 wt. %, ~0.3-~1.8 wt. %, ~0.4-~1.7 wt. %, ~0.5-~1.6 wt. %, ~0.6-~1.5 wt. %, ~0.7-~1.4 wt. %, ~0.8-~1.3 wt. %, ~0.9-~1.2 wt. %, or, e.g., about 1 wt. % of the composition.

In aspects, the coagulation pathway modulating component comprises a therapeutically effective amount of a vitamin K compound. In aspects, the vitamin K compound is present in a sufficient amount to DOS enhance the activity of endogenous protein C; DOS enhance the stability of endogenous protein C; or DOS enhance both the activity and the stability of endogenous protein C. In aspects, the coagulation pathway modulating component consists at least mostly of vitamin K compound(s). In aspects, the coagulation pathway modulating component consists essentially of a vitamin K compound. In aspects, a CP-MC generally only comprises, essentially consists of, or consists of vitamin K compound(s), e.g., vitamin K1.

In aspects, a single dose of composition comprises about 0.5-about 10 mg of a CP-MC, e.g., ~1 mg, ~2 mg, ~3 mg, ~4 mg, ~5 mg, ~6 mg, ~7 mg, ~8 mg, or ~9 mg of a CP-MC.

In aspects, a single dose/application of composition comprises about 5-about 50 mg of a CP-MC (e.g., VKC(s), such as, e.g., ~5-~45 mg, ~5-~40 mg, ≥~~~35 mg, ~5-~30 mg, ~5-~25 mg, ~5-~25 mg, ~5-~20 mg, ~5-~15 mg, or, e.g., ~5-~10 mg, or, e.g., between ~10-~50 mg, ~15-~50 mg, ~20-~50 mg, ~25-~50 mg, ~30-~50 mg, ~35-~50 mg, ~40-~50 mg, or, e.g., ~45-~50 mg of a coagulation pathway modulating component per dose. In aspects, the amount per dose is dependent upon the size of the treatment area, the targeted indication, the targeted affect, or any combination thereof.

### NAD Pathway Modulation Component

In aspects, compositions include effective amount(s) of a nicotinamide-adenine dinucleotide (NAD) pathway modulation component (NADP-MC) (To reiterate, herein, “NADP-MC” is NAD pathway modulation component, the “P” in the acronym meaning “pathway”, as opposed to NADP indicating “nicotinamide-adenine dinucleotide phosphate” also commonly represented as “NADP” where the “P” in the acronym means “phosphate”. Where, specifically, an aspect of the invention is directed to or is specifically related to nicotinamide-adenine dinucleotide phosphate, such will be indicated (e.g., “NADP” may be used alone). In aspects, the NADP-MC directly or indirectly increases one or more aspects of an NAD pathway in subject(s) (e.g., detectably or significantly raises an amount of, activity of, or both amount and activity of an NAD pathway constituent, in subject(s)). In aspects, a NADP-MC directly or indirectly stabilizes (e.g., decreases the rate of degradation of or breakdown of) one or more constituents of an NAD pathway, increases the activity of one or more constituents of an NAD pathway, and the like, such that the component “enhances” certain characteristics of an NAD pathway or characteristics of one or more NAD pathway constituents. In aspects, a NADP-MC is suitable for topical application.

In aspects, an NAD pathway modulation component (NADP-MC) can modulate one or more NAD-related pathways. In aspects, an NADP-MC can detectably or significantly affect one or more constituents of one or more NAD pathways. In aspects, such modulation is direct. In aspects, modulation is indirect (e.g., by detectably or significantly impacting one element which then affects another, etc.) For example, an NADP-MC, such as, e.g., a nicotinate component, e.g., a nicotinate compound constituent, a niacinamide component, e.g., a niacinamide compound constituent, or both, can detectably or significantly increase the amount of nicotinamide-adenine dinucleotide (NAD+), nicotinamide-adenine dinucleotide phosphate (NADP+), or both in subject(s). In aspects, the NADP-MC DOS modulates redox reactions, bioenergetic and signaling pathways (e.g., TCA cycle, pentose phosphate cycle, etc.), ADP-ribosylation reactions, protein deacetylation reactions, fatty acid biosynthesis, metabolic pathway reactions, immune response reactions, or any combination thereof in subject(s).

In aspects, an NADP-MC or a constituent thereof, such as, e.g., a nicotinate compound constituent, detectably or significantly (DOS) increases the amount of niacin (vitamin B3, nicotinic acid) in the body. In aspects, an effective amount of a NADP-MC DOS widens blood vessels, lowers blood fats, and breaks up clotting protein(s). In aspects, an NADP-MC, such as, e.g., a nicotinate compound constituent or a niacinamide compound constituent, DOS increases the amount of poly(ADP-ribose) polymerase-1 (PARP-1) in the body of subject(s). In aspects, an NADP-MC, such as, e.g., a niacinamide compound constituent, detectably or significantly increases the amount of methyl-nicotinamide in the body of subject(s). In aspects, an NADP-MC, such as, e.g., a niacinamide compound constituent, increases the amount of methyl-nicotinamide in the body. In aspects, an NADP-MC, such as, e.g., a niacinamide compound constituent, modulates end-product inhibition on SIRT1 deacetylase activity in subject(s). In aspects, an NADP-MC, such as, e.g., a niacinamide compound constituent, detectably or significantly increase NAD+ levels in the body of subject(s). In aspects, the NADP-MC detectably or significantly reduces trans epidermal water loss. In aspects, the NADP-MC detectably or significantly improves, e.g., increases, the moisture content of the skin, e.g., the horny layer of the skin. In aspects, the NADP-MC detectably or significantly increases protein (e.g., keratin) synthesis. In aspects, the NADP-MC detectably or significantly stimulates (e.g., increases) ceramide synthesis. In aspects, the NADP-MC detectably or significantly increases the speed of differentiation of keratinocytes. In aspects, the NADP-MC detectably or significantly improves the surface structure of the skin, such as, e.g., by detectably or significantly smoothing out or reducing the number of wrinkles. In aspects, the NADP-MC detectably or significantly inhibits photocarcinogenesis. In aspects, the NADP-MC detectably or significantly reduces inflammation related to acne, rosacea, or both. In aspects, the NADP-MC, detectably or significantly reduces relative risk of non-melanoma skin cancer. In aspects, an NADP-MC detectably or significantly affects an endogenous factor, protein, or other physiological element. In aspects, such an element can impart a physiological affect which detectably or significantly affect an NAD pathway constituent, thereby modulating the NAD pathway. Therefore, in aspects, by affecting one or more such endogenous elements, any NAD pathway in which such an element participates can be directly or indirectly modulated by an NAD pathway modulator provided by compositions of the invention.

In aspects, an NADP-MC also imparts activity upon other physiological processes which are processes separate from or, e.g., not directly related to, specific NAD-related pathways (non-NAD pathway modulation activity), such as, e.g., pathways related to DNA damage repair or cancer prevention. In aspects, such pathways may or may not comprise, at least in part, a relationship to NAD. In aspects, an NADP-MC can affect one or more other endogenous process constituents in addition to affecting one or more NAD pathway constituents, and thus directly or indirectly modulate one or more non-NAD pathways or physiological processes separate from specific NAD pathways in a recipient of the composition(s) herein. In aspects, an NAD pathway component can affect a constituent of an NAD pathway which also participates in, e.g., is also a constituent in, an endogenous process unrelated to NAD, and therefore either directly or indirectly an NAD pathway component can impart activity upon other non-NAD pathways.

In aspects, a NADP-MC is, comprises, or mostly comprises an NAD+ precursor, such as, e.g., a niacinamide compound, or a salt or prodrug thereof. Additional examples of NADP-MC constituents are described below. In aspects, the NADP-MC consists essentially of one or more niacinamide compounds.

In aspects, an NADP-MC can comprise one or more constituents. In aspects, the NADP-MC comprises a single constituent. In aspects, the NADP-MC comprises two or more constituents, such as, e.g., 2, 3, 4, 5 or more constituents which each alone or together impart an NAD pathway modulation effect. In aspects, two or more constituents of an NADP-MC can act synergistically in their provision of an NAD pathway modulation effect, such that the presence of two or more constituents impart an effect which is DOS greater than the effect either constituent would provide alone. In aspects where an NADP-MC comprises two or more constituents, any first constituent does not significantly negatively impede the activity of any second constituent, such as, e.g., it does not DOS reduce or negatively impact the activity of any second constituent whereby the second constituent fails to demonstrate a detectable effect in a majority of subjects in an appropriately conducted and controlled clinical trial due to the interference of the first constituent. Further, in aspects, a constituent of an NADP-MC (e.g., any one of its constituents) does/do not significantly negatively impede the activity of any other component or ingredient of a composition, such as, e.g., an NADP-MC does not reduce or negatively impact the activity of a CP-MC, such as, e.g., it does not reduce or negatively impact the activity of a CP-MC (e.g., any one or more of its constituents) such that a CP-MC fails to demonstrate a detectable effect in a majority of subjects in an appropriately conducted and controlled clinical trial due to the interference of the NADP-MC.

In aspects, an NADP-MC detectably or significantly increases the amount of nicotinamide-adenine dinucleotide (NAD), e.g., NAD+ (oxidized NAD) or, e.g., NADH (reduced form), nicotinamide-adenine dinucleotide phosphate (NADP), e.g., NADP+ (oxidized NADP) or, e.g., NADPH (reduced form), or both NAD and NADP. In aspects, an NADP-MC increases the amount of NAD, nicotinamide-adenine dinucleotide phosphate (NADP), or both, in subject(s) by at least ~0.2%, ≥~0.4%, ≥~0.6%, ≥~0.8%, ≥~1%, ≥~1.5%, ≥~2.25%, ≥~2.75%, ≥~3%, ≥~3.5%, ≥~4%, ≥~4.5%, ≥~5%, ≥~5.5%, ≥~6%, ≥~6.5%, ≥~7%, ≥~7.5%, ≥~9%, ≥~10%, ≥~11%, ≥~12.5%, or, e.g., ≥~15%, or by any other suitable detectable, physiologically effective, or significant amount.

In aspects, an NADP-MC, such as, e.g., a nicotinate component, e.g., a nicotinate compound constituent, detectably or significantly increases the amount of niacin (vitamin B3) in the body of subject(s).

In aspects, an NADP-MC, such as, e.g., a nicotinate compound constituent, detectably or significantly increases the amount of poly(ADP-ribose) polymerase-1 (PARP-1) in the body. In aspects, an NADP-MC, such as, e.g., a nicotinate compound constituent, detectably or significantly increases the amount of a poly(ADP-ribose) polymerase-1 (PARP-1) in the body by at least ~0.15%, ≥~0.25%, ≥~0.4%, ≥~0.8%, ≥~1.1%, ≥~1.5%, ≥~2.25%, ≥~2.5%, ≥~3.3%, ≥~3.5%, ≥~4%, ≥~5%, ≥~5.5%, ≥~6%, ≥~7%, ≥~7.5%, ≥~8%, ≥~8.5%, ≥~9%, ≥~9.5%, or, e.g., ≥~10%, or by another suitable and detectable, physiologically effective, or significant amount.

In aspects, an NADP-MC detectably or significantly reduces trans epidermal water loss. In aspects, an NADP-MC reduces trans epidermal water loss by at least ~0.1%, ≥~0.2%, ≥~0.5%, ≥~0.75%, ≥~1%, ≥~1.5%, ≥~2%, ≥~2.5%, ≥~3%, ≥~3.5%, ≥~4%, ≥~4.5%, ≥~5%, ≥~5.5%, ≥~6%, ≥~6.5%, ≥~7%, ≥~7.5%, ≥~8%, ≥~8.5%, ≥~9%, ≥~9.5%, or, e.g., ≥~10%, or by another suitable and detectable, physiologically effective, or significant amount.

In aspects, an NADP-MC detectably or significantly improves, e.g., increases the moisture content of the skin, e.g., the horny layer of the skin. In aspects, an NADP-MC increases moisture content of the skin by at least ~0.2%, ≥~0.3%, ≥~0.6%, ≥~0.8%, ≥~1%, ≥~2.25%, ≥~2.75%, ≥~3.3%, ≥~4%, ≥~5%, ≥~5.5%, ≥~7%, ≥~8%, ≥~10%, ≥~12.5%, ≥~15%, or by any other suitable and detectable, effective, or significant amount.

In aspects, an NADP-MC detectably or significantly increases protein (e.g., keratin) synthesis. In aspects, an NADP-MC increases protein (e.g., keratin) synthesis by at least ~0.1%, ≥~0.2%, ≥~0.5%, ≥~0.75%, ≥~1%, ≥~1.5%, ≥~2%, ≥~2.5%, ≥~3%, ≥~3.5%, ≥~4%, ≥~4.5%, ≥~5%, ≥~5.5%, ≥~6%, ≥~6.5%, ≥~7%, ≥~7.5%, ≥~8%, ≥~8.5%, ≥~9%, ≥~9.5%, or, e.g., ≥~10%, or by any other suitable and detectable, effective, or significant amount.

In aspects, an NADP-MC detectably or significantly stimulates (e.g., increases) ceramide synthesis. In aspects, an NADP-MC increases ceramide synthesis by at least ~0.25%, ≥~0.33%, ≥~0.5%, ≥~0.75%, ≥~1.5%, ≥~2%, ≥~2.5%, ≥~3.5%, ≥~4.5%, ≥~5%, ≥~6%, ≥~7.5%, ≥~9%, ≥~10%, ≥~12%, ≥~14%, or ≥~15% or by any other suitable and detectable, effective, or significant amount.

In aspects, an NADP-MC detectably or significantly increases the speed of differentiation of keratinocytes. In aspects, an NADP-MC detectably or significantly improves the surface structure of the skin, such as, e.g., by detectably or significantly smoothing out or reducing the number of wrinkles. In aspects, an NADP-MC reduces the number of wrinkles, depth of one or more wrinkles, or average depth of two or more wrinkles, or any combination of any two or more thereof in an identified area of wrinkle-affected skin by at least ~0.1%, ≥~0.2%, ≥~0.5%, ≥~0.75%, ≥~1%, ≥~1.5%, ≥~2%, ≥~2.5%, ≥~3%, ≥~3.5%, ≥~4%, ≥~4.5%, ≥~5%, ≥~5.5%, ≥~6%, ≥~6.5%, ≥~7%, ≥~7.5%, ≥~8%, ≥~8.5%, ≥~9%, ≥~9.5%, or, e.g., ≥~10% or other suitable and effective, detectable, or significant amount, over a correspondingly wrinkle-affected, but untreated, area of skin in subject(s) or other suitable control(s).

In aspects, an NADP-MC detectably or significantly inhibits photocarcinogenesis. In aspects, an NADP-MC detectably or significantly reduces inflammation, e.g., skin disease-associated inflammation, such as rosacea-associated inflammation. In aspects, the NADP-MC, detectably or significantly reduces relative risk of non-melanoma skin cancer.

In aspects, compositions provided by the invention comprise an NADP-MC which comprises one or more constituents. An NADP-MC constituent can be any constituent which, when provided in effective amounts, imparts a detectable or significant effect on one or more NAD pathway constituents, such as e.g., proteins (including enzymes) or other element participating in one or more NAD-related activities, or which impart a detectable or significant measurable affect attributable to such a compound.

In aspects, an NADP-MC can comprise an early-stage precursor of an NAD pathway modulator. E.g., in aspects, an NADP-MC can comprise an effective amount of tryptophan, which can in effective amount and context DOS increase NAD+ in subject(s) (as indicated earlier, “in subject(s)” meaning in a particular subject or in a significant population of subject as determined through suitable clinical study/studies). In other aspects, most, generally all, essentially all, substantially all, or all of the NADP-MC is DOS more efficient than tryptophan in enhancing NAD+ in subject(s). NADP-MC constituent(s) also can comprise, e.g., enzyme(s) involved in NAD+ biosynthesis or an enzymatically active fragment or variant thereof, such as nicotinamide mononucleotide adenylyltransferase or nicotinamide phosphoribosyltransferase (see, e.g., US20150265642). Other examples of such polypeptides/proteins include, e.g., nicotinate phosphoribosyl transferase 1 (NPT1), pyrazinamidase/nicotinamidase 1 (PNC1), nicotinic acid mononucleotide adenylyltransferase 1 (NMA1), nicotinic acid mononucleotide adenylyltransferase 2 (NMA2), nicotinamide N-methyltransferase (NNMT), nicotinamide phosphoribosyl transferase (NAMPT or NAMPRT), nicotinate/nicotinamide mononucleotide adenylyl transferase 1 (NMNAT-1), and nicotinamide mononucleotide adenylyl transferase 2 (NMNAT-2); see, e.g., U.S. Pat. No. 7,977,049. In aspects, compositions comprise nucleic acid constructs and related compositions (cells, vectors, etc.) that encode for one or more of such enzyme(s), or a variant/fragment thereof.

In aspects, compositions comprise an NADP-MC which represents about 0.1-about 6 wt. % of the composition, such as, e.g., between ~0.2-~5.8 wt. %, ~0.4-~5.6 wt. %, ~0.8-~5.2 wt. %, ~1-~5 wt. %, ~1.2-~5 wt. %, ~1.4-~5 wt. %, ~1.6-~5 wt. %, ~1.8-~5 wt. %, ~2-~5 wt. %, ~2.2-~5 wt. %, ~2.4-~5 wt. %, ~2.6-~5 wt. %, ~2.8-~5 wt. %, or, e.g., about 3-about 5 wt. %, such as, e.g., ~1-~4.8 wt. %, ~1-~4.6 wt. %, ~1-~4.4 wt. %, ~1-~4.2 wt. %, ~1-~4 wt. %, ~1-~3.8 wt. %, ~1-~3.6 wt. %, or, e.g., ~1-~3.4 wt. %, such as for example between ~1.5-~4.5 wt. %, ~2-~4 wt. %, ~2.5-~3.5 wt. %, as in, e.g., about 3.25 wt. %.

In aspects, a single dose of a composition comprises about 1 mg to about 20 mg of an NADP-MC, such as, e.g., ~1 mg-~18 mg, ~1 mg-~16 mg, ~1 mg-~14 mg, ~1 mg-~12 mg, or, e.g., ~1 mg-~10 mg, of an NADP-MC, such as, e.g., ~2 mg-~20 mg, ~4 mg-~20 mg, ~6 mg-~20 mg, ~8 mg-~20 mg, or, e.g., ~10 mg-~20 mg of an NADP-MC.

In aspects, a single dose of composition comprises about 16-164 mg of an NADP-MC, such as, e.g., between ~26-~164 mg, ~36-~164 mg, ~46-~164 mg, ~56-~164 mg, ~66-~164 mg, ~76-~164 mg, ~86-~164 mg, ~96-~164 mg, ~106-~164 mg, ~116-~164 mg, ~126-~164 mg, ~136-~164 mg, ~146-~164 mg, or, e.g., ~156-~164 mg, such as, e.g., between ~16-~154 mg, ~16-~144 mg, ~16-~134 mg, ~16-~124 mg, ~16-~114 mg, ~16-~104 mg, ~16-~94 mg, ~16-~84 mg, ~16-~74 mg, ~16-~64 mg, ~16-~54 mg, ~16-~44 mg, ~16-~34 mg, or, e.g., ~16-~24 mg of an NADP-MC per dose. In aspects, the amount per dose is dependent upon the size of the treatment area, the targeted indication, the targeted affect, or any combination thereof.

In cases, a NADP-MC comprises a niacinamide compound component (a collection of one or more niacinamide compounds, each comprising a niacinamide-like core/structure, or a composition comprising such compound(s) that is an ingredient). In aspects, the NADP-MC comprises a nicotinate compound component (a collection of one or more nicotinate/niacin/nicotinic acid compounds, each comprising a nicotinate structure/core, or a composition comprising such compound(s) that acts as an ingredient in a composition). In aspects, the NADP-MC comprises both a niacinamide and a nicotinate compound component. The structure of nicotinic acid/a nicotinate core and the structure of niacinamide/a niacinamide core are provided below as Formulas XXIX and XXX, respectively:

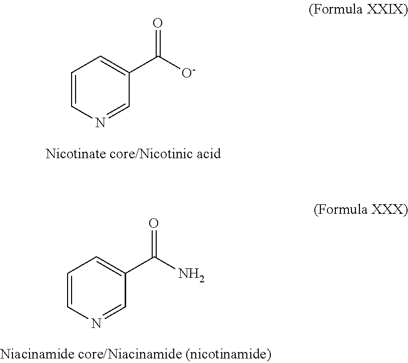 [SMILES: O=C([O-])c1cccnc1 ; NC(=O)c1cccnc1]

Related compounds/derivatives can, in cases, be generated by removal of one or a few of the atoms shown in the structures presented above (e.g., one or both of the hydrogens in the amine in a nicotinamide core can be removed when the remainder of the nicotinamide compound is conjugated to another compound, as in the case of N-Nicotinoyl tyramine or a hydrogen can be removed from the ring nitrogen to form a derivative such as nicotinamide riboside). Each type of such NADP-MC constituent is described in more detail below.

In aspects, the NADP-MC is at least mostly or generally made up of a niacinamide compound component. In aspects, a NADP-MC that is mostly or at least generally made up of a niacinamide compound component. In aspects, the NADP-MC comprises a niacinamide compound component which represents about 86-about 98 wt. % of the NADP-MC, such as, e.g., between ~86-~97 wt. %, ~86-~96 wt. %, ~86-~95 wt. %, ~86-~94 wt. %, or ~86-~93 wt. %, e.g., ~87-~98 wt. %, ~88-~98 wt. %, ~89-~98 wt. %, ~90-~98 wt. %, or ~91-~98 wt. %, such as, e.g., between ~87-~97 wt. %, ~88-~96 wt. %, ~89-~95 wt. %, ~90-~94 wt. %, ~91-~93 wt. %, or, e.g., ~92 wt. % of the NADP-MC.

In aspects, a NADP-MC comprises a nicotinate compound component and a niacinamide compound component, wherein the nicotinate compound component and niacinamide compound component are present in a ratio of about 1:4 to about 1:40. In aspects, the nicotinate compound component and niacinamide compound component are in a ratio of about 1:8 to about 1:20. In aspects, the nicotinate compound component and niacinamide compound component are in a ratio of about 1:10 to about 1:15.

Compositions can include any suitable combination(s) of such NADP-MC constituent(s). E.g., in aspects a composition comprises nicotinamide and nicotinic acid. In aspects, a composition comprises two or all of nicotinamide, nicotinic acid, and nicotinic acid riboside. In some embodiments, a composition comprises two or all of nicotinic acid, nicotinamide mononucleotide, and NAD+/NAD. In some embodiments, a composition comprises two or all of nicotinamide, nicotinamide mononucleotide, and NAD+/nicotinic acid. In aspects, a composition comprises two or all of nicotinic acid adenine dinucleotide, nicotinic acid mononucleotide, and nicotinic acid. In cases, a composition comprises nicotinamide riboside and nicotinic acid. In some embodiments, the composition comprises one or both of nicotinamide riboside and nicotinamide mononucleotide, and nicotinic acid. In some embodiments, the composition comprises both nicotinamide riboside and nicotinamide mononucleotide. In some embodiments, the composition comprises nicotinamide riboside, nicotinic acid, and nicotinamide. In some embodiments, the composition comprises nicotinamide riboside and nicotinic acid riboside. In some embodiments, the composition comprises nicotinamide riboside and nicotinic acid mononucleotide. In some embodiments, the composition comprises nicotinamide in combination with inositol hexanicotinate or nicotinic acid riboside. In some embodiments, the composition comprises inositol hexanicotinate, and nicotinamide mononucleotide. In some embodiments, the composition comprises nicotinamide riboside and inositol hexanicotinate. In some embodiments, the composition comprises nicotinamide riboside, nicotinamide mononucleotide, and inositol hexanicotinate. In some embodiments, the composition comprises nicotinamide riboside or nicotinamide in combination with inositol hexanicotinate. A variety of these and other combinations of such active ingredients have been described in the art (see, e.g., WO2020206160). In embodiments, the composition comprises methyl nicotinate or tocopheryl nicotinate in combination with niacinamide. In embodiments a NADP-MC is mostly, generally only, substantially only, essentially only, or entirely made up of a combination of methyl nicotinate or tocopheryl nicotinate in combination with niacinamide. Additional NAD-related compounds (e.g., NAD pathway anabolites/catabolites) that may be adaptable to or applied in aspects are described, e.g., Makarov M V et al. Biochem Soc Trans. 2019; 47 (1):131-147. The processing of such compounds to NAD+/NAD/NADP/NADPH in subjects are referred to as pathways and also are described in Marakov et al. and other available references. Nicotinate and niacinamide compounds and related compounds can be considered forms of vitamin B3. In aspects, the vitamin B3 content of a composition is the greatest (highest concentration) vitamin component of a composition. In aspects, the amount of vitamin B3 compound content (vitamin B3 compound(s) (VB3C(s)) is at least 1.5×, at least 2×, at least 2.5×, or at least 3× greater than any other vitamin compound(s) in the composition. In aspects, a composition comprises both VKC(s) and VB3C(s) and the ratio of VB3C(s) to VKC(s) is at least about 1.75:1, at least about 2.25:1, at least about 2.75:1, or at least about 3.1:1 (e.g., ~3.25:1), such as about 1.5:1 to about 6:1, about 1.75:1 to about 5.25:1, about 2:1 to about 4:1, or about 2.5:1 to about 3.5:1. In aspects, the V3BC content of a composition makes up ≥50% of the total vitamin content of the composition, such as ≥60%, ≥65%, ≥70%, or ≥75% of the vitamin content of the composition.

In aspects, the NADP-MC constituent(s) do not cause a significant amount of headache, abdominal pain, diarrhea, dyspepsia, nausea, vomiting, rhinitis, pruritus and rash, risk or occurrence of hypotension, fatigue, glucose intolerance/insulin resistance, heartburn, blurred or impaired vision, macular edema, risk or occurrence of liver disease/failure, or risk or occurrence of hepatitis

### Nicotinate Compounds

In aspects, the NADP-MC comprises, mostly comprises, generally consists of, essentially consists of, or consists of an effective amount of nicotinate compound(s) (or a nicotinate compound component). A nicotinate compound is either pharmaceutically or cosmetically effective (and, of course, acceptable/suitable) and present in an effective amount capable of DOS modulating an NAD pathway, such as, e.g., modulating one or more NAD pathway components. In aspects, a nicotinate compound is a pyridinemonocarboxylate. In aspects, a nicotinate compound component comprises nicotinic acid or a conjugate base of nicotinic acid.

In aspects, a nicotinate compound is a nicotinate compound comprising an ionic bonded conjugate, such as a salt, such as, e.g., is present as aluminum nicotinate. Other known nicotinate salt forms include niacin ammonium salt; niacin calcium salt; niacin cobalt (2+) salt; niacin copper (2+) salt; niacin iron (2+) salt; niacin lithium salt; niacin lithium salt, hemihydrate; niacin magnesium salt; niacin manganese (2+) salt; niacin potassium salt; niacin sodium salt; niacin tartrate; niacin tosylate; and niacin zinc salt. Other related compounds include niacin hydrochloride. In aspects, a nicotinate compound can be a nicotinate derivative in which a nicotinate/nicotinic acid core is attached to one or more suitable groups through covalent bond(s), such as, e.g., is present in tocopheryl nicotinate. Sometimes niacinamide is considered a derivative of niacin in the art, but, herein, niacinamide and related compounds are considered a separate class of compounds (described below) are considered separate from niacin and other derivatives comprising a niacin/nicotinic acid core structure.

In aspects, a nicotinate compound component (comprising one or more nicotinate(s)) causes or induces/promotes DOS vasodilation in subject(s). In aspects, a nicotinate compound component or constituent thereof causes DOS more vasodilation than any niacinamide compound in the composition, DOS more vasodilation than is caused by any niacinamide compound component of the composition, or both.

In aspects, a nicotinate compound is a nicotinate compound that is recognized in the art to cause flushing (a “flushing” nicotinate) (flushing being understood as a significant warmth, redness, itching or tingling associated with the compound(s)) or that causes a significantly similar amount of flushing in subject(s). In aspects, a nicotinate compound is a nicotinate compound recognized in the art as not causing flushing or that causes a significantly reduced level of or frequency of flushing compared to a flushing nicotinate (a “non-flushing” nicotinate), such as, e.g., inositol hexanicotinate. Non-flushing nicotinates and related compounds/mimetics are described in, e.g., WO 2008/016968 and WO 2011/163619. In aspects, non-flushing nicotinates still induce/promote or cause DOS vasodilation in subject(s) (in aspects not to a degree that causes flushing). In aspect(s) compositions comprising a nicotinate compound component cause significant flushing in subject(s) for less than 2 weeks, less than 10 days, less than 1 week, less than 5 days, less than 3 days, or less than 2 days, on average, in a significant number of subject(s), or both (or even in most subjects, generally all subjects, or all subjects, e.g., as determined in a clinical study). In aspects, the amount/concentration of nicotinate(s) in the composition and other characteristics of the composition also can impact the degree of vasodilation/flushing arising from nicotinate compound(s) of the composition and, in aspects, compositions are formulated with a concentration of nicotinate(s) or otherwise (e.g., by comprising a delayed release matrix/formulation), so as to DOS reduce flushing as compared to another formulation lacking the characteristic(s) of the formulation.

In aspects, a nicotinate compound is a nicotinate compound comprising two or more nicotinate subunits, such as, e.g., chromium nicotinate (which comprises 3 nicotinate subunits in ionic association with a chromium (Cr³⁺) ion) or inositol nicotinate (which comprises 6 nicotinate subunits covalently bound to an inositol core).

In aspects, a nicotinate compound is an esterified nicotinate. In aspects, the nicotinate compound is a methyl nicotinate, inositol nicotinate, vitamin E (tocopherol) nicotinate, benzyl nicotinate, myristyl nicotinate, ethyl nicotinate, butoxyethyl nicotinate, isopropyl nicotinate, thurfyl nicotinate, aluminum nicotinate, xanthinol niacinate, hexyl nicotinate, nicotinic acid riboside, nicotinic acid adenine dinucleotide, O-ethylnicotinate riboside, triacetyl-O-ethylnicotinate riboside, or O-methylnicotinate riboside (see, e.g., Yang et al., J. Med. Chem., 2007, 50 (26), 6458-6461 and WO2020081923), or other known or otherwise suitable nicotinate, additional examples of which are provided herein (see also, e.g., U.S. Pat. Nos. 3,890,333 and 4,556,715, which describe nicotinate derivatives). Nicotinate conjugates/derivatives can comprise other fatty acid conjugates, alkyl conjugates, metal salt forms, etc.

In aspects, the nicotinate compound is a suitable nicotinic acid/nicotinate prodrug (e.g., a nicotinic-acid ester), a hydrate (e.g., cytosinium nicotinate hydrate), salt (e.g., an ammonium salt, sodium salt, etc.), a nicotinic acid solvate, isomer (e.g., an inositol niacinate isomer, see, e.g., Marszałł M P et al. J Pharm Biomed Anal. 2006 Apr. 11; 41 (1):329-32 and US20090326013), or a polymorph (e.g., inositol nicotinate polymorph A) of a nicotinate/nicotinic acid/niacin compound, etc.

In aspects, an NADP-MC comprises a nicotinate mimetic, which in aspects has one or more detectably or measurably different characteristics (e.g., in size, shape, level of activity, or the like) as a nicotinate compound yet is capable of exerting a physiological functionally similar or equivalent effect as a nicotinate compound (e.g., a nicotinate mimetic). Niacin mimetics are known in the art (see, e.g., US20160030441; Goel H et al. Curr Atheroscler Rep. 2016 April; 18 (4):17; Dunbar R L et al. J Lipid Res. 2017 April; 58 (4):783-797; and Tufts University, “Novel Non-Flushing Niacin Derivatives and Mimetics,” licensing opportunity, published: 23/12/2020 (citing U.S. Pat. Nos. 8,377,971, 8,889,720, 9,193,708, 9,511,060, 8,450,316, 8,937,063, and 9,212,142)). Additional exemplary nicotinate compounds include, e.g., lithium nicotinate, benzyl nicotinate, nicotinyl alcohol, Acipimox, myristyl nicotinate, aluminum nicotinate, xanthinol nicotinate, and chromium nicotinate.

In aspects, the nicotinate compound component comprises a nicotinate compound and formulation associated therewith which is suitable for topical administration. Examples of such compositions/formulations are described herein, and others are known in the art (see, e.g., US20040081672 and Tashtoush B M, et al. Drug Dev Ind Pharm. 2007; 33:1176-1182).

In aspects, the nicotinate compound is a compound which is characterizable as a non-irritating nicotinate. As used, here, a non-irritating nicotinate is a nicotinate which results in irritation detectably or significantly less than that of methyl nicotinate in a majority of subjects when topically applied over a period (e.g., a period of about 3 days, 1 week, 10 days, 2 weeks, 1 month or longer). In aspects, the nicotinate compound component consists at least mostly of a non-irritating nicotinate compound. In aspects, the active ingredient(s) of a NADP-MC comprise, mostly comprise, generally consist of, essentially consist of, or consist of nicotinic acid ester(s). In aspects, the nicotinic acid ester results in a DOS greater effect, a longer sustained effect, or both, in one or more respects when topically applied as compared to nicotinic acid/niacin. In aspects, the nicotinate compound component mostly comprises, generally consists of, or consists essentially of non-irritating nicotinate(s). In aspects, a nicotinate used in compositions comprises, mostly comprises, generally consists of, or essentially consists of methyl nicotinate. In aspects, a non-irritating nicotinate used in compositions comprises, mostly comprises, generally consists of, or consists of tocopheryl nicotinate. In aspects, the NADP-MC or a constituent thereof results in a detectable or significant increase in perfusion, vasodilatory action(s), or other modulation of vasculature/microvasculature when a topic composition comprising the NADP-MC is applied to subject(s) in effective amount(s). In aspects, the nicotinate compound component is at least mostly composed of a non-irritating nicotinate, e.g., a nicotinate which, when delivered in an effective amount, results in no significant increase in inflammation in a population of subjects, as determined by at least one clinical study, such as 1, 2, 3, 4, or 5 or more clinical studies. In aspects, the nicotinate compound component is at least mostly composed of a non-irritating nicotinate, e.g., a nicotinate which, when delivered in an effective amount, results in a level of inflammation which is not deemed an impediment to FDA approval or approval by a corresponding regulatory authority (such as a regulatory authority outside of the United States.) In aspects, the nicotinate compound component comprises a nicotinate compound which is at least mostly composed of a nicotinate which, when delivered in an effective amount, results in DOS less frequent irritation, DOS reduced level of irritation, or both, than that of methyl nicotinate.

In aspects, compositions comprise ~0.005 wt. %-3 wt. % nicotinate compound(s) or ~0.005 wt. %-2 wt. % or, e.g., ~0.01 wt. %-~1 wt. % or ~0.1 wt. %-~1.75 wt. % nicotinate compound(s). In aspects, compositions comprise 0.1 wt. %-1.5 wt. % or 0.1 wt.-1 wt. % nicotinate compound(s). In aspects, compositions comprise 0.05 wt. %-0.75 wt. % nicotinate compound(s), such as 0.09 wt. %-0.66 wt. % nicotinate compound(s), such as 0.1 wt. %-0.75 wt. % or 0.1 wt. %-0.5 wt. % nicotinate compound(s), e.g., about 0.15 wt. %-about 0.35 wt. % or 0.2 wt. %-about 0.3 wt. %. In aspects, compositions contain less than ~1 wt. %, less than ~0.75 wt. %, less than ~0.65 wt. %, less than ~0.5 wt. %, less than ~0.1 wt. %, or less than ~0.05 wt. % nicotinate compound(s).

In aspects, the NADP-MC comprises a nicotinate compound component, which constitutes about 0.01-about 1

In aspects, the NADP-MC comprises a nicotinate compound component, which constitutes about 0.01 wt. %-about 1.5 wt. % of the composition, such as between ~0.02 wt. %-~1.5 wt. %, ~0.03 wt. %-~1.5 wt. %, ~0.04 wt. %-~1.5 wt. %, ~0.06 wt. %-~1.5 wt. %, ~0.07 wt. %-~1.5 wt. %, ~0.08 wt. %-~1.5 wt. %, ~0.09 wt. %-~1.5 wt. %, such as, e.g., about 0.1 wt. %-1.5 wt. %, such as for example ~0.01 wt. %-~1.4 wt. %, ~0.01 wt. %-~1.3 wt. %, ~0.01 wt. %-~1.2 wt. %, ~0.01 wt. %-~1.1 wt. %, or, e.g., ~0.01 wt. %-~1 wt. %, e.g., ~0.02 wt. %-~1.4 wt. %, ~0.04 wt. %-~1.3 wt. %, ~0.06 wt. %-~1.2 wt. %, ~0.08 wt. %-~1.1 wt. %, or about ~0.1 wt. %-~1 wt. % of the composition. In aspects, the nicotinate compound is methyl nicotinate or tocopheryl nicotinate. In aspects, the nicotinate compound component is mostly, generally consists of, consists essentially of, or consists of tocopheryl nicotinate.

In aspects, the NADP-MC comprises a nicotinate compound component which represents about 2-about 14 wt. % of the NADP-MC, such as, e.g., between ~2-~13 wt. %, ~2-~12 wt. %, ~2-~11 wt. %, ~2-~10 wt. %, or ~2-~9 wt. %, e.g., ~3-~14 wt. %, ~4-~14 wt. %, ~5-~14 wt. %, ~6-~14 wt. %, or ~7-~14 wt. %, such as, e.g., between ~3-~13 wt. %, ~4-~12 wt. %, ~5-~11 wt. %, ~6-~10 wt. %, ~7-~9 wt. %, or, e.g., ~8 wt. % of the NADP-MC. In aspects, the nicotinate compound is mostly, generally, essentially, or only tocopheryl nicotinate.

In aspects, a single application of composition comprises about 1-about 13 mg of a nicotinate compound, such as, e.g., comprising between ~2-~13 mg, ~3-~13 mg, ~4-~13 mg, ~5-~13 mg, ~6-~13 mg, ~7-~13 mg, ~8-~13 mg, ~9-~13 mg, ~10-~13 mg, or, e.g., ~11-~13 mg, such as, e.g., ~1-~13 mg, ~1-~11 mg, ~1-~10 mg, ~1-~9 mg, ~1-~8 mg, ~1-~7 mg, ~1-~6 mg, ~1-~5 mg, ~1-~4 mg, ~1- or ~3 mg of a nicotinate compound per application. In aspects, the nicotinate compound is mostly, generally, essentially, or only tocopheryl nicotinate.

### Nicotinamide/Niacinamide Compounds

In aspects, an NADP-MC comprises an effective amount (e.g., a therapeutically effective amount, cosmetically effective amount, or otherwise physiologically effective amount) of one or more niacinamide compound(s) (or, e.g., a niacinamide compound component). In the art, “niacinamide” and “nicotinamide” may be used interchangeably (but should not be confused with “nicotinate” and related terms, such as niacin and nicotinic acid, as discussed above). Niacinamide compounds are characterized by, i.a., comprising a nitrogen in the side chain to the pyrimidine core of the niacinamide core (e.g., forming an amide bond with heterologous group(s) in the case of some derivatives).

Niacinamide compound(s) can comprise derivatives of niacinamide/nicotinamide formed by covalent bonds to one or more suitable conjugate group(s), such as is the case in respect of, e.g., N-Nicotinoyl tyramine, nicotinamide riboside, nicotinamide-adenine dinucleotide (NAD/NAD+), or nicotinamide mononucleotide. Niacinamide compound(s) also or alternatively can comprise non-covalent conjugate compounds, such as those which are formed by ionic bonds or other non-covalent bonds/associations, as is the case with niacinamide ascorbate and niacinamide salicylate. A variety of niacinamide/nicotinamide derivatives and nicotinamide-related compounds are known in the art, such as, e.g., N-Nicotinoyl dopamine (Kim B et al. Exp Dermatol. 2011 November; 20 (11):950-2), and with application of routine experimentation such compounds can be adapted for use in compositions/methods of the invention, where such compound(s) are suitable. See, further, e.g., Peng et al. Synthesis and biological evaluation. Chemistry Central Journal. 11. 10.1186/s13065-017-0338-5; Yang Z et al. J Pestic Sci. 2020; 45 (1):39-44. doi: 10.1584/jpestics.D19-061; WO2017218580; WO2019121548; WO2021125708; US20200352966; US20090105264; U.S. Pat. No. 10,280,190; WO2020257283; WO2020172629; WO2021123388; U.S. Pat. No. 7,902,373; US20120053170; WO2010143803; U.S. Pat. No. 9,051,310; WO2019122084; U.S. Pat. No. 7,141,586; WO2017218580; and WO2021013795. In aspects, niacinamide compound(s) are a prodrug, hydrate, solvate, isomer/enantiomer, or polymorph of one or more of such niacinamide compound(s). In aspects, the niacinamide compound(s) are suitable for topical application. Examples of such compositions are described further here, and others are described in the art, which may be adaptable or applicable to aspects (see, e.g., WO2019121529, US20180000719, WO2014132060, WO2018134714, US20180353497, and U.S. Pat. No. 9,233,061). Optionally, compositions of the invention lack one or more other primary ingredients, active ingredients, or most of the ingredients described in such references. In aspects, an NADP-MC comprises a niacinamide mimetic, which in aspects has one or more detectably or measurably different characteristics (e.g., in size, shape, level of activity, or the like) as a niacinamide compound yet is capable of exerting a physiological functionally similar or equivalent effect, or an improved effect in one or more respects, as a referenced niacinamide compound.

In aspects, compositions comprise a niacinamide compound component which constitutes about 1-about 5%, such as between ~1.2-~5 wt. %, ~1.4-~5 wt. %, ~1.6-~5 wt. %, ~1.8-~5 wt. %, ~2-~5 wt. %, ~2.2-~5 wt. %, ~2.4-~5 wt. %, ~2.6-~5 wt. %, ~2.8-~5 wt. %, such as ~3-~5 wt. %, as in ~1-~4.8 wt. %, ~1-~4.4 wt. %, ~1-~4.2 wt. %, ~1-~4 wt. %, ~1-~3.8 wt. %, ~1-~3.6 wt. %, ~1-~3.4 wt. %, ~1-~3.2 wt. %, or ~1-~3 wt. % of the composition. In aspects, the niacinamide compound component of a composition is mostly, generally, essentially, or only made up of niacinamide.

In aspects, a single dose of a composition comprises about 1 mg to about 20 mg of a niacinamide compound, such as, e.g., ~1 mg-~18 mg, ~1 mg-~16 mg, ~1 mg-~14 mg, ~1 mg-~12 mg, or, e.g., ~1 mg-~10 mg, of a niacinamide compound, such as, e.g., ~2 mg-~20 mg, ~4 mg-~20 mg, ~6 mg-~20 mg, ~8 mg-~20 mg, or, e.g., ~10 mg-~20 mg of a niacinamide compound.

In aspects, a single dose of composition comprises about 15-about 150 mg of a niacinamide compound, such as, e.g., between ~25-~150 mg, ~35-~150 mg, ~45-~150 mg, ~55-~150 mg, ~65-~150 mg, ~75-~150 mg, ~85-~150 mg, ~95-~150 mg, ~105-~150 mg, ~115-~150 mg, ~125-~150 mg, ~135-~150 mg, or, e.g., ~145-~150 mg, such as, ~15-~140 mg, ~15-~130 mg, ~15-~120 mg, ~15-~110 mg, ~15-~100 mg, ~15-~90 mg, ~15-~140 mg, ~15-~80 mg, ~15-~70 mg, ~15-~60 mg, ~15-~50 mg, ~15-~40 mg, ~15-~30 mg, or, e.g., ~15-~20 mg of a niacinamide compound per dose. In aspects, the niacinamide compound is niacinamide. In aspects, the amount per dose is dependent upon the size of the treatment area, the targeted indication, the targeted affect, or any combination thereof.

In aspects, an NADP-MC comprises an amount of a niacinamide compound which represents about 80-about 98% of the NADP-MC, such as ~80-~97%, ~80-~96%, ~80-~95%, ~80-~94%, ~80-~93%, or ~80-~92% of the NADP-MC, such as, e.g., ~82-~98%, ~84-~98%, ~86-~98%, ~88-~98%, ~90-~98%, or, e.g., ~92-~98% of the NADP-MC, such as, e.g., ~82-~97%, ~84-~96%, ~86-~95%, ~88-~94%, or, e.g., between ~90-~93% of the NADP-MC, e.g., about 92% of the NADP-MC.

### Penetration Enhancer Component

In aspects, compositions are adapted for/configured for topical delivery. In aspects, compositions comprise a penetration enhancer component (PEC), comprising one or more penetration enhancer component constituents (PECCs), each of which detectably or significantly (DOS) promotes penetration of one or more other compound(s)/molecule(s) of the composition into/across the skin of subject(s). In aspects, a penetration enhancer component comprises a single constituent (i.e., a single compound/molecule that acts as a penetration enhancer). In aspects, the penetration enhancer component comprises two or more constituents which each alone contribute to a penetration enhancement effect or together impart penetration enhancement effect. In aspects, two or more constituents of penetration enhancer component can act synergistically in their provision of a penetration enhancement effect.

In aspects, a penetration enhancer component detectibly or significantly increases the average amount of one or more other ingredients penetrating the skin per administration/dose (e.g., per application), the average amount of one or more other ingredients penetrating the skin within a given time period, or both. In aspects, presence of a penetration enhancer increases the amount of one or more other ingredients penetrating the skin per dose/application by at least about 1%, such as, e.g., ≥~2.5%, ≥~4%, ≥~5%, ≥~10%, ≥~15%, ≥~25%, ≥~35%, ≥~40%, ≥~50%, ≥~65%, ≥~70%, ≥~75%, ≥~80%, ≥~85%, ≥~90%, ≥~95%, ≥~100% (2×), or even more, such as, e.g., by ≥~125%, ≥~150%, ≥~175%, ≥~200% (3×), ≥~250%, ≥~300%, ≥~350%, ≥~400%, ≥~450%, or ≥~500%, or by any other effective, detectable, or significant amount. In aspects, presence of a penetration enhancer decreases the amount of time it takes for a given amount of one or more other ingredients to penetrate the skin by at least about 1%, such as, e.g., ≥~2%, ≥~3%, ≥~4%, ≥~5%, ≥~10%, ≥~15%, ≥~20%, ≥~25%, ≥~30%, ≥~35%, ≥~40%, ≥~45%, ≥~50%, ≥~55%, ≥~60%, ≥~65%, ≥~70%, ≥~75%, ≥~80%, ≥~85%, ≥~90%, ≥~95%, or even ≥~100% or more (as determined by comparison against an adequate control, such as in a clinical trial performed with and without the penetration enhancer component (PEC)).

In aspects, a penetration enhancer component (PEC) present in a composition allows therapeutically effective, cosmetically effective, or both therapeutically effective and cosmetically effective amount(s) of compound(s) to penetrate the skin within a therapeutically beneficial or cosmetically beneficial time period, which would not otherwise be possible in the absence of the PEC under the conditions of administration. In aspects, a therapeutically beneficial time period is a reasonable amount of time that an average physician or average user would expect for the compound of the composition to become physiologically available to impart an action, such as, e.g., within a matter of minutes or hours as opposed to within a matter of days or weeks (or not at all). In aspects, compositions comprise one or more ingredients which, without the presence of a penetration enhancer component, would be unable to penetrate the skin in therapeutically effective amounts within a therapeutically suitable time period when administered topically. In aspects, compositions comprise one or more ingredients which, without the presence of a penetration enhancer component, would not be physiologically available in therapeutically effective or cosmetically effective amounts.

In aspects, compositions comprise a penetration enhancer component which represents about 0.5 to about 4 wt. % of the composition, such as, e.g., between ~0.5-~3.75 wt. %, ~0.5-~3.5 wt. %, ~0.5-~3.25 wt. %, or, e.g., ~0.5-~3 wt. %, such as ~0.75-~4 wt. %, ~1-~4 wt. %, ~1.25-~4 wt. %, ~1.5-~4 wt. %, ~1.75-~4 wt. %, or ~2-~4 wt. %, such as about ~0.75-~3.75 wt. %, ~1-~3.5 wt. %, ~1.25-~3.5 wt. %, ~1.5-~3.25 wt. %, ~1.75-~3 wt. %, as in, e.g., ~2 wt. %, ~2.25 wt. %, ~2.5 wt. %, ~2.75 wt. %, or, e.g., ~3 wt. % of the composition.

In aspects, a single dose of a composition comprises about 1 mg to about 20 mg of a penetration enhancer component, such as, e.g., ~1 mg-~18 mg, ~1 mg-~16 mg, ~1 mg-~14 mg, ~1 mg-~12 mg, or, e.g., ~1 mg-~10 mg, of a penetration enhancer component, such as, e.g., ~2 mg-~20 mg, ~4 mg-~20 mg, ~6 mg-~20 mg, ~8 mg-~20 mg, or, e.g., ~10 mg-~20 mg of a penetration enhancer component.

In aspects, a single dose/application of a composition comprises about 10 mg to about 100 mg of a penetration enhancer component, such as, e.g., ~10 to ~90 mg, ~10 to ~80 mg, ~10-~70 mg, ~10 to ~60 mg, ~10-~50 mg, ~10 to ~40 mg, ~10 to ~30 mg, or ~10 to ~20 mg, such as, e.g., ~20 to ~100 mg, ~30 to ~100 mg, ~50 to ~100 mg, ~60-~100 mg, ~70-~100 mg, ~80-~100 mg, or, e.g., ~90 to ~100 mg, of a penetration enhancer component per dose. In aspects, the amount per dose is dependent upon the size of the treatment area, the targeted indication, the targeted affect, or any combination thereof.

### Temporary Skin Disruption Penetration Enhancers

In aspects, a penetration enhancer component can comprise a penetration enhancer component characterizable as a temporary skin disruption penetration enhancer component (TSD-PEC). A TSD-PEC is a component capable of causing a DOS reversible and temporary modulation of skin components (e.g., causing rearrangement of tissues that the composition contacts by temporarily, e.g., reversibly, disrupting cell-to-cell (e.g., intercellular) or cell-scaffold attachment), allowing compound(s) (or an increased amount of compound(s)) in an associated composition to pass through skin cell layers (in an otherwise passive manner) or otherwise penetrate the skin. The mechanism of how TSD-PECs work is not critical or limiting. The defining element(s) of a TSD-PEC is the unique capability of such compound(s)/molecule(s) to cause penetration of agents that normally would not penetrate the skin, and, in aspects, the size, weight, and origin/physiochemical properties of the TSD-PEC, which will be exemplified below. A TSD-PEC, in modifying tissue (e.g., skin) in such a way, allows for a compound to penetrate the skin to a sufficient extent that when the compound is topically applied in a therapeutically effective or cosmetically effective amount, a therapeutically effective or cosmetically effective amount of the compound is able to sufficiently penetrate the skin to exert an intended cosmetic or therapeutic effect. In aspects, absent the presence of an effective amount of a TSD-PEC, one, some, or most active ingredients of the composition would be unable to sufficiently penetrate the skin, even if provided topically in therapeutically or cosmetically effective amounts; could not penetrate the skin to a sufficient extent such that a sufficient amount becomes physiologically available to exert a therapeutic effect; could not penetrate the skin to a sufficient extent within a therapeutically beneficial or cosmetically beneficial time period; or a combination of any or all thereof. In aspects, such agents can penetrate the skin without the TSD-PEC, but the presence of the TSD-PEC results in a DOS improvement in the amount/number of agent(s) that penetrate the skin, the time required for agent(s) to penetrate the skin, or both.

In aspects, a TSD-PEC comprises one or more constituents which DOS reduce(s) the strength of binding present between components, e.g., compounds (the same or different compounds, each on the surface of separate cells within the skin) which serve to bind tissue cells together, DOS reduces the number of such bonds between compounds which serve to bind tissue cells together, or both. In aspects, a TSD-PEC can reduce the strength of binding present between compounds which act to bind tissue cells together by at least about 1%, such as, e.g., ≥~5%, ≥~10%, ≥~15%, ≥~20%, ≥~25%, ≥~30%, ≥~35%, ≥~40%, ≥~45%, or, e.g., ≥~50%, or by any other suitable, detectable, or significant amount. In aspects, a TSD-PEC can reduce the number of active bonds between compounds which serve to bind tissue cells together by at least about 1%, such as, e.g., ≥~5%, ≥~10%, ≥~15%, ≥~20%, ≥~25%, ≥~30%, ≥~35%, ≥~40%, ≥~45%, or, e.g., ≥~50%, or by any other suitable, detectable, or significant amount.

In aspects, the effect of a TSD-PEC is temporary, such that, e.g., at least about 20%, ≥~25%, ≥~30%, ≥~35%, ≥~40%, ≥~45%, ≥~50%, ≥~55%, ≥~60%, ≥~65%, ≥~70%, ≥~75%, ≥~80%, ≥~85%, ≥~90%, ≥~95%, or, e.g., even ~100% of any reduction in strength of binding present between compounds, or reduction in the number of active bonds between compounds, induced by the presence of a TSD-PEC, is reversed within a period of about 48 hours following the cessation of the application of the TSD-PEC, such as, e.g., within a period of ~44 hours, ~40 hours, ~36 hours, ~32 hours, ~28 hours, ~24 hours, ~20 hours, ~16 hours, ~12 hours, ~8 hours, or, e.g., ~4 hours, ~2 hours, or, e.g., ~1 hour after cessation of the application of the TSD-PEC.

In aspects, a TSD-PEC DOS enhances the uptake of ingredient(s) of the composition, such as, e.g., providing for an increase of at least about 1%, ≥~3%, ≥~5%, ≥~10%, ≥~15%, ≥~25%, ≥~35%, ≥~40%, ≥~50%, ≥~65%, ≥~70%, ≥~75%, ≥~80%, ≥~85%, ≥~90%, or, e.g., or ≥~95% or in the amount of at least one ingredient topically applied as an ingredient in a composition comprising a TSD-PEC over the amount of the same ingredient(s) topically applied as an ingredient in a composition not comprising a TSD-PEC.

In aspects, ingredient(s) in a composition with a TSD-PEC that DOS penetrate the skin (e.g., components of a TSD-PEC) has/have a molecular weight of at least about 500 kD, such as, e.g., ≥~520 kD, ≥~540 kD, ≥~560 kD, ≥~580 kD, ≥~600 kD, ≥~620 kD, ≥~640 kD, ≥~660 kD, ≥~680 kD, ≥~700 kD, ≥~720 kD, ≥~740 kD, ≥~760 kD, ≥~780 kD, ≥~800 kD, ≥~820 kD, ≥~840 kD, ≥~860 kD, ≥~880 kD, ≥~900 kD, ≥~920 kD, ≥~940 kD, ≥~960 kD, ≥~980 kD, or, e.g., ≥~1000 kD.

The size of a TSD-PEC can, in cases, impact the effectiveness of the TSD-PEC in terms of the size of associated compound(s)/molecule(s) that a TSD-PEC can “assist” in penetrating the skin. In aspects, the TSD-PEC has an average molecular weight of about 1,000-about 30,000 Daltons, such as, e.g., ~1,000 to ~28,000 Daltons, ~1,000 to ~26,000 Daltons, ~1,000 to ~24,000 Daltons, ~1,000 to ~22,000 Daltons, or, e.g., between ~1,200 to 30,000 Daltons, ~1,400 to ~30,000 Daltons, ~1,600 to ~30,000 Daltons, or ~1,800 to ~30,000 Daltons, as in, e.g., ~1,100-~29,000 Daltons, ~1,200-~28,000 Daltons, ~1,300-~27,000 Daltons, ~1,400-~26,000 Daltons, ~1,500-~25,000 Daltons, ~1,600-~24,000 Daltons, ~1,700-~23,000 Daltons, ~1,800-~22,000 Daltons, ~1,900-~21,000 Daltons, or, e.g., about ~2,000-~20,000 Daltons (about 20 kDa).

In aspects, a TSD-PEC or constituent thereof is a peptidyl fragment of extracellular matrix material. In aspects, the TSD-PEC or constituent thereof is saccharidyl (e.g., is a polysaccharide compound). In aspects, a TSD-PEC or constituent thereof is degradable. In aspects, a TSD-PEC or constituent is bioabsorbable. In aspects, the TSD-PEC or constituent thereof can, independent of its penetration enhancement properties, provide one or more DOS beneficial effects to skin, such as, e.g., providing a DOS moisturizing effect, a DOS anti-aging (e.g., anti-wrinkle) effect, or can, e.g., DOS speed up wound healing.

In aspects, the TSD-PEC or constituent thereof is an extracellular matrix component which can be grouped into several classes of biomolecules based upon their structure and function within the extracellular matrix. In aspects, the TSD-PEC or constituent thereof is an extracellular matrix protein, such as, e.g., collagen family proteins (such as collagen) and elastin family proteins (such as elastin). In aspects, the TSD-PEC or constituent thereof is a specialized protein such as, e.g., fibrillin, fibronectin, laminin, merosin, tenascin, or vitronectin. In aspects, the TSD-PEC or constituent thereof can be a proteoglycan or heparan sulfate-containing protein. In aspects, the TSD-PEC or constituent thereof is a non-sulfate glycosaminoglycan (GAG). In aspects, TSD-PECs or constituents thereof are fragments of such biomolecules. Further descriptions of suitable TSD-PECs (specifically so-called “decoy” compounds) can be found in, e.g., US2019/0105261, US20190008795, and WO 2017/180788 (each to WAUGH/Illustris Pharmaceuticals, Inc.) and related patent applications/patents. In aspects a TSD-PEC is composed of a hyaluronic acid (HA) fragment, a collagen fragment, fibronectin fragment, elastin fragment, a derivative of any thereof, or a combination of any thereof. In aspects, some, most, generally all, substantially all, or essentially all of the TSD-PEC of a composition is composed of sodium hyaluronate, comprising any suitable type of hyaluronic acid ingredient(s) (examples of which are provided herein).

In aspects, a penetration enhancer component, e.g., a TSD-PEC, e.g., a “decoy molecule” component, consists at least mostly of an HA fragment. In aspects, a penetration enhancer component, e.g., a TSD-PEC, e.g., a “decoy molecule” component, consists essentially of an HA fragment. In aspects, the HA is a hydrolyzed sodium hyaluronic acid. In aspects, such a hydrolyzed sodium hyaluronic acid is the type sometimes referred to in the art as a “mini-HA.” In aspects, the HA is a hydrolyzed sodium hyaluronic acid has a molecular weight of about 1,000 to about 30,000 Daltons, such as, e.g., between ~1,000 to ~28,000 Daltons, ~1,000 to ~26,000 Daltons, ~1,000 to ~24,000 Daltons, ~1,000 to ~22,000 Daltons, or, e.g., between ~1,200 to 30,000 Daltons, ~1,400 to ~30,000 Daltons, ~1,600 to ~30,000 Daltons, or ~1,800 to ~30,000 Daltons, as in, e.g., ~1,100-~29,000 Daltons, ~1,200-~28,000 Daltons, ~1,300-~27,000 Daltons, ~1,400-~26,000 Daltons, ~1,500-~25,000 Daltons, ~1,600-~24,000 Daltons, ~1,700-~23,000 Daltons, ~1,800-~22,000 Daltons, ~1,900-~21,000 Daltons, or, e.g., about ~2,000-~20,000 Daltons.

In aspects, a penetration enhancement component (PEC) comprises a TSD-PEC, e.g., a “decoy molecule” component, the TSD-PEC represents about 5 wt. %-about 25 wt. % of the PEC, such as, e.g., e.g., ~6-~25 wt. %, ≥~~~25 wt. %, ~8-~25 wt. %, ~9-~25 wt. %, or, e.g., ~10-~25 wt. %, such as, e.g., ~5-~20 wt. %, ~5-~18 wt. %, ~5-~16 wt. %, ~5-~14 wt. %, or, e.g., ~5-~12 wt. %, e.g., between ~5.5-~20 wt. %, ~6-~18 wt. %, ~6.5-~16 wt. %, ~7-~14 wt. %, ~7.5-~12 wt. %, or, e.g., ~8 wt. %, ~9 wt. %, ~10 wt. %, ~11 wt. %, or ~12% of the PEC is made up of a TSD-PEC. In aspects, a TSD-PEC, e.g., a “decoy molecule” component, can represent about 0.05 wt. %-about 1 wt. % of a composition, e.g., ~0.05-~0.8 wt. %, ~0.05-~0.6 wt. %, or, e.g., ~0.05-~0.4 wt. %, such as, e.g., ~0.06-~1 wt. %, ~0.07-~1 wt. %, ~0.08-~1 wt. %, ~0.09-~1 wt. %, or ~0.1-~1 wt. %, such as, e.g., about 0.06-~0.8 wt. %, ~0.08-~0.6 wt. %, or, e.g., ~0.1-~0.4 wt. % of the composition, e.g., ~0.2 wt. % or ~0.3 wt. % of the composition.

In specific aspects, a TSD-PEC constituent, such as, e.g., a hydrolyzed sodium hyaluronic acid having a molecular weight of about 1,000 to about 30,000 Daltons, can be present in amount of about 0.05 wt. % to about 1.5 wt. % of the composition, such as, e.g., between ~0.05-~1.4 wt. %, ~0.05-~1.3 wt. %, ~0.05-~1.2 wt. %, or, e.g., ~0.05-~1 wt. %, such as, e.g., between ~0.06-~1.5 wt. %, ~0.07-~1.5 wt. %, ~0.08-~1.5 wt. %, ~0.09-~1.5 wt. %, or ~0.1-~1.5 wt. %, as in, e.g., between ~0.06-~1.4 wt. %, ~0.07-~1.3 wt. %, ~0.08-~1.2 wt. %, ~0.09-~1.1 wt. %, or, e.g., ~0.1-~1 wt. % of the composition.

In specific aspects, a TSD-PEC constituent, such as, e.g., a hydrolyzed sodium hyaluronic acid having a molecular weight of about 1,000 to about 30,000 Daltons, can be present in an amount of about 5 wt. %-about 20 wt. % of the penetration enhancer component, such as, e.g., between ~5-~18 wt. %, ~5-~16 wt. %, ~5-~14 wt. %, or, e.g., between ~5-~13 wt. %, such as, e.g., between ≥~~~20 wt. %, ~7-~20 wt. %, or, e.g., ~8-~20 wt. %, such as, for example, ~6-~19 wt. %, ~6.5-~18 wt. %, ~7-~17 wt. %, ~7.5-~16 wt. %, or ~8-~15 wt. %, e.g., ~8-~14 wt. %, or, ~8 to 13 wt. %, such as, e.g., ~9 wt. %, ~10 wt. %, ~11 wt. %, or, e.g., ~12 wt. %, of the penetration enhancer component.

In aspects, a single dose of composition comprises less than 1 mg of a TSD-PEC constituent per dose, such as between about 0.01 mg to about 1 mg of a TSD-PEC constituent per dose, e.g., ~0.1 mg, ~0.2 mg, ~0.3 mg, ~0.4 mg, ~0.5 mg, ~0.6 mg, ~0.7 mg, ~0.8 mg, or about 0.9 mg of a TSD-PEC constituent per dose.

In aspects, a single dose of composition comprises about 1 mg to about 13 mg of a TSD-PEC constituent per dose, such as, e.g., between ~2-~12 mg, ~2-~11 mg, ~2-~10 mg, ~2-~9 mg, ~2-~8 mg, ~2-~7 mg, ~2-~6 mg, ~2-~5 mg, ~2-~4 mg, or, e.g., ~2-~ 3 mg, such as ~3-~13 mg, ~4-~13 mg, ~5-~13 mg, ≥~~~13 mg, ≥~~~13 mg, ~8-~13 mg, ~9-~13 mg, ~10-~13 mg, ~11-~13 mg, or, e.g., ~12-~13 mg of a TSD-PEC per dose. In aspects, the TSD-PEC is a fragment of hyaluronic acid. In aspects, the TSD-PEC is “mini-HA”. In aspects, the amount per dose is dependent upon the size of the treatment area, the targeted indication, the targeted affect, or any combination thereof.

### Other Penetration Enhancer Compounds

In aspects, a composition/penetration enhancer component can comprise a penetration enhancer constituent that is not characterizable as a TSD-PEC (a “non-decoy” penetration enhancer).

In aspects, a non-TSD-PEC is any therapeutically effective, pharmaceutically acceptable and cosmetologically acceptable penetration enhancer capable of DOS the amount of compound penetrating the skin, rate of a compound penetrating the skin, depth of penetration into the skin, or any combination thereof of one or more other composition ingredients when the composition is topically applied.

In aspects, the non-TSD-PEC is selected from a group comprising but not limited to, alcohols (e.g., ethanol, decanol, glycols such as propylene glycol or ethoxydiglycol), fatty alcohols, sulfoxides (e.g., dimethylsulfoxide), Azones (e.g., laurocapram), pyrrolidones (e.g., 2-pyrrolidone, 2P), dimethyl isosorbide (sometimes referred to as DMI), isopropyl myristate, eucalyptol, surfactants, terpenes (e.g., menthol, thymol, carvacrol, menthone, cinole) and terpenoids, fatty acids (e.g., oleic acid, capric acid, lauric acid, myristic acid, palmitic acid, stearic acid, palmitoleic acid, linoleic acid, alpha-linolenic acid, lignoceric acid, ricinoleic acid, arachidonic acid), urea, etc.

In aspects, the non-TSD-PEC is a compound derived from a bicyclic compound comprising two fused furan rings. In aspects, the non-TSD-PEC is derived from isosorbide. In aspects, the compound is an isosorbide ether. In aspects, the isosorbide ether is dimethyl isosorbide. In aspects, the dimethyl isosorbide is 2,5-dimethylisosorbide.

In aspects, a non-TSD-PEC, such as, e.g., dimethyl isosorbide (a single non-TSD-PEC constituent), can represent about 20 wt. % to about 40 wt. % of the penetration enhancer component, such as, e.g., between about 20 wt. %-about 38 wt. %, ~20 wt. %-~36 wt. %, or ~20 wt. %-~34 wt. %, e.g., ~21 wt. %-~40 wt. %, ~22 wt. %-~40 wt. %, ~23 wt. %-~40 wt. %, ~24 wt. %-~40 wt. %, or, e.g., ~25 wt. %-~40 wt. %, such as, for example, ~21 wt. %-~39 wt. %, ~22 wt. %-~38 wt. %, ~23 wt. %-~37 wt. %, or, e.g., ~24 wt. %-36 wt. %, or, e.g., ~25 wt. %-~35 wt. % of the penetration enhancer component.

In aspects, a non-TSD-PEC, such as, e.g., dimethyl isosorbide (a single non-TSD-PEC constituent), can be present in an amount of about 0.1 wt. %-about 5% of the composition, such as, e.g., between ~0.1~4.5 wt. %, ~0.1 wt. %-~4 wt. %, ~0.1 wt. %-~3.5 wt. %, or, e.g., ~0.1 wt. %-3 wt. %, such as, e.g., ~0.2 wt. %-~5 wt. %, ~0.3 wt. %-~5 wt. %, ~0.4 wt. %-~5 wt. %, or, e.g., ~0.5-~5 wt. %, such as, e.g., ~0.3 wt. %-~3.5 wt. % or, e.g., between about 0.5-about 3 wt. % of the composition.

In aspects, the non-TSD-PEC is an alcohol. In aspects, the non-TSD-PEC is a glycol. In aspects, the non-TSD-PEC is ethoxydiglycol.

According to certain aspects, the composition can be formulated such that the formulation itself provides penetration enhancement effects. That is, in aspects, compositions provided by the invention are formulated as a gel, a hydrogel, an organogel, a bigel, an emulgel, an emulsion, a microemulsion, a nanoemulsion, as multiple emulsions, or, e.g., as liquid crystals. In aspects, vesicular delivery system(s) are used to provide penetration enhancement effects. In aspects, compositions comprise delivery systems such as, e.g., liposomes, ethosomes, transfersomes, or niosomes.

In aspects, any one or more penetration enhancement ingredients or technologies can be combined to provide penetration enhancing effect.

In aspects, a non-TSD-PEC, such as, e.g., ethoxydiglycol (a single non-TSD-PEC constituent), can be present in an amount of about 0.1 wt. %-about 3.5% of the composition, such as, e.g., between ~0.2~3.5 wt. %, ~0.3 wt. %-~3.5 wt. %, ~0.4 wt. %-~3.5 wt. %, or ~0.5 wt. %-~3.5 wt. %, or, e.g., ~0.1 wt. %-~3%, ~0.1 wt. %-~2.5 wt. %, or, e.g., ~0.1-~2 wt. %, such as for example between ~0.2-~3 wt. %, ~0.3-~2.5 wt. %, or, e.g., ~0.5-~2 wt. % of the composition.

In aspects, a non-TSD-PEC, such as, e.g., ethoxydiglycol, can be present in an amount of about 50 wt. %-about 70 wt. % of the penetration enhancer component (PEC), such as, e.g., b~52 wt. % to ~70 wt. %, ~54 wt. % to ~70 wt. %, or, e.g., ~56 wt. % to 70 wt. %, such as, e.g., ~50 wt. %-~69 wt. %, ~50 wt. %-~68 wt. %, ~50 wt. %-~67 wt. %, ~50-~66 wt. %, or, e.g., ~50 wt. %-~65 wt. %, such as, for example, ~51 wt. % to ~69 wt. %, ~52 wt. %-~68 wt. %, ~53 wt. % to ~67 wt. %, ~54 wt. %-~66 wt. % wt. %, or, e.g., ~55 wt. % to ~65 wt. % of the penetration enhancer component.

In aspects, a single dose of a composition comprises about 1 to about 10 mg, such as ~2-~10 mg, ~4-~10 mg, or ~6-~10 mg of a non-TSD-PEC.

In aspects, a single dose of a composition comprises, e.g., about 8 to about 88 mg of a non-TSD-PEC, such as, e.g., between ~8-~78 mg, ~8-~68 mg, ~8-~58 mg, ~8-~ 48 mg, ~8-~38 mg, ~8-~28 mg, or, e.g., ~8-~18 mg, such as, e.g., ~18-~88 mg, ~28-~88 mg, ~38-~88 mg, ~48-~88 mg, ~58-~88 mg, ~68-~88 mg, or, e.g., ~78-~88 mg, of a non-TSD-PEC per dose. In aspects, the non-TSD-PEC can comprise a single constituent. In aspects, the single non-TSD-PEC constituent is ethoxydiglycol. In aspects, the amount per dose is dependent upon the size of the treatment area, the targeted indication, the targeted affect, or any combination thereof.

In aspects, the penetration enhancer component is at least mostly of ethoxydiglycol. In aspects, the penetration enhancer component consists essentially of ethoxydiglycol. In aspects, a composition is free of any TSD-PEC. In aspects, a composition is free of any TSD-PEC.

### Exemplary Amounts of Composition by Application

In aspects, different amounts of a composition can be utilized for different applications. In aspects, for example, a smaller amount of an inventive composition may be applied to, e.g., bruising related to a blunt trauma, compared to a larger amount of an inventive composition being applied to a larger affected area, such as an area receiving Quo® treatment. In aspects, the amount of composition applied is determined by the affected area. In aspects, about 0.5 mL of a composition, such as, e.g., about 0.25 mL to about 1.5 mL of inventive composition(s) herein can be used to treat an area of about 0.5 cm² to about 20 cm², such as, e.g., ~0.5 cm²-~18 cm², ~0.5 cm²-~16 cm², ~0.5 cm²-~14 cm², ~0.5 cm²-~12 cm², ~0.5 cm²-~10 cm², ~0.5 cm²-~8 cm², ~0.5 cm²-~6 cm², or, e.g., ~0.5 cm²-~4 cm², such as, e.g., ~1 cm²-~20 cm², ~1.5 cm²-~20 cm², ~2 cm²-~20 cm², ~2.5 cm²-~20 cm², ~3 cm²-~20 cm², ~3.5 cm²-~20 cm², or, e.g., ~4 cm²-~20 cm², such as for example ~1 cm²-~15 cm², ~2 cm²-~10 cm², ~3 cm²-~5 cm², or, e.g., ~4 cm².

In aspects, depending on the area to be treated, an amount of about 0.1 mL to about 20 mL, such as, e.g., an amount of between about 0.5 mL to about 15 mL, can be applied depending on the area to be treated. In certain aspects, a single bruise, e.g., an area as small as, for example, about 1 cm² may be treated, such as, e.g., a single bruise resulting from a single injection or a blunt force trauma. In alternative aspects, for example, an area as large as, for example, about 100 cm² or more may be treated, such as, for example, an area receiving Quo® treatment. Therefore, in aspects, depending upon the size of the targeted area of treatment, in aspects, as much as, e.g., 1.2 times, 1.5 times, 2 times, 3 times, 5 times, 10 times, or more composition can be applied to treat a particular indication compared to the amount applied to treat a different indication. In aspects, the total amount of composition applied per dose, and, therefore, the total amount of each ingredient or component within a composition applied per dose, can vary based upon the target area to be treated. Further, in aspects, the number of times per day a composition is applied will impact the total amount of composition and, correspondingly, the total amount of any individual ingredient or component, applied per day. Yet further, in aspects, the treatment period (e.g., the number of days of treatment) will impact the total amount of composition and, correspondingly, the total amount of any individual ingredient or component, applied per treatment period. In aspects, total amounts of composition ingredients/components applied per dose, per day, or, e.g., per treatment period, can be calculated based upon their representative amounts in the composition and the total amount of composition applied.

### Relationship Between Ingredients

In aspects, compositions of the invention can be characterized on the basis of the relationship between two or more ingredients or components of the composition. The proper balance of component(s) of compositions of the invention can be associated with advantageous and unexpected properties in terms of the characteristics or performance of a composition.

In one exemplary aspect, compositions comprise an NADP-MC (e.g., nicotinate compound(s) and nicotinamide compound(s) and a CP-MC (e.g., vitamin K compound(s), such as phytonadione). In some such aspects, the ratio of the NADP-MC to a PCAM, PSAM, or PCPSAM (e.g., a vitamin K compound such as, e.g., vitamin K1) is about 0.25:1-about 6:1, such as, e.g., ~0.5:1-5:1, ~0.75:1-~4:1, or, e.g., ~1:1-~4:1, e.g., ~1.2:1-~5.8:1, ~1.4:1 to ~5.6:1, ~1.6:1 to ~5.4:1, ~1.8:1 to ~5.2:1, or, e.g., ~1.5:1 to ~5:1, ~2:1 to ~5:1, ~2.5:1-~4.5:1, or, e.g., ~3:1-~4:1, such as, ~3.25:1, e.g., about 2.5:1-about 4:1, e.g., 3:1-about 3.5:1.

In aspects, the ratio of at least one NADP-MC constituent to at least one coagulation pathway modulating component constituent (e.g., nicotinate compound:PCAM, PSAM or PCPSAM; or, e.g., tocopheryl nicotinate:vitamin k compound (such as, e.g., vitamin K1)); is about 1:12-about 1:2, ~1:10 to ~1:2, ~1:8 to ~1:2, ~1:6 to ~1:2, ~1:5.5 to ~1:2.5, ~1:5 to ~1:3, or, e.g., about 1:4. In aspects, the composition comprises a nicotinate compound and the ratio of the nicotinate compound component to the PCAM is about 1:6-about 1:2, such as, e.g., about 1:5.5-about 1:2.5, or, e.g., about 1:5-about 1:3.

In aspects, the ratio of at least one NADP-MC constituent to (−) at least one coagulation pathway modulating component constituent (e.g., niacinamide compound: PCAM, PSAM, or PCPSAM (such as, e.g., a vitamin K compound or, specifically, vitamin K1) is about 1:1-about 6:1, such as ~1.5:1 to ~5.5:1, ~2:1 to ~5:1, ~2.5:1 to ~4.5:1, ~2.5:1 to ~4:1, ~2.5:1 to ~3.5:1, ~3:1 to ~3.5:1, or, e.g., ~3:1.

In aspects, the NAD pathway enhancer component comprises a nicotinate compound component and a niacinamide compound component, wherein the nicotinate compound component and niacinamide compound component are present in compositions in a ratio of about 1:4-about 1:300, such as ~1:4 to ~1:250, ~1:4 to ~1:200, ~1:4 to ~1:150, ~1:4 to ~1:100, ~1:4 to ~1:80, ~1:4 to ~1:60, ~1:4 to ~1:40, ~1:5 to ~1:30, ~1:6 to ~1:28, ~1:7 to ~1:26, ~1:6 to ~1:24, ~1:8 to ~1:20, ~1:10 to ~1:15, ~1:10 to ~1:14, or, e.g., about 1:12.

In aspects, the penetration enhancer component comprises two or more constituents. In aspects, the penetration enhancer component comprises a TSD-PEC/decoy constituent (e.g., a mini-hydrolyzed sodium hyaluronic acid compound) and a non-TSD-PEC (e.g., ethyoxydiglycol) constituent. In aspects, the ratio of decoy constituent-to-a non-decoy constituent such as ethyoxydiglycol are present in the compositions in an amount of about 1:1-about 1:15, such as ~1:2 to ~1:15, ~1:3 to ~1:15, ~1:4 to ~1:15, ~1:5 to ~1:15, ~1:6 to ~1:15, or ~1:7 to ~1:15, such as, e.g., ~1:2 to ~1:14, ~1:2 to ~1:13, ~1:2 to ~1:12, ~1:2 to ~1:11, ~1:2 to ~1:10, ~1:2 to ~1:9, ~1:2 to ~1:8, or, e.g., ~1:2 to ~1:7, such as, between ~1:2 to ~1:12, ~1:3 to ~1:11, ~1:4 to ~1:10, ~1:5 to ~1:9, ~1:6 to ~1:8, or, e.g., about 1:7. In aspects, the ratio of decoy constituent-to-total non-decoy constituent such as dimethyl isosorbide are present in the compositions in an amount of about 1:1 to about 1:10, such as ~1:1-~1:8, ~1:1-~1:6, or ~1:1-~1:4, e.g., ~1:1.5-~1:10, ~1:2-~1:10, ~1:2.5-~1:10, or ~1:3-~1:10, such as, for example, ~1:1.5-~1:8, ~1:2-~1:6, ~1:2.5-~1:5, or, e.g., ~1:3-~1:4.

In aspects, the penetration enhancer component comprises two or more non-TSD-PEC constituents (e.g., ethyoxydiglycol and dimethylisosorbide). In aspects, the ratio of a TSD-PEC/decoy constituent (e.g., a mini-hydrolyzed sodium hyaluronic acid compound)-to-the total of the TSD-PEC/decoy constituents is about 1:5 to about 1:15, such as, e.g., ~1:6-~1:15, ~1:7-~1:15, ~1:8-~1:15, or, e.g., ~1:9-~1:15, such as, e.g., ~1:5-~1:14, ~1:5-~1:13, ~1:5-~1:12, or, e.g., ~1:5-~1:11, such as, for example, ~1:6-~1:14, ~1:7-~1:13, ~1:8-~1:12, or, e.g., ~1:9-~1:11.

In aspects, the ratio of the penetration enhancer component to the CP-MC is about 0.2:1-about 4:1, such as between ~0.4:1 to ~4:1, ~0.6:1 to ~4:1, ~0.8:1-4:1, ~1:1 to ~4:1, ~1.2 to ~4:1, ~1.4:1 to ~4:1, ~1.6:1 to ~4:1, or ~1.8:1 to ~4:1, such as ~0.2:1 to ~3.8:1, ~0.2:1 to ~3.6:1, ~0.2:1 to ~3.4:1, ~0.2:1 to ~3.2:1, ~0.2:1-3:1, ~0.2:1 to ~2.8:1, ~0.2:1 to ~2.6:1, ~0.2:1 to ~2.4:1, ~0.2:1 to ~2.2:1, or, e.g., ~0.2:1 to ~2:1, such as between ~1:1 to ~3:1, e.g., ~2:1, ~2.5:1, ~2.7:1, or, e.g., ~3:1.

In aspects, the ratio of the penetration enhancer component to the NAD pathway enhancer component is about 1:0.8-about 1:2.4, such as ~1:0.8 to ~1.2.2, ~1:0.8 to ~1:2, or, e.g., ~1:0.8 to ~1:1.8, such as ~1:1 to ~1:2.4, ~1:1.2 to ~1:2.4, ~1:1.4 to ~1:2.4, or, e.g., between 1:1.4-about 1:1.8, such as about 1:1.6.

### Other Ingredients

Compositions of the invention can comprise any other suitable ingredient(s), such as a carrier (e.g., water), excipients (e.g., preservatives, antioxidants, fragrance agent(s), coloring agent(s), texturizers, the like), or agents that impart other therapeutic or cosmetic effects (independently of or in addition to the effects of a CP-MC, NADP-MC, or both).

In aspects, compositions comprise amino acid(s) or an amino acid component comprising one or more amino acid constituents. In aspects, compositions comprise tryptophan, which, as noted above, can also be considered an element of a NADP-MC. In aspects, compositions comprise an effective amount of one or more other amino acids other than tryptophan. In aspects, compositions are configured for/adapted to topical application and the amino acid is an amino acid that exhibits DOS skin modulating effects. In aspects, the composition comprises one or more basic amino acids. Amino acids that meet one or both of these criteria include, e.g., lysine (Lys), arginine (Arg), and histidine (His). In aspects, the one or more amino acids detectably or significantly increase collagen levels. In aspects, the one or more amino acids detectably or significantly exhibit antioxidant activity. In aspects, the one or more amino acids detectably or significantly increase skin hydration. In aspects, the one or more amino acids detectably or significantly enhance skin healing. In aspects, the one or more amino acids detectably or significantly reduce wrinkles, e.g., reduce fine lines in the skin. In aspects, the one or more amino acids detectably or significantly protect against UV damage. In aspects, the one or more amino acids detectably or significantly enhance activity of the coagulation pathway modulating component.

In aspects, the one or more amino acids detectably or significantly demonstrate a combination of any two or more the following activities: increasing collagen levels, exhibiting antioxidant activity, increasing skin hydration, enhancing skin healing, reducing wrinkles/fine lines, protecting against UV damage, and enhancing activity of the coagulation pathway modulating component.

In aspects, compositions comprise an amount of one or more amino acids selected from (e.g., ≥2, ≥3, ≥5, most, generally all, or all of) arginine, histidine, lysine, methionine, proline, glutamine, Glycine, isoleucine, leucine, proline, tyrosine, and tryptophan. In aspects the one or more amino acids is selected from arginine, histidine, lysine, tryptophan, and combinations of any or all thereof. In aspects, amino acids in compositions comprise modified amino acids or amino acids outside of the twenty typical amino acids expressed in most human proteins. In aspects, some, most, or all of the amino acids in the composition are naturally occurring amino acids, such as the examples provided above.

In aspects, compositions are topical compositions that comprise one or more peptides (compounds comprising 2-40, typically 2-20, e.g., 2-15 or 2-12 amino acid residues, optionally conjugated to other groups/moieties) that induce detectable or significant skin modulation or that enhance the efficacy/effects of one or more aspects of compositions that are applied topically. In one aspect, compositions comprise matricin peptides, peptides that trigger signaling cascade(s), or both. Examples of such peptides include, e.g., carnosine, N-acetylcarnosine, copper tripeptide, trifluoroacetyl-tripeptide-2, tripeptide-10 citrulline, acetyl tetrapeptide-5, acetyl tetrapeptide-9, acetyl tetrapeptide-11, tetrapeptide PKEK, tetrapeptide-21, hexapeptide-11, hexapeptide-14, palmitoyl pentapeptide-4, palmitoyl tripeptide-3/5, palmitoyl tetrapeptide-7, palmitoyl hexapeptide-12, palmitoyl oligopeptide, palmitoyl tripeptide-1, and pentamide-6. In aspects, compositions comprise carrier peptides (peptides that facilitate delivery or stabilization of other elements, such as mineral atoms/ions (e.g., copper or manganese). Examples of such carrier peptides include copper tripeptide and manganese tripeptide-1. In aspects, such peptide(s) include enzyme inhibiting peptides, such as soybean peptide, silk fibroin peptide, or black rice oligopeptides. In aspects, such peptides include structural peptides, such as keratin peptide. In aspects, such peptides include neurotransmitter-inhibiting peptides or peptide mimetics, such as acetyl hexapeptide-3, pentapeptide-18, pentapeptide-3, or tripeptide-3. Details concerning such peptides are provided in, e.g., Schaegen, S. Cosmetics, 2017, 4, 16: doi: 10.3390/cosmetics4020016. Additional examples of peptides suitable and useful for topical application in the treatment of skin disorders, improving skin condition, treatment of skin pain/discomfort, or promotion of cosmetic effects are described in, e.g., WO2016105365, US20120083452, US20070110693, WO2019149450, U.S. Pat. No. 10,463,593, US20030134781, U.S. Pat. Nos. 8,314,065, 11,052,032, 10,668,000, 8,188,053, 8,580,920, US20120083452, U.S. Pat. No. 9,597,274, WO2016105365, U.S. Pat. No. 8,809,276, US20060018851, U.S. Pat. No. 9,687,560, WO2020018926, WO2016033314, U.S. Pat. Nos. 10,603,265, 7,214,655, US20090285770, U.S. Pat. No. 7,025,951, WO2018014936, WO2020018926, and WO2017009487. Such peptides can be linear peptides or cyclic peptides, as appropriate. Such peptides can be derivatized or non-derivatized. Effects of such peptides can include skin color modulation (darkening or lightening, smoothing, skin tightening, reduction of wrinkles/fine lines, etc.). Other peptides can promote the uptake of other agents into the skin. Other topical active peptides can treat diseases or conditions, such as reducing inflammation.

In aspects, compositions comprise an effective amount of one or more peptides that impact neuromodulation properties/functions such as DOS being, comprising an active part/mimetic of (e.g., comprising an active component of SNAP-25), or modulating a neurotransmitter (e.g., inhibiting SNAP-25). For example, in aspects a composition can comprise an effective amount of a SNAP-25 fragment peptide, an analog/fragment, or derivative of any thereof, or a combination of any or all thereof. Known analogs of the SNAP-25 peptide include, e.g., Argireline (Arg0, acetyl hexapeptide-8), Ag1, Arg2, and Arg3. See, e.g., Lim, S. H et al. Sci Rep 8, 1596 (2018). Lubrizol Corporation markets a peptide with similar properties described as an “amplified Argireline peptide” that may be adaptable or applicable in compositions of the invention.

In aspects, compositions comprise an effective amount of one or more antioxidants. For example, in aspects, compositions can comprise an effective amount of resveratrol compound(s). In aspects, compositions can comprise an effective amount of sesquiterpene lactone compound(s). For example, in aspects, compositions can comprise an effective amount of one or more of resveratrol compound(s), an Arnica composition, such as, e.g., an Arnica composition comprising one or more sesquiterpene lactone compound(s), or a combination thereof.

In aspects, compositions comprise an effective amount of one or more exfoliants. In aspects, an exfoliant of the composition can be any pharmaceutically acceptable and cosmetologically acceptable exfoliant. In aspects, an exfoliant of the composition is suitable for topical application. In aspects, an exfoliant of the composition comprises, mostly comprises, or generally consists of, essentially consists of, or consists of salicylic acid.

In aspects, compositions comprise an effective amount of one or more emollients. In aspects, an emollient of the composition can be any pharmaceutically acceptable and cosmetologically acceptable emollient. In aspects, an emollient of the composition is suitable for topical application. In aspects, compositions comprise an effective amount of a squalane emollient. In aspects, the squalane emollient can be a hemisqualane.

In aspects, compositions comprise an amount of one or more fatty acid(s) that are suitable for imparting one or more DOS properties on the composition or ingredients there. In one aspect, fatty acid compounds make up at least about 0.5%, such as at least about 0.75%, or at least about 1 wt. % of the composition, such as, e.g., 2~1 wt. % (e.g., ~1% to ~10%, ~1% to ~7.5%, ~1% to ~5%, or ~1% to ~3.5%), such as ≥~1.5 wt. %, ≥~1.75 wt. %, ≥~2 wt. %, ≥~2.25 wt. %, ≥~2.5 wt. %, ≥~2.75 wt. %, ≥~3 wt. %, ≥~2.5 wt. %, ≥~3 wt. %, ≥~3.5 wt. %, ≥~3.75 wt. %, or ≥~4 wt. % of the composition (e.g., about. In aspects, most, generally all, or all of the fatty acids are medium chain or long chain fatty acids. In aspects, most, generally all, or all of the fatty acids have a chain length of about 7-21 carbons, such as 8-20 carbons, 8-18 carbons, 10-18 carbons, 12-18 carbons, 10-16 carbons, or 12-16 carbon atoms.

In aspects, compositions comprise one or more solvent compound(s)/ingredient(s), which can be any one or more cosmetically acceptable solvents or carriers such as water, oils, or alcohols. Oils can include any type of suitable oil such as coconut oil, olive oil, soybean oil, avocado oil, vegetable oil, hydrogenated vegetable oil, shea butter, and palm oil. Alcohols can include any type of suitable alcohol such as glycerin, propanediol, propanediol 1,3, butylene glycol, hexylene glycol, phenoxyethanol, dipropylene glycol, and propylene glycol. Solvents can also include synthetic compounds such as dimethyl isosorbide, ethoxydiglycol, diethylene glycol monethyl ether, and combination products such as caprylic triglyceride or capric triglyceride.

In aspects, at least about 25 wt. % of a composition provided by the invention is composed of water. In aspects, ≥~28 wt. %, ≥~29 wt. %, ≥~30 wt. %, ≥~31 wt. %, ≥~32 wt. %, ≥~33 wt. 5, ≥~34 wt. %, ≥~35 wt. %, ≥~36 wt. %, ≥~37 wt. %, ≥~38 wt. %, ≥~39 wt. %, or, e.g., ≥~40 wt. % or more of a composition is composed of water (e.g., about 25 wt. %-90 wt. %, about 28 wt. %-80 wt. %, about 30 wt. %-75 wt. %, about 35 wt. %-70 wt. %, about 40 wt. %-65 wt. %, or about 40 wt. %-60 wt. % of the composition is composed of water, which can be a purified water, deionized water, etc.).

In aspects, compositions can comprise ~5 wt. % to ~35 wt. % of one or more of humectant/moisturizing agents or conditioning agents, such as e.g. ~10 wt. % to ~35 wt. %, ~15 wt. % to ~35 wt. %, ~20 wt. % to ~35 wt. %, ~25 wt. % to ~35 wt. %, ~30 wt. % to ~35 wt. %, such as ~5 wt. % to ~30 wt. %, ~10 wt. % to ~30 wt. %, ~15 wt. % to ~30 wt. %, ~20 wt. % to ~30 wt. %, ~25 wt. % to ~30 wt. %, ~5 wt. % to ~25 wt. %, ~10 wt. % to ~25 wt. %, ~15 wt. % to ~25 wt. %, ~20 wt. % to ~25 wt. %, ~5 wt. % to ~20 wt. %, ~10 wt. % to ~20 wt. %, ~15 wt. % to ~20 wt. %, ~5 wt. % to ~15 wt. %, ~10 wt. % to ~15 wt. %, or ~5 wt. % to ~10 wt. %. In further aspects, compositions can comprise ~10 wt. % to ~22 wt. %, such as ~12 wt. % to ~20 wt. % of one or more of humectant/moisturizing agents or conditioning agents.

In aspects, compositions can comprise less than about 0.1 wt. % of one or more humectant/moisturizing agents or conditioning agents, such as for example between about 0.001 wt. %-~0.01 wt. %, or, e.g., 0.01 wt. %-about 1 wt. %, such as 0.01 wt. % to about 0.9 wt. %, 0.8 wt. %, 0.7 wt. %, 0.6 wt. %, or, e.g., 0.5 wt. %. In aspects, compositions can comprise ~0.1 wt. % to ~5 wt. % or ~0.1 wt. % to ~3 wt. %~0.1 wt. % to ~2 wt. %, or ~0.1 wt. %-~1 wt. %, such as ~1 wt. %-5 wt. %, ~1 wt. %-4 wt. %, ~1 wt. %-3 wt. %, ~0.2-~2 wt. %, or ~0.5 wt. %-~1.5 wt. % of one or more of humectant/moisturizing agents or conditioning agents, such as e.g. ~0.1 wt. % to ~4 wt. %, ~0.1 wt. % to ~3 wt. %, ~0.1 wt. % to ~2 wt. %, ~0.1 wt. % to ~1 wt. %, or, e.g., ~0.1 wt. % to ~0.5 wt. %, ~0.5 wt. %-~2.5 wt. %, ~0.5 wt. %-~2 wt. %, or ~0.5 wt. %-~1.5 wt. % of one or more humectant/moisturizing agents or conditioning agents. In aspects, such one or more humectant/moisturizing agents can be present in amount representing about 1 wt. % to about 5 wt. % of the composition, such as, for example, ~1 wt. %-~5 wt. %, ~1 wt. %-~4 wt. %, or ~1 wt. %-~3 wt. %.

In certain aspects, composition scan comprise between about 0.001 wt. %-about 2 wt. % of one or more humectant/moisturizing agents, such as, e.g., ~0.001 wt. %-~1.9 wt. %, ~0.001 wt. %-~1.8 wt. %, ~0.001 wt. %-~1.7 wt. %, ~0.001 wt. %-~1.6 wt. %, or, e.g., 0.001 wt. %-~1.5 wt. %.

In aspects, humectant/moisturizing agents or conditioning agents of a composition/method can be any cosmetically suitable humectant/moisturizing agents or conditioning agents such as alpha-hydroxy acids, salicylic acid, glycerin, hyaluronic acid, urea, panthenol, sodium lactate, glycol, amino acids, fatty acids, butylene glycol, hydrolyzed sodium hyaluronate, lactic acid, lecithin, methyl nicotinate, myristyl alcohol, niacinamide, phytonadione, propanediol, sodium PCA, sodium stearoyl glutamate, sorbitan stearate, squalene, stearyl alcohol, tocopherol, tocopheryl nicotinate, xanthan gum, Glycyrrhiza glabra (licorice) root extract, and Panax notoginseng root extract.

In aspects, compositions can comprise less than about 0.5 wt. % of one or more emollients, such as, e.g., ≤0.45 wt. %, ≤0.4 wt. %, or ≤0.35 wt. %, of one or more emollients, such as between about 0.0001 wt. %-about 0.5 wt. %, ~0.0001 wt. %-~0.45 wt. %, ~0.0001-~0.4 wt. %, or, e.g., ~0.0001 wt. %-~0.35 wt. % of one or more emollients.

In aspects, compositions can comprise ~0.001 wt. %-~2 wt. %, or ~0.001 wt. %-~1.5 wt. %, ~0.01 wt. %-~2 wt. %, ~0.01 wt. %-~1.5 wt. %, or, e.g., ~0.01 wt. %-~1 wt. % of one or more emollients, e.g., ~0.1 wt. % to ~5 wt. % of one or more of emollients, such as e.g. ~0.1 wt. % to ~4 wt. %, or ~0.1 wt. % to ~3 wt. %, ~0.1 wt. % to ~2 wt. %, ~0.1 wt. % to ~1 wt. %, or, e.g., ~0.5 wt. %-2 wt. % of one or more emollients.

In aspects, compositions can comprise ~1 wt. %-~5 wt. % of one or more emollients, e.g., ~1 wt. %-~4 wt. %, or ~1 wt. %-~3 wt. % of the composition emollient(s). In other aspects, compositions can comprise ~5 wt. % to ~25 wt. % of one or more emollients, such as e.g. ~7 wt. % to ~25 wt. %, ~10 wt. % to ~25 wt. %, ~15 wt. % to ~25 wt. %, ~20 wt. % to ~25 wt. %, ~5 wt. % to ~20 wt. %~7 wt. % to ~20 wt. %, ~10 wt. % to ~20 wt. %, ~15 wt. % to ~20 wt. %, ~5 wt. % to ~15 wt. %, ~7 wt. % to ~15 wt. %, ~10 wt. % to ~15 wt. %, or ~5 wt. %-~10 wt. %. In further aspects, compositions can comprise ~12 wt. % to ~18 wt. % such as ~13 wt. % to ~17 wt. % of one or more emollients. In aspects the emollient can be any cosmetically suitable emollient such as petrolatum, zinc oxide, paraffin, mineral oil, glycerin, ethylhexylglycerin, glyceryl stearate, glyceryl stearate citrate, beeswax, olive oil, coconut oil, shea butter, cocoa butter, fatty acids, lecithin, cocoa butter, butyl stearate, diglycol laurate, C12-15 alkyl benzoate, caprylic triglyceride, capric triglyceride, cetyl alcohol, cetearyl alcohol, cetyl ricinoleate, diheptyl succinate, ethylhexyl olivate, myristyl alcohol, and stearyl alcohol.

In aspects, compositions can comprise ~3% to ~20% of one or more stabilizers/emulsifiers used for stabilizing one or more ingredients, oil in water emulsions, water in oil emulsions and pH levels. In aspects, stabilizers can also include preservatives, antioxidants, and chelating agents.

In aspects, compositions can comprise less than about 0.5 wt. % of one or more stabilizers, such as, e.g., ≤0.45 wt. %, ≤0.4 wt. %, or ≤0.35 wt. %, of one or more stabilizers/emulsifiers, such as between about 0.0001 wt. %-about 0.5 wt. %, ~0.0001 wt. %-~0.45 wt. %, ~0.0001-~0.4 wt. %, or, e.g., ~0.0001 wt. %-~0.35 wt. % or ~0.0001 wt. %-~0.1 wt. % of one or more stabilizers/emulsifiers, such as, e.g., ~0.001 wt. %-~2.5 wt. %, ~0.001 wt. %-~2 wt. %, or, e.g., ~0.001 wt. %-~1.5 wt. % of one or more stabilizers.

In aspects, the one or more stabilizers are present in an amount of between about 0.01 wt. % to about 1 wt. %, or, e.g., 0.1 wt. %-about 5 wt. %, such as e.g. ~0.1 wt. % to ~4 wt. %, ~0.1 wt. % to ~3 wt. %, ~0.1 wt. % to ~2 wt. %, ~0.1 wt. % to ~1 wt. %, or, e.g., ~0.1 wt. % to ~0.5 wt. %, ~0.1 wt. % to ~0.1 wt. %, ~0.5 wt. %-~2.5 wt. %, or, e.g., ~0.5 wt. %-~2 wt. %, or ~0.2-~2 wt. %, or ~0.5 wt. %-~1.5 wt. % of the composition.

In aspects, the one or more stabilizers are present in an amount such as, e.g., ~1 wt. %-~5 wt. % or ~1 wt. %-~4 wt. %, such as ~1 wt. %-~3 wt. % of the composition. In aspects, the one or more stabilizers are present in an amount such as e.g. ~5 wt. % to ~20 wt. %, such as ~7 wt. % to ~20 wt. %, such as ~10 wt. % to ~20 wt. %, such as ~15 wt. % to ~20 wt. %, such as ~5 wt. % to ~17 wt. %, such as ~8 wt. % to ~17 wt. %, such as ~10 wt. % to ~17 wt. %, such as ~13 wt. % to ~17 wt. %, such as ~5 wt. % to ~14 wt. %, such as ~7 wt. % to ~14 wt. %, such as ~10 wt. % to ~14 wt. %, such as ~5 wt. % to ~12 wt. %, such as ~7 wt. % to ~12 wt. %, such as ~10 wt. % to ~12 wt. %, such as ~5 wt. % to ~10 wt. %, or such as ~7 wt. % to ~10 wt. %. In further aspects, the one or more stabilizers are present in an amount such as ~4 wt. % to ~8 wt. %, such as ~5 wt. % to ~6 wt. % of the composition.

In aspects the stabilizer can be any cosmetically suitable stabilizer, preservative, antioxidant, or chelating agent such as acetic acid, borax, caprylic/capric triglyceride, carbomer, cetearyl alcohol, cetyl alcohol, cetyl hydroxyethylcellulose, cetyl ricinoleate, citric acid, glyceryl stearate, glyceryl stearate citrate, ethylhexylglycerin, Sensive PA20, lactic acid, lecithin, myristyl alcohol, Optiphen, Optiphen Plus, Peg-100 stearate Pentaerythrityl Tetra-di-t-butyl Hydroxyhydrocinnamate, phenethyl alcohol, phenoxyethanol, potassium sorbate, propanediol, salicylic acid, sclerotium gum, sodium olivate, sodium stearoyl glutamate, sodium hydroxide, sodium lactate, sodium phytate, sorbitan stearate, stearyl alcohol, Siligel, and xanthan gum. Herein, use of the term “carbomer” is used to describe a group of acid-based polymers, anionic in nature, and characterizable as high-molecular-weight, synthetic homo- and copolymers of acrylic acid cross linked with a polyalkenyl polyether (e.g., allyl sucrose or allyl pentaerythritol). While technically the term “carbomer” is a tradename for poly(acrylic acid) or PAA, as has become common in the art, herein the term is used to describe polymers having the formula (CH2-CHCO2-H) n, polymers often sold as ingredient products branded as a “Carbopol”. One of skill in the art will recognize this type of compound as commonly used in the art as, e.g., a thickening agent for water phases, such compounds available in a wide variety of forms, each varying slightly from one another, though all characterizable as described above.

In aspects, compositions can comprise ~0.001 wt. %-~2.5 wt. %, ~0.001 wt. %-~2 wt. %, or, e.g., ~0.001 wt. %-~1.5 wt. %, such as, e.g., ~0.01 wt. % to ~1 wt. %, ~0.1 wt. % to ~5 wt. % or ~0.5 wt. % to ~10 wt. % of one or more thickening agents, such as e.g. about ~0.1 wt. % to ~4 wt. %, about ~0.1 wt. % to ~3 wt. %, ~0.1 wt. % to ~2 wt. %, or about~0.1 wt. % to ~1 wt. %, or ~0.1 wt. % to ~0.5 wt. %, ~0.5 wt. %-~2.5 wt. %, ~0.5 wt. %-~2 wt. %, or, e.g., ~0.5 wt. %-~1.5 wt. %. In aspects, one or more thickening agents can be present in compositions in an amount representing about 1 wt. % to about 5 wt. %, ~1 wt. %-~4 wt. %, or, e.g., ~1 wt. %-~3 wt. % of the composition. In aspects, compositions can comprise ~0.8 wt. % to ~10 wt. %, of one or more thickening agents, such as ~1 wt. % to ~10 wt. %, such as ~2 wt. % to ~10 wt. %, such as ~5 wt. % to ~10 wt. %, such as ~8 wt. % to ~10 wt. %, such as ~0.8 wt. % to ~8 wt. %, such as ~1 wt. % to ~8 wt. %, such as ~2 wt. % to ~8 wt. %, such as ~5 wt. % to ~8 wt. %, such as ~0.8 wt. % to ~5 wt. %, such as ~1 wt. % to ~5 wt. %, such as ~2 wt. % to ~5 wt. %, such as ~3 wt. % to ~5 wt. %, such as ~0.8 wt. % to ~3 wt. %, such as ~1 wt. % to ~3 wt. %, such as ~0.8 wt. % to ~3 wt. %. In aspects, compositions can comprise ~1 wt. % to ~7 wt. %, such as ~2 wt. % to ~6 wt. % of one or more thickening agents.

In aspects the thickening agent can be any cosmetically suitable thickening agent such as waxes such as cetyl alcohol, glyceryl stearate, stearyl alcohol, behenyl alcohol, sorbitan stearate, and stearic acid, gums such as guar gum and xanthan gum, caprylic/capric triglyceride, carbomer, cetearyl alcohol, cetyl hydroxyethylcellulose, magnesium aluminum silica, pullulan, sclerotium gum, silica, and Siligel.

In aspects, compositions can comprise ~0.1 wt. % to ~5 wt. % of one or more texture enhancers, such as e.g. ~0.1 wt. % to ~4 wt. %, ~0.1 wt. % to ~3 wt. %, ~0.1 wt. % to ~2 wt. %, ~0.1 wt. % to ~1 wt. %, e.g., ~0.5 wt. %-~2 wt. % or ~0.4 wt. %-~1 wt. % of one or more texture enhancers. In aspects, compositions can comprise ~1 wt. % to ~8 wt. %, such as, e.g., ~1 wt. %-~7 wt. %, ~1 wt. %-~6 wt. %, or, e.g., ~1 wt. %-~5 wt. % of one or more texture enhancer(s).

In aspects, compositions can comprise ~1 wt. % to ~20 wt. % of one or more texture enhancers, such as e.g. ~2 wt. % to ~20 wt. %, such as ~5 wt. % to ~20 wt. %, such as ~7 wt. % to ~20 wt. %, such as ~10 wt. % to ~20 wt. %, such as ~15 wt. % to 20 wt. %, such as ~2 wt. % to ~18 wt. %, such as ~5 wt. % to ~18 wt. %, 7 wt. % to ~18 wt. %, such as ~10 wt. % to ~18 wt. %, such as ~15 wt. % to 18 wt. %, such as ~2 wt. % to ~15 wt. %, such as ~5 wt. % to ~15 wt. %, 7 wt. % to ~15 wt. %, or such as ~10 wt. % to ~15 wt. %. In further aspects, compositions can comprise ~5 wt. % to ~10 wt. %, such as ~7 wt. % to ~8 wt. % of one or more texture enhancers.

In aspects, the texture enhancer can be any cosmetically suitable texture enhancer such as C12-15 alkyl benzoate, capryloyl glycerin/sebacic acid copolymer, cyclomethicones, diheptyl succinate, ethoxydiglycol, isoamyl laurate, maltodextrin, polyisobutene, polyvinylpyrrolidone, polyvinylpyrrolidone copolymer, propylene glycol stearate, polyquaternium-10, and quaternium-18 hectorite.

In aspects, compositions herein comprise an effective amount of one or more ingredients which provide a detectable or significant cosmetic effect, e.g., a detectable or significant skin smoothing, wrinkle reduction, fine line reduction, erythema reduction, or, e.g., luminosity enhancement effect. In aspects, compositions herein comprise an effective amount of one or more ingredients which provide a detectable or significant reduction in one or more dermatologic diseases or conditions or, e.g., one or more symptoms related to the one or more dermatologic conditions, e.g., seborrheic keratoses (including, e.g., related scaly patches of skin, reddened skin, dandruff, skin dryness, skin flaking, skin peeling, itching etc.), petechia, psoriasis (including, e.g., related skin rash(es) such as skin plaque(s), rash(es) on one or more nails, rash(es) associated with one or more joints, joint pain, skin dryness, skin fissures, skin flaking, skin peeling, skin bumps, thick skin, skin redness, itching, etc.), hyperkeratotic skin, ecchymosis, acne, rosacea, an eczema-related condition (e.g., atopic dermatitis, contact dermatitis, neurodermatitis, dyshidrotic eczema, nummular eczema, seborrheic dermatitis, or statis dermatitis), or, e.g., actinic keratosis (including, e.g., related rough or scaly skin patches, itching, etc.) In aspects, compositions provided by the invention comprise an effective amount of one or more ingredients which provide a detectable or significant reduction in the healing time of skin wound(s) or skin wound-related symptom(s). In aspects, a skin wound can be a scratch, an abrasion, a cut, a blister, a sore, a broken pustule, etc. In aspects, a skin would-related symptom can include an open or otherwise exposed area of a layer of skin which under healthy conditions would not otherwise be exposed to the outside environment, (e.g., exposure of a layer of skin other than the epidermis). In aspects, compositions provided by the invention comprise an effective amount of one or more ingredients which provide a detectable or significant reduction in the healing time, e.g., the time required for a detectable or significant (or, e.g., measurable) reduction in the size, coloration, or pain related to a bruise, hematoma, purpura, or ecchymosis, or, e.g., also or alternatively, the number of bruises or hematomas associated with an injury. In aspects, compositions provided by the invention comprise an effective amount of one or more ingredients which provide detectable or significant prevention of bruising, such as, e.g., bruising associated with one or more medical procedures. In aspects, one or more compounds capable of inducing such effects described in this paragraph are described in the following paragraph.

In aspects, compounds capable of inducing one or more effects described in the preceding paragraph include effective amount(s) of one or more retinoid compounds (compounds derived from retinol (vitamin A) or showing structural, functional, or both structural and functional similarities to retinol. In aspects, a retinoid compound can be any suitable retinoid compound capable of being formulated for safe administration, such as, e.g., safe topical application. In aspects, a retinoid compound is a natural retinoid. In aspects, a retinoid compound is a monoaromatic compound obtained via modification of polar groups at the end and side chain of the polyene vitamin that do not act selectively. In aspects, a retinoid compound is a monoaromatic retinoid. In aspects, a retinoid compound is a synthetic compound wherein the cyclohexene ring is replaced by a benzene ring. In aspects, a retinoid compound is a synthetic analogue of vitamin A. In aspects, a retinoid compound is a polyaromatic retinoid formed via cyclization of polyene side chain and characterized by selective activity toward receptor(s). In aspects, exemplary retinoid compounds can be, e.g., retinol (all-trans retinol), or a derivative thereof. In aspects, a retinoid compound can be any compound having a detectable or significant affinity for retinoid acid receptors and retinoid X receptors. In aspects, a retinoid compound can be, e.g., retinoic acid, e.g., all-trans retinoic acid (tretinoin), retinyl esters (e.g., retinyl acetate, retinyl palmitate), retinaldehyde (retinoic aldehyde), adapalene (naphthalenecarboxylic acid), or, e.g., tazarotene. In aspects, a retinoid compound can be, e.g., retinal, isotretinoin, acitretin, etretinate, 3-dehydroretiol (vitamin A2), 13-cis-retinol, arotinoid, or, e.g., adapalene. Retinol compounds such as these are described in, e.g., Zasada and Budzisz, “Retinoids: active molecules influencing skin structure formation in cosmetic and dermatological treatments,” Postepy Dermatol Alergol, 2019 August; 36 (4):392-397, which, like other cited references herein, is incorporated herein by reference in its entirety.

In aspects, compounds capable of inducing one or more effects described above include effective amount(s) of one or more endocannabinoid compounds, i.e., compounds directly or indirectly detectably or significantly modulating endocannabinoid system-related pathway gene expression. In aspects, compositions or methods can involve/include effective amounts of one or more of endocannabinoids, phytocannabinoids, and cannabinoid receptor agonists and antagonists, inverse agonists, or mimetics of any one or more thereof. In aspects, compound(s) in compositions or methods include compounds that modulate genes in an endocannabinoid or related pathway/system, which are known in the art (see, e.g., Zimmer A. Handb Exp Pharmacol. 2015; 231:129-83). In aspects, compositions comprise an effective amount of one or more endocannabinoid compound(s) or compounds that enhance the activity/action of endocannabinoid compound(s) or provide similar effects (e.g., anti-inflammatory effects) as endocannabinoid compound(s). Such compounds can be from cannabis (e.g., CBD), can mimic cannabinoid compounds, or can be compounds that are considered endocannabinoid compounds in broad construction but that can be or are sourced (when sourced naturally) from other sources than cannabis. In aspects, either of such type of compounds detectably or significantly modulates a CB1 receptor or a CB2 receptor. Examples of such compounds include, e.g., curcumin, B-caryophyllene, N-palmitoylethanolamide (PEA), honokiol, magnalol, 7-hydroxyflavone, triptolide, ginkolide, docosahexaenoic acid (DHA), eicosapentaenoic acid (EPA), rutamarin, eugenol, menthol, camphor, methyl salicylate, cannabidiol, disophenol, isomenthone, menthone, limonene, salvinorin A, triterpene alcohols, or triterpendiol monoesters including faradiol esters, myristicin, sabinen, terpineol, a-pinene, limonen, and terpinene. In aspects, compositions comprise an anti-inflammatory endocannabinoid system-related pathway compound such as, e.g., a compound affecting PPARg, PPARa, and PPARb, such as, e.g., cannabidiol, ajulemic acid, B-caryophyllene, apigenin, daidzein, genestein, quercetin, astaxanthin, beta carotene, lycopene, N-acetyl L-cysteine (NAC), diosphenol, isomenthone, menthone, limonene, rosmarinic acid, t-reservatrol, triptolide, myristicin, honokiol, magnalol, carvacrol, thymol, eugenol, DHA, EPA, a-Lipoic acid, curcumin, ginkolide, methyl salicylate, camphor, cinnamaldehyde, or capsaicin. Additional examples of such compounds include, e.g., epigallocatechin gallate, (−)a-bisabolol, lycopene, N-acetyl L-cysteine, perilloxin, perilla anthocyanin, t-reservatrol, verbascoside, echinoscoside, carnosine, pycnogenol, triptolide, a-pinene, actanol, linalool, octyl acetate, bornyl acetate, incensole, linalool, sesqueterpene lactones, propofol, honokiol, magnolol, eugenol, diosphenol, isomenthone, eucalyptol, menthol, and methyl salicylate. Still other examples of such compounds can include, e.g., ferulic acid, chlorogenic acid, cafeic acid, quinic acid, capsanthin, carnosine, L-ergothioneine, cannabidiol, 3,3-diindolylmethane (DIM), tetrahydrocurcurmin, t-resveratrol, carvacrol, thymol, allicin, camphor, eucalyptol, camphene, β-pinene, borneol, thujone, krill oil, fish oil, menthol, methyl salicylate, carvacrol, thymol, or linalool. Additional examples of such compounds, and classes of other and examples of other related compounds that can make up part of a composition of the invention or can be used in methods of the invention are described in, e.g., WO2020094555, WO2019236607, EP3684353A1, U.S. Pat. Nos. 10,143,639, 9,724,311, US20180369171, US20150104503, and US20200155635, as well as in Río C D et al. Biochem Pharmacol. 2018 November; 157:122-133; Bíró T, et al. Pharmacol Sci. 2009 August; 30 (8):411-20; Mounessa J S, et al. J Am Acad Dermatol. 2017 July; 77 (1):188-190; Gupta A K, Talukder M. J Cosmet Dermatol. 2021 September; 20 (9):2703-2711; Marks D H, Friedman A. Skin Therapy Lett. 2018 November; 23 (6):1-5; Avila C, et al. J Am Acad Dermatol. 2020 May; 82 (5):1205-1212; Osafo N et al. Mol Biol Rep. 2021 April; 48 (4):3665-3680; Martinelli G, et al. Planta Med. 2021 Apr. 13. doi: 10.1055/a-1420-5780; Fowler C J. Curr Drug Targets CNS Neurol Disord. 2005 December; 4 (6):685-96; Joshi N, Onaivi E S. Adv Exp Med Biol. 2019; 1162:1-12; and Lu H C, Biol Psychiatry Cogn Neurosci Neuroimaging. 2021 June; 6 (6):607-615. In aspects, compositions or methods comprise effective amount(s) of other bioactive materials, such as microbiome compositions having detectable or significant effects on any of the conditions described herein, examples of which compositions are known in the art (see, e.g., Yang E J, et al. Dermatol Ther. 2019 November; 32 (6): e13101).

### Optionally Excluded/Included Ingredients

In aspects, compositions of the invention lack one or more ingredients, such as ingredients that may change the physiochemical properties, performance characteristics, or other characteristics/activities of a composition. In other aspects, compositions can contain one or some of the compositions/compounds described in this section as optional ingredient(s).

In aspects, compositions provided by the invention contain less than 5%, less than 2%, less than 1%, less than 0.5%, less than 0.25%, or lack/essentially lack any vitamin ingredient derived from a vitamin that has a molecular weight of about 135-400 g/Mol. In aspects, compositions contain less than 5%, less than 2%, less than 1%, less than 0.5%, less than 0.25%, or lack/essentially lack any vitamin ingredient that has a molecular weight of about 135-400 g/Mol. In aspects, compositions contain less than 5%, less than 2%, less than 1%, less than 0.5%, less than 0.25%, or lack/essentially lack any vitamin ingredient derived from a vitamin that has a molecular weight of greater than about 550 g/Mol. In aspects, compositions contain less than 5 wt. %, less than 2 wt. %, less than 1 wt. %, less than 0.5 wt. %, less than 0.25 wt. %, or lack/essentially lack any vitamin ingredient that has a molecular weight of ≥~550 g/Mol.

In certain aspects, compositions do not contain more than about 0.5 wt. %, such as, e.g., does not contain ≥~0.6 wt. %, ≥~0.7 wt. %, ≥~0.8 wt. %, ≥~0.9 wt. %, ≥~1 wt. %, ≥~1.1 wt. %, ≥~1.2 wt. %, ≥~1.3 wt. %, ≥~1.4 wt. %, or, e.g., ≥~1.5 wt. % of any hyaluronic acid derivative, such as any hyaluronic acid ester. In aspects, compositions do not contain more than about 1 wt. % of any hyaluronic acid derivative, such as any hyaluronic acid ester. In aspects, compositions are free of any hyaluronic acid derivative, such as any hyaluronic acid ester.

In aspects, compositions do not comprise more than about 0.2 wt. % of any hyaluronic acid ingredient or composition having an average molecular weight of greater than about 25,000 Da, such as, e.g., do not comprise more than ~0.3 wt. %, ~0.4 wt. %, ~0.5 wt. %, ~0.6 wt. %, ~0.7 wt. %, ~0.8 wt. %, ~0.9 wt. %, ~1 wt. %, ~1.1 wt. %, ~1.2 wt. %, ~1.3 wt. %, ~1.4 wt. %, ~1.5 wt. %, ~1.6 wt. %, ~1.7 wt. %, ~1.8 wt. %, ~1.9 wt. %, or, e.g., does not comprise more than ~2 wt. % of any hyaluronic acid ingredient or composition having an average molecular weight of greater than about 35,000 Da. In aspects, compositions do not comprise more than about 1 wt. %, such as, e.g., more than 0.5 wt. %, of any hyaluronic acid composition having an average molecular weight of greater than about 25,000 Da. In aspects, compositions are free of any hyaluronic acid composition or ingredient having an average molecular weight of greater than about 25,000 Da.

In aspects, compositions of the invention do not contain more than about 2 wt. %, more than ~1 wt. %, more than ~0.5 wt. %, more than ~0.2 wt. %, more than ~0.1 wt. %, more than ~0.05 wt. %, or more than ~0.01 wt. % of, or are free/essentially free of one or more or all vitamin A composition(s)/compound(s) such as a retinol, retinoic acid, isotretinoin, etc.

In aspects, compositions of the invention do not contain more than about 2 wt. %, more than ~1 wt. %, more than ~0.5 wt. %, more than ~0.2 wt. %, more than ~0.1 wt. %, more than ~0.05 wt. %, or more than ~0.01% of, or are free/essentially free of vitamin E in any free (non-conjugated/bound) form.

In aspects, compositions of the invention do not contain more than about 2 wt. %, more than ~1 wt. %, more than ~0.5 wt. %, more than ~0.2 wt. %, more than ~0.1 wt. %, more than ~0.05 wt. %, or more than ~0.01 wt. % of, or are free/essentially free of vitamin C in any free (non-conjugated/bound) form.

In aspects, compositions of the invention do not contain more than about 2 wt. %, more than ~1 wt. %, more than ~0.5 wt. %, more than ~0.2 wt. %, more than ~0.1 wt. %, more than ~0.05 wt. %, or more than ~0.01 wt. % of, or are free/essentially free of any SARM1 inhibitor.

In aspects, compositions of the invention do not contain more than about 2 wt. %, more than ~1 wt. %, more than ~0.5 wt. %, more than ~0.2 wt. %, more than ~0.1 wt. %, more than ~0.05 wt. %, or more than ~0.01 wt. % of, or are free/essentially free of caffeine, emu oil, or both.

In aspects, compositions provided by the invention are detectably or significantly free of iron. In aspects, an NAD pathway modulation compound can be the only component or ingredient in a composition which comprises a metal ion or metallic compound.

In aspects, compositions provided by the invention are detectably or significantly free of, or are free of, one or more growth hormone compounds. In aspects, compositions are free/essentially free of growth hormone proteins/peptides.

In aspects, compositions provided by the invention are detectably or significantly free of, or are free of, one or more chitosan compounds. In aspects, compositions are free/essentially free of chitosan.

In aspects, compositions provided by the invention are detectably or significantly free of, or are free/essentially free of, one or more psyllium compounds. In aspects, compositions are free of psyllium.

In aspects, compositions provided by the invention are detectably or significantly free of, or are free of, one or more whole-cell products or whole cells (meaning an intentional incorporation of whole cells or whole cell products as an ingredient). In aspects, compositions are free of whole cells.

In aspects, no more than about 2 wt. %, such as, e.g., ≤~1.95 wt. %, ≤~1.9 wt. %, ≤~1.85 wt. %, ≤~1.8 wt. %, ≤~1.75 wt. %, ≤~1.7 wt. %, ≤~1.65 wt. %, ≤~1.6 wt. %, ≤~1.55 wt. %, ≤~1.5 wt. %, ≤~1.45 wt. %, ≤~1.4 wt. %, ≤~1.35 wt. %, ≤~1.3 wt. %, ≤~1.25 wt. %, ≤~1.2 wt. %, ≤~1.15 wt. %, ≤~1.1 wt. %, ≤~1.05 wt. %, ≤~1 wt. %, ~0.95 wt. %, ~0.9 wt. %, ≤~0.85 wt. %, ≤~0.8 wt. %, ≤~0.75 wt. %, ≥~0.7 wt. %, ~0.65 wt. %, ~0.6 wt. %, ≤~0.55 wt. %, or, e.g., ≤~0.5 wt. % of a composition is made up of any cellulose compound. In aspects, no more than about 1 wt. %, ~0.75 wt. %, or ~0.5 wt. % of the composition is made up of any cellulose compound.

In aspects, no more than about 2 wt. %, such as, e.g., ≤~1.95 wt. %, ≤~1.9 wt. %, ≤~1.85 wt. %, ≤~1.8 wt. %, ≤~1.75 wt. %, ≤~1.7 wt. %, ≤~1.65 wt. %, ≤~1.6 wt. %, ≤~1.55 wt. %, ≤~1.5 wt. %, ≤~1.45 wt. %, ≤~1.4 wt. %, ≤~1.35 wt. %, ≤~1.3 wt. %, ≤~1.25 wt. %, ≤~1.2 wt. %, ≤~1.15 wt. %, ≤~1.1 wt. %, ≤~1.05 wt. %, ≤~1 wt. %, ~0.95 wt. %, ≤~0.9 wt. %, ≤~0.85 wt. %, ≤~0.8 wt. %, ≤~0.75 wt. %, ≤~0.7 wt. %, ≤~0.65 wt. %, ≥~0.6 wt. %, ≤~0.55 wt. %, or, e.g., ≤~0.5 wt. % of a composition is made up of any non-vitamin antibacterial agent compound. In aspects, no more than about 1 wt. %, ~0.75 wt. %, or ~0.5 wt. % of the composition is made up of any non-vitamin antibacterial agent compound.

In aspects, composition(s) lack an amount of one or more minerals that detectably or significantly aid in the intended effect of the composition, such as copper, zinc, or both. E.g., in aspects, compositions comprise no more than about 2 wt. %, such as, e.g., ≤~1.95 wt. %, ≤~1.9 wt. %, ≤~1.85 wt. %, ≤~1.8 wt. %, ≤~1.75 wt. %, ≤~1.7 wt. %, ≤~1.65 wt. %, ≤~1.6 wt. %, ≤~1.55 wt. %, ≤~1.5 wt. %, ≤~1.45 wt. %, ≤~1.4 wt. %, ≤~1.35 wt. %, ≤~1.3 wt. %, ≤~1.25 wt. %, ≤~1.2 wt. %, ≤~1.15 wt. %, ≤~1.1 wt. %, ≤~1.05 wt. %, ≤~1 wt. %, ≤~0.95 wt. %, ≤~0.9 wt. %, ≤~0.85 wt. %, ~0.8 wt. %, ~0.75 wt. %, ~0.7 wt. %, ~0.65 wt. %, ~0.6 wt. %, ≤~0.55 wt. %, or, e.g., ≤~0.5 wt. % of zinc. In aspects, compositions comprise no more than about 2 wt. %, such as, e.g., ≤~1.95 wt. %, ≤~1.9 wt. %, ≤~1.85 wt. %, ≤~1.8 wt. %, ≤~1.75 wt. %, ≤~1.7 wt. %, ≤~1.65 wt. %, ≤~1.6 wt. %, ≤~1.55 wt. %, ≤~1.5 wt. %, ≤~1.45 wt. %, ≤~1.4 wt. %, ≤~1.35 wt. %, ≤~1.3 wt. %, ≤~1.25 wt. %, ≤~1.2 wt. %, ≤~1.15 wt. %, ≤~1.1 wt. %, ≤~1.05 wt. %, ≤~1 wt. %, ≤~0.95 wt. %, ≥~0.9 wt. %, ≥~0.85 wt. %, ~0.8 wt. %, ~0.75 wt. %, ≤~0.7 wt. %, ≤~0.65 wt. %, ~0.6 wt. %, ~0.55 wt. %, or, e.g., ≤~0.5 wt. % of copper. In aspects, compositions can comprise compound(s) which comprise a copper or zinc component (e.g., a nicotinate salt such as niacin copper (2+) salt), but such minerals alone do not represent such amounts of a composition.

In aspects, no more than about 2 wt. %, such as, e.g., ≤~1.95 wt. %, ≤~1.9 wt. %, ≤~1.85 wt. %, ≤~1.8 wt. %, ≤~1.75 wt. %, ≤~1.7 wt. %, ≤~1.65 wt. %, ≤~1.6 wt. %, ≤~1.55 wt. %, ≤~1.5 wt. %, ≤~1.45 wt. %, ≤~1.4 wt. %, ≤~1.35 wt. %, ≤~1.3 wt. %, ≤~1.25 wt. %, ≤~1.2 wt. %, ≤~1.15 wt. %, ~1.1 wt. %, ≤~1.05 wt. %, ≤~1 wt. %, ≤~0.95 wt. %, ≤~0.9 wt. %, ≤~0.85 wt. %, ≤~0.8 wt. %, ~0.75 wt. %, ~0.7 wt. %, ≥~0.65 wt. %, ≤~0.6 wt. %, ≤~0.55 wt. %, or, e.g., ≤~0.5 wt. % of a composition is made up of any cellulose compound or any non-vitamin bacterial agent.

In certain aspects, compositions do not contain more than about 0.5 wt. %, such as, e.g., does not contain ≥~0.6 wt. %, ≥~0.7 wt. %, ≥~0.8 wt. %, ≥~0.9 wt. %, ≥~1 wt. %, ≥~1.1 wt. %, ≥~1.2 wt. %, ≥~1.3 wt. %, ≥~1.4 wt. %, or, e.g., ≥~1.5 wt. % of any tripeptide or hexapeptide originating from the fragmentation of matrix proteins exhibiting DOS biological activity. In aspects, compositions do not contain more than about 1% of any tripeptide originating from the fragmentation of matrix protein proteins exhibiting DOS biological activity. In aspects, compositions are free of any such tripeptides. In aspects, compositions do not contain more than about 1% of any hexapeptide originating from the fragmentation of matrix proteins exhibiting DOS biological activity. In aspects, compositions are free of any such hexapeptides. In aspects, compositions to not contain more than about 0.5 wt. %, such as, e.g., does not contain ≥~0.6 wt. %, ≥~0.7 wt. %, ≥~0.8 wt. %, ≥~0.9 wt. %, ≥~1 wt. %, ≥~1.1 wt. %, ≥~1.2 wt. %, ≥~1.3 wt. %, ≥~1.4 wt. %, or, e.g., ≥~1.5 wt. % of any tripeptide/hexapeptide combination product wherein the peptides originate from the fragmentation of matrix proteins exhibiting DOS biological activity. In aspects, compositions do not contain more than about 1% of any tripeptide/hexapeptide combination product wherein the peptides originate from the fragmentation of matrix proteins exhibiting DOS biological activity. In aspects, compositions are free of such tripeptide/hexapeptide combination product(s). In aspects, compositions comprise less than 1% of a component characterizable as “TriHex™” technology (also referred to as “TriHex Technology™”; ALASTIN Skincare, Inc. Carlsbad, CA). In aspects, compositions are free of any component characterizable as “TriHex™” technology (or “TriHex Technology™”).

In further aspects, compositions provided by the invention do not contain more than about 2 wt. %, more than ~1 wt. %, more than ~0.5 wt. %, more than ~0.2 wt. %, more than ~0.1 wt. %, more than ~0.05 wt. %, or more than ~0.01 wt. % of, do not comprise any detectable or significant amount of (e.g., do not comprise sufficient amount so as to impart a detectable or significant effect), or are free/essentially free of any one or more of the following: (S)-Equol, 1,2-propyleneglycol, 1 cetyl ester, 1-(3,7,11-trimethyldodecyl) azacyclohaptane-2-one, 1,2,6-hexanetriol, 1,2-propanediol, 1,3-butanediol, 1,3-butylene glycol, 1,3-dioxolane, 1,3-PDTA ferric complex salts, 1,3-propanediol, 1,4-glucopyranoside, 1,8-cineole, polysorbate 80, isopropyl alcohol (or alternatively ethanol), 11-trans conjugated linoleic acid and 10-trans, 12-cis conjugated linoleic acid isomers (conjugated linoleic acid, CLA), 12-octadecadiynoic acid, 194N-alkyltaurines. 1-dodecylazacycloheptane-2-one (e.g., Azone®), 1-farnesylazacycloheptan-2-one, 1-geranylazacycloheptan-2-one, 1-geranylazacyclopentan-2,5-dione, 1-geranylgeranylazacycloheptan-2-one, 1-hexyl-4-carboxy-2-pyrrolidone, 1-hexyl-4-methoxycarbonyl-2-pyrrolidone, 1-lauryl-2-pyrrolidone, 1-lauryl-4-carboxy-2-pyrrolidone, 1-lauryl-4-ethoxycarbonyl-2-pyrrolidone, 1-methyl-2-pyrrolidone, 1-methyl-4-methoxycarbonyl-2-pyrrolidone, 2-(n-nonyl)-acetylacetone iron complex salt, 2,4-dihydroxy acetophenone (resacetophenone), 2,6-dihydroxy acetophenone, 2-acetylpyridine N-oxide, 2-bromo2-nitropropane-1,3-diol, 2-chloronicotinamide, 2-ethylhexaneic acid, 2-ethylhexyl-2-cyano-3,3-diphenylacrylate, 2-ethylhexyl isononoate, 2-ethyl-hexyl salicylate, 2-ethylhexyl stearate, 2-mercaptonicotinic acid, 2-phenoxyethanol, 2-phenylbenzimidazole-5-sulfonic acid, 2-pyrrolidone, 3,4-dihydroxycinnamic acid, 3-amino-1-(m-(trifluoromethyl)phenyl)-2-pyrazoline, 4,5-dehydro docosahexaenoic acid, 5,6-dehydro arachidonic acid, 5-HETE-lactone, 6,9-diepoxy-6,9-phenylimino-delta 6,8-prostaglandin I, 6-aminonicotinamide, 7-dehydrocholesterol, 9,12-octadecadiynoic acid, abrasives, absorbents, acai berry oil, acetamide MEA, acetate, acetate/crotonate copolymer, acetone insoluble, acetyl dipeptide 1 cetyl ester, acetyl dipeptide 3 aminohexanoate, acetyl dipeptide-1, acetyl glucosamine, acetyl group, acetylated derivatives, acidity regulators, Acorns gramineus, acrylamide copolymer, acrylamide/sodium acrylate copolymer, acrylate copolymer, acrylate/ammonium methacrylate copolymer, acrylates/C10-30 alkyl acrylate crosspolymer, acrylic acid polymers, acrylic/acrylate copolymer, acryloyl methyl taurate-vinylpyrrolidone copolymers, actiplex (extract of various plants), actiplex 2789 (extract of various plants), acyl amino, acyl-derivatives, adenosine diphosphate (ADP), adenosine triphosphate (ATP), adenosine, adipic acid/dimethylaminohydroxypropyl diethylenetriamine copolymer, adipic acid/epoxypropyl diethylenetriamine copolymer, adrenergic nerve inhibitors, adriamycin iron complex, Aesculus hippocastanum (Horse Chestnut) bark extract, Aesculus hippocastanum bark, agar, agarose, alchemilla, alcohols (e.g. α-terpineol, terpinen-4-ol, carvol, etc.), alcohols (octyldodecanol), algae extract, algin, alginate salts, aliphatic alcohols, alkanones, alkenyl acids, alkyl benzoate, alkyl fatty acid esters such as ethyl acetate, alkyl galactmanans (available under the trade name N-Hance® from Hercules), alkyl polysaccharide (APS), alkyl resins, alkyl sulfuric acid salts, alkyl-2-(N,N-disubstituted amino)-alkanoate ester, alkylene glycols, alkylenediamine-N,N′-disuccinic acid iron (III) complex, alkylmethyl siloxanes, allantoin, allantoin ascorbate, methyl nicotinate, allyl stearate/VA copolymer, almond oil, aloe vera, alpha hydroxy acids, alpha-Lipoic acid, alpha-adrenergic receptor antagonist, alpha-Pentyl-3-(2-quinolinylmethoxy)-benzenemethanol, alpha-spinasterol, alumina flake, alumina, aluminium starch, aluminum starch octenylsuccinate, amides, amidobetaines, amidosulfobetaines, Amilite® GCS-11 (Sodium cocoyl glycinate), amine oxides, amino acid amides, amino acids, aminoalkyl methacrylate copolymer RS 2.0, aminobenzoic acid, aminoethylacrylate phosphate/acrylate copolymer, Minofect® (surfactin peptide-amide/ester), aminofoam WOR, aminoglycoside-iron complex, aminopropyl ascorbyl phosphate, Amisoft® CS11-F, Amisoft® LS1 1-F, Amisoft® MS11-F, Amisoft® CS-11 (F) (Sodium cocoyl glutamate), Amisoft® GS-11 P (F) (Sodium stearoyl glutamate/Sodium cocoyl glutamate (mix), Amisoft® HS-1 1 P (F) (Sodium stearoyl glutamate), Amisoft® LS-11 (F) (Sodium lauroyl glutamate), Amisoft® MS-1 1 (F) (Sodium myristoyl glutamate), ammonium acrylate copolymer, ammonium acryloyl dimethyl taurate/vinyl pyrolidone copolymer, ammonium cocoyl sulfate, ammonium lactate, ammonium laureth sulfate, ammonium lauryl sulfate, ammonium vinyl acetate/acrylate copolymer, amni visnaga extract, amorolfine, AMP acrylate/diacetoneacrylamide copolymer, AMPD acrylate/diacetoneacrylamide copolymer, amphoteric surfactants, amyl dimethyl PABA, amylopectins, amylose, analgesics, andrographolide (Andrographis paniculata), androsterone, anesthetics, anhydrous zeolites, anhydroxylitol, anionic inorganic species (e.g., chloride, bromide, iodide, carbonate, preferably chloride), anionic surface-active agents, anionic surfactants (amino acid amides), anionic surfactants (esters of alpha-hydroxycarboxylic acids), anionic surfactants (fatty acids), anionic surfactants (phosphate based), anionic surfactants (surfactin), anthemis nobilis, anthracycline-iron complex, anthroquinone, anti-acne agents, anti-atrophy actives, antibacterials, anti-caking agents, anti-cellulite agents, anti-dandruff actives, antifoaming agents, antifungals, anti-inflammatory agents, antimicrobial agents (e.g., iodopropyl butylcarbamate), anti-oxidants (e.g. alpha-tocopherol, butylated hydroxyanisole (BHA) antiperspirant actives, anti-tack agents, apigenin, Apricosal (AFL-3607/E), apricot kernel oil, arabinans, arabinogalactans, arabinose, arabinoxylans, arbutin, arginine ascorbate, Argireline® (acetyl hexapeptide-3), Aristoflex® AVC (Clariant), arlacel, Arlasilk™ EFA (Phospholipid EFA), Arlasilk™ PTC (Phospholipid PTC), Arlasolve™ 200N (lsoceteth-20), aromatic hydrocarbon resin, Artemisia dracunculus, Ascophyllum Nodosum, ascorbate, ascorbic acid, ascorbic acid derivatives, ascorbyl dipalmitate, ascorbyl esters of fatty acids, ascorbyl glucosamine, ascorbyl glucoside, ascorbyl palmitate, ascorbyl phosphates, ascorbyl sorbate, ascorbyl tetraisopalmitate, asiaticoside (Centella asiatica extract), aspergillus orizae extract (aspergillus orizae), astaxanthin (Haematococcus algae), avobenzene, avocado butter, avocado oil, azaftig, azelaoyl bisdipeptide 10, B1 to B12 compounds, Baeomycesic acid, baicalein, basil, BaSO4, borosilicate, BAY 108888, bayberry wax, BB-20 (Beheneth-20), BC-20TX (Ceteth-20), beads, bearberry extract (Arctostaphylos uva ursi), beech oil, beeswax, behentrimonium chloride, behenyl alcohol, bentonite clay, benzalkonium chloride, benzodiazepines, benzoic acid, benzophenone-3, benzophenone-4, benzyl alcohol, benzyl niacin ascorbate, benzyl niacin lactate, benzyl niacin mandelate, benzyl nicotinate, benzyl or short chain alkyl, berberine, bergamot, beta hydroxy acids, beta-adrenergic receptor agonists, beta-alkyloxy alkane sulfonates, beta-cyclodextrin, betamethasone 17-valerate, beta-sitosterol, bifonazole, bile salts, biliverdin-iron complex, binders, buffering agents, biodegradable cyclic urea (e.g., 1-alkyl-4-imidazoline-2-one), biotin, biotinyl group, bisabolol, bismuth oxychloride, black tea extract (Andrographis paniculata), Blanose® 7HF bleomycin-iron complex, block copolymers, block polymers of ethylene oxide and propylene oxide (for example, those available from BASF Wyandotte under the trade name “Pluronic”®), block polymers of ethylene oxide and/or propylene oxide, blood microcirculation improvement agents (vasodilatory or vasoconstrictive), BO-15V (Oleth-15), boron nitride, Boswellia (EUK) dry, Boswellia (frankincense), Boswellia extract (Boswellia serrata), Boswellia serrata (β-boswellic acids), Boswellin® CG, botanical extracts, brassicasterol, brazil nut oil, Brij® 35 (Laureth-23), Brij® 56 (Ceteth-10), Brij® 58 (Ceteth-20), Brij® 58P (Ceteth-20), Brij® 76 (Steareth-10), Brij® 30, Span® 20, Brij® 93 (polyoxyethylene (2) oleyl ether), Brij® 96 (polyoxyethylene (20) oleyl ether), Brij® 99 (polyoxyl (10) oleyl ether), broken seed nut shells, bromfenac, bromide, bromocresol green (tetrabromo-m-cresolsulfonphthalein sulfone), bulking agents, butaconazole, butanediol, butenafine, butyl acetate, butyl ester of ethylene/maleic anhydride copolymer, butyl ester of PVM/MA copolymer, butyl rubber, butylated hydroxytoluene (BHT), C10-C18 alkyl triglycerides, C11-15 pareth-12, C11-15 pareth-15, C11-15 pareth-20, C12-15 alkyl benzoate, C12-C13 pareth-23, C12-C18 alkyl triglycerides, C30-45 alkyl methicone, CaCO3, caffeine, calamine, Calamintha sylvatica, calcipotriol, calcium carbonate, calcium channel blockers, calcium citrate, calcium disodium edetate, calcium gluconate, calcium hydroxycitrate, calcium lactate, calcium phosphate, calcium salts, calcium sulfate, calcium, calcium/sodium PVM/MA copolymer, Camelina sativa oil (family Brassicaceae, e.g. Camelina sativa, Gold of Pleasure, False Flax, etc.), camellia seed oil, camp-dependent protein kinase activators, campesterol, camphor (D-camphor), candelilla wax, canola oil, capaicin, capric acid (decanoic acid), capric triglyceride, caprylic triglycerides, caprylyl glycol, capsaicin (sabinsa), capsaicinoids USP, capsaicin (sigma) 8-methyl-N-vanillyl-trans-6-nonenamide, capsicum annum, capsicum oleoresin, carageenans, caraway, Carbopol®, Carbopol® Aqua-SF1, Carbopol®980, carbowax, carboxy, carboxylic acid polymers, carboxymethyl cellulose, carboxymethyl guar gum, carboxymethyl(hydroxypropyl), carnitine, carnosine ascorbate, carnosine, niacinamide, carotenes, carotenoids (pro-vitamin A), carrageenan, carrot seed oil, casein, cashew nut oil, CaSO4, castor oil, cationic guar gum, cationic surfactants (PEG alkyl amines), cationic surfactants (synthetic phospholipids), cationic, cedarwood, cellulose acetate butyrate, cellulose acetate, cellulose ethers including methyl cellulose, cellulose, Centella asiatica extract, ceramics, Cercidiphylum japonicum, ceresin, cetalkonium chloride, ceteareth-20, ceteareth-25, ceteareth-30, cetearyl glucoside, ceteth-10 phosphate, ceteth-10, ceteth-15, ceteth-20, cethethyldimonium bromide, cethylpyridinium chloride, cetyl alcohol, cetyl dimethicone, cetyl dimethicone copolyol, cetyl dimethyl carboxymethyl betaine, cetyl ester, cetyl hydroxyethycellulose, cetyl lactate, cetyl palmitate, cetyl propionate, cetyl trimonium chloride, cetylpyridinium chloride, cetyltrimethyl ammonium bromide, CF-600, chamomile extract, chamomile, cha-plu, chelating agents, chenopodium, cherry kernel oil, chia oil, chitosan, chitosan ascorbate, chitosan azelate, chitosan glycolate, chitosan lactate, chitosan mandelate, chitosan pyrrolidone carboxylate, chitosan salicylate chitosan, chloride, chlorinated rubber, chlorogenic acid (Vaccinium vulgaris), chlorphenesin, cholecalciferol (vitamin D3), cholesterol sulphate, cholesterol, cholin, cholinergic modulators, chondroitin sulfate, chondroitin ascorbate, chondroitin, chitins, chromium hydroxide, chromium oxide, chromium picolinate, ciclopirox olamine, ciclopirox, Cimicifuga racemose, cineol, cinnamon bark, cinnamon, cinnamyl-3,4-dihydroxy-a-cyanocinnamate, cinoxate, cithrol 10 MS (PEG-20 stearate), Citrus aurantium, clays, clobetasol propionate, clotrimazole, clove, cobalamins (vitamin B12), cocamidopropyl betaine, coco dimethyl carboxymethyl betaine, coco dimethyl sulfopropyl betaine, cocoa butter, cocoamidopropyl betaine, cocobetaine, cocodimonium hydroxypropyl oxyethyl cellulose, coconut alkyl triethylene glycol ether sulfate, coconut oil fatty acid, cocotrimonium chloride, cocoyl sarcosine, cognis hydagen CMF (chitosan & water), Coleus forskohlii extract, collagen and elastin synthesis boosters, colloidal oat protein, colloidal oatmeal, colloids, colominic acid ([poly-N acetyl-neuraminic acid]), colorants, concentrates of plant extracts, conditioning agents, conjugated estrogen, copolymer, copolymers Carbopol®, copper adenosine triphosphate, copper peptides (GHK-Cu), cordia schomburgkii extract, corn oil, corn starch/acrylamide/sodium acrylate copolymer, cosmetic astringents, cosmetic biocides, Cosmoperine®, cotton fibers, cottonseed oil, coumarin esculoside (esculin), coumaroyl dipeptide 3, creatine ascorbate, creatine, Crithmum maritimum Extract, Crithmum maritimum, crodafos MCA (cetyl phosphate), crodafos SG (PPG-5 ceteth-10), crodet S40LD (PEG-40 stearate), cromul EM1207 (steareth-21), crosslinked dextrans, crosspolymer (available under the trade name Stabilez® 60), crosspolymer-6, curdlan, cyanidin (Vaccinium myrtillus), cyclic amides, cyclic oligosaccharides, cycloamyloses, cyclodextrin glycosyltransferase (CGTase), cyclodextrins, cyclohexanediaminetetraacetic acid iron complex salts, cyclomethicone, cyclo-oxygenase (for example, COX-1 or COX-2), cyclopentadecalactone, cypress, D&C Red No. 27, D-alpha-tocopherol, D-alpha-tocopheryl acetate, D-alpha-tocopheryl succinate (or acetate), darbepoetin (Aranesp), darutoside (Siegesbeckia orientalis extract), darutoside 0, decaglyn 1-L (polyglyceryl-10 laurate), decanediol, decanoic acid (capric acid), decyl glucoside, decyl oleate, decyl stearate, deferiprone iron complex, dehydroacetic acid, dehydroepiandrosterone (DHEA), delta-5-avennasterol, denaturants, derivatives of pentapeptides, dermatin sulfate, dermofeel G 10L (polyglyceryl-10 laurate), dermofeel G 6CY (polyglyceryl-6 caprylate), desferrioxamine-iron complex, desquamation actives, detersive surfactants, devil's claw extract, dextrans, D-glucosamine, DHC-30 (Dihydrocholeth-30), DHEA, dialkyl sulfoxides, diamyl ester of sodium sulfosuccinic acid, diazolidinyl urea, dibenzoylmethane derivatives, dibutyl adipate, dicetyidimonium chloride, dicetyl dipeptide 9, dicetyl phosphate, diclofenac, diethanolamine laureth sulfate, diethanolamine lauryl sulfate, diethanolamine p-methoxy cinnamate, diethanolamine, diethyl sebacate, diethylene glycol monomethyl ether, diethylene glycol, diethylene glycolamine/epichlorohydrin/piperazine-copolymer, diethylenetriaminepentaacetic acid iron complex salts, diethylpolysiloxane, diethyltoluamide, dihexyl ester of sodium sulfosuccinic acid, dihexyldecyl adipate, dihydric alcohols, dihydrogenated tallow benzylmonium chloride, dihydroxyacetone, diisopropyl adipate, diisopropyl sebacate, dilauryidimonium chloride, dilauryl citrate, dill, dimerized rosin, dimethicone crosspolymer, dimethicone/vinyl dimethicone crosspolymer, dimethiconol, dimethylacetamide, dimethylformamide (DMF), dimethylpolysiloxane, dimethylpolysiloxane-diphenylpolysiloxane, dinitrosyl dithiolato iron complex, dioctyl esters of sodium sulfosuccinic acid, diosgenin (trigonella foenumgraecum, fenugreek), diosmin (Citrus sinensis), dioxybenzone, dipalmitylamine, dipeptide 1, dipeptide 10, dipeptide 11, dipeptide 12, dipeptide 15, dipeptide 16, dipeptide 17, dipeptide 18, dipeptide 19, dipeptide 2, dipeptide 20, dipeptide 3, dipeptide 4, dipeptide 5, dipeptide 6, dipeptide 7, dipeptide 8 HCL, dipeptide 8, dipeptide 9, dipeptide carnosine (beta-ala-his), dipeptide diamino butyroyl benzylamide diacetate, diphenylpolysiloxane, dipropylene glycol, disodium cocoyl glutamate, disodium edetate, disodium EDTA, disodium laureth sulfosuccinate, disodium lauryl sulfosuccinate, disodium N-octadecyl sulfosuccinamate, disoyadimonium chloride, distearyl citrate, di-tail phospholipids, ditallowdimonium chloride, dithiocarbamate iron complex, dithiocarboxy-iron complex, DL-alpha-tocopherol, DL-alpha-tocopheryl acetate (or succinate), D-limonene, DMSO, DNA Ascorbate, dodecanedioic acid/cetearyl alcohol/glycol copolymer, dodecanoic acid (laurie acid), dodecyltrimethylammonium chloride, Dow Corning™ 1503 Dow Corning™ 2501 Wax, Dow Corning™ Xiameter, Dow Corning™ 1503, D-panthenol drug astringents, Echinacea angustafolia, Echinacea pupurea, Ecklonia cava extract, econazole nitrate, econazole, edemine (lecithin and escin (triterpenic saponin from Aesculus hippocastanum)), edetate, EDTA, EDTA-iron complex, egg oil, egg powder, egg protein, eicosatriynoic acid, elaidoic acid, elaidoyl group, ellagic acid (Punica granatum), elubiol, Em300, emblica (Phyllanthus emblica) extract, emblica, emollients, Empilan® NP20 (Nonoxynol-20), Emulgin BA 25 (Beheneth-25), Emulgin CS-50 (Ceteareth-50), Emulmetik 300 (Em300), Emulmetik 900 (Em900), Emulmetik 950 (Em950), EMULPUR® IP (EMP IP), emulsan, emulsifying wax, emulsions of nanoparticles, EMULTOP® IP (EMT IP), Enteromorpha compressa extract, Ep130P, Epikuron 130P (Ep130P), Epikuron 145V (Ep145V), epoetin (e.g., procrit, epogen, and eprex), epoxidized soybean oil, ergocalciferol (vitamin D2), erythritol, threitol, erythromycin sulphate, erythromycin, erythropoietin stimulating agents, erythropoietin, escin, esculin, eserine, ester oils (cetearyl isononanoate), esters of alpha-hydroxycarboxylic acids, esters of dimerized rosin, esters of hydrogenated modified rosin; esters of modified wood rosin, esters of tocopherol, estradiol benzoate, estradiol cypionate, estradiol dipropionate, estradiol enanthate, estradiol, estriol, estrofurate, estrogen-like compounds, estrone sulfate, estrone, ethanol, ethanol, ethers (dicarprylyl ether), ethinyl estradiol, ethosulfate, ethoxydiglycol, ethoxylated, ethoxylated aromatic alcohols, ethoxylated carboxylic acids ethoxylated fatty alcohols, ethoxylated fatty alcohols, salt (magnesium chloride, sodium chloride), ethoxylated glycerides, ethoxylated non-aromatic alcohols, ethoxylated PPG acyl ethers, ethyl cellulose, ethyl ester of PVM/MA copolymer, ethyl hydroxyethyl cellulose, ethyl lactate, ethyl lactyle retinoate, ethyl oleate, ethyl paraben, ethyl 4-bis(hydroxypropyl)aminobenzoate, ethyl, ethylene glycol, ethylene glycol monomethyl ether, ethylene oxide, ethylhexyl hydroxystearate, ethylhexyl lactate, ethylhexyl methoxycinnamate, ethylhexyl salicylate, ethylhexylglycerin, ethylparaben, ETYA, eucalyptus, eugenol, Eusolex 4360 (Benzophenone-3), Euxyl® K500, EVA resins, evening primrose oil, excipients, emollients, extract of Arnica (Arnica montana), extract of climbing ivy (Hedera helix), extract of marigold (Calendula officinalis), extract of meadowsweet (Filipendula ulmaria), extract of orthosiphon (Ortosifon stamincus), extract of red vine (Vitis-vinifera) leaves, extract of rosemary (Rosmarinus officinalis), extract of Ruscus (Ruscus aculeatus), extract of sage (Salvia officinalis), extract of St. Johns-wart (Hypericum perforatum), extract of the plant of genus Tephrosia, extracts of Centella Asiatica, fatty acid esters, fatty acids, fatty alcohols (e.g., monohydric alcohols), fenamate, fenticonazole, fenugreek fibers, ferric acetate, ferric albuminate, ferric ammonium citrate, ferric ammonium oxalate, ferric ammonium sulfate, ferric ascorbate, ferric aspartate complex, ferric aspartate, ferric chloride complex, ferric chloride, ferric choline citrate, ferric citrate, ferric fluoride, ferric formate, ferric fumarate, ferric gluconate, ferric glycerophosphate, ferric Glycine sulfate complex, ferric hydroxide saccharate, ferric hydroxide, ferric hydroxypyrone complexes, ferric hypophosphite, ferric manganese citrate, ferric manganese peptonate, ferric manganese saccharate, ferric nitrate, ferric oxide hydrate, ferric oxide saccharated, ferric oxyhydride-dextran complex, ferric oxyhydroxide, ferric peptonate, ferric phosphate, ferric potassium oxalate, ferric potassium tartrate, ferric pyrophosphate soluble, ferric pyrophosphate, ferric quinine citrate, ferric saccharate, ferric sesquichloride, ferric sodium citrate, ferric sodium edetate, ferric sodium oxalate, ferric sodium pyrophosphate, ferric subcarbonate, ferric subsulfate, ferric succinate complex, ferric succinate, ferric sulfate, ferric tartrate, ferric trisglycinate, ferrlecit, ferroGlycine sulfate, ferrous acetate, ferrous ammonium sulfate, ferrous bisglycinate, ferrous carbonate mass, ferrous carbonate saccharated, ferrous carbonate, ferrous chloride, ferrous cholinisocitrate, ferrous citrate, ferrous formate, ferrous fumarate, ferrous fumarate complex, ferrous gluconate, ferrous glutamate, ferrous hydroxide polymaltose complex ferrous hydroxide saccharate complex, ferrous hydroxide saccharate, ferrous hydroxide, ferrous iodide, ferrous lactate, ferrous malate, ferrous nitrate, ferrous oxalate, ferrous oxide, ferrous phosphate, ferrous pyrophosphate, ferrous saccharate complex, ferrous succinate complex, ferrous succinate, ferrous sucrose, ferrous sulfate complex, ferric gluconate complex, ferrous sulfate heptahydrate, ferrous sulfate, ferrous tartrate, ferrozine-iron complex, Ferrum vitis, filbert (hazelnut) oil, film formers, Flamingia macrophylla, flavonoid, flavourants, flaxseed saccharide (acidic), fluconazole, flutrimazole, folic acid, forskohlin extract (from Coleus forskohlii plant), fougere, fragrances, frankincense, frescolat MGA, Fructose-1,6-diphosphate, Fucus vesiculosus extract, Fucus vesiculosus, fumed silica, furcellarans, galactoarabinan, galactoglucomannans, galactomainans, galactosamine, galanga extract (Kaempferia galanga), galanga, galangol, gamma tocopherol, gamma-cyclodextrin (eight units), Garcinia cambogia extract, Gatuline®, gelatin, gellan gum, gellan, Genapol C200 (Coceth-20), Genapol LA030 (Laureth-3), Genapol LA070 (Laureth-7), Genapol T800 (Ceteareth-80), Geogard 221, geranium, Germaben® M-E, Germall II, ginger root extract (Zingiber officianalis), ginger, glass flake, glerecyth-7, Glucamate™ DOE-120 (PEG-120 methyl glucose dioleate), Glucamate™ SSE-20 (PEG-20 methyl glucose sesquistearate), glucomannans, glucosamine ascorbate, glucosamine, glucose glutamate, glutathione, glutathione ascorbate, glycereth-26, glycerol, glycerol tri-, di-, and monoesters, glycerol, polyethylene glycol, glyceryl aminobenzoate, glyceryl ascorbate, glyceryl esters, glyceryl hydrogenated rosinate, glyceryl monoethyl ether, glyceryl rosinate, glyceryl stearate, glyceryl triacetyl hydroxystearate, glyceryl triacetyl ricinoleate, glyceryth-31, Glycine soja, Glycine, glycogens, glycol ether diaminetetraacetic acid iron complex salts, glycol ethers, glycolate, glycolic acid, glycolipids glycols, glycosidic derivatives, glycosphingolipids, glycyrrhetinic acid, Glycyrrhiza inflata, glydant plus, glygeryth-12, GMS-SE, GMS-SE3.04, gossypol, gouania blanchetiana extract, grape seed extract, grape seed oil, graphite, griseofulvin, guar gum, guar hydroxypropyltrimonium chloride, gum arabic, gum ghatti, gum karaya, gum tragancanth (tragacanthin), Gymnema sylvestre extract, gymnemic acid, haloprogin, hardened tallow fatty acid, HCl salt, Heliopsis extract, Heliopsis helianthoides var. scabra, helioxine, hemp oil, heparin, heptanediol, Herbailia® Butcher's Broom, herbal fragrance, Herbalia® Centella, Herbalia® Horse Chestnut, Herbalia® Red Clover, hesperedin (Citrus sinensis), hexadecyl stearate, hexadecyultrimethylammonium chloride, hexamidine compounds, hexane soxhlet extraction of roots, hexanediol, hexanoic acid (caproic acid), hexanoyl dipeptide 3 norleucine acetate,

In further aspects, compositions provided by the invention also or alternatively do not contain more than 2%, more than 1%, more than 0.5%, more than 0.2%, more than 0.1%, more than 0.05%, or more than 0.01% of, do not comprise any detectable or significant amount of (e.g., do not comprise sufficient amount so as to impart a detectable or significant effect), or are free/essentially free of any one or more of hexyl laurate, hexylresorcinol, hibiscus extracts, hickory nut oil, high alkyl betaines, high molecular weight polyalkylglycols, high molecular weight, HMG-COA reductase inhibitors, homomenthyl salicylate, homosalate, honokiol, hop, hops tincture (tincture of Humulus lupus), hordenine, hormones, horse chestnut extract (Aesculus hippocastanum extract), horsetail, human growth hormone (HGH), humectants, hyaluronin sodium salt, hyaluronin, hydrocarbon waxes, hydrocarbons (dioctyl cyclohexane), hydrocarbons (e.g., D-limonene, α-pinene, β-carene, etc.), hydrocortisone 17-butyrate, hydrocortisone, hydrogen, hydrogenated C12-C18 alkyl triglycerides, hydrogenated castor oil laurate, hydrogenated castor oil, hydrogenated coconut oil, hydrogenated cottonseed oil, hydrogenated fish oil, hydrogenated lard, hydrogenated menhaden oil, hydrogenated mink oil, hydrogenated orange roughy oil, hydrogenated shark liver oil, hydrogenated starch hydrolysate, hydrogenated tallow, hydrogenated vegetable oil, hydrolyzed inulin, hydrolyzed silk protein, hydrophobically, hydroquinone, hydroquinone derivatives, hydroxide solution, hydroxy alkyl, hydroxy cetyl phosphate, hydroxy citric acid, hydroxy ethyl cellulose, hydroxy ethyl starch, hydroxy propyl cellulose, hydroxy, hydroxyacids, hydroxyalkyl, hydroxycetyl hydroxyethyl dimonium chloride, hydroxycetyl isostearate, hydroxycitric acid, phaseolamin (from Phaseolus vulgaris extract), hydroxyethyl behenamidopropyl dimonium chloride, hydroxyethyl cetyidimonium chloride, hydroxyethyl guar gum, hydroxyethyl tallowdimonium chloride, hydroxyethylacrylate/sodium acryloyldimethyl taurate copolymers, hydroxyethylcellulose, hydroxyl acids, hydroxyphenoxy propionic acid, hydroxypropyl guar, hydroxypropyl inulin, hydroxypropyl methyl cellulose, hydroxypropyl sorbitol, hydroxypropyl starch, hydroxypropylated guar gums, hydroxypyridone-iron (III) complex, hydroxypropyltrimonium chloride, hydroxytetronic acid, hypericin (Hypericum perforatum), ibuprofen, imidazolidinyl urea, indigoid, indomethacin, inhibitors of acc2 acetyl-COA carboxylase enzyme, inosine, inositol, inter alia, inulin, iodine, iron bis glycinate, iron complex cyanides, iron cyclam complex, iron lactoferrin complexes, iron oxides, iron perhaloporphyrin complex, iron phthalocyamine complex, iron polysaccharide, iron porphyrin complex, iron proteinate, iron thiocyanate complex, iron-arene sandwich complexes, iron-dextran complex, iron-sorbitol-citric acid complex, isethionic acid, iso-, neo-, and n-paraffins, isoceteth-20, isoconazole, isodecyl oleate, isohexyl laurate, isohexyl palmitate, isomers, isonicotinic acid, isoparaffin, isopropanol, isopropyl alcohol (or alternatively ethanol), isopropyl ester of PVM/MA copolymer, isopropyl isostearate, isopropyl lanolate, isopropyl lauroyl, isopropyl lauroyl sarcosinate, isopropyl myristate, isopropyl n-butyrate, isopropyl n-decanoate, isopropyl n-hexanoate, isopropyl palmitate, isosorbide dicaprylate, isosteareth-20, isostearic acid, isostearyl isostearate, isothiazolone, jambu oleoresine extract, Japanese pepper extract, jasmine, jeechem HPIB (silicone blend), jeesilc 6056 (polydimethylsiloxane), jojoba butter, jojoba oil, juniper, Kaempferia galanga (Ethyl-p-methoxycinnamate 98%), kaolin. karaya gum, a methacryloyl ethyl betaine/methacrylate copolymer, kathon CG (preservative), keratin sulfate, ketaconazole, ketones (e.g., carvone, pulegone, piperitone, menthone, etc.), ketoprofen, ketorolac, Killitol, kiwi fruit seed oil, kojic acid, kola seed extract, konjac mannan, kukui oil, L threonate, lactamide DEA, lactamide MEA, lactate, Lactobacillus/Streptococcus Thermophilus/Soybean extract ferment, Hibiscus, lady's thistle, laminarans, Laminaria digitata Extract, lanolin, lard, lanolin derivatives, lanolin fatty acid, lanolin wax, lauralkonium chloride, uramidopropyldimethylamine, laurdimonium hydroxypropyl oxyethyl cellulose, laureth 23, laureth-10, laureth-3, laureth-8, lauric acid, lauric monoglyceride sodium sulfate, lauric triglyceride, lauric/palmitic/oleic triglyceride, laurocapram, lauroyl lysine, Laurus nobilis, Laurydone® (Laurie ester of L-pyrrolidone carboxylic acid) lauryl acetate, lauryl alcohol, lauryl amidopropyl betaine, lauryl bis-(2-hydroxyethyl) carboxymethyl betaine, lauryl bis-(2-hydroxyethyl) sulfopropyl betaine, lauryl bis-(2-hydroxypropyl) alpha-carboxyethyl betaine, lauryl dimethyl alphacarboxyethyl betaine, lauryl dimethyl carboxymethyl betaine, lauryl dimethyl sulfoethyl betaine, lauryl ether sulfate, lauryl lactate, lauryl sarcosine, laurylcapram, lauryltrimonium chloride, lavender, lawsone, l-carnitine, lemon balm, lemon, lemongrass, lesquerella oil, leucocyte extract, levan, lgepal CA-720 (Octoxynol-12), lgepal CO-890 (Octoxynol-40), L-glutathione, L-histidine, lidocaine, limonene, linalool, linalyl acetate, lincol ORH 40/S (PEG-40 hydrogenated castor oil), linoleic acid, linoleic triglyceride, linolenic acid, lipex omega, lipex omega Passiflora, lipid 8OH, lipid 9OH, lipid S75, lipid SL 80-3, lipoic acid, lipophilic derivatives of peptides, lipoprotein complexes, lipoxygenase, Lippia alba, incronam 30 (cocamidopropyl betaine), locust bean gum, long chain alkanolamides, isopropanol, luffa particles, lupenol, lutein (Tagetes patula), luteolin, Lutrol® F127 (poloxamer 407), lycopene (Lycopersicum esculentum), lysine ascorbate, lyso-PC, lyso-phospholipids MA, maca IPA extract (isopropyl alcohol soxhlet extraction of Maca Lepidium meyenii), maca, macadamia oil, mackanate DC-50 (dimethicone copolyol sulfosuccinate), magnesium ascorbyl phosphate, magnesium chloride, magnesium gluconate, magnesium lactate, magnesium oxide, magnesium sulfate, magnesium, magnolol, malate, maleated soybean oil, malic acid, mandelic acid, manganese adenosine triphosphate, mangiferin (Mangifera indica), mango butter, mangostin (Garcinia mangostana), mannitol, mannosamine, Maprounea guianensis extract, marilpal 1618/1 1 (Ceteareth-1 1), maringa oil, marjoram, meadowfoam oil, melaleucol (terpinen-4-ol), melarrest-L, melatonin, melilot (Melilotus officinalis extract), melilot extract, Melissa officinalis, Melissa, menthol, menthyl anthranilate, mestranol, metal complexes (e.g., copper complex of the tripeptide his-gly-gly (also known as Iamin), methosulfate, methyl acetate, methyl gluceth-10, methyl gluceth-20, methyl isonicotinic acid, methyl paraben, methyl undecylenoyl dipeptide 16, methyl, methylidine-iron complex, methyliminodiacetic acid iron complex salts, methylparaben, methylpropanediol, methylsulfonylmethane, methylvalerate, MGD-iron complex, ferrioxamine B, ferrous citrate complex, mica, talc, miconazole, microcrystalline wax, microemulsions, micromerol, microthene FN510, miglyol 840 (propylene glycol dicaprylate/dicaprat), milk powder, milk protein, minerals, minoxidil, mitracarpe extract (Mitracarpus scaber), mixed tocopherols, modified carbohydrates, modified cellulose, modified rosin esters, modified starches, modified wood rosin, moisturizers, molecular weight, monasil PCA (polysiloxy carboxylic acid), monasil PLN (polysiloxy linoleyl phospholipid), mono-, di- and tri-ethylene glycol monoalkyl ethers, monocarboxylic acid salts, monoethanolamine cocoyl sulfate, monoethanolamine laureth sulfate, monoethanolamine lauryl sulfate, monoglycerides, monolauryl citrate, monooleate, monoolein, monostearyl citrate, montan wax, Morus alba leaf extract, Morus nigra, Salix nigra, murumuru butter, myristalkonium chloride, myristic acid, Myristidone® (Myristyl ester of L-pyrrolidone), myristyl 3-glyceryl ascorbate, myristyl group, myristyl lactate, myristyl 3-glyceryl ascorbate, Myrj® 59, Myrj® 51 (polyoxyethylene stearate), Myrj® 52 (polyoxyethylene stearate), myrrh, n-(hydroxymethyl)-nicotinamide, quinolinic acid imide, nicotinanilide, n,n-diethylnicotinamide, nabumetone, N-acetyl galactosamine, N-acetyl glucosamine, N-acetyl mannosamine, N-acetyl-D-glucosamine, N-acetyl-glucosamine, N-acyl amino acid compounds, N-acyl glutamic acid diamide, N-acyl-derivatives, naftifine, naproxen, Natrasol® 250 natriuretic peptide, natural mica, natural rubber, natural waxes, shellac, n-benzylnicotinamide, N-cocoalkypyrrolidone, N-cyclohexylpyrrolidone, N-decane, Nordihydroguaiaretic acid (NDGA), N-dimethylaminopropylpyrrolidone, N-dodecane, neatsfoot oil, neem oil, neomycin (e.g. as the sulphate); neomycin sulphate, neroli, n-ethylnicotinamide, neuroruscogenin N-heptane, N-hexadecane, niacin (vitamin B3), niacin ascorbate, niacin ascorbate, niacin esters, niacinamide ascorbate, niacinamide azelate, niacinamide hyaluronate, niacinamide hydroxybenzoate, niacinamide hydroxycitrate, niacinamide lactate, niacinamide lipoate, niacinamide retinoate, niacinamide salicylate, niacinamide, nialamide, niaprazine nicomol, nicotinaldehyde, nicotinamide ascorbate, nicotinoyl dipeptide 22, nicotinoyl dipeptide 23, nicotinoyl dipeptide 24, nicotinoyl dipeptide 26, nicotinyl alcohol esters of carboxylic acids nifenazone, nipaguard DMDMH, nipaguard PDU, nitrate, nitrile rubber, nitrocellulose, n-methyl-nicotinamide, n-methylpyrrolidone, n-methyltyramine, N-nonane, N-octane, n-octanol, non-amino acid based anionic surfactants, nonionic surfactants, non-ionic surfactants, nonoxynol-15, nonoxynol-20, N-tallowalkylpyrrolidone, N-tetradecane, N-tridecane, N-undecane, nutmeg, nystatin (e.g. clotrimazole, ketaconazole and nystatin), nystatin, oat extract, oat flour, oat protein, octadecyltrimethylammonium chloride, octanoic acid (caprylic acid), octenylsuccinate, octisalate, octocrylene, octopamine, Octopirox® (Piroctone olamine), octoxynol-12, octoxynol-16, octyl dimethyl PABA, octyl methoxycinnamate (ethylhexyl p-methoxycinnamate), octyl salicylate oxybenzone (benzophenone-3), octyl salicylate, octylacrylamide/acrylate copolymer, octylacrylamide/acrylate/butylaminoethyl methacrylate copolymer, octyldodecyl myristate, oil control agents, oil-soluble botanical extracts, okra gum, Olea europaea (olive) fruit oil, olealkmonium chloride, olefin sulfonates, oleic acid (octadecenoic acid), oleic canola oil (Brassica campestris, B. napus, B. rapa), oleic/linoleic triglyceride, oleic/palmitic/lauric/myristic/linoleic triglyceride, oleostearine, oleoyl dipeptide 15, oleth-15, oleth-20, oleth-201.0, oleuropein (Olea europaea), oleyl adipate, oleyl betaine, oleyl dimethyl gamma-carboxypropyl betaine, oleyl myristate, oleyl oleate, oleyl stearate, oligomer olefins, olive husk oil, olive oil (Olea europaea), omental lipids, opacifying agents, oramix CG 110 (caprylyl/capryl glucoside), oramix NS-10 (decyl glucoside), organic acids, organic carboxylic acid salts (including mono-, di- and tri-C1-C₁₈ carboxylic acid salts), organic or inorganic salts, O-substituted ascorbic acid, oxiconazole, oxides (e.g., cyclohexene oxide, limonene oxide, α-pinene oxide, cyclopentene oxide), oxidized starch, ozokerite, P9641 (Laureth-9), P9769 (Laureth-10), PABA (4-Aminobenzoic acid extra pure), paeonol, palm kernel oil fatty acid, palm kernel oil, palmitate, palmitic acid (hexadecanoic acid), palmitoyl, palmitoyl dipeptide 10, palmitoyl dipeptide 13, palmitoyl dipeptide 17, palmitoyl dipeptide 5 diaminobutyroyl hydroxythreonine, palmitoyl dipeptide 5 diaminohydroxybutyrate, palmitoyl dipeptide 7, palmitoyl group, palmitoyl tetrapeptide-7, palmitoyl tripeptide, palmitoyl-gly-his-lys, palmitoyl-lys-thr-thr-lys-ser, panthenol, pantothenic acid (vitamin B5), pantothenic acid, pantothenyl, paper mulberry extract (Broussonetia kazinoke), parabens, paraffin wax, paraffins, particulate materials, Passiflora, passionflower oil (family Passiflora, Passiflora incarnata), patchouli, pationic 138C (sodium lauryl lactylate), PBC-34 (PPG-4 ceteth-20), PDpoietin, pea protein, peach kernel oil, peanut oil, Pecosil® PS-100 (Dimethicone PEG-7 phosphate), pectic acids, pectin, PEG non-sorbitan sugar esters, PEG-120 methyl glucose dioleate, PEG-15 cocamine, PEG-15 cocoyl quaternium 4, PEG-2 oleamonium chloride, PEG-2 stearalkonium 4, PEG-20 stearate, PEG-4, PEG-40 stearate, PEG-400, PEG-5 cocamine, PEG-5 pentaerythritol ether, PEG-5 Stearmonium chloride, PEG-6, PEG-620.0, PEG-6204, PEG-6303, PEG-645.52, Pelargonium inquinans, pengawar djambi oil, pentadesma butter, pentaerythritol hydrogenated rosinate, pentaerythritol rosinate, pentaerythritol, pentanediol, pentapeptide lys-thr-thr-lys-ser, pentapeptide, pentylene glycol, peppermint, peptide CK (arg-lys-arg), peptide CK+ (ac-arg-lys-arg-NH2), peptide E, arg-ser-arg-lys, peptides, perfumes, petrolatum, pH adjusters, phaseolamin, phenanthrolene iron complex, phenoxetol nipa, phenoxy ethanol, phenylbutazone, phenylethyl alcohol, phosphate, phosphatidyl choline, phosphodiesterase-5 inhibitors, phospholipids, shea butter, Phospholipon® 80 H, hydrogenated, Phospholipon® 90 NG, phosphonate, photostabilizers, photosterols, phthalic acid amide, phthalic anhydride/glycidyl decanoate copolymer, phthalic/trimellitic/glycol copolymer, Phyto-Age™, phytonadione, phytosan, phytosterols, pinene, Piper nigrum (tetrahydropiperine), piperine, piroctone olamine, pistachio nut oil, plant derivatives, plant extracts, plant oils, plant seed extracts, plant tissue extracts, Plantacare® 1200UP (C8-16 mainly C12 glucoside/Lauryl glucoside), Plantacare® 2000UP (C8-16 mainly C8 glucoside/decyl glucoside), Plantacare® 810UP (C8-16 mainly C8 glucoside/capryly glucoside), Plantacare® 818UP (C8-16 mainly C12 glucoside/Coco glucoside), Plantactiv® Aesculus, Plantactiv® Centella (madecassic acid, asiatic acid, asiaticoside), Plantapon® ACG 35 (disodium cocoyl glutamate), Plantapon® ACG 50 (sodium cocoyl glutamate), Plantapon® S (sodium cocoyl hydrolyzed wheat protein glutamate), PMX-0245 (n=6), PMX-200 100 cs, PMX-200 350 cs, poloxamer 182, poloxamer 184, Brij® 30 (polyoxyethylene (4) lauryl ether), poloxamer 231, Poly(propylene glycol)-block-poly(ethylene glycol)-block-poly(propylene glycol)), Polyacrylamide, polyacrylamide polymers, polyacrylamidomethylpropane sulfonic acid, polyacrylate, polyacrylate polymer, polyacrylics, polyalcohols, polyalkylene glycols, polyamide, polyamide-3, polyaminodisuccinic acid iron complex, polyaminopolycarbonate iron complexes, polybutylene terephthalate, polyethylacrylate, polychloroprene rubber, polydextrose, polydimethylsiloxane, polydimethylsiloxane 2.0, polyethylene balls, polyethylene glycol, polyethylene glycol (PEG-6), polyethylene glycol 400 monolaurate, polyethylene glycol hydrogenated rosinate, polyethylene glycol rosinate, polyethylene oxide condensates of alkyl phenols, polyethylene, polyglucopyranosyl iron complex, polyglycerins, polyglycerol esters, polyglyceryl sorbitol, polyglyceryl-3-beeswax, polyglyceryl-6-pentastearate, polyglycosylated glycerides, polyhexamethylenebiguanide hydrochloride, polyhydric alcohols, polyhydroxy acids, polyisobutylenes, polymethylsilsesquioxane, polyol ester rosinate, polyols, polyoxyethylene, sorbitan monostearate, polyphenols, polypropylene glycol, polypropylene, polyquaternium-1, polyquaternium-10, polyquaternium-11, polyquaternium-12, polyquaternium-13, polyquaternium-14, polyquaternium-15, polyquaternium-2, Polyquaternium-22, polyquaternium-28, Polyquaternium-37, polyquaternium-39, polyquaternium-4, polyquaternium-47, polyquaternium-5, polyquaternium-6, polyquaternium-7, polyquaternium-8, polyquaternium-9, polysorbate, polysorbate 20, polysorbate 80, polysorbates, polyterpene resins, polyvinyl acetate, polyvinyl alcohol, polyvinyl butyral, polyvinyl ethers, polyvinyl imidazolinium acetate, polyvinyl methyl ether, porous silica, Porphyra umbilicalis extract, Porphyra umbilicalis, porphyrinato iron (III) complex, posaconazole, potassium, potassium channel activators, potassium cocoyl sulfate, potassium hydroxide, potassium laureth sulfate, potassium lauryl sulfate, potassium lauryl wheat amino acids, potato starch, Potentilla erecta extract, PPG-5-Ceteth-20, PPG-8, pregnenolone, preservative, prickly ash tincture, processing aids, procol CS-20 (Ceteareth-20), procol CS-30 (Ceteareth-30), procol IS-20 (Isosteareth-20), procol LA-4 (Laureth-4), procol OA-20 (Oleth-20), procol OA-20SP, procol OA-5 (Oleth-5), procol OA-5SP (Oleth-5 Special), procol SA-20 (Steareth-20), progesterone, propanol, propellants, propoxylated POE ethers, propyl paraben, propylene glycol, propylparaben, prostaglandin, protachem AWS-100 (PPG-5 ceteth-20, protachem DGS (PEG-2 stearate), protachem SMO (Sorbitan oleate), protachem SMP (sorbitan palmitate), protachem SMS (sorbitan stearate), protamate 1000 DPS (PEG-20 stearate), protamate 1540-DPS (PEG-40 stearate), protamate 200 DPS (PEG-4 stearate), protamate 200-OC (PEG-4 oleate), protamate 300 DPS (PEG-6 stearate), protamate 400-DO (PEG-8 dioleate), protamate 6000-DS (PEG-150 distearate), protasorb L-20 (Polysorbate-20), protasorb O-20 (Polysorbate-80), protasorb P-20 (polysorbate-40), protasorb S-20 (polysorbate 60), proteol OAT, Proteol™ APL (Sodium cocoyl apple amino acids), Proteol™ LW 30 (sodium lauryl wheat amino acids), Proteol™ O.A.T. (sodium lauryl oat amino acids), protopectins, protox C-15 (PEG-15 cocamine), protox C-5 (PEG-5 cocamine), pro-vitamin A, pro-vitamin D3, pro-vitamins, psyllium, psyllium husk powder, psyllium seed gum, PT-40 polyol soluble liquorice extract, PT-40, p-toluidine iron complex, P-U polyol soluble liquorice extract, purified seaweeds (granulated spirulina), purpuromycin PVM, PVM/MA copolymer, PVP, PVP/dimethylaminoethylmethacrylate copolymer, PVP/eicosene copolymer, PVP/ethyl methacrylate/methacrylic acid copolymer, PVP/hexadecene copolymer, PVP/PVA copolymers, PVP/VA copolymer, PVP/vinyl acetate/itaconic acid copolymer, pycnogenol (pine bark extract), pyridoxal, pyridoxal-5-phosphate (P5P), pyridoxamine, pyridoxine (vitamin B6), pyridoxine azelate, pyridoxine salicylate, pyridoxine, pyroxidine, pyroxidine, pyrrolidone derivatives, biodegradable pyrrolidone derivatives (e.g., fatty acid esters of N-(2-hydroxyethyl)-2-pyrrolidone), pyruvate salts, pyruvic acid, pyruvic acid, pyruvate salts, quaternium-16, quaternium-18, quercetin, questice CQ U/A (menthyl PCA), quinacetophenone, quinestrol, radical scavengers, raffinose, Randia armata extract, rapeseed oil, resacetophenone resins, retinaldehyde, retinoic acid, retinoids, retinol acetate, retinol palmitate, retinol, retinyl acetate, retinyl palmitate, retinyl propionate, rhamsan, riboflavin (vitamin B2), rice bran husk, rice bran oil, rice flour, rice starch, rilopirox, rose hip oil, rose, roselle tea extract, rosemary extract CG (extract of Rosmarinus officinalis longa), rosemary, rosewood, rosin ester resins, rosins, rosmarinic acid (Rosmarinus officinalis), ruscogenin, Ruscus (butcher's broom), rutin, Cosmoperine® tetrahydropiperine, Piper nigrum, saccharated iron oxide, safflower oil, sage, salicylates (including their methyl, ethyl, and propyl glycol derivatives), salmeterol (as salmeterol xinafoate), Sambucus nigra flower, sandalwood, saperconazole, sarcosinate, scleroglucan, sea kelp (spirulina) powder, sebum control agents, seeds, Sepigel™ 305, SepiWhite™ MSH (Undecylenoyl phenylalanine), sequestrants, sericoside (Terminalia sericea extract), sericoside, sertaconazole, sesame oil, SHA, Sharon™ Biomix II, shea butter, shellac, short chain alcohols, silicone elastomer silicone oils (cyclomethicone), silicone surfactants, silicone wax, silicones, silk fibers, sisterna SP01-C (sucrose distearate), sisterna SP70-C (sucrose stearate), skin and hair conditioning agents, skin bleaching agents, skin cleansers, skin lightening agents, skin penetration enhancers, skin protectant drug actives, skin protectants, skin soothing ingredients, skin whitening agents, skin-conditioning agents (e.g. humectants and occlusive agents), SL 80, sodium, sodium 3-dodecylaminopropane sulfonate, sodium acrylate/vinyl alcohol copolymer, sodium acrylates/acrylnitrogens copolymer, sodium alginate, sodium and ammonium salts of lauryl sulfate, sodium ascorbyl phosphate, sodium benzoate, sodium carrageenan, sodium cholate, sodium cocoyl apple amino acids, sodium cocoyl glutamate, sodium cocoyl glycinate, sodium cocoyl isethionate (tauranol paste), sodium cocoyl isethionate powder, sodium cocoyl methyl taurate, sodium cocoyl sulfate, sodium dehydroacetate, sodium deoxycholate, sodium dodecyl benzene sulfonate. sodium dodecyl sulphate (SDS), sodium ferrous gluconate complex, sodium hyaluronate, sodium laurate, sodium laureth sulfate, sodium laureth sulphate (EMAL® 228 D/JM), sodium lauroyl glutamate, sodium lauroyl sarcosinate, sodium lauroyl sulfate, sodium lauryl oat amino acids, sodium lauryl sarcosinate, sodium lauryl sulfate, sodium lauryl wheat amino acids, sodium methyl paraben, sodium oleate, sodium polyacrylate, sodium potassium aluminosilicate, sodium propyl paraben, sodium stearyl phthalamate, sodium tridecyl benzene sulfonate, sodium cocoyl isethionate, solan E50 (PEG-75 lanolin), solubilizers, soluble ferrous salts, soluble vitamins (including vitamin C), somepan T25 (sodium cocoyl methyl taurate), sophora, sorbic acid or parabens, sorbitan ester surfactants (acylated sorbitan esters), sorbitan ester surfactants (PEG sorbitan esters), sorbitan laurate, sorbitol, sorbitol, glucitol, sorghum oil, soy extract soy isoflavones CG, soy lecithin, soy lecithin extract, soya fibers, soya flour, soya protein, soyatrimonium chloride, soybean fibers, soybean lecithin, soybean oil, Span® 20 (sorbitan monolaurate), Span® 40 (sorbitane monopalmitate), Span® 80 (sorbitane monooleate), Span® 85 (sorbitane trioleate), Span®60 (sorbitane monostearate), spearmint, sweet fennel, Spilanthes acmella, spilanthes CO2 extract (supercritical CO2 extract of Spilanthes oleracea), Spilanthes supercritical CO2 extract, spilanthol, Spondias mombin extract, squalene, Stabileze® QM, stachylose, starch diethylaminoethyl ether, starch Hydroxypropyltrimonium chloride, stearamidopropyl dimethylamine, steardimonium hydroxyethyl cellulose, stearic acid, stearic triglyceride, stearoxytrimethylsilane, stearyl bis-(2-hydroxypropyl) carboxymethyl betaine, stearyl dimethicone, stearyl dimethyl sulfopropyl betaine, stearyl glycrrhetinate, stearyl octyl dimonium methosulfate, stearylvinyl ether/maleic anhydride copolymer, sterol esters, stigmasterol, strogen, styrene-butadiene rubber, succinamates, succinic acid, succinoglycan, sucrose benzoate/sucrose acetate isobutyrate copolymer, sucrose benzoate/sucrose acetate isobutyrate/butyl benzyl phthalate copolymer, sucrose benzoate/sucrose acetate isobutyrate/butyl benzyl phthalate/methyl methacrylate copolymer, sucrose laurate, sucrose myristate, sucrose, sugar amines, sugar ester surfactants (non-sorbitan sugar esters), sugars, sulconazole, sulfate, sulfide group, sulfobetaines, sulfonate, sulfonated polymers, sulfoxides, sulindac, sulisobenzone, sunflower seed oil, superoxide scavengers, surfac OP30 (Octoxynol-30), surfac OP5 (Octoxynol-5), surfactants (cationic, anionic, non-ionic, amphoteric, or zwitterionic), surfactin, surfhope C-1215L (sucrose laurate), surfhope C-1216 (sucrose myristate), surfhope C-1416 (sucrose myristate), surfhope C-1615 (sucrose palmitate), surfhope C-1715 (sucrose oleate), surfhope C-1715L (sucrose oleate), surfhope C-1815 (sucrose stearate), suspensions of nanoparticles, Suttocide® A, sweet almond oil, sweet orange, sympatens-AIC/200 (isoceteth-20), sympatens-AS/1000G (steareth-100), sympatens-BS/300G (PEG-30 stearate), sympatens-BS/500G (PEG-50 stearate), sympatens-NP/090 (nonoxynol-9), sympatens-NP/150 (nonoxynol-15), symperonic PE/L44 (poloxamer 124), synephrine, synovea DOI, synperonic PE/F68 (poloxamer 188), synperonic PE/F87 (poloxamer 237), synperonic PE/L64 (poloxamer 184), synthetic beeswax, synthetic candelilla wax, synthetic carnauba, synthetic mica, synthetic polymers, tacalcitol (e.g., cholecalciferol), talc, tall oil, tallow alkyl hexaoxyethylene sulfate, tallow alkyl triethylene glycol ether sulfate, tallow fatty acid, tallow, tanning actives, tapioca starch, tarragon extract, tasmanian pepper extract, tauranol I-78-6 (sodium cocoyl isethionate), tauranol ws conc. (sodium methyl cocoyl taurate), taurate copolymers, tautomers, T-butyl alcohol, tea tree, TECA (titrated extract of Centella asiatica), terconazole, tergitol 15-S-20, Tergitol® 15-S-12 (C11-15 pareth-12), Tergitol® 15-S-15 (C11-15 pareth-15), Tergitol® 15-S-20 (C11-15 pareth-20), Tergitol® 15-S-40 (C11-15 pareth-40), Tergitol® 15-S-5 (C11-15 Pareth-5), Tergitol® 15-S-7 (C11-15 Pareth-7), Tergitol® NP-10 (Nonoxynol-10), Terminalia catappa leaf extract, Terminalia catappa leaf, ternafine, terpenes, terpinol, tertiary amine oxides, tertiary phosphine oxides, testosterone, testosterone-like compounds, tetradecyltrimethylammonium bromide, tetrahydrocurcumin, tetrahydrocurcuminoids, tetrapeptide gly-gln-pro-arg, tetrapeptides, tetrasodium EDTA, tetrasodium N-(1,2-dicarboxyethyl)-N-octadecylsulfosuccinamate, THC CG (Tetrahydrocurcuminoids), THC ultra pure (tetrahydrocurcuminoids), theobromine (or salts thereof such as aminophylline), theophylline, thiamin (vitamin B1), thiocolchicoside, thionicotinamide, thyme, tightening agents, tin oxide, tinosorb S, tioconazole, titanium dioxide, titanium oxide, tocopherol acetate, tocopherol sorbate, tocopherol succinate, tocotrienol, tolnaftate, tosylate, Totarol® (Totara-8,11,13-trien-13-ol), transdermal permeabilization agents, transferrin-iron complex, triacetin, tribehenin, tricaprin, tricaprylin, triclocarban, triclosan, triethanolamine, triethanolamine laureth sulfate, triethanolamine lauryl sulfate, triethanolamine salicylate, triethyl citrate, triethyl citrate polyamide-3, triethylamine laureth sulfate, triethylamine lauryl sulfate, triethylene glycol, triglycerides (caprylic capric triglycerides), triheptanoin, trihydroxymethoxystearin, trihydroxystearin, triisononanoin, triisostearin, trilaurin, trilinolein, trimethylsiloxysilicate, trimyristin, trioctanoin, triolein, tripalmitin, tripeptide, tripeptide gly-gly-his, tripeptide gly-his-lys, tripeptide his-gly-gly, tripeptide/tetrapeptide3, triphenylmethane, trisebacin, tristearin, triton X-100 (Octoxynol-9), triton X-165 (Octoxynol-16), triton X-405 (Octoxynol-40), tritons (quaternary ammonium compounds), triundecanoin, trolamine salicylate, TWEEN® 20 (polyethylene glycol sorbitan monolaurate, Tween® 20, oil (e.g., ylang, eucalyptus, peppermint), TWEEN® 60 (polyethylene glycol sorbitan monostearate, TWEEN® 80 (polyethylene glycol sorbitan monooleate; polyoxyethylene (20) sorbitan monooleate), Myrj® 45 (polyoxyethylene (8) stearate), tyramine, tyrosinase inhibitor, ultramarines, ultrez 10, ultrez 21, Ultrez®20, undecyclenic acid, unisex bouquet (AFL-3607/A), unisol S-22 (3-benzylidene camphor), urea, ursolic acid, UV actives, UVA/UVB sunscreens, valerian, val-thr-leu peptide, val-tyr-pro, various clays, various hydroxy acids (alpha hydroxy acids, beta hydroxy acids, and polyhydroxy acids), various plant gums, various tritons (quaternary ammonium compounds), vaseline solid paraffin, V-CP vitamin C dipalmitate, vegetable oil, vegetable squalene, vericonazole, vinyl acetate/crotonic acid copolymer, vinyl acetate/crotonic acid/methacryloxybenzophenone-1 copolymer, vinyl acetate/crotonic acid/vinyl neodecanoate copolymer, visnadine, vitamin A compounds, vitamin B compounds, vitamin C compounds, vitamin D compounds, vitamin E compounds, vitamin F compounds, vitamin H compounds, volpo CS20 (Ceteareth-20), volpo CS25 (ceteareth-25), volpo L23 (C12-13 pareth-23), volpo N 10 (Oleth-10), vorconazole, walnut oil, Walteria indica extract, water soluble gums, wheat flour, wheat germ oil, wheat protein, wheat starch, whelan, white clay, white tea extract, witch hazel extract, xanthine dyes, ximenia oil, xylans, xylitol, xylitylglucoside, xyloglucans, yellow dock extract (Rumex crispus and Rumex occidentalis), ylang ylang, yohimbine compounds, zadoary, zanthoxylum clava herculin, zanthoxylum piperitum, zeolites, zinc acetate, zinc adenosine triphosphate, zinc carbonate, zinc oxide, zwitterionic surfactants, α-(1,4) glycosidic linkages, α-amylase, α-cyclodextrin (six units), α-lipoic acid, β-carotene, β-cyclodextrin, or any derivates of any of the above or combinations of any such compounds or derivates.

### Means Plus Function Aspects

In aspects, known equivalents of components described herein also are within the scope of aspects of the invention. Such equivalents can be described as “means” for performing relevant function(s) associated with such components (e.g., modulating coagulation in the case of a CP-MC, enhancing NAD/NADP pathway activity in the case of a NADP-MC, and enhancing penetration of one or more ingredient(s) of a composition in respect of a penetration enhancer component, if present). Thus, in aspects, the invention provides compositions comprising means for modulating a coagulation pathway (comprising any one or more of the CP-MC compound(s) described herein of equivalents thereof) and means for modulating a NAD pathway (comprising any one or more of the NADP-MC constituent(s) described herein or equivalents thereof). In aspects, compositions comprise means for promoting NAD pathway-associated vasodilation (e.g., comprising nicotinate compound(s) and equivalents thereof) and means for non-vasodilating NAD pathway modulation (comprising niacinamide compound(s) or equivalents thereof). In aspects, compositions also comprise a means for enhancing penetration of composition ingredient(s). In aspects, such compositions comprise means for enhancing penetration of compound(s) that typically do not penetrate the skin, enhancing the amount/number of compound(s) penetrating the skin, enhancing the speed at which compound(s) penetrate the skin or any combination of any or all thereof (e.g., comprising a TSD-PEC or an equivalent thereof). Other ingredients described herein also can represent means for performing associated functions and such means (including equivalents thereof) can be included in such compositions of the invention. Examples of such other means include, e.g., a means for carrying/delivering ingredients (e.g., comprising water or other solvate(s) and their equivalents), means for emollience, and the like. In one exemplary aspect, the invention provides a composition comprising (1) means for modulating a protein C pathway (which can include direct modulation of protein C, modulation of protein S, or both), (2) means for promoting NAD-pathway-associated vasodilation, and (3) means for non-vasodilating modulation of the NAD pathway, optionally including (4) means for enhancing penetration of one or more ingredients that form parts of any or all of (1)-(3).

### General Characteristics of Compositions

Compositions of the invention can, in aspects, be further characterized on the basis of one or more characteristics of the composition.

In one respect, compositions can be characterized on the basis of the type of physiological effect(s) the composition promotes/causes (and often an attendant regulatory classification). According to aspects, compositions are cosmetic compositions, which cause a significant impact in terms of the appearance of subject(s) (e.g., the appearance of skin), but which are not intended to treat or prevent a disease or condition (e.g., do not result in any physiological effect that would be considered treating or preventing a disease). According to aspects, compositions herein are pharmaceutical compositions that are suitable for therapeutic uses (prevention or treatment of disease(s) or other medical condition(s)). In aspects, administration of composition(s) results in a DOS improvement in one or more conditions of the skin in a majority of subjects, a significant number of subjects, or both, in one or more adequate and controlled clinical trial(s). In aspects, such one or more conditions of the skin are, e.g., bruising, hyperkeratosis, or both.

Compositions also can be characterized based on the form of the composition. Compositions can take the form of, e.g., a solution or other dispersion (e.g., a suspension).

Compositions can also be characterized on the basis of intended form of administration/application. For example, in aspects, compositions are adapted/configured for topical application. In aspects, compositions are in the form of a gel, cream, lotion, ointment, etc.

In aspects, compositions may be formulated in a conventional manner using one or more physiologically acceptable carriers and optionally including excipients or auxiliaries which can, e.g., facilitate processing of the active compounds into preparations, promote pharmaceutical properties of the formulation/composition (e.g., in terms of stability, purity (lack of contaminants), etc.), or improve the form/delivery or use of the composition. Any of the well-known techniques, carriers, and excipients may be used as suitable and as understood in the art. Examples of well-known excipients and auxiliaries known in the art may be found, for example, in Remington: The Science and Practice of Pharmacy, Nineteenth Ed (Easton, Pa.: Mack Publishing Company, 1995); Liberman, H. A. and Lachman, L., Eds., Pharmaceutical Dosage Forms, Marcel Decker, New York, N. Y., 1980; and Pharmaceutical Dosage Forms and Drug Delivery Systems, Seventh Ed. (Lippincott Williams & Wilkins, 1999).

Compositions of the invention can be formulated for/adapted to/configured for delivery by any suitable route(s) of administration. In aspects, the invention provides combination products comprising two or more compositions of the invention that are in the same or different forms (e.g., a combination of an oral dosage form and a topical dosage form). In aspects, one or more components described herein are delivered in one dosage form and one or more other components are delivered in another dosage form (e.g., a CP-MC can be delivered by injection and an NADP-MC can be delivered topically; a CP-MC can be delivered topically, and an NADP-MC can be both administered topically and orally; etc.).

Routes of administration/dosage forms may include topical, oral, intraoral, rectal, transdermal, buccal, intranasal, pulmonary, subcutaneous, intramuscular, intradermal, sublingual, intracolonic, intraocular, intravenous, or intestinal administration. Certain excipients, auxiliaries, or other secondary (e.g., non-primary, such as, e.g., non-CP-MC, non-NADP-MC, and, e.g., non-PEC-MC components of such formulations are known in the art (see, e.g., Fingl et al., in The Pharmacological Basis of Therapeutics, Ch. 1, p. 1, 1975 and other references cited herein).

Composition can be, where suitable, administered as (and formulated as) tablets, capsules (each of which includes sustained release or timed-release formulations), pills, powders, granules, elixirs, gels (e.g., in situ gels), microspheres, crystalline complexes, liposomes, micro-emulsions, tinctures, suspensions, syrups, aerosol sprays, and emulsions. Compositions can be administered by oral, intravenous (bolus or infusion), intraperitoneal, or subcutaneous, transdermal, or intramuscular route (and comprise attendant “form”). Cosmetic formulations typically are topically applied, but other formulations/compositions for therapeutic or dietary supplement applications can include oral dosage forms, and pharmaceutical applications can include, e.g., injectable dosage forms, intranasal dosage forms, needle free injection dosage forms, inhalation dosage forms, etc. Exemplary forms of pharmaceutical products that can be applied or adapted to compositions of the invention include, e.g., aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

Compositions can comprise any suitable amount of one or more actives that is/are effective for intended use. Oral dosage forms can deliver, e.g., about 0.001 to about 1000 mg/kg of active ingredient(s) body weight, preferably between about 0.01 to about 100 mg/kg of body weight per day, and most preferably between about 0.6 to about 20 mg/kg/day. Compositions can be modified release formulations. E.g., a composition can be administered by a depot formulation/composition that will allow sustained release of active(s) (e.g., primary active(s)) over a period of days/weeks/months as desired. Compositions can be delivered via transdermal routes, e.g., using transdermal skin patches. In aspects, compositions can be administered using a skin patch, e.g., a needleless skin patch or a microneedle skin patch. In aspects, compositions can be administered using a mechanical delivery mechanism other than a skin patch. In aspects, compositions are applied directly to the skin without the aid of a patch or other mechanical delivery system. Dosage forms suitable for administration may contain from about 0.1 mg to about 500 mg of active ingredient per dosage unit. In these pharmaceutical compositions, the active ingredient can be present in an amount of about 0.5 to about 95 wt. % of the composition.

Component(s) of compositions can be administered in a mixture with suitable pharmaceutical diluents, excipients, or carriers (collectively referred to herein as pharmaceutical carriers) suitably selected with respect to the intended form of administration, that is, oral tablets, capsules, elixirs, aerosol sprays generated with or without propellant and syrups, and consistent with conventional pharmaceutical practices.

For instance, for oral administration in the form of a tablet or capsule, component(s) can be combined with an oral, non-toxic, pharmaceutically acceptable, inert carrier such as but not limited to, lactose, starch, sucrose, glucose, methyl cellulose, magnesium stearate, dicalcium phosphate, calcium sulfate, mannitol, sorbitol, etc.; for oral administration in liquid form, the oral drug components can be combined with any oral, non-toxic, pharmaceutically acceptable inert carrier such as, but not limited to, ethanol, glycerol, water, etc., Moreover, when desired or necessary, suitable binders, lubricants, disintegrating agents, and coloring agents can also be incorporated into the mixture. Suitable binders include, but are not limited to, starch, gelatin, natural sugars such as, e.g., but not limited to, glucose or beta-lactose, corn sweeteners, natural and synthetic gums such as acacia, tragacanth, or sodium alginate, carboxymethylcellulose, polyethylene glycol, waxes, etc. Lubricants used in these dosage forms also can include, e.g., sodium oleate, sodium stearate, magnesium stearate, sodium benzoate, sodium acetate, sodium chloride, etc. Disintegrants that can be present include, but are not limited to, starch, methyl cellulose, agar, bentonite, xanthan gum, etc.

Compositions may also be administered in the form of mixed micellar or liposome delivery systems, such as, e.g., small unilamellar vesicles, large unilamellar vesicles, and multilamellar vesicles. Liposomes can be formed from a variety of phospholipids, such as cholesterol, stearylamine, phosphatidyicholines, etc. Permeation enhancer(s) may be added to DOS enhance drug absorption.

Compositions can include pharmaceutically acceptable polymers as carriers that impart function properties, such as drug targeting, modified release, etc. Such polymers can include, e.g., polyvinyl-pyrrolidone, pyran copolymer, polyhydroxypropyl-methacrylamide-phenol, polyhydroxyethylaspartamidephenol, or polyethyleneoxidepolylysine substituted with palmitoyl residues, etc. Furthermore, the composition of the present invention may be combined with a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels, etc.

Compositions can include capsules, tablets, liquid dosage forms, and the like. Gelatin capsules may contain the active ingredient and powdered carriers, such as, e.g., lactose, starch, cellulose derivative, magnesium stearate, and stearic acid. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

Water, a suitable oil, saline, aqueous dextrose (glucose), and related sugar solutions and glycols such as propylene glycol or polyethylene glycols are suitable carriers for solutions, such as parenteral solutions. Compositions, such as, e.g., compositions in the form of suspensions or solutions, can comprise buffering agents/buffers and pH adjusting agents. Compositions can include antioxidizing agents such as sodium bisulfite, sodium sulfite, or ascorbic acid, either alone or combined. Other suitable excipients include citric acid and its salts, chelating agents such as sodium EDTA, and preservatives such as benzalkonium chloride, methyl- or propyl-paraben, chlorobutanol, etc. Suspensions can include cellulose polymers, such as sodium carboxymethyl cellulose, sorbitol solution, etc. Compositions can further comprise viscosity-enhancing agents or viscoelastic agents. Suitable preservatives that can be included in compositions include mercury-containing substances such as merfen and thiomersal; stabilized chlorine dioxide; and quaternary ammonium compounds such as benzalkonium chloride, cetyltrimethylammonium bromide and cetylpyridinium chloride.

Compositions can include binders/carriers, such as cellulose/cellulose derivative polymers (e.g., carboxymethylcellulose, methylcellulose, hydroxypropylmethylcellulose, hydroxyethylcellulose, hydroxypropylcellulose ethylcellulose, and microcrystalline cellulose); microcrystalline dextrose; amylose; magnesium aluminum silicate; polysaccharide acids; bentonites; gelatin; polyvinylpyrrolidone; polyvinylpyrrolidone/vinyl acetate copolymer; povidone and related compositions; starches (e.g., pregelatinized starch); sugars (e.g., dextrose, lactose, etc.); polyethylene glycol; etc. Dispersing agents that can be in compositions include, e.g., hydrophilic polymers, electrolytes, Tween® 60 or 80, PEG(s) (polyethylene glycol(s)), polyvinylpyrrolidone, cellulose/cellulose derivative polymers, poloxamers (e.g., Pluronics F68®, F88®, and F108®), poloxamines, xanthan gum, chitosans, alginates, and carbomers, etc., and equivalents thereof. Compositions can include disintegrants, such as starches, cellulose products, etc. Compositions can include lubricants, such as stearic acid, calcium hydroxide, talc, sodium stearyl fumerate, polyethylene glycols, etc. Compositions can include solubilizers (e.g., ethyl oleate, ethyl caprylate, sodium lauryl sulfate, transcutol, PEGs, TPGS and TPGS derivatives, etc.). Compositions can comprise suspending agents, such as PEGs. A polyethylene glycol in such and other respects can have, e.g., a molecular weight of about 300 to about 6000, or about 3350 to about 4000, or about 7000 to about 5400. Other suspending agents include cellulose derivatives (e.g., sodium carboxymethylcellulose, methylcellulose, hydroxypropylmethylcellulose, or hydroxymethylcellulose acetate stearate), polysorbates (e.g., polysorbate-80), gums, such as, e.g., gum tragacanth and gum acacia, guar gum, xanthans, including xanthan gum, polyethoxylated sorbitan monolaurate, polyethoxylated sorbitan monolaurate, povidone, etc. Compositions can include surfactant(s), such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), polyoxyethylene (60) hydrogenated castor oil; etc. Compositions also can comprise wetting agents, such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, etc.

Compositions can be, e.g., solid oral dosage forms, controlled release formulations, fast melt formulations, effervescent formulations, tablets, powders, pills, capsules, delayed release formulations, extended-release formulations, pulsatile release formulations, multiparticulate formulations, and mixed immediate release and controlled release formulations. Compositions can include particles, such as microparticles or nanoparticles containing or being otherwise associated with active ingredient(s). Compositions can, e.g., include biodegradable microparticles, such as are formed by use of biodegradable polymer(s). In aspects, particles are maintained in suspension. In aspects, particles are maintained in suspension for extended periods of time, such as, e.g., for a period of at least about 1 day, ≥~1 week, ≥~2 weeks, ≥~3 weeks, ≥~1 month, ≥~3 months, ≥~6 months, ≥~9 months, ≥~1 year, ≥~18 months, or, e.g., ≥~2 years. In aspects, particles can be resuspended by mixing, e.g., by stirring, shaking, or otherwise mixing composition(s) for a period about 10 seconds, ~15 seconds, ~20 seconds, ~30 seconds, ~1 minute, ~2 minutes, ~3 minutes, ~4 minutes, or, e.g., about ~5 minutes. Compositions can comprise diluents, such as, e.g., sugars (including lactose, sucrose, and dextrose), polysaccharides (including dextrates and maltodextrin), polyols (including mannitol, xylitol, and sorbitol), cyclodextrins, etc. In some embodiments, compositions are characterizable as self-emulsifying drug delivery systems (SEDDS) (see, e.g., U.S. Pat. Nos. 5,858,401, 6,667,048, and 6,960,563). In embodiments, transdermal formulations described herein are administered using a variety of devices which have been described in the art. For example, such devices include, but are not limited to, U.S. Pat. Nos. 3,598,122; 3,598,123; 3,710,795; 3,731,683; 3,742,951; 3,814,097, 4,031,894; 4,060,084; 4,069,307; 4,077,407; 4,201,211; 4,230,105; 4,292,299; 5,336,168; 5,665,378; 5,837,280; 5,869,090; 6,923,983; 6,929,801; and 6,946,144). In aspects, compositions are suitable for intramuscular, subcutaneous, or intravenous injection. Examples of such compositions can include physiologically acceptable sterile aqueous or non-aqueous solutions, dispersions, suspensions or emulsions, and sterile powders for reconstitution into sterile injectable solutions or dispersions. In embodiments, delivery systems are employed, such as, for example, liposomes, micelles, exosomes, and emulsions. In certain embodiments, compositions provided herein can also include an mucoadhesive polymer, selected from among, for example, carboxymethylcellulose or a carbomer (acrylic acid polymer), poly(methylmethacrylate), polyacrylamide, polycarbophil, acrylic acid/butyl acrylate copolymer, sodium alginate, or dextran. In embodiments, compositions are administered topically and can be formulated into a variety of topically administrable compositions, such as solutions, suspensions, emulsions, lotions, gels, pastes, medicated sticks, balms, creams, or ointments. Such pharmaceutical compounds can contain, e.g., solubilizers, stabilizers, tonicity enhancing agents, buffers, or preservatives.

In aspects, a composition is a multi-phase composition. In aspects, a composition is, comprises, mostly comprises, generally consists of, or consists essentially of an emulsion, such as an oil-in-water emulsion.

Compositions can have any suitable pH characteristics dependent upon the nature of the composition. In aspects in which the composition is formulated for topical application, the composition can be acidic. In aspects, the composition can have a pH of about 4-6.75, such as about 4.25-about 6.5, such as about 4.5-about 6, or such as about 4.75-5.25.

In aspects, compositions, such as topical compositions, can be characterized on the basis of viscosity. In aspects, compositions have a viscosity of, e.g., ~10,000 to ~50,000 centipoise/centipoises (cps), such as ~12,500 to ~42,500 cps, such as ~15,000 to ~35,000 cps (e.g., as determined using a Brookfield Viscometer, RVT spindle C, @ 10 rpm and room temperature, over typical testing times and conditions/settings).

In aspects, compositions are provided in a form suitable for topical application such as, e.g., as a gel, ointment, cream, liniment, lotion, paste, jelly, foam, oil, shake lotion, tincture, or topical solutions such as drops, rinses, or sprays. In aspects, a solid or semi-solid form can be applied topically such as, e.g., a solid for such as is used for deodorant-like products or a powder. In aspects, compositions are formulated for delivery via a patch, tape, or film. In aspects, the compositions are classified as a cream. In aspects, compositions are formulated for topical delivery wherein delivery comprises rubbing or massaging the composition into the skin of the recipient (e.g., an individual). In aspects, compositions can be suitably applied by rubbing or massaging the topical composition into the skin for a period of less than about 2 minutes, such as, e.g., a period of ≤~1.75 minutes, ≤~1.5 minutes, ≤~1.25 minutes, ≤~1 minute, ≤~0.75 minutes, ~0.5 minutes, or, e.g., ≤~0.25 minutes.

In aspects, compositions provided by the invention comprise one or more ingredients which, when the composition is applied topically, are DOS absorbed into the skin. In aspects, the composition is at least mostly, e.g., is substantially, absorbed into the skin within less than about 16 hours of application, such as, e.g., ≤~15 hours, ≤~14 hours, ≤~13 hours, ≤~12 hours, ≤~11 hours, ≤~10 hours, or ≤~8 hours of application, such as, e.g., is at least substantially absorbed into the skin within ≤~7.5 hours, ≤~7 hours, ≤~6.5 hours, ≤~6 hours, ≤~5.5 hours, ≥~5 hours, ≥~4.5 hours, ≤~4 hours, ~3.5 hours, ~3 hours, ≥~2.5 hours, ≤~2 hours, ≤~1.5 hours, ≤~1 hour, or, e.g., ~0.5 hours (30 minutes) or ~0.25 hours (15 minutes) or ~0.1 hours (6 minutes). In aspects, at least about 30%, ~35%, ~40%, ~45%, ~50% or more, such as, e.g., ~60%, ~70%, ~80%, or at least about 90% of the composition is absorbed within about 1 hour of application, e.g., within ~55 minutes, ~50 minutes, ~45 minutes, ~40 minutes, ~35 minutes, ~30 minutes, ~25 minutes, ~20 minutes, ~15 minutes, or, e.g., ~10 minutes of administration.

### Methods of Modulating Conditions in Subject(s)

Another facet of the invention is embodied in methods of modulating the condition of a subject comprising administering effective amounts of a CP-MC and a NADP-MC. In aspects, the invention provides methods of modulating condition(s) of the skin of a subject comprising the administration of a composition described herein in a sufficient amount and a sufficient number of times to DOS modulate one or more conditions of the skin of the subject. In aspects, the invention provides methods of DOS inducing, enhancing, or promoting one or more cosmetic effects in a subject's skin. In aspects, a cosmetic effect is, e.g., skin smoothing, wrinkle reduction, fine line reduction, erythema reduction, luminosity enhancement, etc. or combinations thereof. In aspects, methods comprise administering both a vasodilating/nicotinate NADP-MC (e.g., a NADP-MC comprising effective amount(s) of one or more nicotinate(s) or nicotinate-related compound(s) (e.g., nicotinate mimetic(s))) and a non-vasodilating/nicotinamide NADP-MC (comprising effective amount(s) of one or more nicotinamide compound(s) or nicotinamide-related compound(s) (e.g., nicotinamide mimetic(s)). In aspects, effects of applying the method comprise visible changes in one or more conditions of the skin. In aspects, such changes are cosmetic, modulating the appearance of the skin, but without treating or preventing a condition that is characterized as a disease. In aspects, such changes are therapeutic, treating or preventing an aspect of a disease, such as a dermatologic disease, examples of which are described elsewhere herein. Methods comprise delivery of effective amount(s) of such components, either together (as in the formulations/compositions described above) or separately but in close enough time to exert a combined impact on subject, which is greater than the impact of either agent administered separately with a significant gap in administration of each other (e.g., more than 0.5 days, more than 1 day, more than 2 days, more than 3 days, more than 4 days, more than 5 days, or more than 1 week). Methods can include repeat administration of compositions or components to the subject for a period of time sufficient to DOS modulate physiological conditions, such as changing the appearance of skin or reducing one or more symptoms or causes of disease in the subject, as is further exemplified/discussed here. In aspects, methods comprise concurrent co-administration of different compositions comprising component(s). In aspects, methods comprise separate administration of such different compositions, but within, e.g., less than ~0.5 days, less than ~8 hours, less than ~5 hours, less than ~3 hours, less than ~2 hours, less than ~1 hour, or less than ~30 minutes of each other (typically, on average, generally, in a significant number of cases, etc.).

In aspects, the invention provides methods of administering compositions described herein to treat one or more skin conditions wherein the composition is adapted to, and the amount of composition applied to a recipient is established such that, the method is effective in more than about 40% of treated subjects, such as, e.g., ≥~45%, ≥~50%, ≥~55%, ≥~60%, ≥~65%, ≥~70%, ≥~75%, ≥~80%, ≥~85%, ≥~90%, or, e.g., ≥~95% of treated subjects.

Subjects treated by application of methods (e.g., administration of compositions of the invention) typically are mammals, such as humans. In aspects, subjects have a normal level of protein C (e.g., a protein C plasma level that is 70-140% of average protein C plasma level). In aspects, subject have a normal level of protein S. In aspects, methods comprise evaluating patients for protein C or protein S levels in cases, e.g., where there are possible indicators of protein C deficiency (or in all cases).

With respect to topical application of products, compositions comprising components, such as multi-component compositions, can be applied to any suitable part of the skin. In aspects, topical compositions are mostly, generally, essentially, or entirely not applied to open wounds of significant size in the performance of the method (e.g., most, generally all, substantially all, or all areas treated comprise open wounds of no more than 5 cm, no more than 2 cm, or no more than 1 cm in size). In aspects, methods do not comprise treatment of open wounds. In aspects, methods to not comprise application of a composition directly to an open wound. Examples of such methods can include reduction of bruising/contusion or other hematomas in subject(s). In aspects, such methods comprise treatment of, e.g., seborrheic keratoses. In aspects, methods include treatment of petechia, purpura, ecchymosis, or a combination thereof.

In aspects, methods comprise treatment of bruises or other skin conditions (e.g., edema) in association with performance of an activity that causes such skin conditions, such as, e.g., a medical, aesthetic, or other procedure, e.g., an injection procedure, such as injection of a dermal filler (e.g., Restylane), injection of a collagenase, such as collagenase Clostridium histolyticum-aaes (e.g., in the Qwo® cellulite treatment—Endo International), and the like. Uncontradicted, references to treatment provide implicit support for treating or preventing a condition. E.g., methods can comprise application of compositions prior to an injection, surgery, mesotherapy, botulinum toxin (“botox”) injection, stretch wires, blepharoplasty, rhinoplasty, liposuction, contact physical activity (e.g., contact sports, such as boxing/kickboxing/MMA), or other procedure/activity, after an injection or other procedure/activity, or both. In aspects, methods can cause a significant reduction in average bruise size, bruise number, bruise color or combination thereof. In aspects, methods/compositions can make such improvements within 5 days, 4 days, or 3 days after initial administration of composition(s). In aspects, methods can reduce bruising/bruises to less than a significant number within 8 days, 7 days, 6 days, or 4 days. In aspects, methods can prevent the formation of bruising or can prevent the development of bruising which is DOS less intense (e.g., in size, color, or pain level) compared to the bruising which, on average, develops in untreated individuals experiencing/undergoing the same treatment or activity (e.g., level or intensity of activity).

In aspects, methods of using a composition to treat a skin-associated disease or medical condition of a subject comprise administering composition(s) for a period of time prior to (before) the formation of a skin condition treatable by the compositions herein, such as a skin wound or bruise. In aspects, such methods are applicable in circumstances when such a wound is expected to occur or is likely to occur, such as bruising in subjects having a condition wherein bruising is common or likely to occur (e.g., the frequency of bruising in such a subject is known to be higher than the frequency of burning in a healthy individual). In aspects, provision of the composition can be over a period of about 1 week, ~6 days (144 hours), ~5 days (120 hours), ~4 days (96 hours), ~3 days (72 hours), ~2 days (48 hours), ~1 day (24 hours), ~20 hours, ~18 hours, ~16 hours, ~14 hours, ~12 hours, ~10 hours, ~8 hours, ~6 hours, ~4 hours, ~2 hours, or, e.g., about 1 hours prior to the formation of a wound or bruise on the skin of the subject. In aspects, methods of using a composition to treat a skin-associated disease or medical condition of a subject comprise administering the composition for a period of time promptly, e.g., immediately following (after) the formation of a wound or bruise on the skin of the subject. Here, “promptly” or “immediately” mean within no more than about 1 day, such as no more than about 22 hours, ~20 hours, ~18 hours, ~16 hours, ~14 hours, ~12 hours, ~10 hours, ~8 hours, ~6 hours, ~4 hours, or ~2 hours or less after the first visible signs of the formation of a wound or bruise on the skin. In aspects, provision of the composition can continue for a period of time after the prompt/immediate provision of the composition, such as is described elsewhere herein. In aspects, methods of using a composition to treat a skin-associated disease or medical condition of a subject comprises administering the composition for a period of time both prior to (before) and promptly/immediately following (after) the formation of a wound or bruise. In aspects, a composition can be provided for a period of at least about 1 hour, ~6 hours, ~12 hours, ~24 hours (1 day), or more, such as ~2, ~3, ~4, ~5, ~6, or ~7 days (1 week) or more prior to (before) the formation of a wound or bruise on the skin of the subject and within no more than about 1 day, such as no more than about 22 hours, ~20 hours, ~18 hours, ~16 hours, ~14 hours, ~12 hours, ~10 hours, ~8 hours, ~6 hours, ~4 hours, or ~2 hours or less after the first visible signs of the formation of a wound or bruise on the skin. In aspects, provision of the composition can further continue for a period of time after the prompt/immediate provision of the composition, such as is described elsewhere herein.

In specific aspects, surgical procedures with which use of compositions may be associated include but may not be limited to cellulite treatment procedures such as, e.g., Qwo® treatment (collagenase Clostridium histolyticum-aaes injection), Cellfina treatment, Velashape treatment, Emtone treatment, Thermage treatment, or Vaser treatment; liposuction procedures; dermatological construction procedures such as implant, reconstruction, or reduction procedures, etc. In certain specific aspects, methods provided by the invention include methods of treating one or more skin-related conditions resulting from Qwo® treatment or a similar treatment. In specific aspects, methods of using a composition to treat a skin-associated disease or medical condition associated with the performance of a medical or aesthetic procedure on a subject, e.g., those described herein and, e.g., specifically in this paragraph, comprises administering the composition for a period of time both prior to (before) and following (after) the performance of the medical procedure. In aspects, a composition can be provided for a period of at least about 1 hour, ~6 hours, ~12 hours, ~24 hours (1 day), or more, such as ~2, ~3, ~4, ~5, ~6, or ~7 days (1 week) or more prior to (before) performance of the medical procedure and for a period of at least about 1 hour, ~6 hours, ~12 hours, ~24 hours (1 day), or more, such as ~2, ~3, ~5, ~6, or ~7 days (1 week), ~2 weeks, ~3 weeks, 4 weeks (1 month) or longer, following (after) the performance of the medical procedure, such as until any skin-associated disease or medical condition associated with the performance of the medical or aesthetic procedure is resolved or reaches a state whereby a subject, physician, or both determine that sufficient resolution has occurred.

In aspects, methods provided by the invention comprise applying a composition topically at least about once per day (1×/day), such as, e.g., at least ~2×/day (on average every 12 hours), ~3×/day (on average every 8 hours), ~4×/day (on average every 6 hours), ~5×/day (on average every 5 hours), or ~6×/day (on average every 4 hours). In aspects, methods provided by the invention comprise applying a composition topically for a period of at least about one day (24 hours), ≥~2 days (48 hours), ≥~3 (72 hours) days, ≥~4 (96 hours) days, ≥~5 (120 hours) days, ≥~6 (144 hours) days, or at least ~7 days (1 week). In certain aspects, methods provided by the invention comprise applying a composition topically ≥~1×/day for a period of ≥~3 days. In certain aspects, methods provided by the invention comprise applying a composition topically ≥~1×/day for a period of ≥~7 days (1 week). In certain aspects, methods provided by the invention comprise applying a composition topically ≥~1×/day for a period of ≥~2 weeks (14 days). In certain aspects, methods provided by the invention comprise applying a composition topically ≥~1×/day for a period of ≥~4 weeks (1 month). In certain aspects, methods provided by the invention comprise applying a composition topically ≥~1×/day for a period of ≥~12 weeks (3 months).

In aspects, e.g., when compositions are applied 3×/day, methods include daily application of about 1 to about 20 mg, such as, e.g., ~5-~20 mg, ~10-~20 mg, or, e.g., ~15-~20 mg of a coagulation pathway modulating component.

In aspects, when compositions are applied 3×/day, methods include daily application of composition comprising the daily application of about 15-about 150 mg of a coagulation pathway modulating component, such as, e.g., between ~15-~140 mg, ~25-~140 mg, ~35-~140 mg, ~45-~140 mg, ~55-~140 mg, ~65-~140 mg, ~75-~140 mg, ~85-~140 mg, ~95-~140 mg, ~105-~140 mg, ~115-~140 mg, ~125-~140 mg, or, e.g., ~135-~140, such as, for example, ~15-~140 mg, ~15-~130 mg, ~15-~120 mg, ~15-~110 mg, ~15-~100 mg, ~15-~90 mg, ~15-~80 mg, ~15-~70 mg, ~15-~60 mg, ~15-~50 mg, ~15-~40 mg, ~15-~30 mg, or, e.g., ~15-~20 mg of coagulation pathway modulating component per day. In aspects, compositions are applied once per day or twice per day, e.g., in the same total amount or the same type of amounts described above. In aspects, methods comprise delivery of an effective amount of vitamin K compound(s) (VKC(s)) to subjects.

In aspects, when compositions are applied 3×/day, daily application of composition comprises about 1 to about 20 mg, such as, e.g., ~5-~20 mg, ~10-~20 mg, or, e.g., ~15-~20 mg of VKC(s).

In aspects, when compositions are applied 3×/day, methods include daily application of about 15-about 150 mg of VKC(s), such as, e.g., between ~15-~140 mg, ~25-~140 mg, ~35-~140 mg, ~45-~140 mg, ~55-~140 mg, ~65-~140 mg, ~75-~140 mg, ~85-~140 mg, ~95-~140 mg, ~105-~140 mg, ~115-~140 mg, ~125-~140 mg, or, e.g., ~135-~140, such as, for example, ~15-~140 mg, ~15-~130 mg, ~15-~120 mg, ~15-~110 mg, ~15-~100 mg, ~15-~90 mg, ~15-~80 mg, ~15-~70 mg, ~15-~60 mg, ~15-~50 mg, ~15-~40 mg, ~15-~30 mg, or, e.g., ~15-~20 mg of VKC(s). In aspects, compositions comprise a single VKC. In aspects, the vitamin K compound is vitamin K1. Exemplary amounts for once daily or twice daily administration can be easily derived from such amounts. This principle is applicable to any similar part of this disclosure, even made in connection with other components/types of ingredients. In aspects, such compositions are applied once daily or twice a day, either at the same amount or achieving the same total amount as indicated above.

In aspects, e.g., when the composition is applied 3×/day, the total daily dose of an NADP-MC is about 1 to about 50 mg, such as, e.g., ~10-~50 mg, or, e.g., ~20-~50 mg of an NADP-MC.

In aspects, when compositions are applied 3×/day, methods include daily application of about 40-about 500 mg, such as, e.g., between ~90-~495 mg, ~140-~495 mg, ~190-~495 mg, ~240-~495 mg, ~290-~495 mg, ~340-~495 mg, ~390-~495 mg, or, e.g., ~440-~495 mg, such as, e.g., ~40-~450 mg, ~40-~400 mg, ~40-~350 mg, ~40-~300 mg, ~40-~250 mg, ~40-~200 mg, ~40-~150 mg, ~40-~100 mg, or, e.g., ~40-~50 mg of NADP-MC per day. Exemplary amounts for once daily or twice daily administration can be easily derived from such amounts. This principle is applicable to any similar part of this disclosure, even made in connection with other components/types of ingredients. In aspects, such compositions are applied once daily or twice a day, either at the same amount or achieving the same total amount as indicated above.

In aspects, e.g., where compositions are applied 3×/day, compositions administered in methods provide a total daily dose of about 1 to about 5 mg, such as, e.g., ~2-~5 mg, ~3-~5 mg, or, e.g., ~4-~5 mg, of a nicotinate compound.

In aspects, when compositions are applied 3×/day, methods include daily application of about 3-about 38 mg of a nicotinate compound, such as, e.g., between ~3-~36 mg, ~3-~34 mg, ~3-~32 mg, ~3-~30 mg, ~3-~28 mg, ≥~~~26 mg, ≥~~~24 mg, ~3-~22 mg, ~3-~20 mg, ~3-~18 mg, ~3-~16 mg, ~3-~14 mg, ~~~12 mg, ≥~~~10 mg, ~3-~8 mg, or, e.g., ≥~~~6 mg, such as, e.g., between ~5-~38 mg, ~10-~38 mg, ~15-~38 mg, ~20-~38 mg, ~25-~38 mg, ~30-~38 mg, or, e.g., ~35-~38 mg of nicotinate compound per day. In aspects, the nicotinate compound is tocopheryl nicotinate. Exemplary amounts for once daily or twice daily administration can be easily derived from such amounts. This principle is applicable to any similar part of this disclosure, even made in connection with other components/types of ingredients. In aspects, such compositions are applied once daily or twice a day, either at the same amount or achieving the same total amount as indicated above.

In aspects, e.g., where a composition administered 3×/day, administration of compositions provides about 1 to about 50 mg, such as, e.g., ~10-~50 mg, ~20-~50 mg, or, e.g., ~30-~50 mg of a niacinamide compound per day.

In aspects, when compositions are applied 3×/day, methods include daily application of about 40-450 mg of a niacinamide compound, such as, e.g., between ~50-~450 mg, ~75-~450 mg, ~100-~450 mg, ~125-~450 mg, ~150-~450 mg, ~175-~450 mg, ~200-~450 mg, ~225-~450 mg, ~250-~450 mg, ~275-~450 mg, ~300-~450 mg, ~325-~450 mg, ~350-~450 mg, ~375-~450 mg, ~400-~450 mg, or, e.g., ~425-~450 mg, such as, e.g., ~40-~400 mg, ~40-~375 mg, ~40-~350 mg, ~40-~325 mg, ~40-~300 mg, ~40-~275 mg, ~40-~250 mg, ~40-~225 mg, ~40-~200 mg, ~40-~175 mg, ~40-~150 mg, ~40-~125 mg, ~40-~100 mg, ~40-~75 mg, or, e.g., ~40-~50 mg of a niacinamide compound per day. In aspects, the niacinamide compound is niacinamide. Exemplary amounts for once daily or twice daily administration can be easily derived from such amounts. This principle is applicable to any similar part of this disclosure, even made in connection with other components/types of ingredients. In aspects, such compositions are applied once daily or twice a day, either at the same amount or achieving the same total amount as indicated above.

In aspects, when the composition is applied 3×/day, the total daily dose of a penetration enhancer component is about 1 to about 50 mg, such as, e.g., ~10-~50 mg, ~20-~50 mg, or, e.g., ~30-~50 mg of a penetration enhancer component.

In aspects, when compositions are applied 3×/day, methods include daily application of about 30 to about 300 mg/day, such as, e.g., between ~30 to ~275 mg, ~30 to ~250 mg, ~30-~225 mg, ~30-~200 mg, ~30 to ~175 mg, ~30 to ~150 mg, ~30-~125 mg, ~30-~100 mg, ~30-~75 mg, or, e.g., ~30 to ~50 mg, such as, e.g., ~30-~300 mg, ~50-~300 mg, ~75 to ~300 mg, ~100-~300 mg, ~100 to ~300 mg, ~125 to ~300 mg, ~150-~300 mg, ~175-~300 mg, ~200-~300 mg, ~225-~300 mg, ~250-~300 mg, or, e.g., ~275-~300 mg of a penetration enhancer component per day. Exemplary amounts for once daily or twice daily administration can be easily derived from such amounts. This principle is applicable to any similar part of this disclosure, even made in connection with other components/types of ingredients. In aspects, such compositions are applied once daily or twice a day, either at the same amount or achieving the same total amount as indicated above.

In aspects, when the composition is applied 3×/day, the total daily dose of a TSD-PEC is about 1 to about 5 mg, such as, e.g., ≥~~~5 mg, ~3-~5 mg, or, e.g., ~4-~5 mg of a TSD-PEC.

In aspects, when compositions are applied 3×/day, methods include daily application of about 2 to 26 mg/day, such as, e.g., between ~2-~24 mg, ~2-~22 mg, ~2-~20 mg, ~2-~18 mg, ~2-~16 mg, ~2-~14 mg, ~2-~12 mg, ~2-~10 mg, ~2-~8 mg, ~2-~6 mg, or, e.g., ≥~~~4 mg, such as, ~4-~26 mg, ~6-~26 mg, ~8-~26 mg, ~10-~26 mg, ~12-~26 mg, ~14-~26 mg, ~16-~26 mg, ~18-~26 mg, ~20-~26 mg, ~22-~26 mg, or, e.g., ~24-~26 mg of a TSD-PEC per day. In aspects, the TSD-PEC is a fragment of hyaluronic acid. In aspects, the TSD-PEC is a “mini-HA.” Exemplary amounts for once daily or twice daily administration can be easily derived from such amounts. This principle is applicable to any similar part of this disclosure, even made in connection with other components/types of ingredients. In aspects, such compositions are applied once daily or twice a day, either at the same amount or achieving the same total amount as indicated above.

In aspects, when applied 3×/day, a composition provides about 1 to about 30 mg, such as, e.g., ~10-~30 mg, or, e.g., ~20-~30 mg of a TSD-PEC per day.

In aspects, when compositions are applied 3×/day, methods include daily application of about, e.g., 24 to about 264 mg of a non-TSD-PEC per day, such as, e.g., between ~24 to 244 mg, ~24 to 224 mg, ~24 to 204 mg, ~24 to 184 mg, ~24 to 164 mg, ~24 to 144 mg, ~24 to 124 mg, ~24 to 104 mg, ~24 to 84 mg, ~24 to 64 mg, or, e.g., ~24 to 44 mg per day, such as, e.g., ~44 to 264 mg, ~64 to 264 mg, ~84 to 264 mg, ~104 to 264 mg, ~124 to 264 mg, ~144 to 164 mg, ~184 to 264 mg, ~204 to 264 mg, ~224-264 mg, or, e.g., ~244-264 mg of a non-TSD-PEC per day. In aspects, the non-TSD-PEC comprises a single constituent. In aspects, the single constituent is ethoxydiglycol. Exemplary amounts for once daily or twice daily administration can be easily derived from such amounts. This principle is applicable to any similar part of this disclosure, even made in connection with other components/types of ingredients. In aspects, such compositions are applied once daily or twice a day, either at the same amount or achieving the same total amount as indicated above.

According to aspects, administration of compositions provided by the invention is initiated under the supervision of a licensed medical professional such as, e.g., a general practitioner, oncologist, dermatologist, plastic surgeon, etc. According to alternative aspects, continued administration of compositions after the initiation of administration under the supervision of a licensed medical professional, for a defined period of time (e.g., for the shelf life of a single product, until all of a provided amount of product is used according to a provided administration protocol, etc.) is user-directed (e.g., continued use can comprise the user self-administering the composition over such a defined period of time. According to alternative aspects, administration of compositions provided by the invention is user-initiated, wherein no supervision of a licensed medical professional occurs prior to the initiation of use of composition(s) herein. In aspects, continued use can further comprise user-directed administration, without the oversight of a licensed medical professional.

In aspects, potential subjects can, if not identified or appropriately handled, experience an unwanted or negative side effect from use of the composition(s) herein, such as, e.g., an allergic reaction to a composition ingredient (an adverse event). In aspects, an undesirable or negative side effect from use of the composition could be any side effect known in the art related to topically applied products, such as, e.g., a contact dermatitis, stinging, burning, irritation, dryness, redness, swelling, or more severe reactions such as itching/swelling of the face, tongue, or throat, dizziness, or trouble breathing. In aspects, such events can be rare, e.g., occurring in less than 1 out of every ~100, ~500, ~1000, ~5000, or ~10,000 or more people or at a level that is not considered significant or that does not pose an impediment to regulatory authority approval or regulatory authority approval without restriction (e.g., without any “black box” warning). However, in aspects, methods provided by the invention can comprise excluding a subject from using the composition(s) if the subject is known to have an allergy to any one or more ingredients of the composition(s). In aspects, methods comprise determining prior to administration of a composition whether or not a potential subject is at risk for such an undesirable side effect (adverse event). In aspects, such a pre-determination can be accomplished by, e.g., reviewing subject medical history, conducting a subject interview, administering and reviewing the results of a subject questionnaire, administering one or more allergy tests (screening for allergies to one or more ingredients of the composition), or the like. Examples of such conditions can include, e.g., vitamin K allergy. In aspects, methods include screening for vitamin K allergy, monitoring for development of vitamin K allergy during application of a VKC-containing composition, or both. In aspects, subjects do not include patients known or identified as having, or methods comprise screening potential patient candidates for, one or more conditions/states that may interfere with efficacy of use of methods/compositions, such as use of warfarin therapy, deep vein thrombosis, pulmonary embolism, pregnancy, heparin therapy, recent blood clot diagnosis/treatment, hyperlipidemia, sepsis, renal disease/renal function issues, liver disease, obesity, recent surgery, congestive heart failure, chronic obstructive pulmonary disease (COPD), inflammatory bowel disease, lupus, cancer, smoking, or respiratory stress disease. In aspects, subjects are screened for, and methods not applied to (or are adjusted for), subjects taking a medication that may interfere with an active ingredient of the composition (NADP-MC or CP-MC constituents). E.g., in methods comprising delivery of compositions comprising VKCs methods can include determining if subjects are using or have recently used cephalosporins (such as, e.g., Cefamandole (Mandol), Cefoperazone (Cefobid), Cefmetazole (Zefazone), or Cefotetan (Cefotan)) and adjusting the method or not applying the method based on such information. Such aspects also can similarly include screening for use of Phenytoin (Dilantin); use/significant use of Orlistat (Xenical, Alli) or Olestra; or use of cholesterol-lowering medications such as bile acid sequestrants and similar drugs (e.g., Cholestyramine (Questran), Colestipol (Colestid), or Colsevelam (Welchol)). In aspects, methods provided by the invention do not comprise administration of composition(s) herein to treat a mineral deficiency, such as, e.g., an iron deficiency. In aspects, subjects receiving compositions via a method described herein do not have a mineral deficiency, such as, e.g., they are not iron deficient.

In specific aspects, the invention provides methods of modulating the condition of the skin of a subject comprising administering an effective amount of a composition herein in a sufficient amount, a sufficient number of times, or both, to detectably or significantly modulate one or more conditions of the skin of the subject, wherein the method comprises determining if a potential subject has one or more risk factors relating to the ingredients of the composition prior to administering the composition. In aspects, the method comprises modifying one or more aspects of the method based on such risk factors, such as, e.g., reducing the amount of composition applied, reducing the frequency of dosing of the composition, reducing the period of time over which a composition is applied, or a combination of any or all thereof.

In aspects, the method comprises monitoring the subject for the development or continuation of such risk factors, such as, e.g., being alert to the development of one or more symptoms related to an allergic reaction (e.g., a new skin condition such as redness, itching, hives, or the like) after administration of a composition has begun. In aspects, the method comprises modifying one or more aspects of the method if development of any one or more such symptoms occur(s), such as, e.g., reducing the amount of composition applied, reducing the frequency of dosing of the composition, reducing the period of time over which a composition is applied, or a combination of any or all thereof. In aspects, the method comprises stopping administration of a composition if one or more undesirable symptoms or adverse events occurs at any point over a course of composition administration (e.g., over the course of about 1 hours, ~1 day, ~1 week, ~1 months, or e.g., ~3 months or any course shorter than, longer than, or within such exemplified courses of administration.)

In aspects, use of a composition described herein results in a detectable or significant improvement in one or more conditions of the skin in a majority of subjects, a significant number of subjects, or both, in a controlled clinical trial. In aspects, such a condition is cosmetic. In aspects, such a condition is therapeutic/prophylactic (preventative-reducing the likelihood of occurrence of a condition, preventing a condition from occurring, reducing the severity of a condition on onset, delaying time to onset of condition, reducing the period a condition is experienced once experienced, or a combination thereof). In aspects, methods are also or alternatively performed to supplement the diet of individuals/subjects.

In aspects, cosmetic applications of methods/compositions include reducing the appearance of fine lines, wrinkles, skin discoloration, skin darkness, smoothing the skin, or any combination thereof. In aspects, application of methods/compositions provide DOS improvement in any one or more of such conditions.

In aspects, use of a composition described herein results in a detectable or significant improvement in hyperkeratosis in a subject, in a majority of subjects, a significant number of subjects, or both, in one or more controlled clinical trial(s). In aspects, methods/compositions are applied to treat eczema (e.g., chronic eczema), actinic keratosis, seborrheic keratosis, or epidermolytic hyperkeratosis. In aspects, a composition can be used to treat/reduce one or more aspects of psoriasis, such as hyperkeratotic skin associated with psoriasis. In aspects, methods comprise treatment of skin associated with acne, rosacea, and the like. In other aspects, methods exclude the treatment of skin associated with acne or rosacea. In aspects, methods exclude treatment of psoriasis.

In aspects, a subject has one or more conditions treatable by use of composition(s) provided by the invention. In aspects, a subject has been diagnosed with or has one or more symptoms of hyperkeratotic skin. In aspects, the invention provides a method of using a composition described herein to improve the appearance of hyperkeratotic skin, e.g., to smooth the skin. In aspects, use of a composition described herein results in a detectable or significant improvement in hyperkeratosis in a majority of subjects, a significant number of subjects, or both, in a controlled clinical trial. In aspects, a detectable or significant difference in skin smoothness can be measured within about 24 hours of topical application of the composition to the skin, such as within about 22 hours, ~20 hours, ~18 hours, ~16 hours, ~14 hours, ~12 hours, ~10 hours, ~8 hours, ~6 hours, ~4 hours, or even less, such as within ~2 hours of topical application of the composition to the skin.

In aspects, a subject has been diagnosed with a bruising-related condition or has one or more visible bruises on the skin. In aspects, the invention provides a method of using a composition described herein to improve the appearance of the bruise, e.g., to reduce the size, improve the appearance of, or resolve the bruise/bruising. In aspects, use of a composition described herein results in a detectable or significant improvement in bruising in a majority of subjects, a significant number of subjects, or both, in a controlled clinical trial. In aspects, detectable or significant difference in bruise size, bruise coloration (e.g., a lightening of a deep black, purple, or red color typical of a hematoma/significant bruise), or both can be measured within 1 week (7 days), such as within about 6 days (144 hours), ~5 days (120 hours), ~4 days (96 hours), ~3 days (72 hours), ~2 days (48 hours), ~1 day (24 hours), or even less, such as within ~12 hours of topical application of the composition to the skin.

In aspects, a subject suffers from bruising associated with a skin-associated disease or medical condition. In aspects, the invention provides a method of using a composition described herein to improve the appearance of the disease- or medical condition-associated bruise/bruising, e.g., to reduce the size, improve the appearance of, or resolve the bruise/bruising. In aspects, use of a composition described herein results in a detectable or significant improvement in bruising in a majority of subjects, a significant number of subjects, or both, in a controlled clinical trial. In aspects, detectable or significant difference in bruise size, bruise coloration (e.g., a lightening of a deep black, purple, or red color typical of a hematoma/significant bruise (e.g., reduction of bruising associated with a level of 3-5 in a bruise visibility scale (see, e.g., Schafide et al., Journal of Forensic Nursing: Jan. 3, 2021—Volume 17—Issue 1—p 24-33) by 1 or more points), or both can be measured within about 1 week (7 days), such as within about 6 days (144 hours), ~5 days (120 hours), ~4 days (96 hours), ~3 days (72 hours), ~2 days (48 hours), ~1 day (24 hours), or even less, such as within ~12 hours of topical application of the composition to the skin. In aspects, methods comprise treatment of bruises having a bruise visibility score of 3-5 or patients comprising a significant number of such bruises, which have bruises with a visibility score that averages in such range, or both.

In aspects, a subject has been diagnosed with psoriasis or has one or more clinical symptoms of psoriasis. In aspects, the invention provides a method of using a composition described herein to improve or resolve one or more psoriasis-related symptoms. In aspects, a psoriasis-related symptom can include a skin rash such as a skin plaque, a rash on one or more nails, a rash associated with one or more joints, joint pain, skin dryness, skin fissures, skin flaking, skin peeling, skin bumps, thick skin, skin redness, itching, etc. In aspects, use of a composition described herein results in a detectable or significant improvement in one or more psoriasis-related symptoms in a majority of subjects, a significant number of subjects, or both, in a controlled clinical trial. In aspects, detectable or significant difference in one or more psoriasis-related symptoms can be measured within about 15 weeks, ~14 weeks, ~13 weeks, ~12 weeks, ~11 weeks, ~10 weeks, ~9 weeks, ~8 weeks, ~7 weeks, ~6 weeks, ~5 weeks, ~4 weeks, ~3 weeks, or, e.g., about 2 weeks, (14 days), ~1 week (7 days), ~6 days (144 hours), ~5 days (120 hours), ~4 days (96 hours), ~3 days (72 hours), ~2 days (48 hours), ~1 day (24 hours), or even less, such as within ~12 hours of topical application of the composition to the skin.

In aspects, a subject has been diagnosed with an eczema-related condition or has one or more clinical symptoms of an eczema-related condition. In aspects, the eczema-related condition can be atopic dermatitis, contact dermatitis, neurodermatitis, dyshidrotic eczema, nummular eczema, seborrheic dermatitis, or statis dermatitis. In aspects, a subject has been diagnosed with seborrheic dermatitis or has one or more clinical symptoms of seborrheic dermatitis. In aspects, the invention provides a method of using a composition described herein to improve or resolve one or more seborrheic dermatitis-related symptoms. In aspects, a seborrheic dermatitis-related symptom can include scaly patches of skin, reddened skin, dandruff, skin dryness, skin flaking, skin peeling, itching etc. In aspects, use of a composition described herein results in a detectable or significant improvement in one or more seborrheic dermatitis-related symptoms in a majority of subjects, a significant number of subjects, or both, in a controlled clinical trial. In aspects, detectable or significant difference in one or more seborrheic dermatitis-related symptoms can be measured within about 8 weeks, such as ~7 weeks, ~6 weeks, ~5 weeks, ~4 weeks, ~3 weeks, or, e.g., about 2 weeks, (14 days), ~1 week (7 days), ~6 days (144 hours), ~5 days (120 hours), ~4 days (96 hours), ~3 days (72 hours), ~2 days (48 hours), ~1 day (24 hours), or even less, such as within ~12 hours of topical application of the composition to the skin.

In aspects, a subject has been diagnosed with actinic keratosis or has one or more clinical symptoms of actinic keratosis. In aspects, the invention provides a method of using a composition described herein to improve or resolve one or more actinic keratosis-related symptoms. In aspects, an actinic keratosis-related symptom can include rough or scaly skin patches, itching etc. In aspects, use of a composition described herein results in a detectable or significant improvement in one or more actinic keratosis-related symptoms in a majority of subjects, a significant number of subjects, or both, in a controlled clinical trial. In aspects, detectable or significant difference in one or more actinic keratosis-related symptoms can be measured within about 12 weeks, such as within ~11 weeks, ~10 weeks, ~9 weeks, ~8 weeks, ~7 weeks, ~6 weeks, ~5 weeks, ~4 weeks, ~3 weeks, or, e.g., about 2 weeks, (14 days), ~1 week (7 days), ~6 days (144 hours), ~5 days (120 hours), ~4 days (96 hours), ~3 days (72 hours), ~2 days (48 hours), ~1 day (24 hours), or even less, such as within ~12 hours of topical application of the composition to the skin.

In aspects, a subject has been diagnosed with a wound to (e.g., on or within) the skin or has one or more clinical symptoms of a skin wound. In aspects, the invention provides a method of using a composition described herein to improve or resolve one or more skin wounds or skin wound-related symptoms. In aspects, a skin wound can be a scratch, an abrasion, a cut, a blister, a sore, a broken pustule, etc. In aspects, a skin would-related symptom can include an open or otherwise exposed area of a layer of skin which under healthy conditions would not otherwise be exposed to the outside environment, (e.g., exposure of a layer of skin other than the epidermis). In aspects, use of a composition described herein results in a detectable or significant improvement in the size (e.g., length, width, or depth) of a skin wound, or a detectable or significant improvement in the replacement of epidermal cells in the area of broken skin in a majority of subjects, a significant number of subjects, or both, in a controlled clinical trial. In aspects, detectable or significant difference in in the size (e.g., length, width, or depth) of a skin wound, or a detectable or significant improvement in the coverage provided by new epidermal cells within the area of previously broken skin can be measured within about 2 weeks, (14 days), such as, e.g., within ~1 week (7 days), ~6 days (144 hours), ~5 days (120 hours), ~4 days (96 hours), ~3 days (72 hours), ~2 days (48 hours), ~1 day (24 hours), or even less, such as within ~12 hours of topical application of the composition to the skin. As has been previously noted, in certain aspects, compositions are not applied directly to an open wound.

According to some aspects, compositions herein are not utilized to treat skin-associated diseases or medical conditions such as acne, rosacea, or vaginal dryness (e.g., the one or more skin-associated diseases or medical conditions treatable by methods herein does not solely comprise, does not primarily comprise, or does not comprise any one or more of acne, rosacea, or vaginal dryness). In aspects, compositions herein are not utilized to treat dark circles (e.g., dark circles under the eyes).

In aspects, as has been alluded to previously, a composition provided by the invention is used to treat a skin-associated disease or condition associated with the performance of a medical or aesthetic procedure on the subject. For example, in aspects, a composition is used to treat bruising associated with the performance of a medical or aesthetic procedure. In another example, a composition is used to treat a skin wound associated with the performance of a medical or aesthetic procedure (such as a removal of a growth or skin mark, cryogenic treatment of the skin, etc.). According to certain aspects, such a skin-associated disease or condition associated with the performance of a medical or aesthetic procedure can be any skin-associated disease or condition which is treatable by use of a composition described herein, such as, e.g., bruising or an open wound.

In an aspect, methods of the invention include administering a composition comprising an effective amount of vitamin K compound(s) and either assessing a subject for a vitamin K allergy during the course of treatment, screening the subject for vitamin K allergy prior to treatment, or both, and modifying the course/condition(s) of treatment if such an allergy is detected. In aspects, modifying the conditions of treatment comprises not applying the treatment, applying an alternative composition of the invention not comprising vitamin K compound(s), reducing the frequency of treatment, reducing the dose of composition or vitamin K compound(s) administered, advising the subject of risks associated with the allergy and use of the composition, monitoring the subject for allergic reaction events, or any combination thereof.

In aspects, the invention provides methods of modulating skin condition(s) of the skin of a subject comprising administering an effective amount of a composition herein a sufficient number of times and an in a sufficient amount such that the method results in the treatment or prevention of a skin-associated disease or medical condition. In aspects, method(s) comprise repeatedly applying the composition topically 2 or more times, such as, e.g., ≥3 times, ≥4 times, ≥5 times, ≥6 times, ≥7 times, ≥8 times, ≥9 times, ≥10 times, ≥15 times, ≥20 times, ≥25 times, ≥30 times, ≥35 times, ≥40 times, ≥45 times, or, e.g., ≥50 times, over a period, such as, e.g., a period of a single day, ~2 days, ~3 days, ~4 days, ~5 days, ~6 days, ~1 week, ~2 weeks, ~3 weeks, ~4 weeks, ~5 weeks, ~6 weeks, ~7 weeks, ~8 weeks, ~3 months, ~4 months, ~5 months, or, e.g., ~6 months or longer. In aspects, a method (or methods) comprises applying a composition about once per day, about 2×/day, ~3×/day, ~4×/day, or ~5×/day for a sustained period of time, e.g., ~2 days-~1 week, ~2 days-~1 month, ~2 days-~3 months, or, e.g., ~2 days-~6 months. In aspects, compositions are only administered 1× or 2×/day over a course of several days, e.g., ≥~~~30, ~3-~21, ~5-~20, ~5-~15, ~5-~10, or ~2-~14 days.

In certain aspects, methods herein comprise applying a composition provided by the invention topically at least once per day for a period of at least ~1 week, such as, e.g., at least ~2 weeks, at least ~1 month, or at least ~3 months.

In aspects, the invention provides methods of administering composition(s) described herein, e.g., any method described herein, wherein the method comprises administering a composition to several separate areas of skin of a subject, such as, e.g., 2 or more, 3 or more, 4 or more, 5 or more, 6 or more, 7 or more, 8 or more 9 or more, or, e.g., 10 or more separate areas of skin. In certain aspects, compositions are administered to defined, individual lesions, e.g., to a single lesion. In aspects, compositions are administered to one or more lesions or affected areas of skin in addition to some amount of skin immediately surrounding the lesion or affected area of skin, e.g., an area extending to up to about 10 cm, such as, e.g., ~9 cm, ~8 cm, ~7 cm, ~6 cm, ~5 cm, ~4 cm, ~3 cm, ~2 cm, or, e.g., an area extending up to about cm away from the outer edge of a lesion in any one or more directions around the lesion(s). In a specific aspect a lesion is a seborrheic keratosis lesion(s).

In aspects, compositions, when applied as part of a method described herein, do not cause a significant increase in skin flushing, blood pressure, pulse rate, body temperature, or a combination of some or all thereof.

### Methods of Manufacturing

In aspects, the invention provides method(s) of manufacturing compositions described herein. In aspects, a method (e.g., a process) of manufacturing comprises a multi-phase ingredient mixture process comprising at least 4 separate phases of preparation or mixing (e.g., at least 4 separate preparation or mixing steps), such as, e.g., ~4, ~5, ~6, ~7, ~8, ~9, ~10, ~11, or, e.g., ~12 such steps. In aspects, the process comprises one or more heating steps, one or more continuous mixing steps (e.g., two or more constituents are continuously mixed for an extended period of time or for a period of time until such a desired outcome is achieved), one or more cooling steps, or combinations thereof. In aspects, the process comprises one or more heating steps, one or more continuous mixing steps, and one or more cooling steps.

According to aspects, there can be two or more, such as three or more processes, e.g., series of steps, combinations of steps, sequences of steps, suitable for manufacturing compositions of the invention. In aspects, a particular method of manufacturing may be appropriate for small batch sizes, large batch sizes, or both. In certain aspects, a particular method of manufacturing may be different depending on batch size, in some aspects, two or more phases can be performed in a variety of sequences or, e.g., two or more phases can be performed concurrently. In aspects, a multi-phase manufacturing process can comprise multiple phases wherein one or more individual ingredients can be suitably added, e.g., at the discretion of the manufacturer, during different phases of the manufacturing process.

As a simple example of such combinations, utilizing (I), (II), and (III) to indicate three different manufacturing phases and (i) to represent a single ingredient, in aspects, phases (I), (II), and (III) may be performed in that order. In aspects, phase (I) may be performed, followed by phase (III) then phase (II). In alternative aspects, phase (II) may be performed, followed by phase (I) then phase (III) or phase (III) followed by phase (I). In yet an additional alternative embodiment, phases (I), (II), and (III) may be performed concurrently. In certain aspects, ingredient (i) may be added during phase (I), while in alternative aspects, ingredient (i) may be added during phase (II) or phase (III). Such examples are provided purely to aid the reader in understanding that specific embodiments of manufacturing processes described here should not be interpreted as being limited only to such description. Herein, phases are generally referenced by a letter, such as “phase A” or “phase B”. Accordingly, in the example provided by this paragraph, “A”, “B”, and “C” may be substituted for “I”, “II”, and “III” to illustrate the interchangeable process steps suitable in certain scenarios of a manufacturing process (e.g., where the resulting composition is a suitable composition according to one or more characteristics of such composition(s) described herein).

In aspects, when and to what phase an ingredient may be added during the manufacturing process, or, alternatively, when a phase is added to a composition during the manufacturing process, can be dictated by the conditions under which such an ingredient or phase can remain stable or can be suitably mixed with other ingredients of a composition without detrimentally affecting the added ingredient(s) or any other ingredient(s) of the composition (e.g., the stability or efficacy of an ingredient or ingredients). In aspects, characteristics which may affect (a) which ingredients can be combined in a phase, (b) the timing of the addition of an ingredient during the manufacturing process (e.g., the stage of the manufacturing process at which an ingredient is added), (c) which phases can be added a particular time or point (stage) of the manufacturing process, (d) which ingredients can be moved from one phase to another according to the preference of the manufacturer, etc. are influenced by factors such as, e.g., the sensitivity of one or more ingredients to light, the sensitivity of one or more ingredients to acidic or basic conditions, the sensitivity of one or more ingredients to temperature, such as, e.g., the sensitivity of one or more ingredients to heat, the solubility, e.g., the water solubility of one or more ingredients (or, alternatively, the lipid solubility of one or more ingredients), the sensitivity of one or more ingredients or combinations of ingredients to shear stress(es), etc.

As a simple example, an ingredient sensitive to light may be included in the preparation of a phase of manufacturing which is protected from light. In aspects, a manufacturing process may comprise two or more phases in which ingredients are detectably or significantly protected from light. In certain aspects, ingredients benefiting from the protection from light can be added in either phase, depending upon the preference or restrictions of the manufacturer. As another simple example, an ingredient may be sensitive to elevated temperatures and, accordingly, should be added during a phase of manufacturing wherein ingredients are protected from heat. In certain aspects, a manufacturing process may comprise two or more phases in which ingredients are maintained at temperatures sufficient to protect ingredients from detectable or significant degradation from heat, and, one or more such ingredients can be added to either phase of the manufacturing process. These examples are provided simply to demonstrate the flexibility of the manufacturing process and should not be seen as limiting.

In one exemplary embodiment, compositions are made according to a process comprising the following steps. In aspects, one or more ingredients described in this exemplary process may be absent in another exemplary embodiment of compositions made according to a similar process. In aspects, one or more ingredients described in this exemplary process may be added at a different point in time during the manufacturing process (e.g., added as an ingredient to a different phase than that exemplified here.) This exemplary embodiment should not be interpreted as limiting to a specific manufacturing process, but rather the embodiment can be used to exemplify a phased manufacturing process suitable for manufacturing composition(s) provided by the invention.

In aspects, water suitable for formulating is added to a mixing container. In aspects, one or more components is added to the water. In aspects, such an ingredient can be any ingredient in the formulation, such as, for example, if the composition comprises an ingredient known in the art as a carbomer, the one or more components added to the water can be, e.g., a carbomer. In aspects, the ingredient is not a carbomer (such as, e.g., in aspects, the formulation does not comprise a carbomer and the ingredient added is not characterizable as a carbomer.) In aspects, the ingredient, e.g., the carbomer, is slowly added, e.g., in small quantities a time (e.g., in aspects, by “sprinkling” the component into the water). In aspects, the ingredient, e.g., the carbomer, is added to the water with mixing (e.g., mixing techniques standard in the art) until completely hydrated.

In aspects, the mixture is heated, e.g., to a temperature of between about 70-75° C. In aspects, the mixture is heated while being mixed (e.g., while continuously mixing). In aspects, upon heating, one or more additional composition constituents is added, e.g., xanthan gum. In aspects, mixing continues until a uniform mixture is obtained (e.g., a mixture having at least generally, at least essentially, or at least substantially the same composition throughout the mixture). In aspects, remaining “Phase A” ingredients listed in Table 4 or their functional equivalents (in, e.g., aspects where substitute compound(s) are utilized) can be added, one at a time, in the order provided by Table 4 in the Example section herein, such as, e.g., adding 03 before 04, 04 before 05, etc.). In aspects, each ingredient is fully solubilized prior to the addition of the next ingredient. In certain aspects, one or more ingredients can be moved to one or more different phases, if such phases are produced or maintained under conditions capable of maintaining the stability (e.g., the integrity, efficacy, etc.) of the ingredient. In certain aspects, one or more ingredients can be added in a different order, if such a modification of order does not affect the solubility or stability (e.g., the integrity, efficacy, etc.) of the ingredient. In aspects, a temperature of between about 70-75° C. is maintained throughout the addition of “Phase A” ingredients (Phase A ingredients as defined by Table 4 or their known or disclosed functional equivalents). In aspects, one or more additional phases are maintained at less than about 75° C., such as between about 65° C.-75° C., or, e.g., between about 70° C.-75° C.

In aspects, a process can comprise a second set of steps, or a second phase, comprising the combination of a second set of ingredients to form a second mixture (e.g., a “Phase B” mixture). In aspects, Phase B ingredients as defined by Table 4 in the Example section herein are combined to form a Phase B mixture. In aspects, ingredients are combined while continuously mixing. In aspects, ingredients are heated to between about 75-80° C. In aspects, Phase B ingredients are heated to between about 75-80° C. while being continuously mixed, until the target temperature is reached, and the Phase B mixture is uniform. In certain aspects, one or more ingredients of Phase B can be moved to one or more different phases, if such phases are produced or maintained under conditions capable of maintaining the stability (e.g., the integrity, efficacy, etc.) of the ingredient. In certain aspects, one or more ingredients can be added in a different order, if such a modification of order does not affect the solubility or stability (e.g., the integrity, efficacy, etc.) of the ingredient. In aspects, one or more additional phases comprising heating to a temperature of at least about 70° C., such as, e.g., between about 70° C.-80° C., or, e.g., between about 75° C.-80° C.

In aspects, the Phase B mixture can be added to the Phase A mixture, creating a “Phase A/Phase B mixture”. In aspects, the Phase B mixture is added to the Phase A mixture slowly under continuous mixing. In aspects, mixing can be maintained for a period of at least about 1 minute, such as, e.g., at least ~2 minutes, at least ~3 minutes, at least ~4 minutes, or, e.g., at least about 5 minutes, such as, e.g., ~2-~5 minutes. In aspects, exact mixing times will vary depending on the size of the batch of composition (e.g., the volume of composition) being made. In aspects, exact mixing times will vary on the specific equipment used to perform the mixing. In aspects, mixing times are dependent upon the total volume of composition being made and the equipment being used to perform the mixing.

In aspects, the combination Phase A/Phase B mixture can be homogenized. In aspects, the mixture is homogenized for a sufficient period of time so as to establish an at least generally, essentially, or substantially uniform mixture. In aspects, the mixture is at least minimally homogenized or, e.g., fully homogenized, so as to establish an at least generally, essentially, or substantially uniform mixture. In aspects, the mixture is homogenized to an extent such that the total amount of any single ingredient in a sample collected representing at least about 10% (e.g., ~5%, ~10%, ~15%, ~20%, or, e.g., ~25%) of the total mixture varies from the total amount of that ingredient present in any other sample of the mixture representing about 10% of the total mixture by more than about 30%, or, e.g., more than about 25%~20%, ~15%, ~10%, ~5%, or, e.g., by more than ~1%. In aspects, such homogenization can be obtained, e.g., by passing the mixture through the homogenization head of the homogenizer one time. In aspects, such homogenization can be obtained, e.g., by passing the mixture through the homogenization head of the homogenizer at least one time, such as, e.g., at least about 1, ~2, or, e.g., at least ~3 times. In aspects, depending upon the specific equipment used, the mixture may be passed through the homogenization head of the homogenizer any number of times to obtain suitable homogenization, such as about 5, ~10, ~20, ~50, or ~100 or more times as needed to obtain suitable homogenization. In aspects, following a homogenization step, the mixed batch is allowed to cool. In aspects, the mixed batch is allowed to cool to about 30-50° C., such as, e.g., the mixed batch is allowed to cool to a temperature of less than about 50° C., such as, e.g., a temperature of ~45° C., ~44° C., ~43° C., ~42° C., or ~41° C. In aspects, the mixed batch is allowed to cool, such as, e.g., to 40-45° C., while being continuously mixed.

In aspects, a “Phase C” mixture can be made using the ingredients of “Phase C” of Table 4 in the Examples section herein. In aspects, each Phase C ingredient is mixed until completely in solution (e.g., until there are no visible, e.g., detectable or significant particulates). In certain aspects, one or more ingredients of Phase C can be moved to one or more different phases, if such phases are produced or maintained under conditions capable of maintaining the stability (e.g., the integrity, efficacy, etc.) of the ingredient. In certain aspects, one or more ingredients can be added in a different order, if such a modification of order does not affect the solubility or stability (e.g., the integrity, efficacy, etc.) of the ingredient.

In aspects, once the Phase C mixture is established, the mixture can be heated to about 40° C.-50° C., such as, e.g., between about 42° C.-48° C., 44° C.-46° C., or, e.g., about 45° C. In aspects, the mixture is maintained at the target temperature until a point at which the Phase A/Phase B mixture is cooled to its target temperature. In aspects, once the Phase A/phase B mixture is cooled to about, e.g., 40-45° C., the Phase C mixture at a temperature of, e.g., ~45° C., is added to the Phase A/Phase B mixture. In aspects, Phase C is added under continuous mixing to form a “Phase A/B/C mixture”. In aspects, mixing parameters are adjusted to maintain a desired consistent viscosity.

In aspects, continued mixing of the Phase A/B/C mixture continues while the Phase A/B/C mixture is allowed to cool to about 40° C.-50° C., such as, e.g., between about 42° C.-48° C., 44° C.-46° C., or, e.g., about 45° C.

In aspects, one or more additional phases can be prepared. In aspects, one or more additional ingredients can be added to a separate container. In aspects, one or more such an ingredient is Transcutol®. In aspects, one or more additional ingredients can be added to the separate container. In aspects, the one or more additional ingredients is butylene glycol. In aspects, such an additional ingredient is added under continuous mixing. In aspects, the resulting mixture is continuously mixed until the mixture is uniform. In aspects, the resulting mixture (“working/incomplete Phase D mixture”) is then heated.

In aspects, the working Phase D mixture is then heated to a temperature of between about 60° C.-about 70° C., such as, e.g., about 62° C.-about 68° C., about 64° C.-about 66° C., or, e.g., about 65° C. In aspects, heating occurs under continuous mixing. In aspects, once heated, one or more additional ingredients can then be added, such as, e.g., resveratrol. In aspects, the mixture is mixed until a clear solution is achieved. In aspects, this mixture, still a working Phase D mixture, is cooled to a temperature of 40° C.-about 50° C., such as, e.g., about 42° C.-about 48° C., about 44° C.-about 46° C., or, e.g., about 45° C. In aspects, cooling is allowed under continuous mixing. In aspects, once a target temperature (e.g., about 45° C.) is reached, one or more additional components can be added. In aspects, such an additional component is licorice. In aspects, the resulting mixture is further mixed, e.g., continuously mixed, until all components of the working Phase D mixture are in solution, e.g., there are no DOS (or no visible) particulates. In aspects, the working Phase D mixture is allowed to cool to about 30° C.-about 50° C., such as, e.g., about 32° C.-about 48° C., about 34° C.-about 46° C., about 40° C.-about 45° C., or, e.g., about 45° C. In aspects, the mixture is allowed to cool under continuous mixing. In aspects, one or more additional ingredients is added to the working Phase D mixture. In aspects, the ingredient is curcumin. In aspects, the mixture is continuously mixed until curcumin is completely in solution. In aspects, mixing is maintained at the target temperature, e.g., about 40° C. In aspects, this mixture is the finished Phase D mixture (“Phase D” mixture).

In certain aspects, one or more ingredients of Phase D can be moved to one or more different phases, if such phases are produced or maintained under conditions capable of maintaining the stability (e.g., the integrity, efficacy, etc.) of the ingredient. In certain aspects, one or more ingredients can be added in a different order, if such a modification of order does not affect the solubility or stability (e.g., the integrity, efficacy, etc.) of the ingredient.

In aspects, once the Phase A/B/C (also referred to as “Phase A-C”) mixture is at its target temperature, e.g., about 45° C. and the Phase D mixture is at its target temperature of about 40° C., the Phase D mixture is added to the Phase A-C mixture. In aspects, this addition occurs slowly. In aspects, this addition occurs under continuous mixing. In aspects, the resulting mixture (the “Phase A-D” mixture) is continuously mixed and allowed to cool to less than about 45° C., such as, e.g., to a temperature of between ~40° C.-~45° C., or, e.g., to ~30° C.-45° C.

In aspects, “Phase E” ingredients of Table 4 in the Examples section herein are added to the batch with mixing. In certain aspects, “Phase E” ingredients are first blended in a separate container and then added to the batch. In certain aspects, one or more ingredients of a particular phase, such as, e.g., Phase E can be moved to one or more different phases, if such phases are produced or maintained under conditions capable of maintaining the stability (e.g., the integrity, efficacy, etc.) of the ingredient. In certain aspects, one or more ingredients can be added in a different order, if such a modification of order does not affect the solubility or stability (e.g., the integrity, efficacy, etc.) of the ingredient. Once uniform (e.g., once Phase E and all Phase I ingredients are suitably mixed into the batch), in aspects, the batch is cooled to less than about 45° C., such as, e.g., to between ~30° C.-~40° C., such as, e.g., between ~35° C.-~40° C. with mixing.

In aspects, the resulting “Phase A-E” mixture is mixed until uniform and is allowed to cool to a temperature of less than about 40° C., such as, e.g., to between ~25° C.-~40° C., such as, e.g., to ~30° C.-~35° C. In aspects, cooling takes place under continuous mixing. In aspects, the manufacturing process can comprise the preparation of one or more yet further phases. In aspects, in a separate container, “Phase F” ingredients are added (Phase F ingredients according to Table 4 of the Examples section herein). In aspects, Phase F ingredients are combined and mixed until a uniform solution is achieved. In certain aspects, one or more ingredients of Phase F can be moved to one or more different phases, if such phases are produced or maintained under conditions capable of maintaining the stability (e.g., the integrity, efficacy, etc.) of the ingredient. In certain aspects, one or more ingredients can be added in a different order, if such a modification of order does not affect the solubility or stability (e.g., the integrity, efficacy, etc.) of the ingredient. In aspects, one or more Phase F ingredients are detectably or significantly sensitive to light exposure. In aspects, the preparation of Phase F is performed while limiting exposure of such ingredients to significant light exposure (e.g., enough light exposure to significantly impact the performance of such ingredients.) In aspects, once a uniform mixture is achieved, the Phase F mixture is added to the Phase A-E mixture. In aspects, the addition occurs slowly. In aspects, the addition occurs under continuous mixing. In aspects, this step establishes a mixture referenced as the “Phase A-F” mixture.

According to certain aspects, amounts of the same ingredient can be added during two or more phases of a manufacturing process. In aspects, the same amount of a single ingredient can be added during two or more phases of a manufacturing process. In aspects, an ingredient added during two or more phases of a manufacturing process can be added in an amount during one phase which varies from the amount added during at least one other phase. For example, a solubilizing ingredient can be added during two or more phases to solubilize one or more other ingredient in each phase. As one example, dimethyl isosorbide can be added in one phase to solubilize one ingredient (e.g., added in one phase to aid in the solubilization of, e.g., ethyl lactyl retinoate) and added in at least one other phase to solubilize a different ingredient (e.g., added in another phase to aid in the solubilization of, e.g., phytonadione.)

In aspects, the manufacturing process can comprise the preparation of one or more yet further phases. In aspects, one or more additional ingredients are added to a separate container. In aspects, water and, e.g., propanediol, are added and mixed in such a separate container. In aspects, the ingredients are mixed until uniform. In aspects, this establishes a working, or incomplete “Phase G” mixture. In aspects, to the working Phase G mixture, one or more additional ingredients is added. In aspects, the additional ingredient is hyaluronic acid. In aspects, the hyaluronic acid is added slowly, in small quantities at a time (e.g., by “sprinkling”) into the working Phase G mixture. In aspects, the addition is performed under continuous mixing. In aspects, the addition is made in a manner such that the formation of DOS clumping is avoided. In aspects, mixing is continued until a uniform solution is achieved. In aspects, once a uniform solution is achieved, one or more additional ingredients can be added. In aspects, the additional ingredient is niacinamide. In aspects, niacinamide is not added at this stage of manufacturing. In aspects, niacinamide is added under continuous mixing. In aspects, mixing continues until the niacinamide is completely in solution. In aspects, once niacinamide is completely in solution, the remaining ingredients in Phase G per Table 4 are added to the working Phase G mixture, ensuring that each ingredient is in solution prior to the addition of the next. In aspects, once all Phase G ingredients have been added (representing the establishment of the final “Phase G mixture”). In certain aspects, one or more ingredients of Phase G can be moved to one or more different/distinct phases, if such phases are produced or maintained under conditions capable of maintaining the stability (e.g., the integrity, efficacy, etc.) of the ingredient. In certain aspects, one or more ingredients can be added in a different order, if such a modification of order does not affect the solubility or stability (e.g., the integrity, efficacy, etc.) of the ingredient. In aspects, once the Phase A-F mixture is at a temperature of about ~25° C.-~40° C., such as, e.g., ~30° C.-~35° C., and the Phase G mixture is at room temperature or, e.g., a similar temperature, such as, e.g., between ~25° C.-~40° C., or, e.g., ~30° C.-~35° C., the Phase G mixture is added to the Phase A-F mixture. In aspects, the addition occurs under continuous mixing. In aspects, the resulting mixture (“Phase A-G mixture”) is allowed to cool to a temperature of less than about 40° C., such as to a temperature of less than about 35° C., such as, e.g., to between about 20-35° C. In aspects, cooling is permitted under continuous mixing.

In aspects, the manufacturing process can comprise the preparation of one or more yet further phases. In aspects, a mixture of “Phase H” ingredients (Phase H ingredients according to Table 4) is prepared. In certain aspects, one or more ingredients of Phase H can be moved to one or more different phases, if such phases are produced or maintained under conditions capable of maintaining the stability (e.g., the integrity, efficacy, etc.) of the ingredient. In certain aspects, one or more ingredients can be added in a different order, if such a modification of order does not affect the solubility or stability (e.g., the integrity, efficacy, etc.) of the ingredient. In aspects, once the Phase A-G mixture is between about 20° C.-about 40° C., e.g., between ~20° C.-~35° C., or, e.g., between ~25° C.-~30° C., the Phase H mixture is added to the Phase A-G mixture. In aspects, the addition occurs under continuous mixing. In aspects, continuous mixing occurs until a uniform composition is achieved. This establishes the “Phase A-H mixture”. In certain aspects, “Phase H” comprises a single ingredient and, thus, no Phase H mixture is established; rather, the single ingredient is added to the batch (e.g., is added to the Phase A-G mixture.)

In aspects, samples are taken of the phase A-H mixture and the pH of the mixture is evaluated. In aspects, if needed, the pH of the final phase A-H mixture is adjusted to a target/desired pH of between about 4 to about 6, such as, e.g., between about 4.2 to about 6, e.g., between ~4.4-~6, ~4.6-~6, or, e.g., ~4-~5.8, ~4-~5.6, ~4-~5.4, or, e.g., between about 4.25-~5.75, ~4.5-~5.5, or, e.g., ~4.75-~5.25. In aspects, “Phase I” ingredients (Phase I ingredients according to the description of Example 4, Phase I ingredients being optional ingredients, added as needed) are used to adjust pH, such as, e.g., a 50% solution of citric acid, a 20-50% solution of sodium hydroxide, or both, are used to adjust the pH of the phase A-H mixture.

In yet a further example of a suitable manufacturing process of an inventive composition described herein, the following process is provided. Percentage amounts provided are percent by weight of the final composition. Specific ingredients exemplified here can be, in aspects, substituted with other suitable equivalents described herein or, e.g., known in the art. In aspects, one or more such ingredients can be excluded provided that resulting final compositions demonstrate at least generally equivalent, at least substantially equivalent, at least essentially equivalent, or, e.g., the same efficacy as compositions comprising such an exemplified composition.

In aspects, a Phase A component of the composition is prepared comprising deionized water in an amount of between about 1 to about 5 wt. % and arginine, e.g., L-Arginine (e.g., C Grade L-Arginine), in an amount of between about 0.5-about 3%, such as, e.g., ~0.5 wt. %-~3 wt. %, ~0.5 wt. %-~2 wt. %, or, e.g., ~0.1 wt. %-~1 wt. %. In aspects, deionized water is added to a container, e.g., a stock pot. In aspects, the L-Arginine is added with mixing until completely in solution, e.g., until no particulate matter is visible. In aspects, Phase A is heated to between about 40° C. to about 50° C., such as, e.g., ~42° C.-~48° C., ~44° C.-~46° C., or, e.g., ~45° C., with mixing. In aspects, Phase A is held (e.g., is maintained in such a state) for further processing until one or more additional manufacturing steps are completed. In aspects, the Phase A component is maintained until required for addition to (combination with) one or more additional phases or ingredients.

In aspects, a Phase B component of the composition is prepared comprising ethoxydiglycol, e.g., Transcutol CG, in an amount of between about 0.1 wt. % to about 3 wt. %, such as, e.g., ~0.2 wt. %-~2.5 wt. %, or, e.g., ~0.5 wt. %-~2 wt. %; butylene glycol in an amount of between about 1 wt. %-about 5 wt. %, such as, e.g., ~1.5 wt. %-about 4.5 wt. %, e.g., about 1 wt. %-about 4 wt. %; resveratrol, e.g., Respure Synthetic Resveratrol, in an amount of between about 0.0.05 wt. % to about 2 wt. %, such as, e.g., ~0.05 wt. %-~1.5 wt. %, e.g., ~0.1 wt. %-~1 wt. %; Glycyrrhiza glabra (licorice) root extract, e.g., Licorice Extract PT-40, in an amount of between about 0.001 wt. % to about 0.02 wt. %, e.g., ~0.001 wt. %-~0.015 wt. %, e.g., ~0.001 wt. %-~0.01 wt. %; and Curcuma longa (Turmeric) root extract, in an amount of between about 0.0001 wt. % to about 0.001 wt. %, such as, e.g., ~0.0001 wt. %-~0.0005. In aspects, the ethoxydiglycol is added to a suitable container (e.g., a container separately from that maintaining Phase A of the composition.) In aspects, butylene glycol is added while mixing. In aspects, mixing is continued until a uniform mixture is obtained. In aspects, the solution is heated to between about 60° C. and about 70° C., such as, e.g., about 65° C., with mixing. In aspects, the resveratrol is added and mixed until a clear solution is obtained. In aspects, the mixture is cooled to between about 40° C. and about 50° C., such as, e.g., 45° C. In aspects, the licorice ingredient is added, and the composition is mixed until completely in solution, such as, e.g., no particulate matter is visible. In aspects, the mixture is then cooled to between about 35° C. and about 45° C., such as, e.g., about 40° C. with mixing. In aspects, the curcumin ingredient is then added, and the composition is mixed until completely in solution, such as, e.g., no particulate matter is visible. In aspects, the solution is maintained at, e.g., about 40° C. while mixing until one or more additional manufacturing steps are completed. In aspects, the Phase B component is maintained until required for addition to (combination with) one or more additional phases or ingredients.

In aspects, a Phase C component of the composition is prepared comprising dimethyl isosorbide, e.g., Arlasolve DMI-LQ, in an amount of about 0.1 wt. % to about 4 wt. %, such as, e.g., about 0.5 wt. %-about 3 wt. %, and phytonadione (vitamin K1) in an amount of about 0.1 wt. % to about 1.5 wt. %, such as, e.g., about 0.1 wt. %-~1 wt. %. In aspects in a separate container (e.g., a container not containing Phase A or Phase B), dimethyl isosorbide is collected. In aspects, phytonadione is added slowly, with mixing, until a uniform mixture is obtained (e.g., no particulate matter is visible.) In aspects, the Phase C component is maintained until one or more additional manufacturing steps are completed. In aspects, the Phase C component is maintained until required for addition to (combination with) one or more additional phases or ingredients. In aspects, Phase C is detectably or significantly protected from light.

In aspects, a Phase D component of the composition is prepared comprising deionized water in an amount of between about 2 wt. %-about 10 wt. % of the composition; propanediol, e.g., Zemea® Propanediol, in an amount of between about 0.5 wt. % and about 6 wt. %, such as, e.g., ~0.5 wt. %-about 5 wt. %, e.g., ~1 wt. %-~4 wt. %; hyaluronic acid, such as, e.g., hydrolyzed sodium hyaluronate, e.g., Mini HA in an amount of about 0.05 wt. %-about 1.5 wt. %, e.g., ~0.1 wt. %-~1 wt. %; Arnica montana flower extract and maltodextrin, e.g., Arnica montana Herbasec, in an amount of between about 0.0005 wt. % to about 2 wt. %, e.g., ~0.0005 wt. % to about 1.5 wt. %, e.g., ~0.001 wt. %-~1 wt. %; niacinamide, e.g., Niacinamide PC, in an amount of between about 1 wt. % to about 5 wt. %, such as, e.g., ~1 wt. %-~4 wt. %, e.g., ~1 wt. %-~3 wt. %; epigallocatechin gallate (green tea extract), e.g., EGCG 98, in an amount of between about 0.001 wt. % to about 1 wt. %, e.g., ~0.005 wt. %-~0.75 wt. %, e.g., ~0.01 wt. %-~0.5 wt. %; Panax notoginseng Root Extract in an amount of between about 0.05 wt. % to about 1 wt. %, such as, e.g., ~0.01 wt. %-~0.8 wt. %, e.g., ~0.1 wt. %-~0.7 wt. %; and, a combination of water, acetyl hexapeptide-8, potassium sorbate, phenoxyethanol, and acetic acid (e.g., Argireline Peptide Solution NP) in an amount (in total) of about, e.g., 0.01 wt. % to about 0.2 wt. %, e.g., ~0.05 wt. %-~0.15 wt. %, e.g., ~0.1 wt. %. In aspects, the water and propanediol are mixed in a separate container, e.g., a container not maintaining an existing Phase mixture. In aspects, mixing is continued until a uniform mixture is obtained (e.g., no particulate matter is visible). In aspects, the hyaluronic acid ingredient is added. In aspects, the hyaluronic acid ingredient is added slowly, e.g., sprinkled in slowly, while mixing. In aspects, the hyaluronic acid ingredient is added in intervals so as to prevent clumping of the hyaluronic acid ingredient. In aspects, mixing is continued until a clear solution is achieved. In aspects, the Arnica ingredient is added slowly in intervals to facilitate solubilization. In aspects, mixing is continued until a clear, uniform mixture is obtained. In aspects, the niacinamide ingredient is added while mixing until completely in solution (e.g., no particulate matter is visible). In aspects, remaining ingredients are added to the Phase D mixture. In aspects, remaining ingredient are added to the Phase D mixture in the order provided above. In aspects, ingredients of Phase D are added in an alternative order. In aspects, each ingredient is added one at a time. In aspects, each ingredient is added and mixed until completely in solution prior to the addition of the next ingredient. In aspects, the Phase D component is maintained until one or more additional manufacturing steps are completed. In aspects, the Phase D component is maintained until required for addition to (combination with) one or more additional phases or ingredients. In aspects, the Phase D component is maintained under constant mixing until needed for further processing.

In aspects, a Phase E component of the composition is prepared comprising deionized water in an amount representing about 40 wt. % to about 70 wt. %, about 45 wt. % to about 65 wt. % of the composition, and carbomer, e.g., Carbopol Ultrez 30, in an amount representing about 0.05 wt. % to about 1 wt. %, such as, e.g., about 0.05 wt. % to about 0.75 wt. %, e.g., about 0.1 wt. %-about 0.5 wt. % of the composition. In aspects, the deionized water is added to a separate container, which, in aspects, may be a main (or primary) container or “kettle”. In aspects, the carbomer is slowly added, e.g., by sprinkling, into the water under mixing, such as propeller mixing. In aspects, upon the complete addition of the carbomer, mixing is continued until the carbomer ingredient is completely hydrated. In aspects, once completely hydrated, the mixture is heated to a temperature of at least about 70° C., such as between about 70° C. to about 75° C. In aspects, the mixture is heated to no more than about 77° C. In aspects, mixing is continued, using, e.g., propeller mixing and including container/vessel sweeping (gently scraping/sweeping thicker material away from the container/vessel wall). In aspects, the propeller and sweep mixing are continued during the heating process. In aspects, the mixture is recirculated using pump recirculation while heating and mixing continues. According to alternative aspects, mixing can be performed using other types of mixing, e.g., according to the manufacturer's available equipment. In aspects, for example, Ekato PARAVISC mixing is performed. In aspects, mixing is continued during the heating process, and, depending on composition throughput through a mixing head or, e.g., depending on equipment turnover during mixing, pump recirculation may be used. In aspects, this is applicable to any mixing step described herein.

In aspects, a Phase F component of the composition comprises xanthan gum, such as, e.g., Xanthan Gum FNSCP-PC. In aspects, xanthan gum, in an amount representing about 0.1 wt. % to about 0.5 wtis added as Phase F to the Phase E mixture in the main kettle under continued propeller and sweep mixing. Upon the complete addition of Phase F, in aspects mixing is continued until the Phase F ingredient(s) (e.g., xanthan gum) is completely in solution. In aspects, pump recirculation of the mixture is continued. In aspects, the Phase E/F mixture in the main kettle is maintained at a temperature of between about 70° C. to about 75° C. with mixing. In aspects, the type of mixing and the utilization of pump recirculation is, as is stated previously, dependent upon, e.g., the type of equipment utilized. Again, in aspects, sufficient mixing is applied to assure suitable dispersion, hydration, uniformity, or any combination thereof, of ingredient(s).

In aspects, a Phase G component of the composition comprises sodium PCA (the sodium salt of pyrrolidone carboxylic acid (PCA)), also known as pyroglutamic acid, and water, such as, e.g., is provided by a single ingredient, e.g., Ajidew NL-50, in a total amount representing about 0.05 wt. % to about 2.5 wt. %, such as about 0.5 wt. % to about 2 wt. % or, e.g., about 0.1 wt. %-~2 wt. % of the composition; sodium phytate, e.g., Dermafeel PA-12, in an amount representing about 0.01 wt. % to about 0.5 wt. %, such as, e.g., about 0.01 wt. % to about 0.3 wt. %, e.g., about 0.01 wt. % to about 0.1 wt. %; phenoxyethanol and ethylhexylglycerin, e.g., Euxyl PE-9010, in a total amount of about 0.0001 wt. % to about 2 wt. %, such as, e.g., about 0.0001 wt. % to about 1.5 wt. %, or, e.g., about 0.0001 wt. %-about 1 wt. %; and sodium stearoyl glutamate, e.g., Eumulgin SG, in an amount representing about 0.1 to about 3 wt. % of the composition, such as, e.g., about 0.2 to about 2 wt. % of the composition. In aspects, ingredients of Phase G are added to Phase E/F. In aspects, ingredients of Phase G are added to Phase E/F under constant mixing, e.g., propeller mixing. In aspects, ingredients of Phase G are added to Phase E/F under continued propeller mixing and sweep mixing (or other appropriate forms of mixing as described previously). In aspects, each ingredient is added in the order listed. In aspects, one or more ingredients is added in an alternative order. In aspects, each ingredient is added one at a time and mixed until completely in solution (e.g., no particulate matter is visible) prior to the addition of the next ingredient. Upon the addition of all Phase G ingredients (forming a Phase E/F/G component), in aspects mixing is continued for about 3 to about 10 minutes, such as, e.g., ~3 minutes, ~4 minutes, ~5 minutes, ~6 minutes, ~7 minutes, ~8 minutes, ~9 minutes, or, e.g., about 10 minutes, or for a sufficient period of time to ensure complete uniformity. In aspects, such mixing is conducted with pump recirculation of the mixture. In aspects, the Phase E/F/G component is maintained until required for addition to (combination with) one or more additional phases or ingredients. In aspects, the Phase E/F/G component is maintained under constant mixing, in aspects with pump recirculation, and at a relatively constant temperature, until needed for further processing.

In aspects, a phase H component of the composition comprises glyceryl stearate citrate, e.g., Axol C-62 Pellets, in an amount representing about 1 wt. % to about 5 wt. %, such as, e.g., ~1 wt. % to about 4 wt. %, such as, e.g., ~1 wt. %-~3.5 wt. % of the composition; cetearyl alcohol, such as, e.g., Lanette O, in an amount representing about 0.5 wt. % to about 3 wt. %, such as, e.g., ~0.5 wt. % to about 2.5 wt. %, e.g., ~0.5 wt. %-~2 wt. % of the composition; caprylic/capric triglyceride, e.g., Myritol 312, in an amount representing about 1 wt. % to about 7 wt. %, such as, e.g., ~1 wt. %-~6 wt. %, e.g., ~1 wt. %-~5 wt. % of the composition; cetyl ricinoleate, e.g., Naturechem CR, in an amount of about 1 wt. % to about 7 wt. %, such as, e.g., ~1 wt. % to about 6 wt. %, or, e.g., ~1 wt. %-~5 wt. % of the composition; pentaerythrityl tetra-di-t-butyl hydroxyhydrocinnamate, e.g., Tinogard® TT, in an amount of about 0.1 wt. % to about 0.5 wt. %, such as, e.g., ~0.1 wt. %-~0.4 wt. %, e.g., about 0.1 wt. % to about 0.3 wt. % of the composition; salicylic acid, e.g., Salicylic Acid, USP, in an amount of about 0.1 wt. % to about 1 wt. %, such as, e.g., ~0.1 wt. %-~0.8 wt. %, such as, e.g., ~0.1 wt. %-~0.5 wt. % of the composition; myristyl alcohol, cetyl alcohol, and stearyl alcohol, such as, e.g., as is provided by Cetyl Alcohol 98% C16/98, in a total amount of about 0.001 wt. % to about 2.5 wt. %, e.g., ~0.001 wt. %-~2 wt. %, such as, e.g., ~0.001 wt. %-~1.5 wt. % of the composition; ethylhexyl olivate and squalane, such as, e.g., is provided by Botanessential Olive S-EHO, in a total amount representing about 1 wt. % to about 8 wt. %, e.g., ~1 wt. %-~7 wt. %, such as, e.g., ~1 wt. %-~6 wt. % or, e.g., ~1 wt. % to ~5 wt. % of the composition; and diheptyl succinate and capryloyl glycerin/sebacic acid copolymer, such as that provided by Lexfeel N200 MB, in a total amount of about 0.2 wt. % to about 2 wt. %, e.g., ~0.2 wt. %-~1.5 wt. %, such as, e.g., ~0.4 wt. %-~1 wt. % of the composition. In aspects, ingredients of Phase H are combined in a separate container (e.g., a container not containing other ingredients or phase components). In aspects, ingredients of Phase H are added in the order provided above. In aspects, ingredients of Phase H are added in an alternative order. In aspects, once all ingredients are combined, ingredients are heated, with mixing, to a temperature of at least 75° C., such as to a temperature of between about 75° C. to about 80° C. In aspects, the ingredients are not heated to a temperature of more than about 83° C. or about 82° C. In aspects, mixing is continued during heating and until a homogenous solution is obtained. In aspects, once a homogenous solution is obtained, the Phase H component is slowly added to the Phase E/F/G component. In aspects, the Phase H component is added to the Phase E/F/G component with mixing and pump recirculation. In aspects, pump recirculation, if applied, is started once all of Phase H is added. In aspects, upon the complete addition of the Phase H component, mixing is continued for at least an additional 3 minutes, such as, e.g., about 10 minutes, such as, e.g., ~4 minutes, ~5 minutes, ~6 minutes, ~7 minutes, ~8 minutes, or, e.g., ~9 minutes or for a sufficient period of time for a uniform mixture to be obtained. In aspects, time is dependent upon one or more factors including, e.g., batch size, equipment utilized, or e.g., both batch size and equipment utilized. In aspects, once sufficiently mixed, homogenization of the Phase E/F/G/H (Phase E-H) component is performed. In aspects, an emulsification is formed using equipment suitable for ensuring the establishment of a suitable particle size. In aspects, such equipment is homogenizer, homo mixer, or other such equipment known in the art. In aspects, at least one complete turn-over of the batch through the mixer head (homogenizer/homo mixer) is sufficient to homogenize the mixture. In aspects, such turn-over may be describable in other terms, e.g., according to the type of equipment utilized. For example, such turn-over may be such that it is capable of attaining a level of homogenization similar to that attained by the passage of a small volume of composition through a mixer head a number of times at a smaller scale, e.g., a lab scale. In aspects, more than one pass through the homogenizer/homo mixer is utilized. In aspects, after homogenization, mixing is switched to mixing sufficient to ensure proper turnover, such as, e.g., propeller and sweep mixing with (or, e.g., without) pump recirculation. In aspects, mixing is continued while allowing the mixture to cool to a temperature of at least about 67° C., such as at least about 66° C., ~65° C., or, e.g., to a temperature of between about 58° C.-about 67° C., such as, e.g., between about 60° C. to about 65° C. In aspects, the component (e.g., the “batch”) is then maintained under mixing and at the cooled temperature until required for further processing.

In aspects, the Phase A component is added to the Phase E-H component (the batch) in the primary/main container. In aspects, the Phase A component is added slowly, under continuous mixing, e.g., propeller and sweep mixing and pump recirculation. In aspects, once all of the Phase A component has been added, the mixture is mixed for a period of time to ensure complete mixing, e.g., at least an additional 5 minutes, such as, e.g., for an additional about 5 minutes to about 15 minutes, such as, e.g., about 10 minutes, or, e.g., for a sufficient period of time for a uniform mixture to be obtained. In aspects, mixing speed, pump recirculation speed, or both is adjusted one or more times during mixing. In aspects, mixing equipment is adjusted, e.g., mixing speed, pump recirculation speed, or both is/are adjusted to accommodate detectably or significant changes in mixture viscosity. In aspects, mixing is continued, e.g., propeller and sweep mixing and, e.g., pump recirculation, is continued while the mixture is cooled to at least about 48° C., such as to a temperature of between about 42° C. to about 48° C., such as, e.g., to a temperature of ~43° C.-~47° C., such as, e.g., to a temperature of about 45° C.

In aspects, the Phase B composition is then added to the batch. In aspects, the Phase B component is added to the batch under continuous mixing, e.g., propeller and sweep mixing and, e.g., pump circulation. In aspects, upon the complete addition of the Phase B component, mixing is continued for a sufficient period of time to ensure uniformity, such as, e.g., at least an additional about 3 minutes, such as, e.g., about 10 minutes, such as for ~4 minutes to about 7 minutes, or, e.g., ~5 minutes, or, e.g., for a sufficient period of time for a uniform mixture to be obtained. In aspects, the batch is then cooled to a temperature of at least about 37° C., such as to a temperature of between about 27° C. to about 37° C., e.g., ~28° C.-~36° C. or, e.g., about 30° C. to about 35° C. In aspects, cooling of the batch takes place under continuous mixing and pump recirculation.

In aspects, a phase I component is then added to the composition. In aspects, Phase I comprises tocopheryl nicotinate in an amount representing about 0.05 wt. % to about 2 wt. %, such as, e.g., ~0.075 wt. %-~1.5 wt. %, or, e.g., ~0.1 wt. %-~1 wt. % of the composition. In certain aspects, Phase I may contain one or more additional ingredients. In aspects, Phase I is added under suitable mixing, such as, e.g., propeller and sweep mixing with, in aspects, pump recirculation. In aspects, sufficient mixing is performed to obtain a uniform composition. In aspects, mixing is continue for a period of time to maintain uniform mixing. In aspects during mixing, the batch is cooled to between about 20° C. to about 40° C., such as, e.g., ~25° C.-~35° C., ~30° C.-40° C., or, e.g., ~30° C.-~35° C.

In aspects, the Phase C composition is then added to the batch. In aspects, the Phase C component is added to the batch under suitable mixing, such as, e.g., continuous mixing using, for example, propeller and sweep mixing. In aspects, pump recirculation is used. In aspects, upon the complete addition of the Phase C component, mixing is continued for sufficient period of time to obtain composition uniformity, such as, e.g., at least an additional about 8 minutes, such as for between about 8 minutes to about 12 minutes, e.g., for about 10 minutes or for a sufficient period of time for a uniform mixture to be obtained. In aspects, mixing time may vary due to batch size (volume of composition), equipment utilized, and the like.

In aspects, the Phase D composition is then added to the batch. In aspects, the Phase D component is added to the batch while mixing using suitable mixing techniques, such as, e.g., under continuous mixing, using, mixing techniques such as propeller and sweep mixing. In aspects, pump recirculation is utilized. In aspects, upon the complete addition of the Phase D component, mixing is continued for a period of time sufficient to ensure composition uniformity, such as, for example, at least an additional about 8 minutes, such as for between about 8 minutes to about 12 minutes, e.g., for about 10 minutes or for a sufficient period of time for a uniform mixture to be obtained. In aspects, as has been stated, mixing time can depend on certain conditions, such as, for example, batch size (composition volume), equipment utilized, etc.

In aspects, a Phase J component comprises phenethyl alcohol, ethylhexylglycerin and tocopherol, such as, e.g., is provided by Sensiva PA20, in a total amount representing between about 0.0001 wt. % to about 0.5 wt. % of the composition, e.g., ~00001 wt. %-~0.45 wt. % of the composition, such as, e.g., ~0.0001 wt. %-~0.4 wt. % of the composition. In aspects, the Phase J component is added to the batch. In aspects, the Phase J component is added to the batch. In aspects, Phase J is added while mixing using suitable mixing techniques. In aspects, a suitable mixing technique is continuous mixing. In aspects, mixing comprises use of propeller and sweep mixing, such as, e.g., continuous propeller and sweep mixing. In aspects, pump recirculation is applied. In aspects, upon the complete addition of the Phase J component, mixing is continued for a period of time sufficient to ensure composition uniformity, such as, for example, at least an additional about 8 minutes, such as for example between about 8 minutes to about 12 minutes, e.g., for about 10 minutes or for a sufficient period of time for a uniform mixture to be obtained. In aspects, suitable and sufficient mixing time can vary depending on the batch size (composition volume), mixing equipment utilized, etc.

In aspects, the batch is sampled to check the pH of the composition. In aspects at least 2 samples are obtained. In aspects, a sample is taken from the top of the batch and from the bottom of the batch in the container and checked for pH.

In aspects, a Phase K component comprises citric acid, e.g., a 50% solution of citric acid, and sodium hydroxide, e.g., a 20% solution of sodium hydroxide. In aspects, individual components of Phase K, such as, e.g., citric acid or sodium hydroxide, are added to the batch to adjust pH as needed to attain a target pH, such as, e.g., a pH of between about 4- 6.75, such as about 4.25-about 6.5, such as about 4.5-about 6, or such as about 4.75-5.25. In aspects, upon the addition of an amount of a Phase K component, the batch is mixed for at least approximately 15 minutes, such as, e.g., at least about 15 to about 25 minutes, e.g., for about 18 to about 22 minutes or about 20 minutes or a period of time sufficient to ensure composition uniformity before re-sampling for pH testing. In aspects, mixing is accompanied by pump recirculation. In aspects, each time a Phase K ingredient is added, the batch is mixed and resampled for pH testing.

In aspects, upon reaching a target pH, the batch is transferred to a storage or holding container (storage/holding vessel). In aspects, the storage/holding vessel, e.g., the top of the storage/holding vessel, is nitrogen purged, creating a nitrogen blanket over the surface of the composition. In aspects, the composition is stored in the airtight holding vessel for further processing, such as, e.g., until required for final packaging.

In aspects, all steps of the above-described exemplary manufacturing process are performed under aseptic conditions.

### EXAMPLES

The following detailed Examples are provided to assist readers in further understanding aspects of the invention or putting aspects of the invention into practice. The particular materials, methods, steps, and conditions employed/described in the following Examples, and results thereof, are intended to be further illustrative of aspects of the invention. These Examples reflect exemplary embodiments of the invention, and the specific methods, findings, principles of such Examples, and the general implications thereof, can be combined with any other aspect of the invention. However, readers should understand that the invention is not limited by or to any part of these Examples.

### Example 1: Exemplary Formulation and Production Thereof

Three exemplary compositions of the invention (Formulas 1, 2, and 3, respectively) were prepared having the formulas set forth in Table 4 (based on the amount of ingredients thereof present in each such formulation).

TABLE 4
(Exemplary Formulations 1-3)

| PHASE / | | Wt. % of | Wt. % of | Wt. % of |
|---|---|---|---|---|
| ORDER | INCI NAME | Formula 1 | Formula 2 | Formula 3 |
| A-01 | Purified Water | 30-85 | 29-85 | 28-84 |
| A-02 | Magnesium Aluminum Silicate | 0.1-1.5 | 0.1-1.5 | 0.1-1.5 |
| A-03 | Cetyl Hydroxyethylcellulose | 0.1-1 | 0.1-1 | — |
| A-04 | Glycerin | 0.5-3 | 0.5-3 | 0.5-3 |
| A-05 | Xanthan Gum | 0.1-0.5 | 0.1-0.5 | 0.1-0.5 |
| A-06 | Xanthan Gum, Lecithin, Sclerotium | — | — | 0.01-1 |
| | Gum, Pullulan | | | |
| A-07 | Methyl Nicotinate | 0.01-0.1 | — | — |
| A-08 | Sodium PCA, Water | 0.1-2 | 0.1-2 | 0.1-2 |
| A-09 | Niacinamide | 1-3 | 1-3 | 1-3** |
| A-10 | Phenoxyethanol, Ethylhexylglycerin | 0.1-1 | 0.1-1 | 0.1-1 |
| B-01 | Cetyl Ricinoleate | 1-5 | 1-5 | 1-5 |
| B-02 | Ethylhexyl Olivate, Squalane | 1-5 | 1-5 | 1-5 |
| B-03 | Sorbitan Olivate | 1-2 | 1-2 | 1-2 |
| B-04 | Glyceryl Stearate | 1-3 | 1-3 | — |
| B-05 | Glyceryl Stearate, PEG 100 Stearate | — | — | 1-3.5 |
| B-06 | Sorbitan Stearate | — | — | 1.5 |
| B-07 | Sodium Stearoyl Glutamate | 0.2-2 | 0.2-2 | 0.2-2 |
| B-08 | Cetearyl Alcohol | 0.5-2 | 0.5-2 | 0.5-2 |
| B-09 | Diheptyl Succinate, Capryloyl | 0.4-1 | 0.4-1 | 0.4-1 |
| | Glycerin/Sebacic Acid Copolymer | | | |
| B-10 | C12-15 Alkyl Benzoate | 1-4 | 1-4 | 1-4 |
| B-11 | Salicylic Acid | 0.1-0.5 | 0.1-0.5 | 0.1-0.5 |
| B-12 | Pentaerythrityl Tetra-di-t-butyl | 0.1-0.3 | 0.1-0.3 | 0.1-0.3 |
| | Hydroxyhydrocinnamate | | | |
| C-01 | Lactic Acid, Aqua | 0.1-0.5 | 0.1-0.5 | 0.1-0.5 |
| C-02 | Purified Water | 1-2.5 | 1-2.5 | 1-2.5 |
| C-03 | Sodium Lactate, Aqua | 0.5-1.5 | 0.5-1.5 | 0.5-1.5 |
| C-04 | Sodium Phytate | 0.01-0.1 | 0.01-0.1 | 0.01-0.1 |
| C-05 | Arginine | 0.1-1 | 0.1-1 | 0.1-1 |
| D-01 | Ethoxydiglycol | 0.5-2 | 0.5-2 | 0.5-2 |
| D-02 | Resveratrol | 0.1-1 | 0.1-1 | 0.1-1 |
| D-03 | Butylene Glycol | 1-4 | 1-4 | 1-4 |
| D-04 | Glycyrrhiza Glabra (Licorice) Root | 0.001-0.01 | 0.001-0.01 | 0.001-0.01 |
| | Extract | | | |
| D-05 | Curcuma Longa (Turmeric) Root | 0.0001-0.0005 | 0.0001-0.0005 | 0.0001-0.0005 |
| | Extract | | | |
| E-01 | Dimethyl Isosorbide | 0.5-3 | 0.5-3 | 0.5-3 |
| E-02 | Ethyl Lactyl Retinoate | 0.01-0.1 | 0.01-0.1 | 0.01-0.1 |
| F-01 | Dimethyl Isosorbide | 0.5-3 | 0.5-3 | 0.5-3 |
| F-02 | Phytonadione | 0.1-1 | 0.1-1 | 0.1-1 |
| G-1 | Purified Water | 2-6 | 2-6 | 2-6 |
| G-2 | Propanediol | 1-4 | 1-4 | 1-4 |
| G-3 | Hydrolyzed Sodium Hyaluronate | 0.1-1 | 0.1-1 | 0.1-1 |
| G-4 | Epigallocatechin Gallate | 0.01-0.5 | 0.01-0.5 | 0.01-0.5 |
| G-5 | Panax Notoginseng Extract | 0.1-0.7 | 0.1-0.7 | 0.1-0.7 |
| G-6 | Arnica Montana Flower Extract, | 0.001-1 | 0.001-1 | 0.001-1 |
| | Maltodextrin | | | |
| H-1 | Phenethyl Alcohol, | 0 | 0.0001-0.4 | 0.0001-0.4 |
| | Ethylhexylglycerin, alpha-Tocopherol | | | |

| | | | |
|---|---|---|---|
| Total | 100 | 100 | 100 |

**In formula 3, the niacinamide component was moved from phase A to phase G.

These formulations were generated in multiple steps/phases. For example, at least one of these formulations was prepared according to the following steps.

Phase A Steps: Water was added to a mixing container. Ultrez/carbomer was sprinkled into water with typical mixing until completely hydrated, then the mixture was heated to 70-75° C. with mixing. Then, xanthan gum was added, and mixing was continued until a uniform mixture was obtained. The remaining Phase A ingredients were added, one at a time, in the order indicated in Table 4 (03 before 04, 04 before 05, etc.), waiting until each ingredient is in solution before the next one was added. A temperature of 70-75° C. was held throughout this part of the process.

Phase B Preparation and Mixture with Phase A: Next, the Phase B ingredients were combined to form Phase B and the Phase B mixture was heated to 75-80° C. while mixing. Once the Phase B composition was uniform and at temperature, Phase B was slowly added to Phase A with mixing maintained for a few minutes (e.g., 2-5 minutes). The mixture was then subjected to homogenization for one turn of the batch. The mixed batch was thereafter allowed to cool to 60-65° C. while continued mixing was applied.

Phase C Preparation and Addition. Next, the phase C ingredients were mixed completely until completely in solution with no particulates. The mixture was heated to 45° C. and maintained. Once the Phase A-B emulsion was cooled to 60-65C, Phase C was slowly added to the emulsion with continued mixing for a few minutes. Mixing parameters were adjusted to maintain a desired consistent viscosity. While maintaining mixing, the batch was cooled to 45° C.

Phase D and Mixing. Next, Transcutol® was added to a separate container. While mixing, butylene glycol was added and mixed until uniform. The composition was heated to 65° C. while mixing continued. Resveratrol was then added, and the mixture mixed until a clear solution was achieved. This composition was cooled to 45° C., at which time licorice was added and the blend further mixed until completely in solution with no visible particulates. The mixture was then cooled to 40° C. under continued mixing. At this point curcumin was added to the mixture and the mixture further mixed until completely in solution. Mixing was thereafter maintained at 40° C. When the Phase A-C batch was at 45 C and Phase D mixture uniform and at 40° C., Phase D was added to the batch and the resulting mixed batch further mixed for a few minutes and the allowed to cool to 35-40° C.

Addition of Phase E, then Phase F. Once uniform, the batch (containing Phase A-D ingredients) was cooled to 30-35° C. with continued mixing. The Phase E ingredients were added to the batch under continued mixing and the batch was allowed to cool.

In a separate container, the Phase F ingredients were combined and mixed until a uniform solution was achieved. The Phase F mixture was added to the batch when temperature reached 30-35° C., under continued mixing.

Phase G Steps. In a separate container, water and propanediol were added and mixed. Mix until uniform. Hyaluronic acid was slowly sprinkled into this mixture under continued mixing conditions slowly to avoid formation of clumps. Mixing of the ingredients continued until a uniform solution was achieved. Niacinamide was added with mixing until completely in solution. Once Niacinamide is in solution, the remaining ingredients in Phase G were added to the mixture, making sure each ingredient is in solution before the next one is added. Once all of Phase G has been combined and was uniform, and batch was at 30-35° C., Phase G was added to the batch (containing Phase A-F ingredients) with mixing. The resulting combined batch was mixed and permitted to cool to 25-30° C.

Phase H Steps. Once the batch was at 25-30° C., Phase H was prepared and added to the batch with mixing until a uniform combined batch was achieved. A sample was taken, and pH evaluated.

Phase I Steps (Optional). If needed to achieve desired pH (4.75-5.25), a Phase I was prepared and added to the batch to adjust pH. Phase I comprised each of a 50% citric acid solution and a sodium hydroxide solution. A citric acid 50% solution was/can be used to lower pH and a sodium hydroxide solution (20-50%) was/can be used to increase pH.

This Example exemplifies how a multi-ingredient formulation according to the invention can be generated through conventional mixing, heating, and cooling methods, applied across multiple phases/steps, to ensure optimal mixing of ingredients. This Example exemplifies that the invention provides compositions produced by a multi-phase ingredient mixture process, comprising at least four, at least five, or at least six separate phase preparation/mixture steps. In aspects, such steps include heating, continuous mixing, and cooling, as the batch increases in complexity. Thus, this disclosure can be applied to more general amounts of the ingredients described herein provided in the specification to provide compositions defined as product-by-process compositions, inasmuch as such process steps impart beneficial properties to such compositions which are distinct from compositions prepared by other method steps. Such product-by-process compositions are yet another aspect of the invention.

### Example 2: Exemplary Formulation

Fourth and fifth exemplary compositions of the invention (Formula 4 and Formula 5) were prepared having the formulas set forth in Table 5.

TABLE 5
(Exemplary Formulations 4 and 5)

| | Wt. % of | Wt. % of |
|---|---|---|
| INCI NAME | Formula 4 | Formula 5 |
| Water/Aqua | 27-86 | 38-89 |
| Cetyl Ricinoleate | 1-5 | 1-5 |
| Ethylhexyl Olivate | 1-5 | 1-5 |
| C12-15 Alkyl Benzoate | 1-4 | — |
| Butylene Glycol | 1-4 | 1-4 |
| Niacinamide | 1-3 | 1-3 |
| Caprylic/Capric Triglyceride | — | 1-5 |
| Glyceryl Stearate Citrate | — | 1.5-3.5 |
| Propanediol | 1-4 | 1-4 |
| Glycerin | 0.5-3 | — |
| Ethoxydiglycol | 0.5-2 | 0.5-2 |
| Sorbitan Olivate | 1-2 | — |
| Glyceryl Stearate | 1-3.5 | — |
| PEG-100 Stearate | 1-3.5 | — |
| Dimethyl Isosorbide | 0.5-3 | 0.5-3 |
| Phytonadione | 0.1-1 | 0.1-1 |
| Resveratrol | 0.1-1 | 0.1-1 |
| Sorbitan Stearate | 0.5-1.5 | — |
| Maltodextrin | 0.1-1 | 0.1-1 |
| Phenoxyethanol | 0.1-1 | 0.1-1 |
| Magnesium Aluminum Silicate | 0.1-1.5 | — |
| Sodium Lactate | 0.5-1.5 | — |
| Capryloyl Glycerin/Sebacic Acid | 0.4-1 | 0.4-1 |
| Copolymer | | |
| Arginine | 0.1-1 | 0.1-1 |
| Cetearyl Alcohol | 0.5-2 | 0.5-2 |
| Panax Notoginseng Root Extract | 0.1-0.7 | 0.1-0.7 |
| Sodium PCA | 0.1-2 | 0.1-2 |
| Salicylic Acid | 0.1-0.5 | 0.1-0.5 |
| Diheptyl Succinate | 0.4-1 | 0.4-1 |
| Carbomer | — | 0.1-0.5 |
| Xanthan Gum | 0.01-1 | 0.01-1 |
| Pentaerythrityl Tetra-di-t-butyl | 0.1-0.3 | 0.1-0.3 |
| Hydroxyhydrocinnamate | | |
| Hydrolyzed Sodium Hyaluronate | 0.1-1 | 0.1-1 |
| Sodium Stearoyl Glutamate | 0.2-2 | 0.2-2 |
| Tocopheryl Nicotinate | 0.1 | 1 |
| Squalane | 0.1-1 | 0.1-1 |
| Lactic Acid | 0.1-0.5 | — |
| Phenethyl Alcohol | 0.0001-0.35 | 0.0001-0.35 |
| Lecithin | 0.01-1 | — |
| Sclerotium Gum | 0.01-1 | — |
| Ethylhexylglycerin | 0.1-1 | 0.1-1 |
| Pullulan | 0.01-1 | — |
| Cetyl Alcohol | — | 0.001-1.5 |
| Sodium Phytate | 0.01-0.1 | 0.01-0.1 |
| Silica | 0.01-1 | — |
| Epigallocatechin Gallate | 0.01-0.5 | 0.01-0.5 |
| Glycyrrhiza Glabra (Licorice) | 0.001-0.01 | 0.001-0.01 |
| Root Extract | | |
| Arnica Montana Flower Extract | 0.001-1 | 0.001-1 |
| Myristyl alcohol | — | 0.001-1.5 |
| Stearyl Alcohol | — | 0.001-1.5 |
| Curcuma Longa (Turmeric) | 0.0001-0.0005 | 0.0001-0.0005 |
| Root Extract | | |
| Tocopherol | 0.0002 | 0.0001-0.35 |
| Potassium Sorbate | 0.0001-0.1 | 0.0001-0.1 |
| Acetyl Hexapeptide-8 | 0.00005-0.001 | 0.00005-0.001 |
| Acetic Acid | — | — |
| Citric Acid | — | — |
| Sodium Hydroxide | — | — |
| TOTAL: | 100.0 | 100.0 |

### Example 3: Case Study (Subject MW)

This Example demonstrates the application of a composition according to the invention in the treatment of bruising and purpura associated with long-term use of immune suppressants.

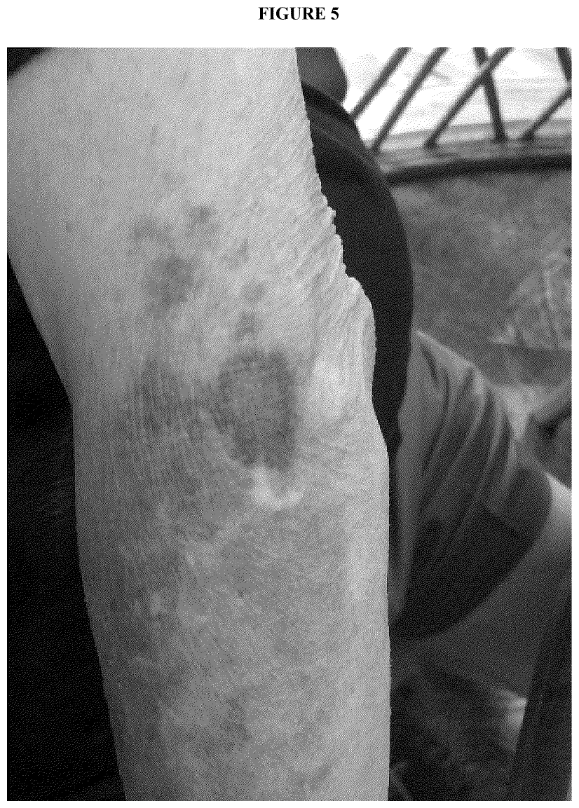

A 70-year-old male taking immune suppressants for a solid organ transplant was provided a test composition according to formulation 4 (Formula 4 of Table 5, Example 2) to apply to bruising and purpura associated with long-term use of immune suppressants. The patient applied approximately 0.2-0.5 mL of the formulation to a fresh bruise (“test bruising”) and fresh purpura (“test purpura”) as soon as the test bruising and purpura first appeared, estimated to be within approximately 0-24 hours of first appearance. The patient continued applying approximately the same amount of composition to the test bruising and purpura twice per day. A second set of bruising (“control bruising”) and purpura (“control purpura”) was selected which were, according to visual observation, of comparable size and which appeared at about the same time as the test bruising and purpura (e.g., appeared within about 12-24 hours) of the appearance of the test bruising and purpura). The patient made visual observations of the test and control bruising and purpura over the course of treatment. Notably, the patient notably was an experienced dermal investigator. Photographs of the test sites over the course of treatment are provided as FIGS. 5-8.

FIG. 5 shows the test bruise, present on the patient's elbow, prior to treatment.

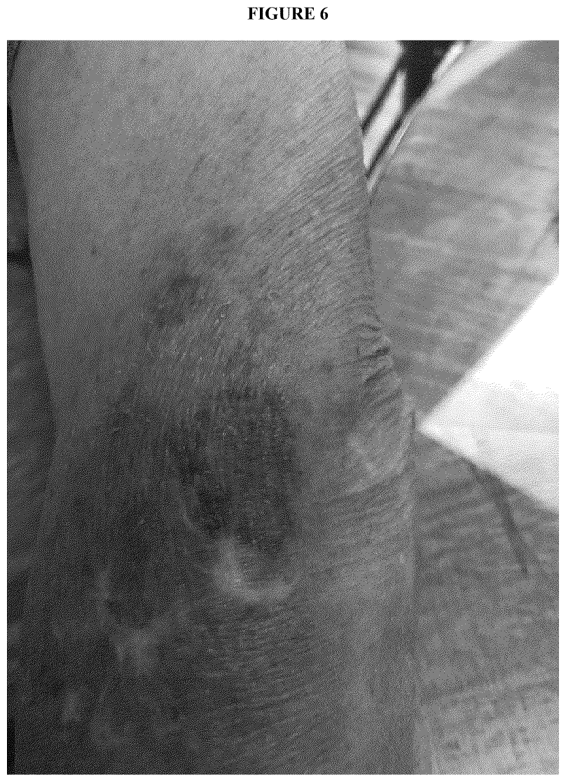

FIG. 6 shows test bruise, present on the patient's elbow, approximately 4 days after initial application.

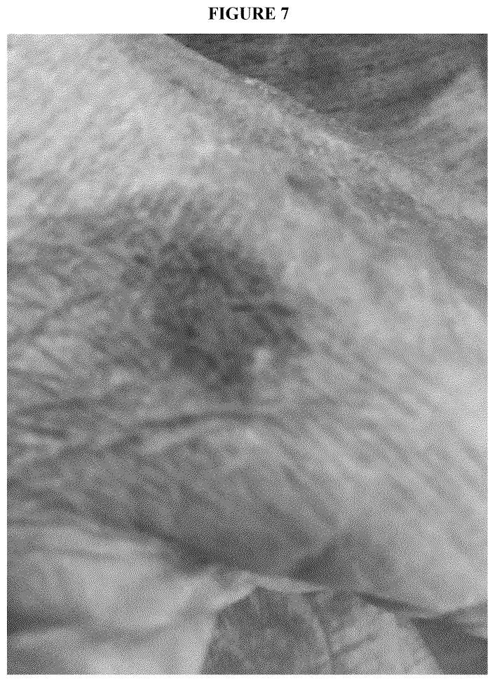

FIG. 7 shows the test purpura, present on the patient's hand, prior to treatment.

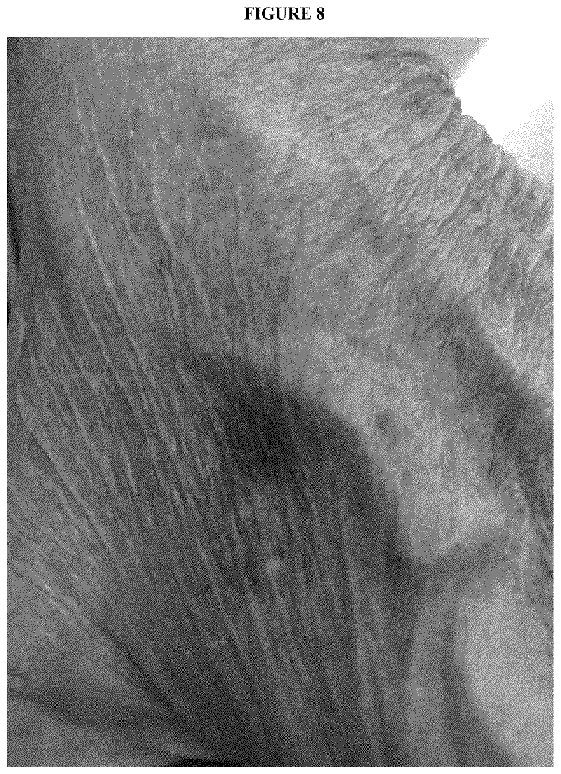

FIG. 8 shows the test purpura, present on the patient's hand, approximately 4 days after initial application.

Initial improvement in the color and intensity of the bruise compared to control was observed within 24 hours of first application. The central region of the bruise was the first region of the bruise to respond. Within the same period (initial 24 hours of first application), the control bruise remained the same or the color of the bruise intensified. Improvement of the treated bruise continued over the course of 5 days at which time the treated bruise was present only as a faint crescent or halo. The control areas faded slowly over the course of over 2 weeks. Purpura responded in a similar fashion resolving within 5 days similarly to bruising.

This experiment was performed a number of additional times in a comparable manner, comparing the treatment of treated bruise(s) to control bruise(s) and treated purpura to control purpura. Detectably faster response to the treatment was observed when composition(s) were applied to bruising or purpura within the first 0-24 hours of their appearance than when composition(s) were applied to older bruises or purpura which had been present for about 36 hours or longer (such as bruises or purpura present for a period of 2-3 days) prior to the administration of composition(s). As noted, the patient was a solid organ transplant recipient, extremely sensitive to bruising/purpura. The patient reported that several other products have been tested over time in attempts to reduce such visible bruising. The patient reported that the product tested in this experiment, the results of which are shown in FIGS. 5-8, illustrate resolution of bruising and purpura within days as opposed to weeks observed either without treatment or with treatment using other products.

Further, while it was observed that treatment of bruise(s) and purpura present for greater than 36 hours prior to initial treatment still demonstrated faster time to complete resolution than untreated bruise(s) and purpura, the time to such complete resolution took longer when treatment was applied to such older bruise(s) and purpura than when treatment was applied to bruise(s) and purpura within 24 hours of their first appearance. The pattern of the clearance of bruising was similar whether the compositions were applied within 24 hours of first appearance or after 24 hours of first appearance of bruising.

This experiment illustrates the effectiveness in treating bruising/purpura using a single inventive test formulation. The product demonstrated successful reduction in resolution time of both bruising and purpura compared to that without treatment experienced and reported by the test subject, a solid organ transplant recipient extremely susceptible to bruising and a common sufferer of bruising therefrom.

### Example 4: Case Study (Subject MW)

This Example demonstrates the application of a composition according to the invention in the treatment of bruising and purpura associated with long-term use of immune suppressants (the same treatment subject of Example 3).

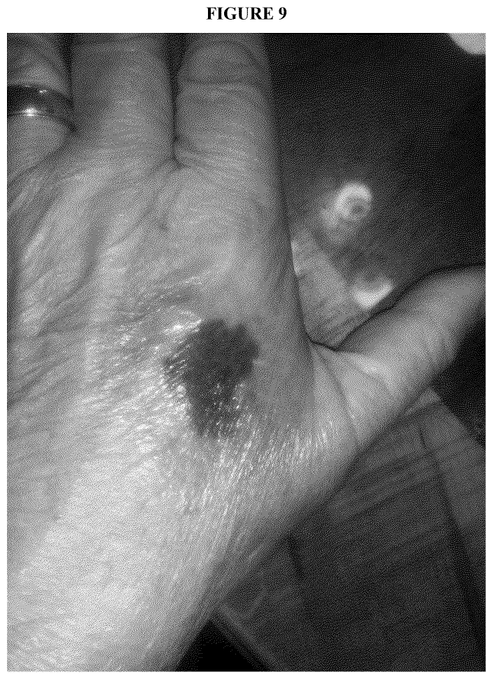

A 70-year-old male taking immune suppressants for a solid organ transplant was provided a test composition according to Formulation 4 (Formula 4 of Table 5, Example 2) to apply to bruising associated with long-term use of immune suppressants. The patient applied approximately 0.2-0.5 mL of the formulation to a fresh bruise (“test bruising”) on the patient's hand as soon as the test bruising first appeared, estimated to be within approximately 0-24 hours of first appearance. The patient continued applying approximately the same amount of composition to the test bruising twice per day. The patient made visual observations of the test bruising over the course of treatment. Notably, the patient notably was an experienced dermal investigator. Photographs of the test site over the course of treatment are provided as FIGS. 9-12.

FIG. 9 shows the test bruise, present on the patient's hand, 2 hours post-first application of Formulation 4.

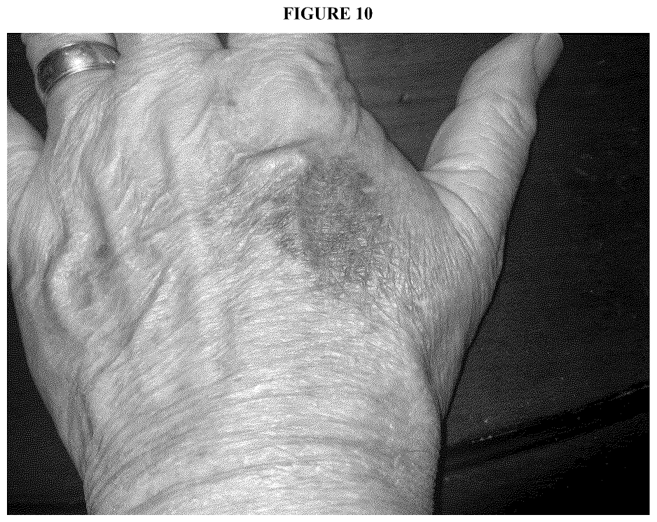

FIG. 10 shows the test bruise, present on the patient's hand, approximately 72 hours (3 days) after initial application.

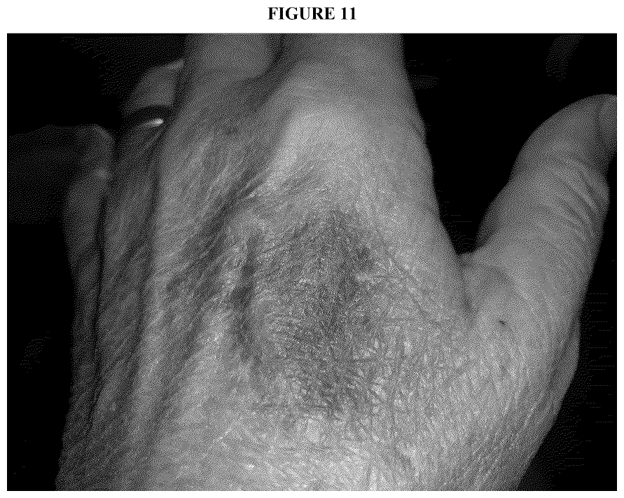

FIG. 11 shows the test bruise, present on the patient's hand, approximately 96 hours (4 days) after initial application.

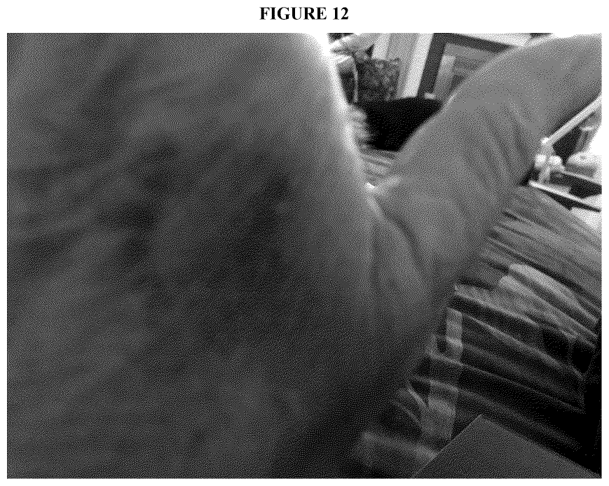

FIG. 12 shows the test bruise, present on the patient's hand, approximately 7 days after initial application where the bruise is almost clear, especially at the center of the bruise.

Initial improvement in the color and intensity of the bruise compared to control was observed within 24 hours of first application. The central region of the bruise was, similar to that reported in Example 4, the first region of the bruise to respond. Improvement of the treated bruise continued over the course of 7 days at which time the treated bruise was almost cleared, with the center of the bruise being essentially cleared based on visual observation.

This experiment illustrates the effectiveness in treating bruising using a single test inventive test formulation. The product demonstrated successful reduction in resolution time of bruising compared to that without treatment experienced and reported by the test subject, a solid organ transplant recipient extremely susceptible to bruising and a common sufferer of bruising therefrom.

### Example 5: Case Study (Subject AR)

This Example exemplifies the use of compositions of the invention to reduce moderate and severe bruising associated with application of an activity, here an aesthetic injection procedure.

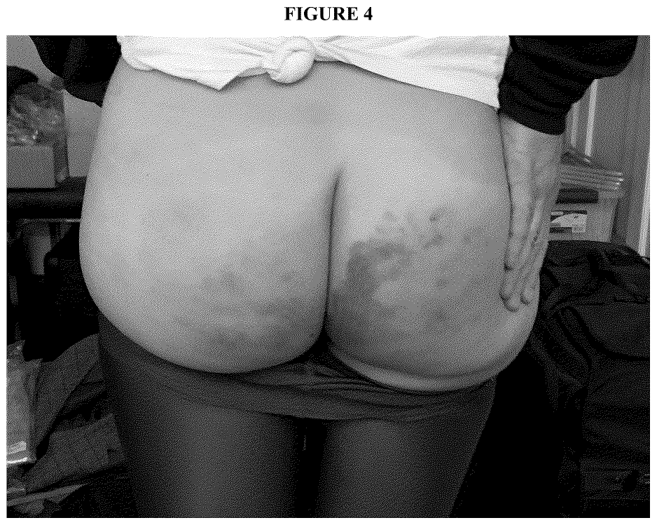

A forty-seven-year-old female in good health underwent cellulite treatment using the Qwo® (collagenase Clostridium histolyticum) injection treatment (Endo International). The Qwo® product/procedure is commercially available and described in the art (see, e.g., Sadick N S, et al. Dermatol Surg. 2019 August; 45 (8):1047-1056 and Quo® prescribing information (available from FDA). The subject underwent the first injection without application of the composition. For the second round of collagenase Clostridium injection, performed about a month after the first treatment, the subject applied a test composition according to Formulation (Formula 2 of Table 4, Example 1) twice a day for two days to the left half of her buttocks and did not apply the composition to the right buttocks. At the beginning of treatment, the amount of bruising on both sides of the buttocks of the subject was similar. However, after only two days post treatment, the subject experienced a dramatic reduction in the amount of dark bruising. The results of this case study are reflected in FIG. 4, which is photograph of the subject taken after two days of treatment with the composition. The difference in the degree of bruising, size of bruise area, number of visible bruises, etc. (e.g., in terms of darkness of bruises, amount of purple coloring, extent of bruised area, etc.) can be clearly seen in this photograph. This Example demonstrates that compositions of the invention are capable of reducing bruising associated with activities, such as application of aesthetic procedures, within a short amount of time (e.g., reduction by a significant amount, such as at least about 20%, at least about 25%, at least about 33%, or at least about 50% of bruising in one or more aspects after only ~two days post treatment.

### Example 6: Case Study (Subject JW)

A 50-year-old Caucasian male with a notable history for mild hypertension and daily aspirin use, resulting in the patient being somewhat prone to bruising, experienced the formation of a highly indurated hematoma caused by a blunt force injury to the patient's upper leg. The hematoma demonstrated deep bleeding, with an about 8 mm to about 12 mm palpated depth with a hard, indurated area. The hematoma was highly circumscribed (well defined).

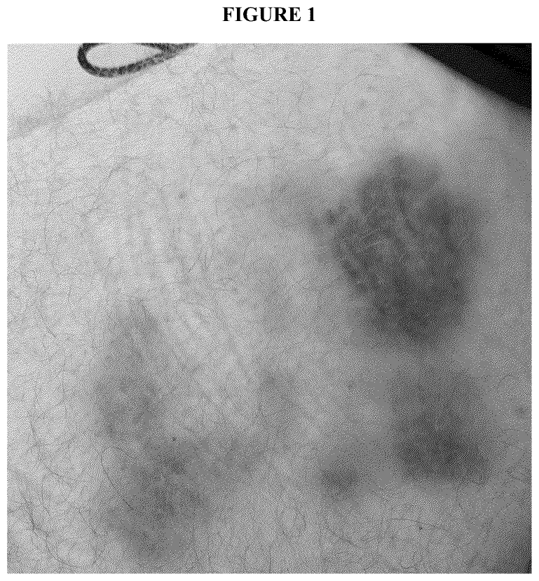

The patient was provided with a test composition according to formulation 5 (Formula 5 of Table 5, Example 2) for product testing purposes and observed by product development personnel over the treatment period. Test Formula 5 was applied twice per day to the highly indurated hematoma and the surrounding associated bruises visible in FIG. 1, a photograph taken upon initial injury and at the beginning of treatment. Of note is the physiological effect demonstrated that because the injury is deep, it takes time for blood to get to the surface (hence later photographs show darker bruising during the healing process.) Treatment involved massaging a small amount of the formulation onto the affected area and allowing the formulation to absorb into the skin before covering with clothing.

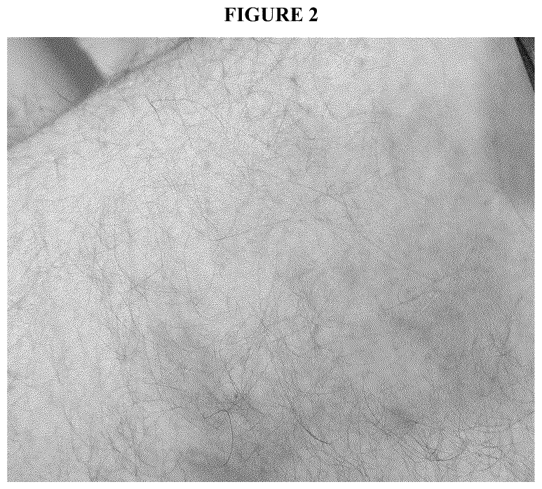

FIG. 2 is a photograph of the treated bruises and hematoma 3 days after treatment began. Within 24-48 hours, much of the simple bruising had resolved.

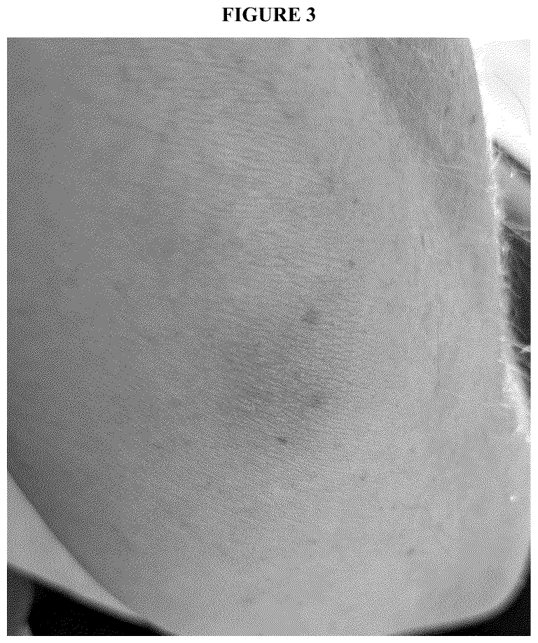

FIG. 3 is a photograph of the treated bruises and hematoma 5 days after treatment began. FIG. 3 illustrates that at day 5 of treatment, the bruising and hematoma was almost completely, based on visible observation, completely resolved.

This experiment illustrates the effectiveness in treating bruising using a single test inventive test formulation. As reported by observational personnel, such bruising/hematoma formation would, under normal circumstances without treatment, normally take 21 days to resolve. Hence in this Example, the inventive formulation demonstrated a successful reduction in resolution time of bruising compared to a similar injury without treatment (an expedited healing time) of almost 75%.

### Example 7: Case Study (Cupping)

A study was conducted to evaluate the effect of inventive composition(s) on somewhat controlled and expected bruising caused by a cupping procedure.

A patient underwent a provider-controlled dry-cupping procedure to both the left and right sides of the patient's spine. The dry cupping procedure comprised the application of suction to a single defined area on either side of the patient's spine using cups, drawing blood and fluids to the targeted area. No blood was removed (e.g., the procedure did not involve wet cupping.) As is known in the art, cupping procedure(s) cause changes in lymphatic and blood flow but does not cause induration (localized hardening of soft tissue).

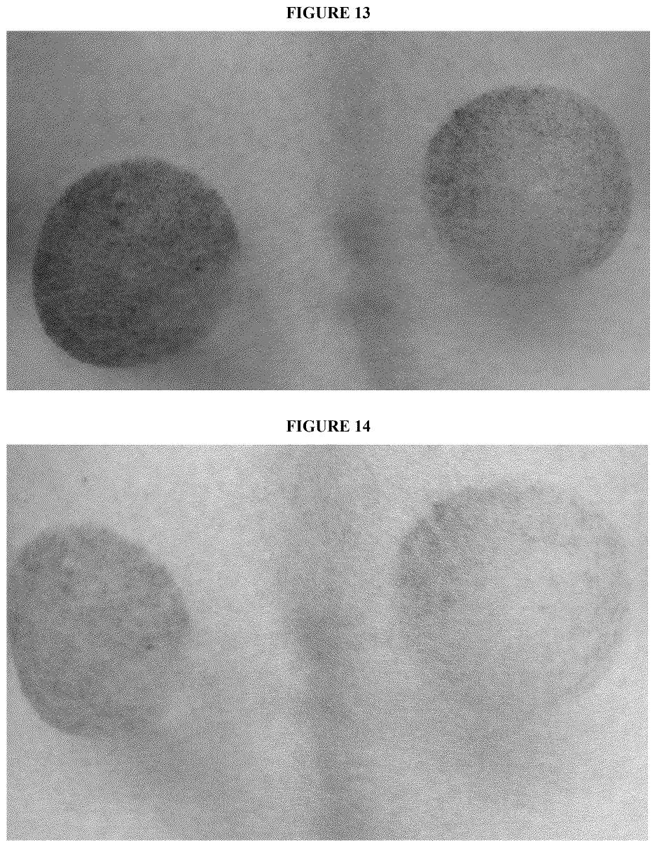

Immediately post-cupping procedure, a control everyday moisturizer composition was applied to the affected area on the left side of the patient's spine. Immediately post-cupping procedure, a composition according to investigative formulation 5 (Formula 5 of Table 5, Example 2) was applied to affected area on the right side of the patient's spine. (Description of left and right sides are provided according to the perspective of an observer, e.g., the left and right sides of photographs 13-15; also, the patient's left and right sides.) Each of the control moisturizer composition and Formula 5 was applied in an amount of about 0.5-1 mL to each respective affected area twice per day for 48 hours. Results are shown in FIGS. 13-15.

FIG. 13 shows each of the two affected areas at 24-hours post-procedure, with the area receiving twice daily application of the control moisturizer on the left and the area receiving twice daily application of Formula 5 on the right. There is clear evidence of clearing of the bruise already visible at 24 hours in the area treated with Formula 5, especially in the central and anatomically lower regions of the bruise.

FIG. 14 shows each of the two affected areas at 36-hours post-procedure. Visible clearing of the bruise continues in the affected area on the right side of the spine, treated with Formula 5, compared to the area treated with the control moisturizer on the left side.

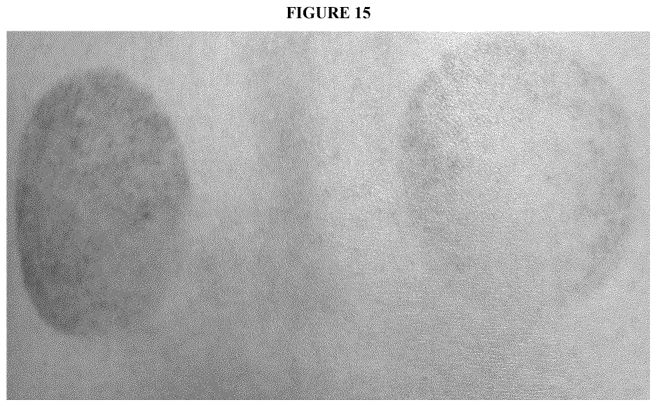

FIG. 15 shows each of the two affected areas at 48-hours post-procedure. Visible clearing of the bruise continues in the affected area on the right side of the spine, treated with Formula 5, compared to the area treated with the control moisturizer on the left side.

This Example shows the remarkable effect of the inventive composition on the time required to visibly observe clearing of bruising caused by a cupping procedure.

### Example 8: Clinical Study

A clinical study was performed using a test formulation product developed according to the principles of Example 1 and other relevant parts of this disclosure.

Twenty-five patients in total were enrolled in the study. Fifteen of the twenty-five patients were treated with the test formulation one day prior to being initially treated with collagenase Clostridium histolyticum-aaes (“CCH”) in either both buttocks or both thigh areas. Ten patients were treated with the test formulation three days prior to similar initial CCH treatment(s). Medical professionals administering the study assessed initial CCH treatment-associated bruising in the subjects as either mild or moderate. Other study demographics are presented in Table 6, below.

TABLE 6
Clinical Study Demographics

| | ALL SUBJECTS | PRE-TREAT 1 DAY | PRE-TREAT 3 DAYS |
|---|---|---|---|
| | (N = 25) | (N = 15) | (N = 10) |
| AGE | 42 yrs. (Mean) | 42 yrs. (Mean) | 42 yrs. (Mean) |
| | 22, 50 (Min., Max) | 22, 50 (Min., Max) | 23, 50 (Min., Max) |
| RACE | African American/Black - 16% (4) | African American/Black - 7% (1) | African American/Black - 30% (3) |
| | Caucasian/White - 64% (16) | Caucasian/White - 80% (12) | Caucasian/White - 40% (4) |
| | *Other - 16% (4) | *Other - 7% (1) | *Other - 30% (3) |
| | Multi-Race - 4% (1) | Multi-Race - 7% (1) | Multi-Race - 0 |
| **ETHNICITY | Hispanic - 36% (9) | Hispanic - 40% (6) | Hispanic - 30% (3) |
| | Not Hispanic - 56% (14) | Not Hispanic - 60% (9) | Not Hispanic - 50% (5) |
| SKIN TYPE | II - 32% (8) | II - 13% (2) | II - 60% (6) |
| | III - 28% (7) | III - 47% (7) | III - 0 |
| | IV - 28% (7) | IV - 40% (6) | IV - 10% (1) |
| | V - 4% (1) | V -0 | V - 10% (1) |
| | VI - 8% (2) | VI -0 | VI - 20% (2) |
| SEVERITY | Mild - 28% (7) | Mild - 27% (4) | Mild - 30% (3) |
| | Moderate - 72% (18) | Moderate - 73% (11) | Moderate - 70% (7) |
| BUTTOCKS | 56% (14) | 47% (7) | 70% (7) |
| THIGHS | 44% (11) | 53% (8) | 30% (3) |

*Other (n = 1, each): Amazonian/Indian/W. African; Middle Eastern; N. African; Dominican; British/Arabic/Venezuelan.
**Data Missing

Study subjects were instructed to apply the test formulation to the affected area of the buttocks or thigh on a daily basis beginning prior to a first CCH treatment according to their assigned test group (e.g., 1 or 3 days prior to first CCH treatment) and continuing post-CCH treatment. Subjects were randomized with respect to which buttock or thigh (right or left) of the CCH-treated buttocks or thighs was to receive administration of the test formulation. As noted, some subjects were instructed to pre-treat the selected CCH-treated area for 3 days and others were instructed to pre-treat the selected CCH-treated area for 1 day (prior to initial CCH administration). CCH treatment that ran concurrently with the study of the test formulation included 3 CCH applications. Trained dermatologists or other trained and qualified medical professionals made bruising assessments at the following timepoints: 72 hours post the first CCH administration (CCH #1), one week post CCH #1, and three weeks post CCH #1; three weeks post CCH treatment two (CCH #2); and four weeks post CCH treatment three (CCH #3). Medical professionals assessed bruising in the study using the following scale.

TABLE 7
Bruising Assessment Scale Used in Clinical Study

| SCORE | CATEGORY | DESCRIPTION |
|---|---|---|
| 0 | No/Almost No | No Bruising or almost no bruising. |
| | Bruising | |
| 1 | Mild Bruising | Mild intensity bruising, pink-light |
| | | red color covering approximately 25% |
| | | of the buttock or thigh. |
| 2 | Moderate Bruising | Mild intensity bruising, deep red to |
| | | purple color, covering approximately |
| | | 50% of the buttock or thigh. |
| 3 | Severe Bruising | Severe bruising, deep purple color |
| | | covering approximately 75% of the |
| | | buttock or thigh. |
| 4 | Very Severe Bruising | Severe bruising, deep to darkest |
| | | purple color covering approximately |
| | | 100% of the buttock or thigh. |

The results of these assessments are presented in Table 8, below.

TABLE 8
Bruising Assessment Measurements

| Visit | All Subjects | Pretreat 1 day | Pretreat 3 days |
|---|---|---|---|
| Statistic | (N = 25) | (N = 15) | (N = 10) |
| Baseline | | | |
| N | 25 | 15 | 10 |
| Mean (SD)* | 0.0 (0.00) | 0.0 (0.00) | 0.0 (0.00) |
| Median | 0.00 | 0.00 | 0.00 |
| Min, Max | 0, 0 | 0, 0 | 0, 0 |
| p-value | N/A | N/A | N/A |
| Treatment 1, 72 Hour | | | |
| N | 24 | 14 | 10 |
| Mean (SD)* | −0.2 (1.01) | −0.1 (1.00) | −0.3 (1.06) |
| Median | 0.00 | 0.00 | −0.50 |
| Min, Max | −2, 2 | −2, 2 | −2, 1 |
| p-value | 0.4259 | 0.7929 | 0.3938 |
| Treatment 1, Week 1 | | | |
| N | 24 | 15 | 9 |
| Mean (SD)* | −0.3 (0.69) | −0.3 (0.72) | −0.2 (0.67) |
| Median | 0.00 | 0.00 | 0.00 |
| Min, Max | −1, 1 | −1, 1 | −1, 1 |
| p-value | 0.0499 | 0.0961 | 0.3466 |
| Treatment 1, Week 3 | | | |
| N | 22 | 14 | 8 |
| Mean (SD)* | 0.0 (0.00) | 0.0 (0.00) | 0.0 (0.00) |
| Median | 0.00 | 0.00 | 0.00 |
| Min, Max | 0, 0 | 0, 0 | 0, 0 |
| p-value | N/A | N/A | N/A |
| Treatment 2, Week 3 | | | |
| N | 22 | 14 | 8 |
| Mean (SD)* | −0.0 (0.21) | −0.1 (0.27) | 0.0 (0.00) |
| Median | 0.00 | 0.00 | 0.00 |
| Min, Max | −1, 0 | −1, 0 | 0, 0 |
| p-value | 0.3287 | 0.3356 | N/A |
| Treatment 3, Week 4 | | | |
| N | 21 | 14 | 7 |
| Mean (SD)* | −0.3 (0.56) | −0.4 (0.63) | −0.1 (0.38) |
| Median | 0.00 | 0.00 | 0.00 |
| Min, Max | −1, 1 | −1, 1 | −1, 0 |
| p-value | 0.0300 | 0.0548 | 0.3559 |

*Note:
Mean values generated by a statistician and represent average(s)s across all subjects based on the assessment of bruising severity on each side of the treated area using the 5-point scale.

As can be seen from the data presented in Table 8, there was a statistically significant improvement in the overall population in terms of bruising reduction in the treated area of subjects as compared to the untreated area (p=0.03). This data is supportive of the effect of formulations of the invention, such as the test formulation, to reduce bruising in subjects over similar periods of time. Results at one week post initial CCH treatment also were encouraging, suggesting that such formulations may be useful in initial and longer-term reduction of bruising effects.

Subjects also were instructed to make a self-assessment on effects of the test formulation (the “PQ Cream”) and on the product's attributes 72 hours post CCH #1, one week post CCH #1, and three-weeks post CCH #1; three weeks post CCH #2; and four weeks post CCH #3. The results of these assessments are presented in Table 9.

TABLE 9
Clinical Trial Subject Self-Assessment Scores

| 72 Hours PostCCH#1 | | 1 Week Post CCH#1 | | 3 Weeks Post CCH#1 | | 4 Weeks Post CCH#3 | |
|---|---|---|---|---|---|---|---|
| (N = 24) | | (N = 24) | | (N = 22) | | (N = 22) | |
| AGREE | DISAGREE | AGREE | DISAGREE | AGREE | DISAGREE | AGREE | DISAGREE |

SUBJECT SATISFACTION

| | | | | | | | | | |
|---|---|---|---|---|---|---|---|---|---|
| 1. | My skin looks better | 46% | 54% | 46% | 54% | 59% | 41% | 64% | 36% |
| | overall on the PQ | (11) | (13) | (11) | (13) | (13) | (9) | (14) | (8) |
| | Cream-treated side in | | | | | | | | |
| | comparison to the | | | | | | | | |
| | side with no | | | | | | | | |
| | application of cream. | | | | | | | | |
| 2. | My skin is less | 50% | 50% | 42% | 58% | 50% | 50% | 59% | 41% |
| | bruised-looking on | (12) | (12) | (10) | (14) | (11) | (11) | (13) | (9) |
| | the PQ Cream-treated | | | | | | | | |
| | side in comparison to | | | | | | | | |
| | the side with no | | | | | | | | |
| | application of cream. | | | | | | | | |
| 3. | My skin is less | 54% | 46% | 42% | 58% | 50% | 50% | 64% | 36% |
| | discolored-looking | (13) | (11) | (10) | (14) | (11) | (11) | (14) | (8) |
| | on the PQ Cream- | | | | | | | | |
| | treated side in | | | | | | | | |
| | comparison to the | | | | | | | | |
| | side with no | | | | | | | | |
| | application of cream. | | | | | | | | |
| 4. | My skin is less red- | 50% | 50% | 46% | 54% | 59% | 41% | 64% | 36% |
| | looking on the PQ | (12) | (12) | (11) | (13) | (13) | (9) | (14) | (8) |
| | Cream-treated side in | | | | | | | | |
| | comparison to the | | | | | | | | |
| | side with no | | | | | | | | |
| | application of cream. | | | | | | | | |
| 5. | My skin is less | 33% | 67% | 46% | 54% | 55% | 45% | 64% | 36% |
| | yellow-looking on | (8) | (16) | (11) | (13) | (12) | (10) | (14) | (8) |
| | the PQ Cream-treated | | | | | | | | |
| | side in comparison to | | | | | | | | |
| | the side with no | | | | | | | | |
| | application of cream. | | | | | | | | |
| 6. | My skin is less | 46% | 54% | 50% | 50% | 45% | 55% | 59% | 41% |
| | black-looking on the | (11) | (13) | (12) | (12) | (10) | (12) | (13) | (9) |
| | PQ Cream-treated | | | | | | | | |
| | side in comparison to | | | | | | | | |
| | the side with no | | | | | | | | |
| | application of cream. | | | | | | | | |
| 7. | My skin is more | 46% | 54% | 46% | 54% | 50% | 50% | 68% | 32% |
| | even-looking in | (11) | (13) | (11) | (13) | (11) | (11) | (15) | (7) |
| | appearance on the PQ | | | | | | | | |
| | Cream-treated side in | | | | | | | | |
| | comparison to the | | | | | | | | |
| | side with no | | | | | | | | |
| | application of cream. | | | | | | | | |
| 8. | The appearance of | 46% | 54% | 38% | 62% | 50% | 50% | 64% | 36% |
| | my skin is | (11) | (13) | (9) | (15) | (11) | (11) | (14) | (8) |
| | improving quicker | | | | | | | | |
| | on the PQ Cream- | | | | | | | | |
| | treated side in | | | | | | | | |
| | comparison to the | | | | | | | | |
| | side with no | | | | | | | | |
| | application of cream. | | | | | | | | |
| 9. | My skin looks less | 67% | 33% | 63% | 37% | 59% | 41% | 73% | 27% |
| | swollen or puffy on | (16) | (8) | (15) | (9) | (13) | (9) | (16) | (6) |
| | the PQ Cream-treated | | | | | | | | |
| | side in comparison to | | | | | | | | |
| | the side with no | | | | | | | | |
| | application of cream. | | | | | | | | |
| 10. | My skin feels more | 63% | 37% | 67% | 33% | 73% | 27% | 77% | 23% |
| | soothed or calmer | (15) | (9) | (16) | (8) | (16) | (6) | (17) | (5) |
| | on the PQ Cream- | | | | | | | | |
| | treated side in | | | | | | | | |
| | comparison to the | | | | | | | | |
| | side with no | | | | | | | | |
| | application of cream. | | | | | | | | |
| 11. | The overall texture | 63% | 37% | 58% | 42% | 73% | 27% | 73% | 27% |
| | of my skin is more | (15) | (9) | (14) | (10) | (16) | (6) | (16) | (6) |
| | improved on the PQ | | | | | | | | |
| | Cream-treated side in | | | | | | | | |
| | comparison to the | | | | | | | | |
| | side with no | | | | | | | | |
| | application of cream. | | | | | | | | |
| 12. | My skin feels more | 75% | 25% | 79% | 21% | 86% | 14% | 86% | 14% |
| | hydrated on the PQ | (18) | (6) | (19) | (5) | (19) | (3) | (19) | (3) |
| | Cream-treated side in | | | | | | | | |
| | comparison to the | | | | | | | | |
| | side with no | | | | | | | | |
| | application of cream. | | | | | | | | |

PRODUCT ATTRIBUTES

| | | | | | | | | | |
|---|---|---|---|---|---|---|---|---|---|
| 1. | The PQ Cream | 100% | — | 100% | — | 100% | — | 100% | — |
| | absorbs quickly into | (24) | | (24) | | (22) | | (22) | |
| | my skin. | | | | | | | | |
| 2. | The PQ Cream did | 100% | — | 100% | — | 100% | — | 100% | — |
| | not irritate my skin. | (24) | | (24) | | (22) | | (22) | |
| 3. | The PQ Cream has a | 100% | — | 100% | — | 100% | — | 100% | — |
| | pleasant texture and | (24) | | (24) | | (22) | | (22) | |
| | feel. | | | | | | | | |
| 4. | I would continue to | 92% | 8% | 83% | 17% | 86% | 14% | 86% | 14% |
| | use the PQ Cream | (22) | (2) | (20) | (4) | (19) | (3) | (19) | (3) |
| | following this | | | | | | | | |
| | procedure (CCH | | | | | | | | |
| | Treatment). | | | | | | | | |

The data presented in Table 9 supports several characteristics of the test formulation (PQ Cream), which may be applicable to other formulations of the invention. All of the subjected tested found the product to rapidly absorb, be non-irritating, and to have good cosmetic elegance properties. Moreover, an overwhelming majority of subjects (86%) indicated that they would continue to use the product at the end of the clinical study.

Notable results include the finding that (1) a large majority of clinical trial subjects found that the test formulation reduced swelling throughout the study (73% at the end of the study); (2) a large majority of clinical trial subjects found that the test formulation improved skin texture (73% at end of the study); (3) a majority of clinical trial subjects found that treated skin was more even looking/uniform (68% at end of the study); and (4) a large majority of clinical trial subjects found that treated skin was more hydrated (86% at end of the study). Nearly two-thirds of subjects (64%) at end of the study believed that the test formulation was improving the appearance of the skin faster than without the treatment. These results further support the efficacy of the inventive products described in this disclosure, similar to this test formulation, in a wide variety of areas relating to the improvement of skin appearance and condition.

### Listed Exemplary Aspects of the Invention

The following is a non-limiting list of exemplary aspects of the invention, which illustrates embodiments of the invention in a summary form to aid readers in quickly understanding the overall scope of the invention. Similar to patent claims, listed aspects described in the paragraphs of this section may make reference to (depend on/from) one or more other paragraphs. Readers will understand that such references mean that the features/characteristics or steps of such referenced aspects are incorporated into/combined with the referring aspect. E.g., if an aspect in a paragraph (e.g., a paragraph indicated by text at the end of the paragraph as aspect 2) refers to another aspect by one or more aspect numbers (e.g., aspect 1 or “any one of aspects 1-3”), it will be understood to include the elements, steps, or characteristics of such referenced aspects (e.g., aspect 1) in addition to those of the aspect in which the reference is made (e.g., if aspect 2 refers to aspect 1, it provides a description of a composition, method, system, device, etc., including the features of both aspect 1 and aspect 2).

In one aspect, the invention provides a composition suitable for application to a human comprising (1) a physiologically effective amount (e.g., a cosmetically effective amount or therapeutically effective amount) of a coagulation pathway modulation component (CP-MC), wherein the coagulation pathway modulation component is a protein C activity modulator (PCAM), a protein S activity modulator (PSAM), a combination thereof, or a component that modulates both protein C and protein S activity (a PCAM/PSAM), and (2) a physiologically effective amount (e.g., a cosmetically effective amount or therapeutically effective amount) of a nicotinamide-adenine dinucleotide (NAD) pathway modulation component (NADP-MC), which composition optionally is formulated for topical administration (aspect 1).

Another aspect is a composition according to aspect 1, wherein the CP-MC includes an effective amount (e.g., a therapeutically or cosmetically effective amount) of a PCAM/PSAM an amount that detectably or significantly enhances the activity of endogenous protein C, detectably or significantly stabilizes endogenous protein C, or both detectably or significantly enhances the activity of and stabilizes endogenous protein C (aspect 2).

A further aspect is a composition of aspect 2, wherein the coagulation pathway modulation component comprises a vitamin K compound in an amount that detectably or significantly enhances the activity of endogenous protein C, detectably or significantly enhances the stability of protein C, or both detectably or significantly enhances the stability of and stabilizes endogenous protein C (aspect 3).

An aspect is a composition according to any one or more of aspects 1-3, wherein the NADP-MC comprises (1) a niacinamide compound component, (2) a nicotinate compound component, or (3) both a niacinamide compound component and a nicotinate compound component (aspect 4).

Also provided is a composition of any one or more of aspects 1-4, wherein the NADP-MC comprises a nicotinate compound component (aspect 5).

Further provided is a composition of any one or more of aspects 1-5, wherein the coagulation pathway modulation component consists essentially of a PCAM/PSAM (aspect 6).

Additionally provided is a composition of any one of aspects 4-6, wherein the composition comprises a nicotinate compound and ratio of the nicotinate compound component to the PCAM is about 1:6 to about 1:2 (aspect 7).

An aspect is a composition of any one or more of aspects 4-6, wherein the wherein the composition comprises a nicotinate compound and the ratio of the nicotinate compound component to the PCAM is about 1:5.5 to about 1:2.5 (aspect 8).

An aspect is a composition of any one or more of aspects 4-6, wherein the wherein the composition comprises a nicotinate compound and the ratio of the nicotinate compound component to the PCAM is about 1:5 to about 1:3 (aspect 9).

Provided also is a composition of any one or more of aspects 4-9, wherein the nicotinate compound component constitutes about 0.1 wt. %-about 0.5 wt. % of the composition (aspect 10).

Also provided is a composition of aspect 10, wherein the nicotinate compound component constitutes about 0.15 wt. %-about 0.35 wt. % of the composition (aspect 11).

Provided is a composition of aspect 11, wherein the nicotinate compound component constitutes about 0.2 wt. %-about 0.3 wt. % of the composition (aspect 12).

An aspect is a composition of any one or more of aspects 4-12, wherein the nicotinate compound component is at least mostly composed of a non-irritating nicotinate (e.g., a nicotinate that when delivered in an effective amount results in no significant increase in inflammation in a population of subjects, such as in ≥1 clinical studies) or results in inflammation at a level that is not deemed an impediment to FDA approval or corresponding regulatory authority approval (aspect 13).

Further provided is a composition according to aspect 13, wherein the nicotinate compound component consists essentially of one or more non-irritating nicotinates (aspect 14).

Also provided is a composition per aspect 14, wherein the nicotinate compound component consists essentially of tocopheryl nicotinate (aspect 15).

Another aspect is a composition of any one or more of aspects 1-4, wherein NADP-MC comprises a therapeutically effective amount of a niacinamide compound component (aspect 16).

Yet another aspect is a composition of aspect 16, wherein the NADP-MC consists essentially of one or more niacinamide compounds (aspect 17).

Also provided is a composition of any one or more of aspects 5-15, wherein the NADP-MC further comprises a therapeutically effective amount of a niacinamide compound component (aspect 18).

An aspect is a composition of aspect 17 or aspect 18, wherein the coagulation pathway modulation component consists essentially of a PCAM/PSAM and the ratio of the NAD pathway enhancer component to the PCAM/PSAM is about 1.5:1 to about 5:1 (aspect 19).

Further provided is a composition of aspect 19, wherein the ratio of the NADP-MC to the PCAM/PSAM is about 2.5:1 to about 4:1 (aspect 20).

Also provided is a composition of aspect 20, wherein the ratio of the NADP-MC to the PCAM/PSAM is about 3:1 to about 3.5:1 (aspect 21).

Still further provided is a composition of any one or more of aspects 19-21, wherein the NADP-MC comprises a nicotinate compound component and a niacinamide compound component, wherein the nicotinate compound component and niacinamide compound component are present in a ratio of about 1:4 to about 1:40 (aspect 22).

The invention also provides a composition of aspect 22, wherein the nicotinate compound component and niacinamide compound component are in a ratio of about 1:8 to about 1:20 (aspect 23).

Additionally provided is a composition of aspect 23, wherein the nicotinate compound component and niacinamide compound component are in a ratio of about 1:10 to about 1:15 (aspect 24).

Further provided still is a composition of any one or more of aspects 1-24, wherein the composition comprises about 1 wt. % to about 5 wt. % of a niacinamide compound component (aspect 25).

A further embodiment is a composition according to aspect 25, wherein the composition comprises about 1 wt. % to about 4 wt. % of a niacinamide compound component (aspect 26).

Another aspect is a composition of aspect 26, wherein the composition comprises about 1 wt. % to about 3 wt. % of a niacinamide compound component (aspect 27).

Further provided is a composition of any one or more of aspects 1-27, wherein the coagulation pathway modulation component is present in an amount of about 0.05 wt. %-about 2.5 wt. %, such as ~0.05 wt. % to ~1 wt. %, ~0.05 wt. % to ~0.75 wt. %, ~0.05 wt. % to ~0.5 wt. %, ~0.05 wt. % to ~0.25 wt. %, ~0.1 wt. % to ~1.5 wt. %, ~0.15 wt. % to ~1.5 wt. %, ~0.25 wt. % to ~1.5 wt. %, or ~0.5 wt. % to ~1.5 wt. % (aspect 28).

An aspect is a composition of aspect 28, wherein the coagulation pathway modulation component is present in an amount of about 0.75 wt. %-about 1.5 wt. % (aspect 29).

Another aspect is a composition of any one or more of aspects 1-29, wherein the CP-MC consists essentially of a vitamin K compound (aspect 30).

Still a further aspect is a composition of any one or more of aspects 1-30, wherein the CP-MC consists essentially of a vitamin K1 compound (aspect 31).

Also provided is a composition of any one or more of aspects 1-31, wherein the composition comprises an effective amount of one or more penetration enhancers (a penetration enhancer component) (aspect 32).

Still further provided is a composition of aspect 32, wherein the penetration enhancer component makes up about 1.5 wt. % to about 2.5 wt. % of the composition (aspect 33).

Moreover, the invention provides a composition of aspect 33, wherein the penetration enhancer component comprises an effective amount of one or more decoy molecule constituents (forming a decoy molecule component), wherein the decoy molecule component is composed of a sodium hyaluronate component, collagen fragment, fibronectin fragment, elastin fragment, or a combination of any thereof (aspect 34).

Furthermore, provided is a composition of aspect 34, wherein the decoy molecule component has an average molecular weight of about 2,000 Daltons to about 20,000 Daltons (aspect 35).

Additionally provided is a composition of aspect 35, wherein the decoy molecule component consists essentially of a hyaluronic acid fragment (aspect 36).

An additional aspect is a composition of any one or more of aspects 34-36, wherein about 5 wt. % to about 25 wt. % of the one or more penetration enhancers is composed of a decoy molecule component (aspect 37).

Moreover, the invention provides a composition of any one or more of aspects 34-37, wherein the decoy molecule component makes up about 0.1 wt. % to about 0.4 wt. % of the composition (aspect 38).

Another aspect is a composition of aspect 38, wherein the decoy molecule component makes up about 0.2 wt. % to about 0.3 wt. % of the composition (aspect 39).

Still further provided is a composition of any one or more of aspects 1-39, wherein the composition comprises an amount of one or more amino acids selected from arginine, histidine, lysine, methionine, proline, glutamine, Glycine, isoleucine, leucine, proline, tyrosine, and tryptophan, wherein the one or more amino acids detectably or significantly increases collagen levels, exhibits antioxidant activity, increases skin hydration, enhances skin healing, reduces wrinkles/fine lines, reduces dark circles or dark spots, protects against UV damage, enhances activity of the coagulation pathway modulating component, or a combination of any or all thereof (aspect 40).

Additionally provided is a composition of aspect 40, wherein the one or more amino acids is selected from arginine, histidine, lysine, tryptophan, and combinations of any or all thereof (aspect 41).

Furthermore, provided is a composition of any one or more of aspects 1-41, wherein the composition comprises an effective amount of one or more neurotransmitter-inhibiting peptides (aspect 42).

An aspect is a composition of aspect 42, wherein one or more neurotransmitter-inhibiting peptides comprises of a SNAP-25 fragment peptide or an analog thereof (aspect 43).

Further provided still is a composition of aspect 43, wherein the SNAP-25 fragment peptide or analog thereof is acetyl hexapeptide-8, Arg-2, or Arg-3 (aspect 44).

An aspect is a composition of any one or more of aspects 1-44, wherein the composition comprises an effective amount of one or more resveratrol compounds (aspect 45).

Another exemplary aspect of the invention is a composition of any one or more of aspects 1-45, wherein the composition comprises an effective amount of one or more sesquiterpene lactone compounds (aspect 46).

An aspect is a composition of aspect 46, wherein the sesquiterpene lactone compounds are contained in an Arnica composition (aspect 47).

Further provided is a composition of any one or more of aspects 1-47, wherein the composition comprises an effective amount of an exfoliant, such as salicylic acid (aspect 48).

Yet another aspect is a composition of any one or more of aspects 1-48, wherein the composition comprises one or more emollients, such as a squalane emollient, such as hemisqualane (aspect 49).

Moreover, the invention provides a composition of any one or more of aspects 1-49, wherein the composition lacks any vitamin that has a molecular weight of between 135-400 g/Mol or above 550 g/Mol (aspect 50).

The invention also provides a composition of any one or more of aspects 1-50, wherein the most abundant vitamin in the composition forms part of the coagulation pathway modulation component, the NADP-MC, or both (aspect 51).

The invention further provides a composition according to any one or more of aspects 1-51, wherein the composition is a cosmetic composition that modifies the appearance of skin but does not result in any physiological effect that would be considered treating or preventing a disease (aspect 52).

Also provided is a composition of any one or more of aspects 1-52, wherein the composition results in a detectable or significant improvement in one or more conditions of the skin in a majority of subjects, a significant number of subjects, or both, in one or more adequate and controlled clinical trial(s) (aspect 53).

An aspect is a composition of aspect 53, wherein the one or more conditions of the skin is bruising, hyperkeratosis, or both (aspect 54).

An aspect is a composition of aspect 54, wherein a detectable or significant improvement in skin hyperkeratosis can be measured within about 10 hours of topical application of the composition (aspect 55).

An aspect is a composition of aspect 55, wherein a detectable or significant improvement in skin hyperkeratosis can be measured within about 8 hours of topical application of the composition (aspect 56).

The invention further provides a composition of aspect 56, wherein a detectable or significant improvement in skin hyperkeratosis can be measured within about 2, 4, 6, or 8 hours of topical application of the composition (aspect 57).

An aspect is a composition of any one or more of aspects 1-57, wherein the composition is substantially absorbed into the skin within less than 12 hours of application, is at least about 50% absorbed within the skin within about 30 minutes of application or is at least about 50% absorbed in the skin after about 20 minutes of application (aspect 58).

A further aspect is a composition of any one or more of aspects 1-58, wherein at least about 1 wt. %, 1.5 wt. %, 2 wt. %, 2.5 wt. %, 3 wt. %, or more of the composition is composed of one or more fatty acid compounds (aspect 59).

Another aspect of the invention is a composition of any one or more of aspects 1-59, wherein at least about 33 wt. %, such as at least about 40 wt. %, such as at least about 50 wt. % of the composition, or more than 50 wt. % is composed of water (aspect 60).

An additional aspect of the invention is a composition of aspect [0534], wherein the composition is a cream (aspect 61).

An aspect is a composition of any one or more of aspects 1-61, wherein the composition does not contain more than 1 wt. % of any hyaluronic acid derivative, such as any hyaluronic ester, and optionally is free of any such compounds (aspect 62).

A further aspect is a composition of any one or more of aspects 1-62, wherein the composition lacks more than 1 wt. % of (e.g., more than 0.5 wt. % of) or is free of any hyaluronic acid composition having an average molecular weight of greater than about 25,000 Da (aspect 63).

Also provided is a composition of any one or more of aspects 1-63, wherein (a) the composition is free of growth hormone, chitosan, psyllium, or whole cells; (b) the composition lacks an amount of one or more minerals (or any minerals) that detectably or significantly aid in the intended effect of the composition, such as copper, zinc, or both; or (c) the composition has the characteristics of both (a) and (b) (aspect 64).

Moreover, the invention provides a composition of any one or more of aspects 1-[0538], wherein (a) no more than 1 wt. %, 0.75 wt. %, or 0.5 wt. % of the composition is made up of any cellulose compounds, (b) no more than 1 wt. %, 0.75 wt. %, or 0.5 wt. % of the composition is composed of any non-vitamin antibacterial agent, or both (aspects 65).

An aspect is a method of modulating condition(s) of the skin of a subject comprising administering an effective amount of a composition according to any one or more of aspects 1-65 in a sufficient amount and a sufficient number of times to detectably or significantly modulate one or more conditions of the skin of the subject (aspect [0540]).

An aspect is a method of aspect [0540], wherein the method includes/is performed to improve the appearance of hyperkeratotic skin (aspect 67).

Another aspect is a method of any one or both of aspect or aspect [0541], wherein the method results in/is performed to smooth the skin (aspect [0542]).

The invention also provides a method of any one or more of aspects 66-68, wherein a detectable or significant difference in skin smoothness can be measured within 10 hours of topical application of the composition to the skin (aspect 69).

Further provided is a method of aspect 69, wherein a detectable or significant difference in skin smoothness can be measured within ~4, 6, 8, 10, 12, or about 24 hours of topical application of the composition to the skin (aspect 70).

Yet another aspect of the invention is a method of any one or more of aspects 66-70, wherein the method comprises/results in the treatment or prevention of a skin-associated disease or medical condition (aspect 71).

An aspect is a method of aspect 71, wherein the skin-associated disease or medical condition comprises seborrheic dermatitis (aspect 72).

An aspect is a method of aspect 71, wherein the skin-associated disease or medical condition comprises psoriasis (aspect 73).

An aspect is a method of aspect 71, wherein the skin-associated disease or medical condition comprises actinic keratosis (aspect 74).

Furthermore, the invention provides a method of any one or more of aspects 71-74, wherein the skin-associated disease or medical condition to be treated does not solely comprise, does not primarily comprise, or does not comprise any one or more of acne, rosacea, improvement of dark circles, and vaginal dryness (aspect 75).

An aspect is a method of aspect 71, wherein the skin-associated disease or medical condition comprises bruising (aspect 76).

An aspect is a method of aspect 71, wherein the skin-associated disease or medical condition comprises wound healing (aspect 77).

A further aspect is a method of aspect 76 or aspect 77, wherein the skin-associated disease or medical condition is associated with the performance of a medical or aesthetic procedure on the subject (aspect 78).

An aspect is a method of aspect 78, wherein the method comprises administering the composition before the performance of the medical or aesthetic procedure (aspect 79).

An aspect is a method of aspect 78, wherein the method comprises administering the composition after the performance of the medical or aesthetic procedure (aspect 80).

An aspect is a method of any one or more of aspects 66-80, wherein the method comprises administering the composition both before and after the performance of the medical or aesthetic procedure (aspect 81).

Further provided is a method of any one or more of aspects 66-81, wherein the subject has bruising, and the method comprises administering the composition promptly following the formation of a wound or bruise on the skin of the subject (aspect 82).

Also provided is a method of any one or more of or more of aspects 66-82, wherein the method comprises repeatedly applying the composition topically two or more times over a period of time (e.g., over a day, week, month, etc.) (aspect 83). In aspects, a method comprises applying a composition once a day, twice a day, three times a day, four times a day, or five times a day, for one day or over a sustained period of time. In aspects, a composition is only applied once or twice a day, but administration is optionally repeated for several days (e.g., 2-30, 3-21, 5-20, 5-15, 5-10, or 2-14 days). All such aspects can be considered parts of aspect 83.

The invention further provides a method of any one or more of aspects 66-83, wherein the method comprises applying the composition topically at least once per day for a period of at least about 1 week (aspect 84).

An aspect of the invention is a method of any one or more of aspects 66-84, wherein the method comprises applying the composition topically at least once per day for a period of about 2 weeks (aspect 85).

Further provided is a method of any one or more of aspects 66-85, wherein the method comprises applying the composition topically at least once per day for a period of at least about one month (aspect 86).

Also provided is a method of aspect 86, wherein the method comprises applying the composition topically at least once per day for a period of at least about 3 months (aspect 87).

Moreover, provided is a method of any one or more of aspects 66-87, wherein the method is initiated only under the supervision of a licensed medical professional (e.g., the method is initiated under the supervision of a licensed healthcare professional, the performance of the method is monitored by a licensed healthcare professional, or both) (aspect 88).

An aspect is a method per aspect 88, wherein the method comprises determining if a potential subject has one or more risk factors relating to the ingredients of the composition prior to administering the composition and either monitoring the subject for such risk factors, modifying one or more aspects of the method based on such risk factors, or excluding the subject from receiving the treatment based on such risk factors (aspect 89).

Also provided is a method of aspect 88 or aspect 89, wherein the method comprises testing the subject for vitamin K allergy prior to treatment, during treatment, or both, and either not performing the method, stopping the method, or modifying the method in subjects identified as having a vitamin K allergy (aspect 90).

Still also provided is a method of any one or more of aspects 66-90, wherein the method primarily comprises applying the composition directly to the skin without the aid of a patch or other mechanical delivery system (aspect 91).

Another aspect is a method of aspect 91, wherein the method comprises massaging the composition into the treated skin of an individual (aspect 92).

Further provided is a method of any one or more of aspects 66-92, wherein the method does not comprise applying the composition to any open wound (aspect 93).

An aspect is a method of any one or more of aspects 66-93, wherein the composition is adapted such that, and the amount of composition applied to the subject is such that, the method is effective in more than 50% of treated subjects (aspect 94).

An aspect is a method of any one or more of aspects 66-94, wherein the subject does not have a mineral deficiency, such as an iron deficiency; the method is not used to treat a mineral deficiency; or both (aspect 95).

A further aspect is a method of any one or more of aspects 66-95, wherein the method comprises administering the composition to several separate areas of skin of the subject (aspect 96).

Still another aspect is a method of aspect 96, wherein the method comprises administering the composition to defined, individual lesions (optionally including the immediately surrounding area of the skin) (aspect 97).

An aspect is a method of aspect 97, wherein the method comprises administering the composition to individual seborrheic keratosis lesions of the subject (aspect 98).

Another aspect of the invention is a method of any one or more of aspects 66-95, wherein the method comprises detectably or significantly inducing, enhancing, or promoting one or more cosmetic effects in the subject's skin (aspect 99).

Also provided by the invention is a method of aspect 99, wherein the one or more cosmetic effects comprise reduction of fine lines, reduction of wrinkles, reduction of erythema, enhancement of luminosity, or a combination thereof (aspect 100).

Another aspect is a composition according to any one of aspects 1-65 or method of using such a composition, optionally including any other elements of aspects 66-100, wherein the composition does not cause a significant amount of skin flushing, increase in blood pressure, pulse rate increase, increase in body temperature, or combination of some or all thereof (aspect 101).

In aspects, the invention provides a multi-ingredient formulation/composition according to any one or more of aspects 1-65, wherein the composition is made by a process comprising a multi-phase ingredient mixture process comprising at least four separate phase preparation/mixture steps (aspect 102).

Another aspect is a multi-ingredient formulation/composition according to aspect 102, wherein the process comprises a multi-phase ingredient mixture process comprising at least five separate phase preparation/mixture steps (aspect 103).

Another aspect is a multi-ingredient formulation/composition according to any one or more of aspects 102 or 103, wherein the process comprises a multi-phase ingredient mixture process comprising at least six separate phase preparation/mixture steps (aspect 104).

Another aspect is a multi-ingredient formulation/composition according to any one or more of aspects 102-104, wherein the process comprises heating, continuous mixing, and cooling steps (aspect 105).

In aspects, the invention provides a process for manufacturing a composition or multi-ingredient composition according to any one or more of aspects 1-65, 101, 102-105, wherein the process comprises a multi-phase ingredient mixture process comprising at least four separate phase preparation/mixture steps, and wherein the process comprises at least one heating step, at least one continuous mixing step, at least one cooling step, or any combination thereof (aspect 106).

In aspects, the invention provides a method according to any one or more of aspects 66-100, wherein the composition used in the method is a composition according to any one or more of aspects 102-105 (aspect 107).

In aspects, the invention provides a method according to any one or more of aspects 66-101, wherein the composition used in the method is a composition made according to the process of aspect 106 (aspect 108).

In aspects, the invention provides a composition according to any one or more of the composition aspects provided in this section, e.g., aspects 1-65, 101, or, e.g., 102-105, wherein the administration of an effective mount of the composition (e.g., composition/formulation) results in any one or more of the results described in any one or both of Table 8 and Table 9, ±about 25%, ±about 20%, ±about 15%, ±about 10%, ±about 5%, or, e.g., ±about 1% (aspect 109).

In aspects, the invention provides a method according to any one or more of the method aspects provided in this section, e.g., aspects 66-101 and 107-108, wherein the application of the method results in attaining any one or more of the results described in any one or both of Table 8 and Table 9, ±about 25%, ±about 20%, ±about 15%, ±about 10%, ±about 5%, or, e.g., ±about 1% (aspect 110).

All of the original claims of this disclosure are incorporated herein as aspects, which can be combined with any other suitable aspects in this section or any other suitable part of this disclosure.

1. A composition suitable for application to a human comprising (1) a physiologically effective amount of a coagulation pathway modulation component (CP-MC), wherein the coagulation pathway modulation component is a protein C activity modulator (PCAM), a protein S activity modulator (PSAM), a combination thereof, or a component that modulates both protein C and protein S activity (a PCAM/PSAM), and (2) a physiologically effective amount of a nicotinamide-adenine dinucleotide (NAD) pathway modulation component (NADP-MC), wherein the composition is formulated for topical administration, and further wherein the coagulation pathway modulation component comprises an effective amount of a compound that modulates both protein C and protein S activity and which detectably or significantly enhances the activity of endogenous protein C, detectably or significantly stabilizes endogenous protein C, or both detectably or significantly enhances the activity of and stability of endogenous protein C.

2. The composition of claim 1, wherein the coagulation pathway modulation component comprises a dermatologically suitable vitamin K compound in an amount that detectably or significantly enhances the activity of endogenous protein C, detectably or significantly enhances the stability of protein C, or both detectably or significantly enhances the activity of and stability of endogenous protein C.

3. The composition of claim 1, wherein the nicotinamide-adenine dinucleotide pathway modulation component comprises an effective amount of (1) a niacinamide compound component, (2) a nicotinate compound component, or (3) both a niacinamide compound component and a nicotinate compound component.

4. The composition of claim 3, wherein the composition comprises a nicotinate compound component, and the ratio of the nicotinate compound component to the protein C activity modulator is about 1:5 to about 1:3.

5. The composition of claim 4, wherein the nicotinate compound component represents about 0.1 wt. %-about 0.5 wt. % of the composition.

6. The composition of claim 5, wherein the nicotinate compound component is at least mostly composed of a nicotinate that when delivered in an effective amount results in no significant increase in inflammation in a population of subjects in an appropriately controlled clinical study.

7. The composition of claim 6, wherein the nicotinate compound component is mostly composed of tocopheryl nicotinate.

8. The composition of claim 1, wherein the nicotinamide-adenine dinucleotide pathway modulation component is at least mostly composed of a therapeutically effective amount of a niacinamide compound component.

9. The composition of claim 8, wherein the nicotinamide-adenine dinucleotide pathway modulation component is at least mostly composed of one or more niacinamide compounds.

10. The composition of claim 1, wherein the coagulation pathway modulation component is at least mostly composed of a compound that modulates both protein C and protein S activity, and the ratio of the nicotinamide-adenine dinucleotide pathway modulation component to the compound that modulates both protein C and protein S activity is about 1.5:1 to about 5:1.

11. The composition of claim 3, wherein the nicotinate compound component and niacinamide compound component are present in a ratio of about 1:4 to about 1:40.

12. The composition of claim 1, wherein the composition comprises about 1 wt. % to about 5 wt. % of a niacinamide compound component, and the coagulation pathway modulation component is present in an amount of about 0.75 wt. % to about 1.5 wt. % of the composition.

13. The composition of claim 1, wherein the composition comprises a penetration enhancer component that is present in an amount of about 1.5 wt. % to about 4 wt. % of the composition.

14. The composition of claim 13, wherein the penetration enhancer component mostly comprises an effective amount of one or more of a sodium hyaluronate component, collagen fragment, fibronectin fragment, elastin fragment, or a combination of any thereof.

15. The composition of claim 14, wherein the penetration enhancer component is at least mostly composed of hyaluronic acid fragments that have an average molecular weight of about 2,000 Daltons to about 20,000 Daltons.

16. The composition of claim 13, wherein the penetration enhancer component comprises about 0.5 wt. % to about 3 wt. % of a bicyclic compound comprising two fused furan rings that detectably or significantly enhances penetration of one or more components of the composition.
